(19) United States
(12) Patent Application Publication
(10) Pub. No.: US 2025/0333418 A1
(43) Pub. Date: Oct. 30, 2025

(54) PURINE COMPOUNDS, COMPOSITIONS COMPRISING THEM AND USES THEREOF

(71) Applicant: Novo Nordisk A/S, Bagsvaerd (DK)

(72) Inventors: Jeremy Green, Waltham, MA (US); Shawn Gallagher-Duval, Montreal (CA); Alexandre Lemire, Montreal (CA); Yuchen Zhou, Pointe-Claire (CA); Hong Wang, Verdun (CA)

(21) Appl. No.: 19/124,007

(22) PCT Filed: Oct. 26, 2023
(86) PCT No.: PCT/CA2023/051428

§ 371 Date: Apr. 24, 2025

Related U.S. Application Data
(60) Provisional application No. 63/381,036, filed on Oct. 26, 2022.

(51) Int. Cl. C07D 473/16; A61K 31/52; A61K 31/5377; A61K 31/5386; A61K 31/541; A61K 31/55; A61K 31/551; A61K 31/675; C07D 473/04; C07D 473/18; C07D 473/24; C07D 473/28; C07D 473/34; C07D 473/40; C07D 519/00; C07F 9/6561

(52) U.S. Cl. CPC C07D 473/16; A61K 31/52; A61K 31/5377; A61K 31/5386; A61K 31/541; A61K 31/55; A61K 31/551; A61K 31/675; C07D 473/04; C07D 473/18; C07D 473/24; C07D 473/28; C07D 473/34; C07D 473/40; C07D 519/00; C07F 9/65616

(57) ABSTRACT

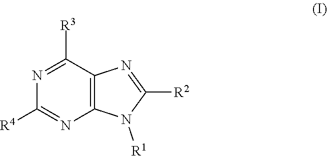

The present document relates to purine compounds, pharmaceutical compositions comprising the same and their use in the treatment or prevention of diseases and disorders associated with the cannabinoid CB₁ receptor. For example, the purine compounds, or a tautomeric form and/or salt thereof, are of the formula (I): wherein R¹ to R⁴ are as defined herein.

### RELATED APPLICATION

The present application claims priority under applicable law to U.S. provisional application No. 63/381,036 filed on Oct. 26, 2022, the content of which is incorporated herein by reference in its entirety for all purposes.

### TECHNICAL FIELD

This disclosure generally relates to compounds, especially purine compounds, pharmaceutical compositions comprising them and their use and methods of use in the treatment and prevention of diseases and disorders.

### BACKGROUND

It is generally known that activation of the cannabinoid CB₁ receptor increases appetite, increases the biosynthesis and storage of lipids, inhibits the actions of insulin and leptin, and promotes inflammation and fibrosis. Research was thus focused on developing CB₁ receptor inhibitors for the potential treatment of obesity and the metabolic disorder associated therewith, referred to as metabolic syndrome. Rimonabant was shown effective in treating metabolic syndrome but caused neuropsychiatric (i.e. CNS-related) side effects, which resulted in its withdrawal from the market.

There remains a need for the development of alternative compounds targeting the CB₁ receptor for the treatment or prevention of disorders associated thereto.

### SUMMARY

According to one aspect, the present technology relates to compounds and their pharmaceutically acceptable salts, their pharmaceutical compositions, uses thereof and methods of treatment comprising their administration. More specifically, the following embodiments are provided:

Embodiment 1. A compound of Formula I:

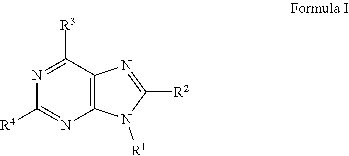

wherein,
R¹ is an optionally substituted C₆aryl group;
R² is an optionally substituted C₆aryl or C₅-C₆heteroaryl group;
R³ is an optionally substituted group selected from R⁷O—, N(R⁸)₂—, C₄₋₁₀heterocycloalkyl, and C₅₋₆heteroaryl;
R⁴ is an optionally substituted group selected from R⁵O— and N(R⁶)₂—;
R⁵ is a group selected from C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl;
R⁶ is H or a group selected from C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl, wherein at least one R⁶ is other than H, or two R⁶ groups are taken together with their adjacent nitrogen atom to form a C₄-C₁₀heterocycloalkyl or C₅-C₆heteroaryl group;
wherein at least one of said alkyl, cycloalkyl, heterocycloalkyl, or heteroaryl in R⁵ or R⁶ is substituted with at least one hydroxyl or hydroxy-substituted C₁-C₄alkyl group and is optionally further substituted with other substituents;
R⁷ is an optionally substituted C₁-C₆alkyl; and
R³ is independently in each occurrence selected from hydrogen, C₁-C₆alkyl, C₃-C₇cycloalkyl, and C₄-C₁₀heterocycloalkyl;
wherein each of the alkyl, cycloalkyl, heterocycloalkyl, aryl, and heteroaryl groups is optionally substituted;
or an isomer or a tautomer thereof, or a pharmaceutically acceptable salt thereof.

Embodiment 2. The compound of embodiment 1, wherein said compound is of Formula II:

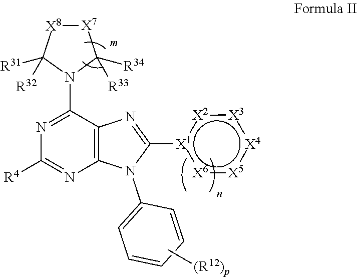

wherein,
R⁴ is as defined in embodiment 1;
X¹ is C and X² to X⁶ are each independently selected from N and CR²¹ and n is 1, wherein at most three of X² to X⁶ are N; or
X¹ is C or N and X² to X⁵ are each independently selected from CR²¹, O, S, N, or NR¹¹, n is zero and X⁶ is absent and replaced by a bond between X¹ and X⁵, wherein at most three of X¹ to X⁵ are other than C or CR²¹;
X⁷ and X⁸ are each independently selected from O, S, SO₂, NR³⁶, or C(R³⁵)₂, wherein when one of X⁷ and X³ is O, S, or NR³⁶, then the other is C(R³⁵)₂;
R⁹ is independently in each occurrence selected from optionally substituted C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, and C₅-C₆heteroarylC₁-C₃alkyl;
R¹⁰ is independently in each occurrence selected from H or a group selected from optionally substituted C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, and C₅-C₆heteroarylC₁-C₃alkyl, or two R¹⁰ groups are taken together with their adjacent nitrogen atom to form an optionally substituted C₄-C₁₀heterocycloalkyl or C₅-C₆heteroaryl;
R¹¹ is independently in each occurrence selected from H or a group selected from optionally substituted C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl;
R¹² is independently in each occurrence selected from F, Cl, CN, NH₂, N(H)C₁-C₃alkyl, N(C₁-C₃alkyl)₂, and C₁-C₃alkyl, and p is 0, 1, 2, or 3, preferably 0 or 1;
R²¹ is independently in each occurrence selected from hydrogen, halogen, OH, OR⁹, CN, NO₂, C(O)R⁹, C(O)N(R¹⁰)₂, C(R¹¹)═NR¹¹, SO₂R⁹, SO₂N(R¹⁰)₂, N(R¹¹)C(O)R⁹, N(R¹¹)SO₂R⁹, N(R¹¹)C(O)N(R¹⁰)₂, N(R¹¹)SO₂N(R¹⁰)₂, N(R¹⁰)₂, P(O)(R¹⁰)₂, P(O)(OR¹⁰)₂, B(OR¹⁰)₂, and optionally substituted C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl groups;
R³¹ to R³⁵ are independently in each occurrence selected from hydrogen, halogen, OH, OR⁹, CN, NO₂, C(O)OH, C(O)OR⁹, C(O)R⁹, C(O)N(R¹⁰)₂, C(R¹¹)═NR¹¹, SO₂R⁹, SO₂N(R¹⁰)₂, N(R¹¹)C(O)R⁹, N(R¹¹)SO₂R⁹, N(R¹¹)C(O)N(R¹⁰)₂, N(R¹¹)SO₂N(R¹⁰)₂, N(R¹⁰)₂, P(O)(R¹⁰)₂, P(O)(OR¹⁰)₂, B(OR¹⁰)₂, and optionally substituted C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl groups;
R³⁶ is selected from hydrogen, C(O)R⁹, C(O)OR⁹, C(O)N(R¹⁰)₂, C(R¹¹)═NR¹¹, SO₂R⁹, SO₂N(R¹⁰)₂, P(O)(R¹⁰)₂, P(O)(OR¹⁰)₂, B(OR¹⁰)₂, and optionally substituted C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl groups;
or two of R³¹ to R³⁶ are taken together with their adjacent atoms to form a cycle, preferably a bridged or spiro heterocycle; and
m is 0, 1, 2, or 3;
wherein each of the alkyl, cycloalkyl, heterocycloalkyl, aryl, and heteroaryl groups is optionally substituted;
or an isomer or a tautomer thereof, or a pharmaceutically acceptable salt thereof.

Embodiment 3. The compound of embodiment 2, wherein m is zero and X⁷ and X⁸ are each independently C(R³⁵)₂, preferably X⁷ is CH₂.

Embodiment 4. The compound of embodiment 3, wherein X³ is C(R³⁵)₂, wherein one of the two R³⁵ is fluorine, C(O)R⁹, C(O)OR⁹, C(O)N(R¹⁰)₂, N(R¹¹)C(O)R⁹, or optionally substituted C₁₋₆alkyl, and the other of the two R³⁵ is hydrogen, fluorine, CN, OH, OR⁹, N(R¹⁰)₂, or optionally substituted C₁₋₆alkyl.

Embodiment 5. The compound of embodiment 3, wherein X³ is C(R³⁵)₂ and the two R³⁵ are taken together with their adjacent carbon atom to form a spiro C₄₋₅heterocycle.

Embodiment 6. The compound of embodiment 2, wherein m is 1 and X⁷ and X⁸ are each independently C(R³⁵)₂, preferably X⁷ is CH₂.

Embodiment 7. The compound of embodiment 6, wherein X³ is C(R³⁵)₂, wherein one of the two R³⁵ is fluorine, C(O)R⁹, C(O)OR⁹, C(O)N(R¹⁰)₂, N(R¹¹)C(O)R⁹, or optionally substituted C₁₋₆ alkyl, and the other of the two R³⁵ is hydrogen, fluorine, CN, OH, OR⁹, N(R¹⁰)₂, or optionally substituted C₁₋₆alkyl.

Embodiment 8. The compound of embodiment 6, wherein X³ is C(R³⁵)₂ and the two R³⁵ are taken together with their adjacent carbon atom to form a spiro C₄₋₅heterocycle.

Embodiment 9. The compound of embodiment 2, wherein m is 2 and X⁷ and X⁸ are each independently C(R³⁵)₂.

Embodiment 10. The compound of embodiment 9, wherein X³ is CH₂ and X⁷ is C(R³⁵)₂, wherein one of the two R³⁵ is fluorine, C(O)R⁹, C(O)OR⁹, C(O)N(R¹⁰)₂, N(R¹¹)C(O)R⁹, or optionally substituted C₁₋₆alkyl, and the other of the two R³⁵ is hydrogen, fluorine, CN, OH, OR⁹, N(R¹⁰)₂, or optionally substituted C₁₋₆alkyl.

Embodiment 11. The compound of embodiment 10, wherein one of the two R³⁵ is a C(O)N(R¹⁰)₂, or N(R¹¹)C(O)R⁹, and the other of the two R³⁵ is OR⁹ or an optionally substituted C₁₋₄alkyl.

Embodiment 12. The compound of embodiment 11, wherein the other of the two R³⁵ is OR⁹ and R⁹ is a C₁₋₆alkyl, preferably C₂₋₄alkyl.

Embodiment 13. The compound of embodiment 11, wherein the other of the two R³⁵ is OR⁹ and R⁹ is selected from methyl, ethyl, n-propyl, isopropyl, i-butyl, and sec-butyl, preferably ethyl or isopropyl, most preferably isopropyl.

Embodiment 14. The compound of embodiment 9, wherein X³ is CH₂ and X⁷ is C(R³⁵)₂ and the two R³⁵ are taken together with their adjacent carbon atom to form a spiro C₄₋₅heterocycle.

Embodiment 15. The compound of embodiment 9, wherein X⁷ is CH₂ and X⁸ is C(R³⁵)₂, wherein one of the two R³⁵ is fluorine, C(O)R⁹, C(O)OR⁹, C(O)N(R¹⁰)₂, N(R¹¹)C(O)R⁹, or optionally substituted C₁₋₆alkyl, and the other of the two R³⁵ is hydrogen, fluorine, CN, OH, OR⁹, N(R¹⁰)₂, or optionally substituted C₁₋₆alkyl.

Embodiment 16. The compound of embodiment 15, wherein one of the two R³⁵ is a C(O)N(R¹⁰)₂, or N(R¹¹)C(O)R⁹, and the other of the two R³⁵ is OR⁹ or an optionally substituted C₁₋₄alkyl.

Embodiment 17. The compound of embodiment 16, wherein the other of the two R³⁵ is OR⁹ and R⁹ is a C₁₋₆alkyl, preferably C₂₋₄alkyl.

Embodiment 18. The compound of embodiment 16, wherein the other of the two R³⁵ is OR⁹ and R⁹ is selected from methyl, ethyl, n-propyl, isopropyl, i-butyl, and sec-butyl, preferably ethyl or isopropyl, most preferably isopropyl.

Embodiment 19. The compound of embodiment 9, wherein X⁷ is CH₂ and X⁸ is C(R³⁵)₂ and the two R³⁵ are taken together with their adjacent carbon atom to form a spiro C₄₋₅heterocycle.

Embodiment 20. The compound of embodiment 2, wherein m is 2 or 3, X⁷ is O, S, SO₂, or NR³⁶, and X³ is C(R³⁵)₂.

Embodiment 21. The compound of embodiment 20, wherein m is 2.

Embodiment 22. The compound of embodiment 20, wherein m is 3.

Embodiment 23. The compound of any one of embodiments 20 to 22, wherein X⁷ is O, SO₂, or NR³⁶.

Embodiment 24. The compound of embodiment 23, wherein X⁷ is NR³⁶ and R³⁶ is selected from hydrogen, C(O)R⁹, C(O)OR⁹, C(O)N(R¹⁰)₂, and optionally substituted C₁₋₆alkyl.

Embodiment 25. The compound of any one of embodiments 20 to 24, wherein one of the two R³⁵ is hydrogen, fluorine, C(O)R⁹, C(O)OR⁹, C(O)N(R¹⁰)₂, N(R¹¹)C(O)R⁹, or optionally substituted C₁₋₆alkyl, and the other of the two R³⁵ is hydrogen, fluorine, OH, OR⁹, N(R¹⁰)₂, or optionally substituted C₁₋₆alkyl, or one of the R³⁵ is taken together with R³³ and their adjacent atoms to form a bridged cycle.

Embodiment 26. The compound of embodiment 25, wherein both R³⁵ are hydrogen.

Embodiment 27. The compound of embodiment 25, wherein one of the two R³⁵ is fluorine, C(O)R⁹, C(O)OR⁹, C(O)N(R¹⁰)₂, N(R¹¹)C(O)R⁹, or optionally substituted C₁₋₆alkyl, and the other of the two R³⁵ is hydrogen.

Embodiment 28. The compound of embodiment 25, wherein one of the R³⁵ together with R³³ form a C₁₋₃alkylene group.

Embodiment 29. The compound of any one of embodiments 2 to 27, wherein R³³ is independently in each occurrence selected from hydrogen, fluorine and optionally substituted C₁₋₆alkyl.

Embodiment 30. The compound of any one of embodiments 2 to 29, wherein R³⁴ is independently in each occurrence selected from hydrogen, fluorine and optionally substituted C₁₋₆alkyl.

Embodiment 31. The compound of any one of embodiments 2 to 30, wherein R³¹ and R³² are each hydrogen atoms.

Embodiment 32. The compound of embodiment 1 or 2, wherein R³ is selected from groups C1-C7, C10, C15, C16, C18-C22, C24-C28, C32-C40, and C47-C69, preferably C1, C16 or C18, more preferably C18.

Embodiment 33. The compound of embodiment 1, wherein said compound is of Formula III:

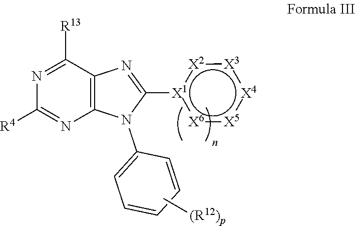

wherein,
R¹³ is an optionally substituted group selected from R⁷O—, N(R³)₂—, and C₅₋₆heteroaryl;
R⁴, R⁷ and R³ are as defined in embodiment 1; and
R¹², X¹ to X⁶, n and p are as defined in embodiment 2;
or an isomer or a tautomer thereof, or a pharmaceutically acceptable salt thereof.

Embodiment 34. The compound of embodiment 33, wherein R¹³ is R⁷O—.

Embodiment 35. The compound of embodiment 34, wherein R⁷ is an optionally substituted C₁-C₄alkyl.

Embodiment 36. The compound of embodiment 33, wherein R¹³ is N(R⁸)₂—.

Embodiment 37. The compound of embodiment 36, wherein one R³ is an optionally substituted C₄-C₁₀heterocycloalkyl and the other R³ is hydrogen or an optionally substituted C₁-C₆alkyl.

Embodiment 38. The compound of embodiment 36, wherein one R³ is an optionally substituted C₄-C₇heterocycloalkyl and the other R³ is hydrogen or an optionally substituted C₁-C₄alkyl.

Embodiment 39. The compound of embodiment 36, wherein one R³ is an optionally substituted C₁-C₆alkyl, and the other R³ is hydrogen or an optionally substituted C₁-C₆alkyl.

Embodiment 40. The compound of embodiment 33, wherein R¹³ is selected from C8, C9, C11-C14, C17, C23, C29-C31, and C41-C46.

Embodiment 41. The compound of any one of embodiments 2 to 40, wherein R¹² is Cl and p is 1, preferably forming a 4-chlorophenyl group.

Embodiment 42. The compound of any one of embodiments 2 to 40, wherein p is zero and R¹² is absent, forming an unsubstituted phenyl group.

Embodiment 43. The compound of any one of embodiments 2 to 42, wherein n is 1, X¹ is C and X² to X⁶ are each independently CR²¹.

Embodiment 44. The compound of any one of embodiments 2 to 42, wherein n is 1, X¹ is C, one of X² to X⁶ is N and the others are CR²¹.

Embodiment 45. The compound of any one of embodiments 2 to 42, wherein n is zero, X⁶ is absent, and X¹ is C.

Embodiment 46. The compound of embodiment 45, wherein one or two of X² to X⁵ is N or NR¹¹ and the others are CR²¹, preferably R¹¹ is a C₁-C₆alkyl.

Embodiment 47. The compound of embodiment 45, wherein one of X² to X⁵ is S and the others are CR²¹.

Embodiment 48. The compound of any one of embodiments 43 to 47, wherein all CR²¹ are CH.

Embodiment 49. The compound of embodiment 43, wherein one CR²¹ is other than CH, preferably R²¹ being selected from halogen, CN, N(C₁₋₆alkyl)₂, and C₁₋₆alkyl, more preferably halogen or CN, most preferably halogen (e.g. Cl).

Embodiment 50. The compound of embodiment 44, wherein one CR²¹ is other than CH, preferably R²¹ being selected from halogen, CN, N(C₁₋₆alkyl)₂, and C₁₋₆alkyl, more preferably halogen, CN, and C₁₋₆alkyl, most preferably CN.

Embodiment 51. The compound of any one of embodiments 45 to 47, wherein one CR²¹ is other than CH, preferably R²¹ being selected from halogen, CN, N(C₁₋₆alkyl)₂, and C₁₋₆alkyl, more preferably CN and C₁₋₆alkyl, most preferably C₁₋₆alkyl.

Embodiment 52. The compound of any one of embodiments 1 to 51, wherein R² is selected from groups B1 to B23 as defined herein.

Embodiment 53. The compound of embodiment 52, wherein R² is selected from groups 1 to B4, for example R² is B1.

Embodiment 54. The compound of embodiment 52, wherein R² is selected from groups B5 to B8, B12 to B19, and B21 to B23, for example R² is B14.

Embodiment 55. The compound of embodiment 52, wherein R² is selected from groups B9 to B11 and B20.

Embodiment 56. The compound of any one of embodiments 1 to 55, wherein R⁴ is an R⁵O— group, wherein R⁵ is as defined in embodiment 1 and is substituted with at least one hydroxyl group or hydroxy-substituted C₁-C₄alkyl group.

Embodiment 57. The compound of embodiment 56, wherein R⁵ is a C₂-C₆alkyl, C₄-C₁₀heterocycloalkyl, or C₄-C₁₀heterocycloalkylC₁-C₃alkyl group substituted with a hydroxy group and optionally other substituents.

Embodiment 58. The compound of embodiment 57, wherein R⁵ is selected from 2-hydroxyethyl, 3-hydroxy-1-propyl, 2-hydroxy-1-propyl, 1-hydroxy-2-propyl, 2-hydroxy-2-methyl-1-propyl, 3-hydroxy-2-methyl-1-propyl, 2-hydroxy-1-methyl-1-propyl, 3-hydroxy-1-methyl-1-propyl, and 2-hydroxy-1,1-dimethyl-1-ethyl.

Embodiment 59. The compound of embodiment 57, wherein R⁵ is C₄-C₆heterocycloalkyl, or C₄-C₆heterocycloalkylC₁-C₃alkyl group substituted with a hydroxy group and optionally other substituents, preferably the C₄-C₆heterocycloalkyl is selected from pyrrolidinyl, imidazolidinyl, piperidinyl, and piperazinyl groups.

Embodiment 60. The compound of any one of embodiments 1 to 55, wherein R⁴ is an N(R⁶)₂-group, wherein R⁶ is as defined in embodiment 1 and at least one R⁶ is substituted with at least one hydroxyl group or hydroxy-substituted C₁-C₄alkyl group and optionally substituted with one or more other substituents.

Embodiment 61. The compound of embodiment 60, wherein one R⁶ is a C₂-C₆alkyl, C₃-C₇cycloalkyl, C₄-C₁₀heterocycloalkyl, C₃-C₇cycloalkylC₁-C₃alkyl, or C₄-C₁₀heterocycloalkylC₁-C₃alkyl group substituted with at least one hydroxyl or hydroxy-substituted C₁-C₄alkyl group, and the other R⁶ is a hydrogen or C₁-C₆alkyl and optionally substituted with one or more other substituents.

Embodiment 62. The compound of embodiment 61, wherein one R⁶ is a C₂-C₆alkyl or C₃-C₇cycloalkylC₁-C₃alkyl group substituted with a hydroxyl or hydroxy-substituted C₁-C₄alkyl group, and the other R⁶ is a hydrogen or C₁-C₆alkyl.

Embodiment 63. The compound of embodiment 60, wherein the two R⁶ groups are taken together with their adjacent nitrogen atom to form a C₄-C₁₀heterocycloalkyl or C₅heteroaryl group substituted with at least one hydroxyl or hydroxy-substituted C₁-C₄alkyl group and optionally substituted with one or more other substituents.

Embodiment 64. The compound of embodiment 63, wherein the two R⁶ groups are taken together with their adjacent nitrogen atom to form a C₄-C₇heterocycloalkyl substituted with at least one hydroxyl or hydroxy-substituted C₁-C₄alkyl group and optionally substituted with one or more other substituents.

Embodiment 65. The compound of embodiment 64, wherein the C₄-C₇heterocycloalkyl group is selected from pyrrolidinyl, imidazolidinyl, piperidinyl, and piperazinyl groups substituted with at least one hydroxyl or hydroxy-substituted C₁-C₄alkyl group and optionally substituted with one or more other substituents.

Embodiment 66. The compound of embodiment 63, wherein the two R⁶ groups are taken together with their adjacent nitrogen atom to form a C₅heteroaryl group substituted with at least one hydroxyl or hydroxy-substituted C₁-C₄alkyl group and optionally substituted with one or more other substituents.

Embodiment 67. The compound of embodiment 66, wherein the C₅heteroaryl group is selected from an imidazole and a pyrrole group substituted with at least one hydroxyl or hydroxy-substituted C₁-C₄alkyl group and optionally substituted with one or more other substituents.

Embodiment 68. The compound of any one of embodiments 1 to 55, wherein R⁴ is selected from groups D5-D9, D12, D13, D19-D21, D23, D25, D27, D28, D30-D32, D36, D37, D43-D49, D52, D53, D67, D71, D75, D76, and D78-D81.

Embodiment 69. The compound of embodiment 68, wherein R⁴ is selected from groups D6-D9, D43-D47, D75 and D76.

Embodiment 70. The compound of embodiment 69, wherein R⁴ is selected from groups D6-D9 and D43-D47, preferably D9.

Embodiment 71. The compound of embodiment 68, wherein R⁴ is selected from groups D12, D19, D21, D23, D30-D32, D36, D37, D48, D49, and D78-D81, preferably D19 or D21.

Embodiment 72. The compound of embodiment 68, wherein R⁴ is selected from groups D5, D13, D20, D52, D53, D67, and D71, preferably D20.

Embodiment 73. A compound of Formula IV:

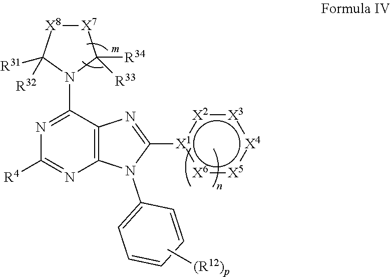

wherein X¹ to X⁸, R¹², R³¹ to R³⁴, m, n and p are as defined in any one of the above embodiments; and
R¹⁴ is N(R¹⁶)₂— or optionally substituted C₅-C₆heteroaryl; and
R¹⁶ is selected from H or a group selected from optionally substituted C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl, or two R¹⁶ groups are taken together with their adjacent nitrogen atom to form an optionally substituted C₄-C₁₀heterocycloalkyl or C₅-C₆heteroaryl;
or an isomer or a tautomer thereof, or a pharmaceutically acceptable salt thereof.

Embodiment 74. The compound of embodiment 73, wherein R¹⁴ is N(R¹⁶)₂—.

Embodiment 75. The compound of embodiment 74, wherein one R¹⁶ is an optionally substituted C₁-C₆alkyl, C₃-C₇cycloalkyl, C₄-C₁₀heterocycloalkyl, C₃-C₇cycloalkylC₁-C₃alkyl, or C₄-C₁₀heterocycloalkylC₁-C₃alkyl group, and the other R¹⁶ is a hydrogen or C₁-C₆alkyl.

Embodiment 76. The compound of embodiment 75, wherein one R¹⁶ is an C₂-C₆alkyl group substituted with one or more substituents, and the other R¹⁶ is a hydrogen or C₁-C₆alkyl.

Embodiment 77. The compound of embodiment 76, wherein said substituent is selected from F, OH, CN, alkoxy, alkylcarbonylamino, akoxycarbonylamino, alkylsulfonamido, benzyl amino, aminocarbonyl, dialkylphosphino, phosphonato, dialkylamino, cycloalkyl, heterocycloalkyl, aryl, and heteroaryl.

Embodiment 78. The compound of embodiment 74, wherein the two R¹⁶ groups are taken together with their adjacent nitrogen atom to form an optionally substituted C₄-C₁₀heterocycloalkyl or C₅heteroaryl group connected through the nitrogen.

Embodiment 79. The compound of embodiment 78, wherein the two R¹⁶ groups are taken together with their adjacent nitrogen atom to form an optionally substituted C₄-C₇heterocycloalkyl connected through the nitrogen.

Embodiment 80. The compound of embodiment 79, wherein the C₄-C₇heterocycloalkyl group is selected from optionally substituted pyrrolidinyl, imidazolidinyl, piperidinyl, and piperazinyl groups.

Embodiment 81. The compound of embodiment 78, wherein the two R¹⁶ groups are taken together with their adjacent nitrogen atom to form an optionally substituted C₅heteroaryl group connected through the nitrogen atom.

Embodiment 82. The compound of embodiment 81, wherein the C₅heteroaryl group is selected from an optionally substituted imidazole and pyrrole groups.

Embodiment 83. The compound of embodiment 73, wherein R¹⁴ is an optionally substituted C₅-C₆heteroaryl.

Embodiment 84. The compound of embodiment 73, wherein R¹⁴ is selected from D3-D5, D11-D16, D19-D28, D30-D32, D36, D37, D39-D41, D48-D54, D56, D57, D63-D67, D71, and D78-D81.

Embodiment 85. A compound of Formula V:

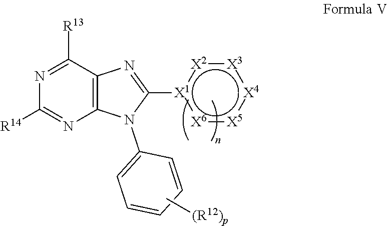

wherein X¹ to X⁶, R¹², R¹³, R¹⁴, n and p are as defined in any one of the above embodiments;
or an isomer or a tautomer thereof, or a pharmaceutically acceptable salt thereof.

Embodiment 86. A compound of Formula VI:

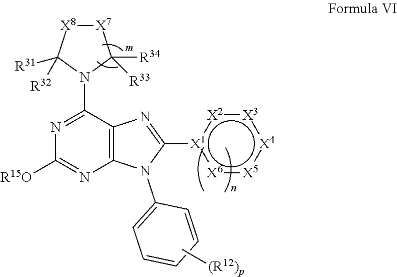

wherein X¹ to X⁸, R¹², R³¹ to R³⁴, m, n and p are as defined in any one of the above embodiments; and
R¹⁵ is a group selected from C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl;
or an isomer or a tautomer thereof, or a pharmaceutically acceptable salt thereof.

Embodiment 87. The compound of embodiment 86, wherein R¹⁵ is an optionally substituted C₁-C₆alkyl, C₄-C₁₀heterocycloalkyl, or C₄-C₁₀heterocycloalkylC₁-C₃alkyl group.

Embodiment 88. The compound of embodiment 87, wherein R¹⁵ is a C₁-C₆alkyl group substituted with one or more substituents, for instance selected from F, OH, CN, alkoxy, alkylcarbonylamino, akoxycarbonylamino, alkylsulfonamido, benzyl amino, aminocarbonyl, dialkylphosphino, phosphonato, dialkylamino, cycloalkyl, heterocycloalkyl, aryl, and heteroaryl.

Embodiment 89. The compound of embodiment 87, wherein R¹⁵ is an optionally substituted C₄-C₆heterocycloalkyl or C₄-C₆heterocycloalkylC₁-C₃alkyl group, preferably the C₄-C₆heterocycloalkyl is selected from pyrrolidinyl, imidazolidinyl, morpholinyl, piperidinyl, and piperazinyl groups.

Embodiment 90. The compound of embodiment 86, wherein the OR¹⁵ group is selected from D6-D10, D17, D29, D33-D35, D43-D47, D55, D68-D70, and D72-D77.

Embodiment 91. A compound of Formula VII:

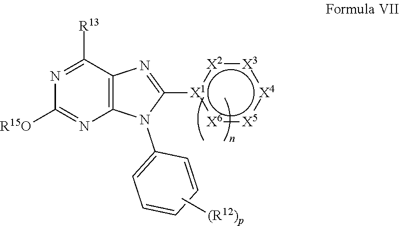

wherein X¹ to X⁶, R¹², R¹³, R¹⁵, n and p are as defined in any one of the above embodiments;
or an isomer or a tautomer thereof, or a pharmaceutically acceptable salt thereof.

Embodiment 92. A compound of Formula VIII:

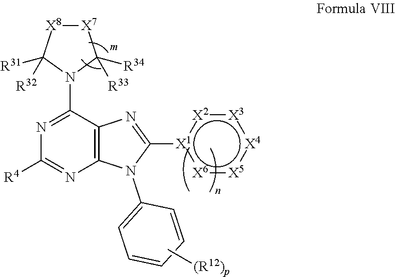

wherein X¹ to X⁸, R¹², R³¹ to R³⁴, m, n and p are as defined in any one of the above embodiments; and
R²⁴ is selected from Cl, CN, C(O)OH, R⁹C(O)N(R¹¹)—, R⁹C(O)NHC(NH)NH—, R⁹S(O)₂—, N(R¹⁰)₂C(O)—, and an optionally substituted C₁-C₆alkyl group, wherein R⁹ to R¹¹ are as previously defined;
or an isomer or a tautomer thereof, or a pharmaceutically acceptable salt thereof.

Embodiment 93. A compound of Formula IX:

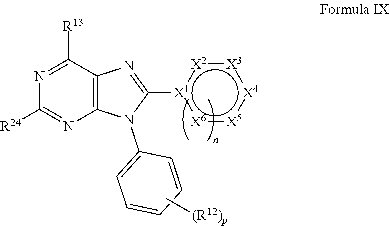

wherein X¹ to X⁶, R¹², R¹³, R²⁴, n and p are as defined in any one of the above embodiments;
or an isomer or a tautomer thereof, or a pharmaceutically acceptable salt thereof.

Embodiment 94. A compound of embodiment 92 or 93, wherein R²⁴ is selected from groups D1, D2, D18, D38, D42, and D58-D62.

Embodiment 95. A compound selected from compounds 1 to 158 and 160 to 240 as defined herein, or an isomer or a tautomer thereof, or a pharmaceutically acceptable salt thereof.

Embodiment 96. The compound of embodiment 95, wherein said compound is selected from Compounds 1-12, 14-28, 30-69, 71-97, 99-102, 104-115, 117-131, 133-137, 139-148, 150-158, 160-175, and 177-240, or an isomer or a tautomer thereof, or a pharmaceutically acceptable salt thereof.

Embodiment 97. The compound of embodiment 95, wherein said compound is selected from Compounds 1-12, 14-28, 31-36, 38-50, 52-56, 60-62, 64-68, 72-96, 99, 101, 102, 108, 110, 113, 114, 117, 119-121, 123-128, 130, 131, 133, 135-137, 140-146, 148, 150-155, 157, 158, 160-174, 177-204, 206, 207, 209-216, 220-237, and 240, or an isomer or a tautomer thereof, or a pharmaceutically acceptable salt thereof.

Embodiment 98. The compound of embodiment 95, wherein said compound is selected from Compounds 6-10, 12, 15-17, 19, 20, 23-26, 32, 33, 35, 36, 38, 41-44, 46-48, 54-56, 62, 65-67, 72-76, 78-83, 85, 86, 88-94, 96, 117, 123, 130, 131, 133, 136, 140-146, 148, 150, 152-154, 157, 158, 161-163, 165-167, 169-171, 173, 174, 177-186, 188, 189, 191, 192, 194-197, 199, 203,206, 207, 211, 213-216, 222-226, 228, 230-232, 234, and 237, or an isomer or a tautomer thereof, or a pharmaceutically acceptable salt thereof.

Embodiment 99. The compound of embodiment 1, wherein said compound is selected from Compounds 5-9, 12, 13, 19, 20, 23-26, 29, 35-38, 40, 41, 43, 46-52, 54-59, 63-67, 72-76, 78-88, 90, 93-101, 103-116, 118-124, 128-133, 135-143, 148, 153-155, 157, 158, 161-163, 165-204, 206-227, 231, 233-238, and 240, or an isomer or a tautomer thereof, or a pharmaceutically acceptable salt thereof.

Embodiment 100. The compound of embodiment 99, wherein said compound is selected from Compounds 5-9, 12, 19, 20, 23-26, 29, 35-38, 40, 41, 43, 46-52, 54-59, 63-67, 72-76, 78-88, 90, 93-97, 99-101, 104-115, 118-124, 128-131, 133, 135-137, 139-143,148,153-155, 157, 158, 161-163, 165-175, 177-204, 206-227, 231, 233-238, and 240, or an isomer or a tautomer thereof, or a pharmaceutically acceptable salt thereof.

Embodiment 101. The compound of embodiment 99, wherein said compound is selected from Compounds 5-9, 12, 19, 20, 23-26, 35, 36, 38, 40, 41, 43, 46-50, 52, 54-56, 64-67, 72-76, 78-88, 90, 93, 94, 96, 99, 101, 108, 110, 113, 114, 119-121,123,124, 128, 130, 131, 133, 135-137, 140-143, 148, 153-155, 157, 158, 161-163,165-174, 177-204, 206, 207, 209-216, 220-227, 231, 233-237, and 240, or an isomer or a tautomer thereof, or a pharmaceutically acceptable salt thereof.

Embodiment 102. The compound of embodiment 99, wherein said compound is selected from Compounds 6-9, 12, 19, 20, 23-26, 35, 36, 38, 41, 43, 46-48, 54-56, 65-67, 72-76, 78-83, 85, 86, 88, 90, 93, 94, 96, 123, 130, 131, 133, 136, 140-143, 148, 153, 154, 157, 158, 161-163, 165-167, 169-171, 173, 174, 177-186, 188, 189, 191, 192, 194-197, 199, 203, 206, 207, 211, 213-216, 222-226, 231, 234, and 237, or an isomer or a tautomer thereof, or a pharmaceutically acceptable salt thereof.

Embodiment 103. A pharmaceutical composition comprising a compound as defined in any one of embodiments 1 to 102, together with a pharmaceutically acceptable carrier, diluent or excipient.

Embodiment 104. Use of a compound as defined in any one of embodiments 1 to 102 or a pharmaceutical composition as defined in embodiment 103 for the treatment of a disorder related to appetite or one of its complications, a disorder related to glucose regulation or one of its complications, a fibrosis related disorder or one of its complications, a disorder related to metabolism or one of its complications, a disorder related to skin and hair growth and healing, a disorder related to the GI tract, a disorder related to obesity or one of its complications, or a combination thereof.

Embodiment 105. The use of embodiment 104, wherein said disorder related to appetite or one of its complications is selected from Prader-Willi Syndrome (PWS), hypothalamic obesity, pro-opiomelanocortin (POMC) deficiency (including POMC obesity, heterozygous POMC deficiency obesity, POMC epigenetic disorders), Leptin receptor (LepR) deficiency, Bardet-Biedl (BB) syndrome, and Alström syndrome.

Embodiment 106. The use of embodiment 104, wherein said disorder related to glucose regulation or one of its complications is selected from diabetes Type I, diabetes Type II, insulin resistance, pre-diabetes, pancreatic diseases (by β-cell protection and/or increased insulin production), and associated nephropathies, neuropathies and retinopathies.

Embodiment 107. The use of embodiment 104, wherein said fibrosis related disorder or one of its complications is selected from progressive fibrosis associated with interstitial lung disease, idiopathic pulmonary fibrosis (IPF), Hermansky-Pudlak syndrome pulmonary fibrosis (HPS-PF), cirrhosis and other liver fibrosis disorders (such as nonalcoholic steatohepatitis (NASH), primary sclerosing cholangitis, primary biliary cholangitis), skin fibrotic disorders (such as scleroderma), fibrotic renal diseases and chronic kidney diseases.

Embodiment 108. The use of embodiment 104, wherein said disorder related to metabolism or one of its complications is selected from metabolic syndrome and hyperlipidemia (e.g. hyper-triglyceridemia, hyper-triglyceridemia in the setting of low HDL-cholesterol, elevation of LDL and/or total cholesterol and/or VLDL and/or elevated Apolipoprotein B, atherosclerotic cardiovascular disease, etc.).

Embodiment 109. The use of embodiment 104, wherein said disorder related to obesity or one of its complications is selected from sleep apnea, snoring, asthma, pulmonary hypoventilation syndrome, dementia, heart disease, hypertension, gallbladder disease, gastrointestinal disorders, menstrual irregularities, degenerative arthritis, venous statis ulcer, coronary artery disease, arterial sclerotic disease, pseudotumor cerebri, osteoarthritis, high cholesterol, and increased incidence of malignancies of the liver, ovaries, cervix, uterus, breasts, prostate, or gallbladder.

Embodiment 110. The use of embodiment 104, wherein said disorder of the skin and hair is selected from alopecia (male pattern baldness and alopecia associated with metabolic syndrome), excessive scar formation (cicatrix and keloid), and scleroderma.

Embodiment 111. The use of embodiment 104, wherein said disorder related to the GI tract is selected from constipation, irritable bowel syndrome, and inflammatory bowel syndrome, including ulcerative colitis and Crohn's disease.

Embodiment 112. A method for the treatment of a disorder selected from disorders related to appetite or their complications, disorders related to glucose regulation or their complications, fibrosis related disorders or their complications, disorders related to metabolism or their complications, disorders related to skin and hair growth and healing, disorders related to the GI tract, disorders related to obesity or their complications, or a combination thereof, comprising administering a compound as defined in any one of embodiments 1 to 102 or a pharmaceutical composition as defined in embodiment 103 to a subject in need thereof.

Embodiment 113. The method of embodiment 112, wherein said disorders related to appetite or their complications are selected from Prader-Willi Syndrome (PWS), hypothalamic obesity, pro-opiomelanocortin (POMC) deficiency (including POMC obesity, heterozygous POMC deficiency obesity, POMC epigenetic disorders), leptin receptor (LepR) deficiency, Bardet-Biedl (BB) syndrome, and Alström syndrome.

Embodiment 114. The method of embodiment 112, wherein said disorders related to glucose regulation or their complications are selected from diabetes Type I, diabetes Type II, insulin resistance, pre-diabetes, pancreatic diseases (by β-cell protection and/or increased insulin production), and associated nephropathies, neuropathies and retinopathies.

Embodiment 115. The method of embodiment 112, wherein said fibrosis related disorders or their complications are selected from progressive fibrosis associated with interstitial lung disease, idiopathic pulmonary fibrosis (IPF), Hermansky-Pudlak syndrome pulmonary fibrosis (HPS-PF), cirrhosis and other liver fibrosis disorders (such as nonalcoholic steatohepatitis (NASH), primary sclerosing cholangitis, primary biliary cholangitis), skin fibrotic disorders (such as scleroderma), fibrotic renal diseases and chronic kidney diseases.

Embodiment 116. The method of embodiment 112, wherein said disorders related to metabolism or their complications are selected from metabolic syndrome and hyperlipidemia (e.g. hyper-triglyceridemia, hyper-triglyceridemia in the setting of low HDL-cholesterol, elevation of LDL and/or total cholesterol and/or VLDL and/or elevated Apolipoprotein B, atherosclerotic cardiovascular disease, etc.).

Embodiment 117. The method of embodiment 112, wherein said disorders related to obesity or their complications are selected from sleep apnea, snoring, asthma, pulmonary hypoventilation syndrome, dementia, heart disease, hypertension, gallbladder disease, gastrointestinal disorders, menstrual irregularities, degenerative arthritis, venous statis ulcer, coronary artery disease, arterial sclerotic disease, pseudotumor cerebri, osteoarthritis, high cholesterol, and increased incidence of malignancies of the liver, ovaries, cervix, uterus, breasts, prostate, or gallbladder.

Embodiment 118. The method of embodiment 112, wherein said disorders of the skin and hair is selected from alopecia (male pattern baldness and alopecia associated with metabolic syndrome), excessive scar formation (cicatrix and keloid), and scleroderma.

Embodiment 119. The method of embodiment 112, wherein said disorders related to the GI tract is selected from constipation, irritable bowel syndrome, and inflammatory bowel syndrome, including ulcerative colitis and Crohn's disease.

Additional objects and features of the present compound, compositions, methods and uses will become more apparent upon reading of the following non-restrictive description of exemplary embodiments and examples section, which should not be interpreted as limiting the scope of the invention.

### DETAILED DESCRIPTION

All technical and scientific terms and expressions used herein have the same definitions as those commonly understood by a person skilled in the art to which the present technology pertains. The definition of some terms and expressions used is nevertheless provided below. To the extent the definitions of terms in the publications, patents, and patent applications incorporated herein by reference are contrary to the definitions set forth in this specification, the definitions in this specification will control. The section headings used herein are for organizational purposes only and are not to be construed as limiting the subject matter disclosed.

Chemical structures described herein are drawn according to conventional standards. Also, when an atom, such as a carbon atom, as drawn seems to include an incomplete valency, then the valency is assumed to be satisfied by one or more hydrogen atoms even though these are not necessarily explicitly drawn. Hydrogen atoms should be inferred to be part of the compound.

The terminology used herein is for the purpose of describing particular embodiments only and is not intended to be limiting. It should be noted that, the singular forms “a”, “an”, and “the” include plural forms as well, unless the content clearly dictates otherwise. Thus, for example, reference to a composition containing “a compound” also contemplates a mixture of two or more compounds. It should also be noted that the term “or” is generally employed in its sense including “and/or” unless the context clearly dictates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the terms “compounds”, “compounds herein described”, “compounds of the present application”, “purines”, “purine compounds” and equivalent expressions refer to compounds described in the present application, e.g. those encompassed by structural Formulae I to IX, optionally with reference to any of the applicable embodiments, and also includes exemplary compounds, such as Compounds 1 to 240, their pharmaceutically acceptable salts and tautomeric forms, as well as solvates, esters, and prodrugs thereof when applicable. When a zwitterionic form is possible, the compound may be drawn as its neutral form for practical purposes, but the compound is understood to also include its zwitterionic form. Embodiments herein may also exclude one or more of the compounds. Compounds may be identified either by their chemical structure or their chemical name. In a case where the chemical structure and chemical name would conflict, the chemical structure will prevail.

Unless otherwise stated, structures depicted herein are also meant to include all isomeric (e.g., enantiomeric, diastereomeric, tautomeric and geometric (or conformational)) forms of the structure when applicable; for example, the R and S configurations for each asymmetric center unless specified. Therefore, single stereochemical isomers as well as enantiomeric, diastereomeric, tautomeric and geometric (or conformational) mixtures of the present compounds are within the scope of the present description. The present compounds unless otherwise noted, also encompasses all possible tautomeric forms of the illustrated compound, if any. The term also includes isotopically labeled compounds where one or more atoms have an atomic mass different from the atomic mass most abundantly found in nature. Examples of isotopes that may be incorporated into the present compounds include, but are not limited to, ²H (D), ³H (T), ¹¹C, ¹³C, ¹⁴C, ¹⁵N, ¹⁸O, ¹⁷O, any one of the isotopes of sulfur, etc. The compounds may also exist in unsolvated forms as well as solvated forms, including hydrated forms. The compounds may exist in multiple crystalline or amorphous forms. In general, all physical forms are equivalent for the uses contemplated herein and are intended to be within the scope of the present invention.

Where a particular enantiomer is preferred, it may, in some embodiments be provided substantially free of the corresponding enantiomer and may also be enantiomerically enriched. “Enantiomerically enriched” means that the compound is made up of a significantly greater proportion of one enantiomer. In certain embodiments the compound is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including high-pressure liquid chromatography (HPLC) or supercritical Fluid Chromatography (SFC) on chiral support, or by the formation and crystallization of chiral salts or be prepared by asymmetric syntheses.

The expression “pharmaceutically acceptable salt” refers to those salts of the compounds of the present description which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the present description, or separately by reacting a free base function of the compound with a suitable organic or inorganic acid (acid addition salts) or by reacting an acidic function of the compound with a suitable organic or inorganic base (base addition salts).

The term “solvate” refers to a physical association of one of the present compounds with one or more solvent molecules, including water and non-aqueous solvent molecules. This physical association may include hydrogen bonding. In certain instances, the solvate will be capable of isolation, for example when one or more solvent molecules are incorporated in the crystal lattice of a crystalline solid. The term “solvate” encompasses both solution-phase and isolable solvates. Exemplary solvates include, without limitation, hydrates, hemihydrates, ethanolates, hemiethanolates, n-propanolates, iso-propanolates, 1-butanolates, 2-butanolate, and solvates of other physiologically acceptable solvents, such as the Class 3 solvents described in the International Conference on Harmonization (ICH), Guide for Industry, Q3C Impurities: Residual Solvents (1997). Accordingly, the compound as herein described also includes each of its solvates and mixtures thereof.

As used herein, the expression “pharmaceutically acceptable ester” refers to esters of the compounds formed by the process of the present description which may hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates of hydroxyl groups, and alkyl esters of an acidic group. Other ester groups include sulfonate or sulfate esters.

The expression “pharmaceutically acceptable prodrugs” as used herein refers to those prodrugs of the compounds formed by the process of the present description which are, within the scope of sound medical judgment, suitable for use in contact with the tissues of humans and lower animals with undue toxicity, irritation, allergic response, and the like, commensurate with a reasonable benefit/risk ratio, and effective for their intended use. The term “prodrug”, as used herein, means a compound which is convertible in vivo by metabolic means (e.g. by hydrolysis) to afford any compound delineated by the formulae of the instant description.

Abbreviations may also be used throughout the application, unless otherwise noted, such abbreviations are intended to have the meaning generally understood by the field. Examples of such abbreviations include Me (methyl), Et (ethyl), Pr (propyl), i-Pr (isopropyl), Bu (butyl), t-Bu (tert-butyl), i-Bu (iso-butyl), s-Bu (sec-butyl), c-Bu (cyclobutyl), Ph (phenyl), Bn (benzyl), Bz (benzoyl), CBz or Cbz or Z (carbobenzyloxy), Boc or BOC (tert-butoxycarbonyl), and Su or Suc (succinimide).

The number of carbon atoms in a hydrocarbon substituent can be indicated by the prefix “Cₓ-C_{y}” or “C_{x-y}” where x is the minimum and y is the maximum number of carbon atoms in the substituent. However, when the prefix “Cₓ-C_{y}” or “C_{x-y}” is associated with a group incorporating one or more heteroatom(s) by definition (e.g. heterocycloalkyl, heteroaryl, etc.), then x and y define respectively the minimum and maximum number of atoms in the group (e.g. in the cycle or cycles), including carbon atoms as well as heteroatom(s).

The term “heteroatom” includes atoms other than carbon and hydrogen, such as, but not limited to nitrogen, oxygen, or sulfur, and includes any oxidized form of nitrogen or sulfur, substituted form of nitrogen, and any quaternized form of a basic nitrogen.

The term “alkyl” as used herein, refers to a saturated, straight- or branched-chain hydrocarbon radical typically containing from 1 to 20 carbon atoms. For example, “C₁₋₈alkyl” contains from one to eight carbon atoms. Examples of alkyl radicals include, but are not limited to, methyl, ethyl, propyl, isopropyl, n-butyl, tert-butyl, neopentyl, n-hexyl, heptyl, octyl radicals and the like.

The term “alkenyl” as used herein, denotes a straight- or branched-chain hydrocarbon radical containing one or more double bonds and typically from 2 to 20 carbon atoms. For example, “C₂₋₈alkenyl” contains from two to eight carbon atoms. Alkenyl groups include, but are not limited to, for example, ethenyl, propenyl, butenyl, 1-methyl-2-buten-1-yl, heptenyl, octenyl and the like.

The term “alkynyl” as used herein, denotes a straight- or branched-chain hydrocarbon radical containing one or more triple bonds and typically from 2 to 20 carbon atoms. For example, “C₂₋₈alkynyl” contains from two to eight carbon atoms. Representative alkynyl groups include, but are not limited to, for example, ethynyl, 1-propynyl, 1-butynyl, heptynyl, octynyl and the like.

The terms “cycloalkyl”, “alicyclic”, “carbocyclic” and equivalent expressions refer to a group comprising a saturated or partially unsaturated (non-aromatic) carbocyclic ring in a monocyclic or polycyclic ring system, including spiro (sharing one atom), fused (sharing at least one bond) or bridged (sharing two or more bonds) carbocyclic ring systems, having from three to fifteen ring members. Examples of cycloalkyl groups include, without limitation, cyclopropyl, cyclobutyl, cyclopentyl, cyclopenten-1-yl, cyclopenten-2-yl, cyclopenten-3-yl, cyclohexyl, cyclohexen-1-yl, cyclohexen-2-yl, cyclohexen-3-yl, cycloheptyl, bicyclo[4.3.0]nonanyl, norbornyl, and the like. The term cycloalkyl includes both unsubstituted cycloalkyl groups and substituted cycloalkyl groups.

The term “C₃₋ₙcycloalkyl” refers to a cycloalkyl group having from 3 to the indicated “n” number of carbon atoms in the ring structure. Unless the number of carbons is otherwise specified, “lower cycloalkyl” groups as herein used, have at least 3 and equal or less than 8 carbon atoms in their ring structure.

As used herein, the terms “heterocycloalkyl”, “heterocyclyl”, and the like are used interchangeably and refer to a chemically stable 3- to 7-membered monocyclic or 7-10-membered bicyclic heterocycloalkyl moiety that is either saturated or partially unsaturated, and having, in addition to carbon atoms, one or more, preferably one to four, heteroatoms, as defined above. As an example, in a saturated or partially unsaturated ring having 1-3 heteroatoms selected from oxygen, sulfur or nitrogen, the nitrogen may be N (as in 3,4-dihydro-2H-pyrrolyl), NH (as in pyrrolidinyl), or NR (as in N-substituted pyrrolidinyl). A heterocycloalkyl can be attached to its pendant group at any heteroatom or carbon atom that results in a chemically stable structure and any of the ring atoms can be optionally substituted. Examples of heterocycloalkyl groups include, but are not limited to, 1,3-dioxolanyl, pyrrolidinyl, pyrrolidonyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, tetrahydrofuranyl, tetrahydropyranyl, tetrahydrothiopyranyl, tetrahydrodithienyl, tetrahydrothienyl, thiomorpholino, thioxanyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, 2H-pyranyl, 4H-pyranyl, dioxanyl, dithianyl, dithiolanyl, dihydropyranyl, dihydrothienyl, dihydrofuranyl, 3-azabicyclo[3.1.0]hexanyl, 3-azabicyclo[4.1.0]heptanyl, quinolizinyl, quinuclidinyl, tetrahydroquinolinyl, tetrahydroisoquinolinyl, decahydroquinolinyl, and the like. Heterocycloalkyl groups also include groups in which a heterocycloalkyl ring is fused to one or more aryl, heteroaryl, or cycloaliphatic rings, such as indolinyl, 3H-indolyl, chromanyl, chromenyl, phenanthridinyl, 2-azabicyclo[2.2.1]heptanyl, octahydroindolyl, or tetrahydroquinolinyl, where the radical or point of attachment is on the heterocycloalkyl ring. A heterocycloalkyl group may be mono- or bicyclic. The term “heterocyclylalkyl” refers to an alkyl group substituted by a heterocycloalkyl, wherein the alkyl and heterocyclyl portions independently are optionally substituted. The term “C₃₋ₙheterocycloalkyl” refers to a heterocycloalkyl group having from 3 to the indicated “n” number of atoms in the ring structure, including carbon atoms and heteroatoms.

As used herein, the term “partially unsaturated” refers to a ring moiety that includes at least one double or triple bond between ring atoms but is not aromatic. The term “partially unsaturated” is intended to encompass rings having one or multiple sites of unsaturation but is not intended to include aryl or heteroaryl moieties, as herein defined.

The term “aryl” used alone or as part of a larger moiety as in “aralkyl”, “aralkoxy”, “aryloxy”, or “aryloxyalkyl”, refers to aromatic groups having 4n+2 conjugated π(pi) electrons, wherein n is an integer from 1 to 3, in a monocyclic moiety or a bicyclic or tricyclic fused ring system having a total of six to 15 ring members, wherein at least one ring in the system is aromatic and wherein each ring in the system contains three to seven ring members. The term “aryl” may be used interchangeably with the expression “aryl ring”. In certain embodiments of the present description, “aryl” refers to an aromatic ring or ring system which includes, but is not limited to, phenyl, biphenyl, naphthyl, azulenyl, anthracyl and the like, which may bear one or more substituents. The term “aralkyl” or “arylalkyl” refers to an alkyl residue attached to an aryl ring. Examples of aralkyl include, but are not limited to, benzyl, phenethyl, and the like. Also included within the scope of the term “aryl”, as it is used herein, is a group in which an aromatic ring is fused to one or more non-aromatic rings, such as indanyl, indenyl, phthalimidyl, naphthimidyl, fluorenyl, phenanthridinyl, or tetrahydronaphthyl, and the like. The term “C₆₋ₙaryl” refers to an aryl group having from 6 to the indicated “n” number of atoms in the ring structure.

The term “heteroaryl”, used alone or as part of a larger moiety, e.g., “heteroaralkyl”, or “heteroaralkoxy”, refers to aromatic groups having 4n+2 conjugated π(pi) electrons, wherein n is an integer from 1 to 3 (e.g. having 5 to 18 ring atoms, preferably 5, 6, or 9 ring atoms; having 6, 10, or 14 π electrons shared in a cyclic array); and having, in addition to carbon atoms, from one to five heteroatoms. The term “heteroatom” is as defined above. A heteroaryl may be a single ring, or two or more fused rings. The term “heteroaryl”, as used herein, also includes groups in which a heteroaromatic ring is fused to one or more aryl, cycloalkyl, or heterocycloalkyl rings. Nonlimiting examples of heteroaryl groups include thienyl, furanyl (furyl), pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, triazinyl, indolyl, 3H-indolyl, isoindolyl, indolizinyl, benzothienyl (benzothiophenyl), benzofuranyl, dibenzofuranyl, indazolyl, benzimidazolyl, benzoxazolyl, benzothiazolyl, benzotriazolyl, pyrrolopyridinyl (e.g. pyrrolo[3,2-b]pyridinyl or pyrrolo[3,2-c]pyridinyl), pyrazolopyridinyl (e.g. pyrazolo[1,5-a]pyridinyl), furopyridinyl, purinyl, imidazopyrazinyl (e.g. imidazo[4,5-b]pyrazinyl), quinolyl (quinolinyl), isoquinolyl (isoquinolinyl), quinolonyl, isoquinolonyl, cinnolinyl, phthalazinyl, quinazolinyl, quinoxalinyl, 4H-quinolizinyl, naphthyridinyl, and pteridinyl carbazolyl, acridinyl, phenanthridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. Heteroaryl groups include rings that are optionally substituted. The term “heteroaralkyl” refers to an alkyl group substituted by a heteroaryl, wherein the alkyl and heteroaryl portions are independently optionally substituted. Examples include, but are not limited to, pyridinylmethyl, pyrimidinylethyl and the like. For instance, the term “C₅₋ₙheteroaryl” refers to a heteroaryl group having from 5 to the indicated “n” number of atoms in the ring structure, including carbon atoms and heteroatoms.

The term “halogen” or “halo” designates a halogen atom, i.e. a fluorine, chlorine, bromine or iodine atom, preferably fluorine or chlorine.

As described herein, compounds of the present description may contain “optionally substituted” moieties. In general, the term “substituted” means that one or more hydrogens of the designated moiety are replaced with a suitable substituent. Unless otherwise indicated, an “optionally substituted” group may have a suitable substituent at any or each substitutable position of the group, and when more than one position in any given structure may be substituted with more than one substituent selected from a specified group, the substituent may be either the same or different at each position. Combinations of substituents envisioned under the present description are preferably those that result in the formation of chemically stable or chemically feasible compounds. The term “chemically stable”, as used herein, refers to compounds that are not substantially altered when subjected to conditions to allow for their production, detection, and, in certain embodiments, their recovery, purification, and use for one or more of the purposes disclosed herein.

Examples of substituents include, but are not limited to halogen (F, Cl, Br, I), OH, CO₂H, alkoxy, oxo, thiooxo, NO₂, CN, CF₃, CHF₂, NH₂, NHalkyl, NHalkenyl, NHalkynyl, NHcycloalkyl, NHaryl, NHheteroaryl, NHheterocycloalkyl, dialkylamino, diarylamino, diheteroarylamino, dicycloalkylamino, diheterocycloalkylamino, N-alkyl-N-arylamino, N-alkyl-N-heteroarylamino, N-alkyl-N-cycloalkylamino, N-alkyl-N-heterocycloalkylamino, O-alkyl, O-alkenyl, O-alkynyl, O-cycloalkyl, O-aryl, O-heteroaryl, O-haloalkyl, O-heterocycloalkyl, C(O)alkyl, C(O)alkenyl, C(O)alkynyl, C(O)cycloalkyl, C(O)aryl, C(O)heteroaryl, C(O)heterocycloalkyl, CO₂alkyl, CO₂alkenyl, CO₂alkynyl, CO₂cycloalkyl, CO₂aryl, CO₂heteroaryl, CO₂heterocycloalkyl, OC(O)alkyl, OC(O)alkenyl, OC(O)alkynyl, OC(O)cycloalkyl, OC(O)aryl, OC(O)heteroaryl, OC(O)heterocycloalkyl, C(O)NH₂, C(O)NHalkyl, C(O)NHalkenyl, C(O)NHalkynyl, C(O)NHcycloalkyl, C(O)NHaryl, C(O)NHheteroaryl, C(O)NHheterocycloalkyl, OCO₂alkyl, OCO₂alkenyl, OCO₂alkynyl, OCO₂cycloalkyl, OCO₂aryl, OCO₂heteroaryl, OCO₂heterocycloalkyl, OC(O)NH₂, OC(O)NHalkyl, OC(O)NHalkenyl, OC(O)NHalkynyl, OC(O)NHcycloalkyl, OC(O)NHaryl, OC(O)NHheteroaryl, OC(O)NHheterocycloalkyl, OP(O)(Oalkyl)₂, OP(O)(OH)₂, OP(O)(Oalkenyl)₂, OP(O)(Oalkynyl)₂, OP(O)(Ocycloalkyl)₂, OP(O)(Oaryl)₂, OP(O)(Oheteroaryl)₂, OP(O)(Oheterocycloalkyl)₂, P(O)(Oalkyl)₂, P(O)(OH)₂, P(O)(Oalkenyl)₂, P(O)(Oalkynyl)₂, P(O)(Ocycloalkyl)₂, P(O)(Oaryl)₂, P(O)(Oheteroaryl)₂, P(O)(Oheterocycloalkyl)₂, P(O)(alkyl)₂, P(O)(alkenyl)₂, P(O)(alkynyl)₂, P(O)(cycloalkyl)₂, P(O)(aryl)₂, P(O)(heteroaryl)₂, P(O)(heterocycloalkyl)₂, NHC(O)alkyl, NHC(O)alkenyl, NHC(O)alkynyl, NHC(O)cycloalkyl, NHC(O)aryl, NHC(O)heteroaryl, NHC(O)heterocycloalkyl, NHCO₂alkyl, NHCO₂alkenyl, NHCO₂alkynyl, NHCO₂cycloalkyl, NHCO₂aryl, NHCO₂heteroaryl, NHCO₂heterocycloalkyl, NHC(O)NH₂, NHC(O)NHalkyl, NHC(O)NHalkenyl, NHC(O)NHalkenyl, NHC(O)NHcycloalkyl, NHC(O)NHaryl, NHC(O)NHheteroaryl, NHC(O)NHheterocycloalkyl, NHC(S)NH₂, NHC(S)NHalkyl, NHC(S)NHalkenyl, NHC(S)NHalkynyl, NHC(S)NHcycloalkyl, NHC(S)NHaryl, NHC(S)NHheteroaryl, NHC(S)NHheterocycloalkyl, NHC(NH)NH₂, NHC(NH)NHalkyl, NHC(NH)NHalkenyl, NHC(NH)NHalkenyl, NHC(NH)NHcycloalkyl, NHC(NH)NHaryl, NHC(NH)NHheteroaryl, NHC(NH)NHheterocycloalkyl, NHC(NH)alkyl, NHC(NH)alkenyl, NHC(NH)alkenyl, NHC(NH)cycloalkyl, NHC(NH)aryl, NHC(NH)heteroaryl, NHC(NH)heterocycloalkyl, C(NH)NHalkyl, C(NH)NHalkenyl, C(NH)NHalkynyl, C(NH)NHcycloalkyl, C(NH)NHaryl, C(NH)NHheteroaryl, C(NH)NHheterocycloalkyl, S(O)alkyl, S(O)alkenyl, S(O)alkynyl, S(O)cycloalkyl, S(O)aryl, S(O)₂alkyl, S(O)₂alkenyl, S(O)₂alkynyl, S(O)₂cycloalkyl, S(O)₂aryl, S(O)heteroaryl, S(O)heterocycloalkyl, SO₂NH₂, SO₂NHalkyl, SO₂NHalkenyl, SO₂NHalkynyl, SO₂NHcycloalkyl, SO₂NHaryl, SO₂NHheteroaryl, SO₂NHheterocycloalkyl, NHSO₂alkyl, NHSO₂alkenyl, NHSO₂alkynyl, NHSO₂cycloalkyl, NHSO₂aryl, NHSO₂heteroaryl, NHSO₂heterocycloalkyl, CH₂NH₂, CH₂SO₂CH₃, alkyl, alkenyl, alkynyl, aryl, arylalkyl, heteroaryl, heteroarylalkyl, heterocycloalkyl, cycloalkyl, carbocyclic, heterocycloalkyl, polyalkoxyalkyl, polyalkoxy, methoxymethoxy, methoxyethoxy, SH, S-alkyl, S-alkenyl, S-alkynyl, S-cycloalkyl, S-aryl, S-heteroaryl, S-heterocycloalkyl, or methylthiomethyl. Each of these substituents may also be further substituted where possible.

The present document therefore to purine compounds as defined herein and in the following paragraphs. When referring to chemical moieties, the recitation of a listing of chemical groups in any definition of a variable includes definitions of that variable as any single group or combination of listed groups. Similarly, the recitation of an embodiment for a variable herein includes that embodiment as any single embodiment or in combination with any other embodiments or portions thereof. As such, the following embodiments are present alone or in combination if applicable.

The present compounds present a purine core structure to which is attached defined substituents. Exemplary compounds as defined herein are illustrated by the General Formula:

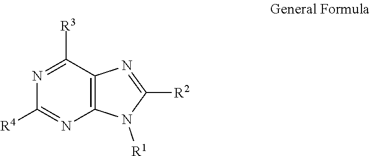

wherein,
R¹ and R² are each independently selected from optionally substituted C₆₋₁₀aryl and optionally substituted C₅₋₁₀heteroaryl;
R³ is selected from an optionally substituted R⁷O—, N(R⁸)₂—, or C₅₋₆heteroaryl group;
R⁴ is selected from Cl, CN, C(O)OH, and an optionally substituted group selected from
R⁹C(O)N(R¹¹)—, R⁹C(O)NHC(NH)NH—, R⁹S(O)₂—, N(R¹⁰)₂C(O)—, R⁵O—, N(R⁶)₂—, C₁-C₆alkyl, C₅-C₆heteroaryl;
R⁵ is a group selected from C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl;
R⁶ is H or a group selected from C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl, wherein at least one R⁶ is other than H, or two R⁶ groups are taken together with their adjacent nitrogen atom to form a C₄-C₁₀heterocycloalkyl or C₅-C₆heteroaryl group;
R⁷ is an optionally substituted C₁-C₆alkyl; and
R³ is independently in each occurrence selected from hydrogen, C₁-C₆alkyl, C₃-C₇cycloalkyl, and C₄-C₁₀heterocycloalkyl;
R⁹ is independently in each occurrence selected from optionally substituted C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, and C₅-C₆heteroarylC₁-C₃alkyl;
R¹⁰ is independently in each occurrence selected from H or a group selected from optionally substituted C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, and C₅-C₆heteroarylC₁-C₃alkyl, or two
R¹⁰ groups are taken together with their adjacent nitrogen atom to form an optionally substituted C₄-C₁₀heterocycloalkyl or C₅-C₆heteroaryl;
R¹¹ is independently in each occurrence selected from H or a group selected from optionally substituted C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl;
wherein each of the alkyl, cycloalkyl, heterocycloalkyl, aryl, and heteroaryl groups is optionally substituted;
or an isomer or a tautomer thereof, or a pharmaceutically acceptable salt thereof.

Non-limiting examples of R¹ comprises groups A1 and A2:

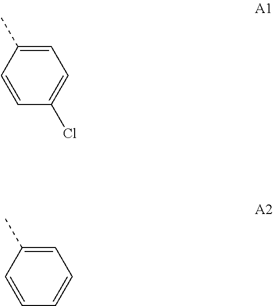

Non-limiting examples of R² comprises groups 1 to B23:

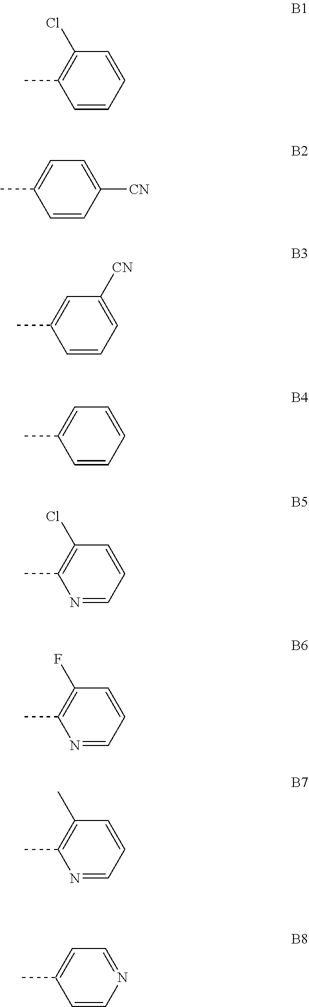

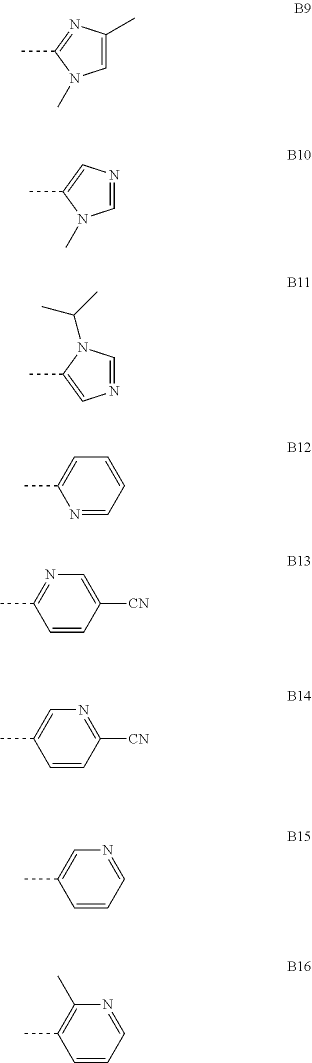

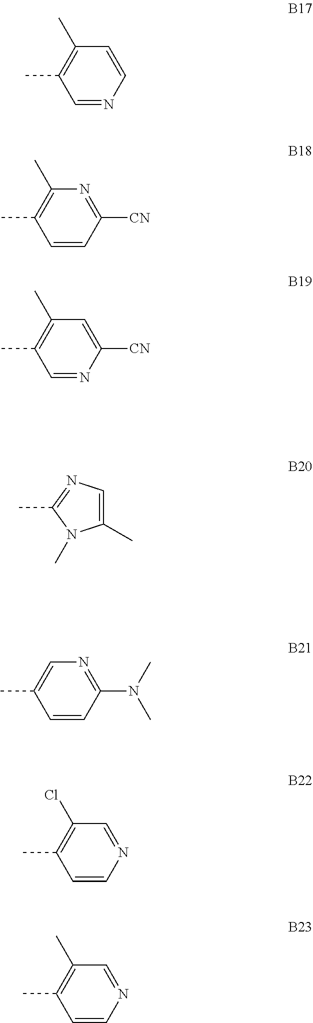

Non-limiting examples of R³ comprises groups C1 to C69:

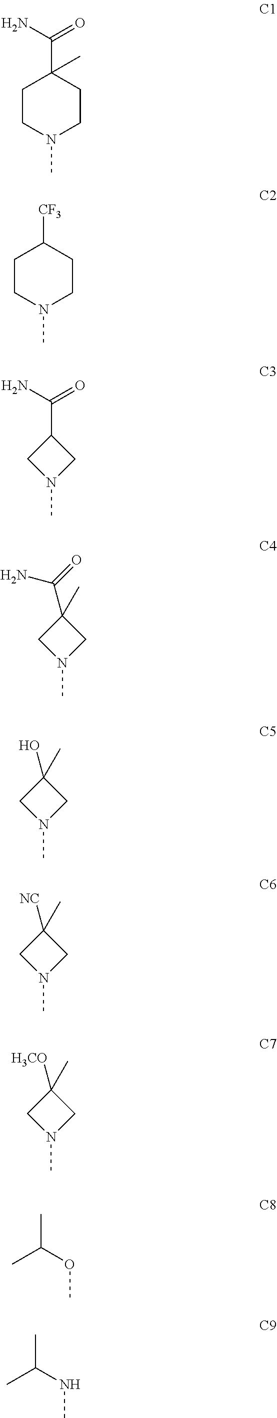

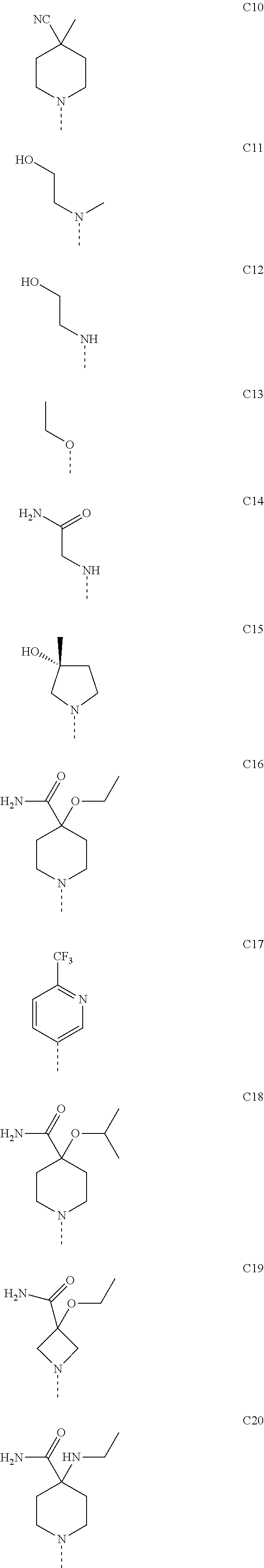

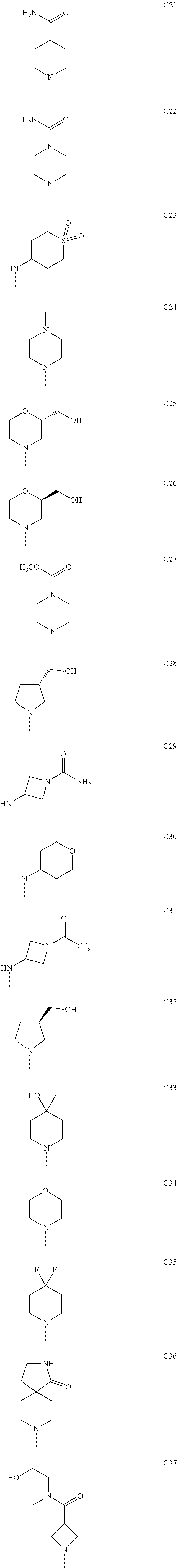

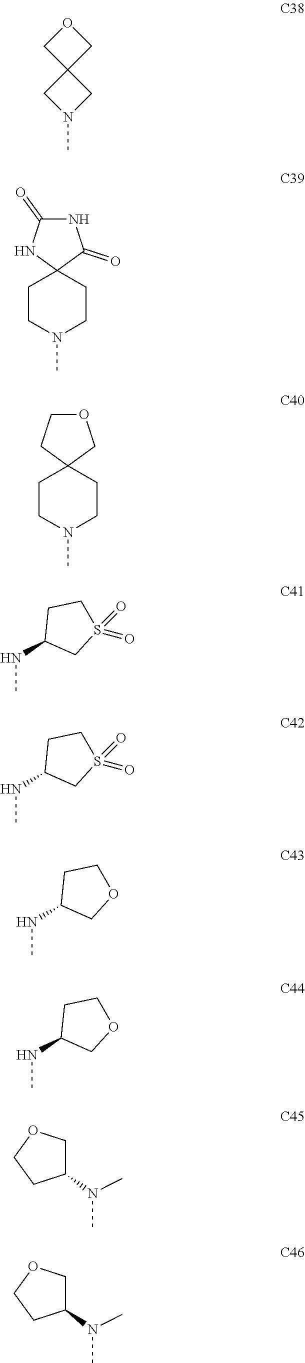

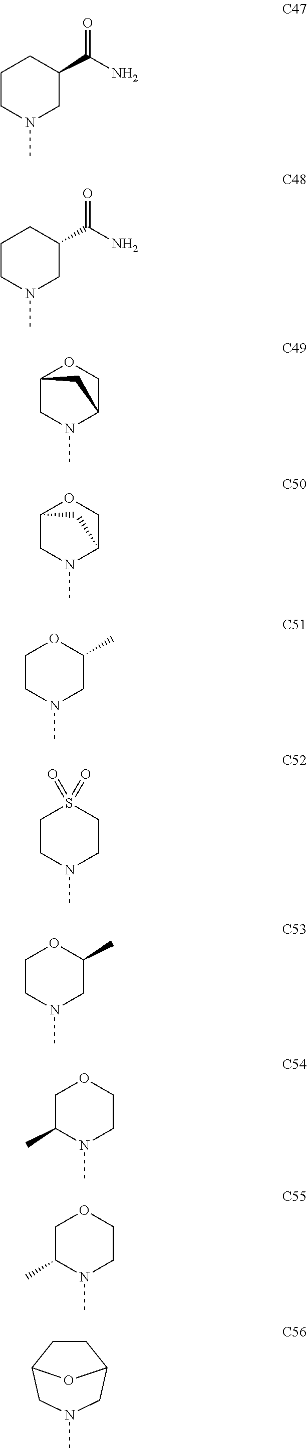

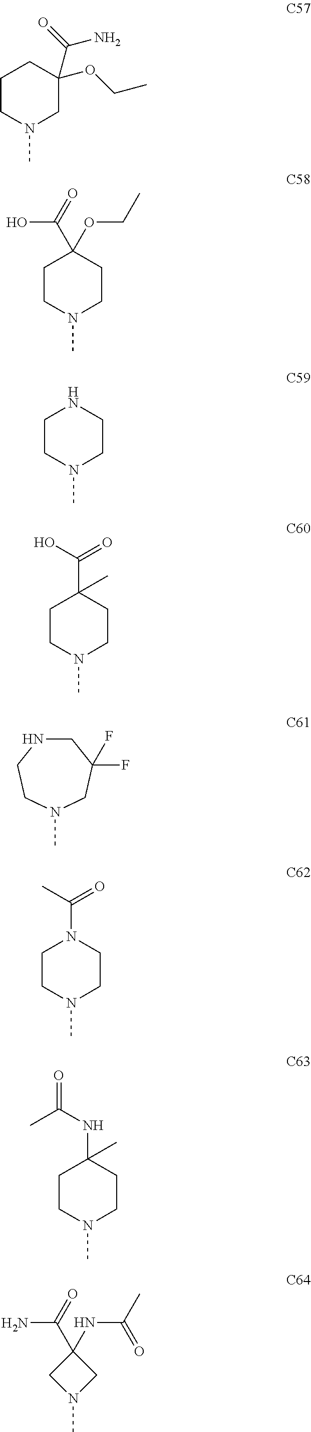

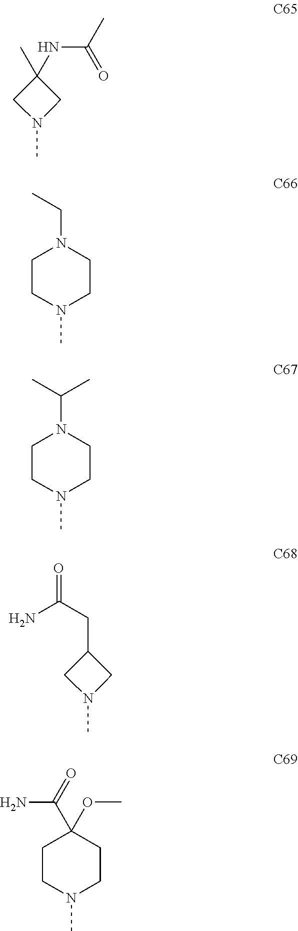

Non-limiting examples of R⁴ comprises groups D1 to D81:

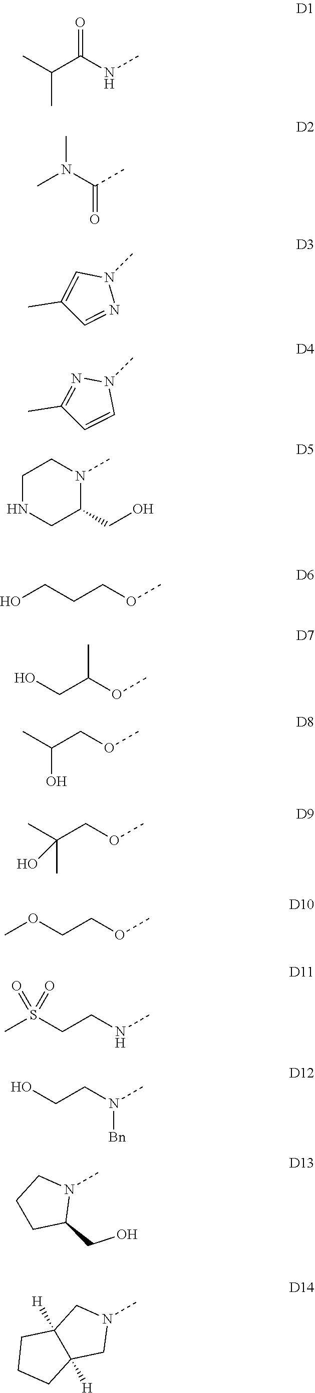

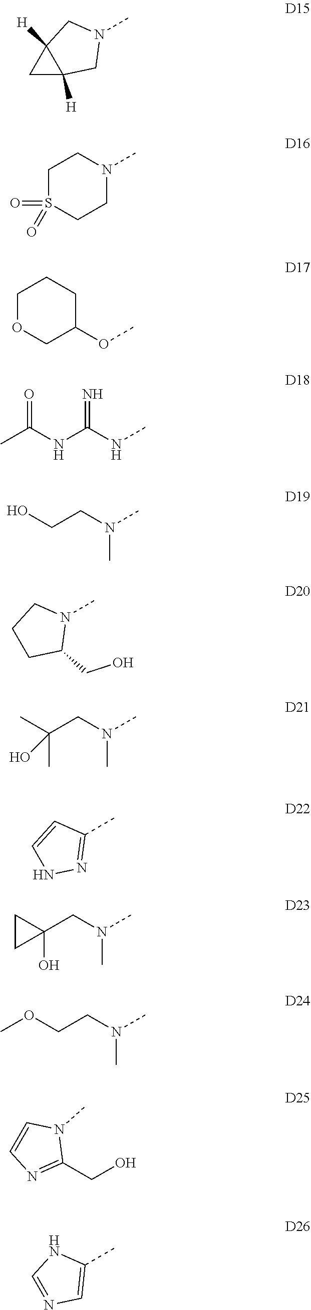

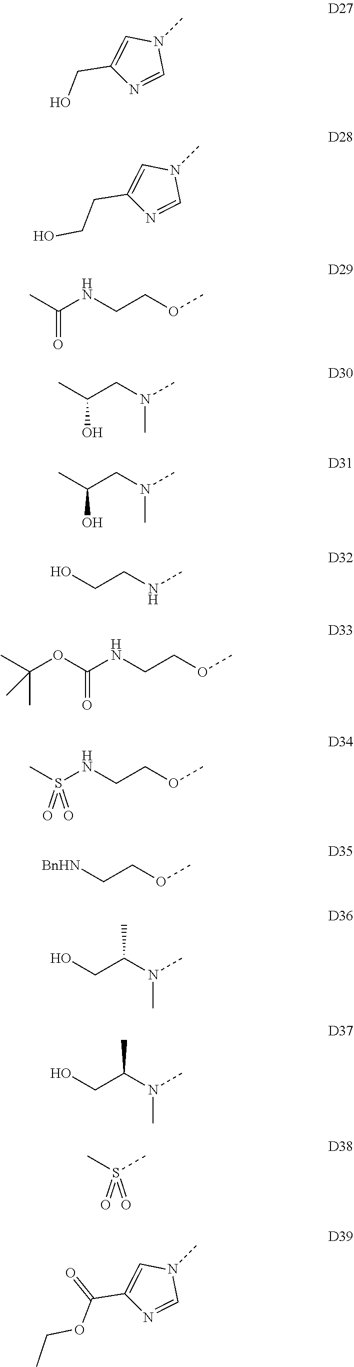

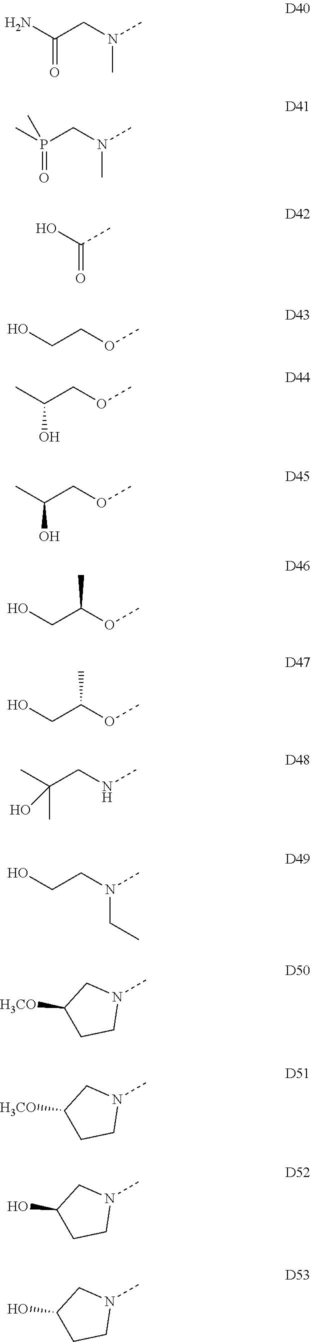

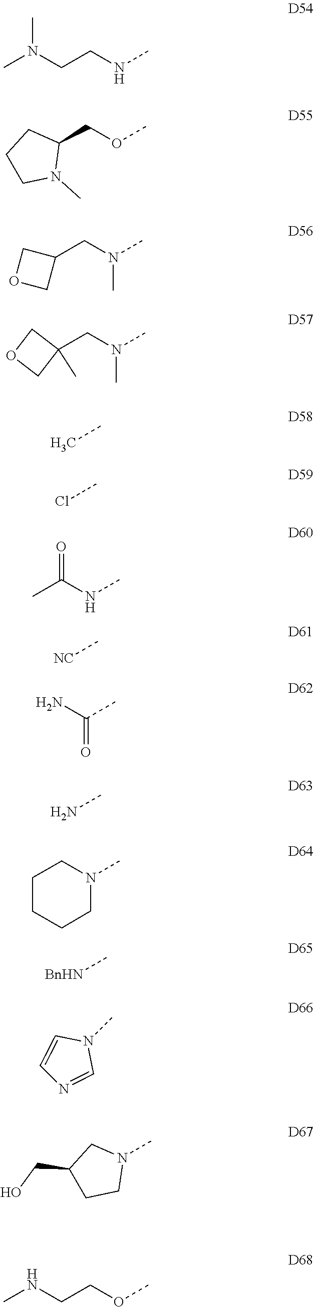

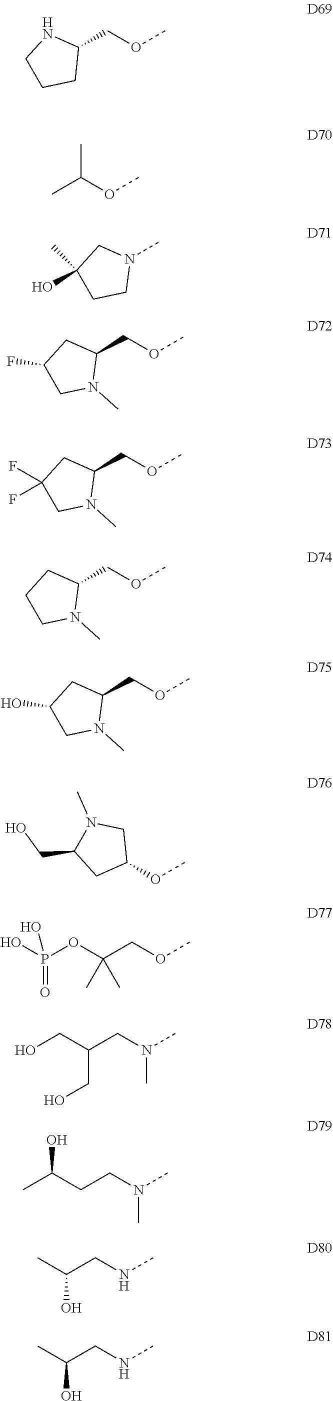

Exemplary compounds as defined herein are also further illustrated by Compounds 1 to 240 as defined in Table 1 hereinbelow, or an isomer or a tautomer thereof, or a pharmaceutically acceptable salt thereof.

TABLE 1
Exemplary compounds with group definitions

| Compound | R¹ | R² | R³ | R⁴ |
|---|---|---|---|---|
| 1 | A1 | B1 | C1 | D1 |
| 2 | A1 | B1 | C1 | D2 |
| 3 | A1 | B1 | C1 | D3 |
| 4 | A1 | B1 | C1 | D4 |
| 5 | A1 | B1 | C1 | D5 |
| 6 | A1 | B1 | C1 | D6 |
| 7 | A1 | B1 | C1 | D7 |
| 8 | A1 | B1 | C1 | D8 |
| 9 | A1 | B1 | C1 | D9 |
| 10 | A1 | B1 | C1 | D10 |
| 11 | A1 | B1 | C1 | D11 |
| 12 | A1 | B1 | C1 | D12 |
| 13 | A1 | B1 | C1 | D13 |
| 14 | A1 | B1 | C1 | D14 |
| 15 | A1 | B1 | C1 | D15 |
| 16 | A1 | B1 | C1 | D16 |
| 17 | A1 | B1 | C1 | D17 |
| 18 | A1 | B1 | C1 | D18 |
| 19 | A1 | B1 | C1 | D19 |
| 20 | A1 | B1 | C1 | D20 |
| 21 | A1 | B1 | C2 | D3 |
| 22 | A1 | B1 | C2 | D4 |
| 23 | A1 | B1 | C2 | D6 |
| 24 | A1 | B1 | C2 | D7 |
| 25 | A1 | B1 | C2 | D8 |
| 26 | A1 | B1 | C2 | D9 |
| 27 | A1 | B1 | C2 | D10 |
| 28 | A1 | B1 | C2 | D11 |
| 29 | A1 | B1 | C2 | D13 |
| 30 | A1 | B1 | C2 | D14 |
| 31 | A1 | B1 | C2 | D15 |
| 32 | A1 | B1 | C2 | D16 |
| 33 | A1 | B1 | C2 | D17 |
| 34 | A1 | B1 | C2 | D18 |
| 35 | A1 | B1 | C2 | D20 |
| 36 | A1 | B2 | C1 | D20 |
| 37 | A1 | B3 | C1 | D20 |
| 38 | A1 | B1 | C2 | D21 |
| 39 | A1 | B1 | C2 | D22 |
| 40 | A1 | B1 | C1 | D21 |
| 41 | A1 | B1 | C1 | D23 |
| 42 | A1 | B1 | C1 | D24 |
| 43 | A1 | B1 | C1 | D25 |
| 44 | A1 | B1 | C1 | D22 |
| 45 | A1 | B1 | C1 | D26 |
| 46 | A1 | B1 | C1 | D27 |
| 47 | A1 | B1 | C1 | D28 |
| 48 | A1 | B4 | C1 | D19 |
| 49 | A1 | B5 | C1 | D19 |
| 50 | A1 | B6 | C1 | D19 |
| 51 | A1 | B7 | C1 | D19 |
| 52 | A1 | B8 | C1 | D19 |
| 53 | A1 | B1 | C2 | D29 |
| 54 | A1 | B1 | C1 | D30 |
| 55 | A1 | B1 | C1 | D31 |
| 56 | A1 | B1 | C1 | D32 |
| 57 | A1 | B9 | C1 | D19 |
| 58 | A1 | B10 | C1 | D19 |
| 59 | A1 | B11 | C1 | D19 |
| 60 | A1 | B1 | C1 | D33 |
| 61 | A1 | B1 | C1 | D34 |
| 62 | A1 | B1 | C2 | D35 |
| 63 | A1 | B12 | C1 | D19 |
| 64 | A1 | B13 | C1 | D19 |
| 65 | A1 | B14 | C1 | D19 |
| 66 | A1 | B1 | C1 | D36 |
| 67 | A1 | B1 | C1 | D37 |
| 68 | A1 | B1 | C1 | D38 |
| 69 | A1 | B1 | C1 | D39 |
| 70 | A1 | B1 | C1 | D40 |
| 71 | A1 | B1 | C1 | D41 |
| 72 | A1 | B1 | C3 | D19 |
| 73 | A1 | B1 | C4 | D19 |
| 74 | A1 | B1 | C5 | D19 |
| 75 | A1 | B1 | C6 | D19 |
| 76 | A1 | B1 | C7 | D19 |
| 77 | A1 | B1 | C1 | D42 |
| 78 | A1 | B1 | C1 | D43 |
| 79 | A1 | B1 | C1 | D44 |
| 80 | A1 | B1 | C1 | D45 |
| 81 | A1 | B1 | C1 | D46 |
| 82 | A1 | B1 | C1 | D47 |
| 83 | A1 | B1 | C1 | D80 |
| 84 | A1 | B1 | C1 | D81 |
| 85 | A1 | B1 | C1 | D48 |
| 86 | A1 | B1 | C8 | D9 |
| 87 | A1 | B1 | C9 | D9 |
| 88 | A1 | B1 | C8 | D47 |
| 89 | A1 | B1 | C10 | D1 |
| 90 | A1 | B1 | C1 | D49 |
| 91 | A1 | B1 | C1 | D50 |
| 92 | A1 | B1 | C1 | D51 |
| 93 | A1 | B1 | C1 | D52 |
| 94 | A1 | B1 | C1 | D53 |
| 95 | A2 | B1 | C1 | D9 |
| 96 | A1 | B1 | C67 | D9 |
| 97 | A1 | B1 | C11 | D9 |
| 98 | A1 | B1 | C12 | D9 |
| 99 | A1 | B1 | C13 | D9 |
| 100 | A1 | B1 | C14 | D9 |
| 101 | A1 | B1 | C8 | D45 |
| 102 | A1 | B1 | C1 | D54 |
| 103 | A1 | B15 | C1 | D9 |
| 104 | A1 | B16 | C1 | D9 |
| 105 | A1 | B17 | C1 | D9 |
| 106 | A1 | B2 | C1 | D9 |
| 107 | A1 | B18 | C1 | D9 |
| 108 | A1 | B19 | C1 | D9 |
| 109 | A1 | B14 | C1 | D9 |
| 110 | A1 | B14 | C1 | D21 |
| 111 | A1 | B14 | C1 | D48 |
| 112 | A1 | B14 | C5 | D9 |
| 113 | A1 | B14 | C6 | D9 |
| 114 | A1 | B14 | C7 | D9 |
| 115 | A1 | B20 | C1 | D19 |
| 116 | A1 | B21 | C1 | D9 |
| 117 | A1 | B1 | C1 | D55 |
| 118 | A1 | B14 | C60 | D9 |
| 119 | A1 | B1 | C15 | D9 |
| 120 | A1 | B14 | C16 | D9 |
| 121 | A1 | B22 | C1 | D9 |
| 122 | A1 | B23 | C1 | D9 |
| 123 | A1 | B1 | C17 | D9 |
| 124 | A1 | B1 | C17 | D21 |
| 125 | A1 | B1 | C17 | D56 |
| 126 | A1 | B1 | C17 | D57 |
| 127 | A1 | B1 | C1 | D58 |
| 128 | A1 | B1 | C3 | D21 |
| 129 | A1 | B14 | C3 | D21 |
| 130 | A1 | B14 | C1 | D20 |
| 131 | A1 | B14 | C1 | D52 |
| 132 | A1 | B14 | C16 | D48 |
| 133 | A1 | B14 | C16 | D20 |
| 134 | A1 | B14 | C16 | D58 |
| 135 | A1 | B14 | C18 | D9 |
| 136 | A1 | B14 | C18 | D20 |
| 137 | A1 | B14 | C3 | D9 |
| 138 | A1 | B14 | C4 | D9 |
| 139 | A1 | B14 | C19 | D9 |
| 140 | A1 | B14 | C3 | D20 |
| 141 | A1 | B1 | C20 | D32 |
| 142 | A1 | B1 | C20 | D43 |
| 143 | A1 | B1 | C20 | D20 |
| 144 | A1 | B1 | C20 | D59 |
| 145 | A1 | B1 | C20 | D60 |
| 146 | A1 | B1 | C20 | D61 |
| 147 | A1 | B1 | C20 | D62 |
| 148 | A1 | B1 | C20 | D19 |
| 149 | A1 | B1 | C20 | D63 |
| 150 | A1 | B1 | C20 | D64 |
| 151 | A1 | B1 | C20 | D65 |
| 152 | A1 | B1 | C20 | D66 |
| 153 | A1 | B1 | C20 | D67 |
| 154 | A1 | B1 | C21 | D20 |
| 155 | A1 | B1 | C21 | D19 |
| 156 | A1 | B1 | C21 | D68 |
| 157 | A1 | B1 | C22 | D19 |
| 158 | A1 | B1 | C1 | D19 |
| 160 | A1 | B1 | C1 | D69 |
| 161 | A1 | B1 | C2 | D19 |
| 162 | A1 | B1 | C2 | D20 |
| 163 | A1 | B1 | C17 | D20 |
| 164 | A1 | B1 | C23 | D66 |
| 165 | A1 | B1 | C17 | D19 |
| 166 | A1 | B1 | C23 | D20 |
| 167 | A1 | B1 | C23 | D19 |
| 168 | A1 | B1 | C24 | D19 |
| 169 | A1 | B1 | C25 | D20 |
| 170 | A1 | B1 | C25 | D19 |
| 171 | A1 | B1 | C26 | D20 |
| 172 | A1 | B1 | C26 | D19 |
| 173 | A1 | B1 | C27 | D19 |
| 174 | A1 | B1 | C28 | D19 |
| 175 | A1 | B1 | C29 | D20 |
| 176 | A1 | B1 | C29 | D19 |
| 177 | A1 | B1 | C16 | D20 |
| 178 | A1 | B1 | C30 | D19 |
| 179 | A1 | B1 | C31 | D19 |
| 180 | A1 | B1 | C32 | D19 |
| 181 | A1 | B1 | C33 | D19 |
| 182 | A1 | B1 | C16 | D19 |
| 183 | A1 | B1 | C34 | D19 |
| 184 | A1 | B1 | C35 | D19 |
| 185 | A1 | B1 | C32 | D19 |
| 186 | A1 | B1 | C36 | D19 |
| 187 | A1 | B1 | C37 | D19 |
| 188 | A1 | B1 | C38 | D19 |
| 189 | A1 | B1 | C8 | D19 |
| 190 | A1 | B1 | C39 | D19 |
| 191 | A1 | B1 | C40 | D19 |
| 192 | A1 | B1 | C41 | D19 |
| 193 | A1 | B1 | C42 | D19 |
| 194 | A1 | B1 | C43 | D19 |
| 195 | A1 | B1 | C44 | D19 |
| 196 | A1 | B1 | C45 | D19 |
| 197 | A1 | B1 | C46 | D19 |
| 198 | A1 | B1 | C21 | D9 |
| 199 | A1 | B1 | C33 | D9 |
| 200 | A1 | B1 | C11 | D19 |
| 201 | A1 | B1 | C47 | D9 |
| 202 | A1 | B1 | C48 | D9 |
| 203 | A1 | B1 | C49 | D9 |
| 204 | A1 | B1 | C50 | D9 |
| 205 | A1 | B1 | C8 | D70 |
| 206 | A1 | B1 | C51 | D9 |
| 207 | A1 | B1 | C16 | D9 |
| 208 | A1 | B1 | C20 | D9 |
| 209 | A1 | B1 | C52 | D9 |
| 210 | A1 | B1 | C53 | D9 |
| 211 | A1 | B1 | C54 | D9 |
| 212 | A1 | B1 | C55 | D9 |
| 213 | A1 | B1 | C69 | D9 |
| 214 | A1 | B1 | C56 | D9 |
| 215 | A1 | B1 | C18 | D9 |
| 216 | A1 | B1 | C57 | D9 |
| 217 | A1 | B1 | C57 | D9 |
| 218 | A1 | B1 | C58 | D9 |
| 219 | A1 | B1 | C59 | D9 |
| 220 | A1 | B1 | C60 | D9 |
| 221 | A1 | B1 | C61 | D9 |
| 222 | A1 | B1 | C62 | D9 |
| 223 | A1 | B1 | C1 | D71 |
| 224 | A1 | B1 | C27 | D9 |
| 225 | A1 | B1 | C16 | D71 |
| 226 | A1 | B1 | C63 | D9 |
| 227 | A1 | B1 | C24 | D9 |
| 228 | A1 | B1 | C1 | D72 |
| 229 | A1 | B1 | C1 | D73 |
| 230 | A1 | B1 | C1 | D74 |
| 231 | A1 | B1 | C18 | D20 |
| 232 | A1 | B1 | C1 | D72 |
| 233 | A1 | B1 | C64 | D9 |
| 234 | A1 | B1 | C64 | D20 |
| 235 | A1 | B1 | C1 | D75 |
| 236 | A1 | B1 | C1 | D76 |
| 237 | A1 | B1 | C3 | D20 |
| 238 | A1 | B1 | C3 | D9 |
| 239 | A1 | B1 | C65 | D77 |
| 240 | A1 | B1 | C66 | D9 |

In a preferred embodiment, group R⁴ comprises a hydroxyl-substituted functional group and the present technology relates to compounds of Formula I:

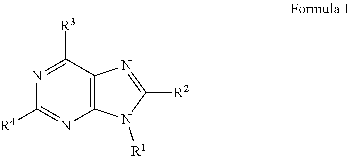

wherein,
R¹ is an optionally substituted C₆aryl group;
R² is an optionally substituted C₆aryl or C₅-C₆heteroaryl group;
R³ is an optionally substituted group selected from R⁷O—, N(R³)₂—, C₄₋₁₀heterocycloalkyl, and C₅₋₆heteroaryl;
R⁴ is an optionally substituted group selected from R⁵O— and N(R⁶)₂—;
R⁵ is a group selected from C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl;
R⁶ is H or a group selected from C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl, wherein at least one R⁶ is other than H, or two R⁶ groups are taken together with their adjacent nitrogen atom to form a C₄-C₁₀heterocycloalkyl or C₅-C₆heteroaryl group;
wherein at least one of said alkyl, cycloalkyl, heterocycloalkyl, or heteroaryl in R⁵ or R⁶ is substituted with at least one hydroxyl or hydroxy-substituted C₁-C₄alkyl group and is optionally further substituted with other substituents;
R⁷ is an optionally substituted C₁-C₆alkyl; and
R³ is independently in each occurrence selected from hydrogen, C₁-C₆alkyl, C₃-C₇cycloalkyl, and C₄-C₁₀heterocycloalkyl;
wherein each of the alkyl, cycloalkyl, heterocycloalkyl, aryl, and heteroaryl groups is optionally substituted;
or an isomer or a tautomer thereof, or a pharmaceutically acceptable salt thereof.

In another embodiment, the compounds are of Formula II:

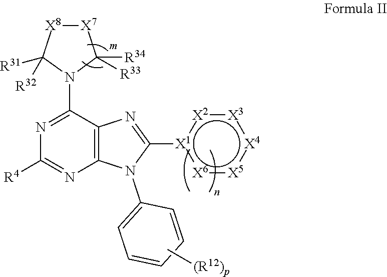

wherein,
R⁴ is as defined in Formula I;
X¹ is C and X² to X⁶ are each independently selected from N and CR²¹ and n is 1, wherein at most three of X² to X⁶ are N; or
X¹ is C or N and X² to X⁵ are each independently selected from CR²¹, O, S, N, or NR¹¹, n is zero and X⁶ is absent and forms a bond between X¹ and X⁵, wherein at most three of X¹ to X⁵ are other than C or CR²¹;
X⁷ and X⁸ are each independently selected from O, S, SO₂, NR³⁶, or C(R³⁵)₂, wherein when one of X⁷ and X⁸ is O, S, or NR³⁶, then the other is C(R³⁵)₂;
R⁹ is independently in each occurrence selected from optionally substituted C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, and C₅-C₆heteroarylC₁-C₃alkyl;
R¹⁰ is independently in each occurrence selected from H or a group selected from optionally substituted C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, and C₅-C₆heteroarylC₁-C₃alkyl, or two R¹⁰ groups are taken together with their adjacent nitrogen atom to form an optionally substituted C₄-C₁₀heterocycloalkyl or C₅-C₆heteroaryl;
R¹¹ is independently in each occurrence selected from H or a group selected from optionally substituted C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl;
R¹² is independently in each occurrence selected from F, Cl, CN, NH₂, N(H)C₁-C₃alkyl, N(C₁-C₃alkyl)₂, and C₁-C₃alkyl, and p is 0, 1, 2, or 3, preferably 0 or 1;
R²¹ is independently in each occurrence selected from hydrogen, halogen, OH, OR⁹, CN, NO₂, C(O)R⁹, C(O)N(R¹⁰)₂, C(R¹¹)═NR¹¹, SO₂R⁹, SO₂N(R¹⁰)₂, N(R¹¹)C(O)R⁹, N(R¹¹)SO₂R⁹, N(R¹¹)C(O)N(R¹⁰)₂, N(R¹¹)SO₂N(R¹⁰)₂, N(R¹⁰)₂, P(O)(R¹⁰)₂, P(O)(OR¹⁰)₂, B(OR¹⁰)₂, and optionally substituted C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl groups;
R³¹ to R³⁵ are independently in each occurrence selected from hydrogen, halogen, OH, OR⁹, CN, NO₂, C(O)OH, C(O)OR⁹, C(O)R⁹, C(O)N(R¹⁰)₂, C(R¹¹)═NR¹¹, SO₂R⁹, SO₂N(R¹⁰)₂, N(R¹¹)C(O)R⁹, N(R¹¹)SO₂R⁹, N(R¹¹)C(O)N(R¹⁰)₂, N(R¹¹)SO₂N(R¹⁰)₂, N(R¹⁰)₂, P(O)(R¹⁰)₂, P(O)(OR¹⁰)₂, B(OR¹⁰)₂, and optionally substituted C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl groups;
R³⁶ is selected from hydrogen, C(O)R⁹, C(O)OR⁹, C(O)N(R¹⁰)₂, C(R¹¹)═NR¹¹, SO₂R⁹, SO₂N(R¹⁰)₂, P(O)(R¹⁰)₂, P(O)(OR¹⁰)₂, B(OR¹⁰)₂, and optionally substituted C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl groups;
or two of R³¹ to R³⁶ are taken together with their adjacent atoms to form a cycle, preferably a bridged or spiro heterocycle; and
m is 0, 1, 2, or 3;
wherein each of the alkyl, cycloalkyl, heterocycloalkyl, aryl, and heteroaryl groups is optionally substituted;
or an isomer or a tautomer thereof, or a pharmaceutically acceptable salt thereof.

According to some examples, m is zero and X⁷ and X⁸ are each independently C(R³⁵)₂, preferably X⁷ is CH₂. For instance, X⁸ is C(R³⁵)₂, wherein one of the two R³⁵ is fluorine, C(O)R⁹, C(O)OR⁹, C(O)N(R¹⁰)₂, N(R¹¹)C(O)R⁹, or optionally substituted C₁₋₆alkyl, and the other of the two R³⁵ is hydrogen, fluorine, CN, OH, OR⁹, N(R¹⁰)₂, or optionally substituted C₁₋₆alkyl. Alternatively, X⁸ is C(R³⁵)₂ and the two R³⁵ are taken together with their adjacent carbon atom to form a spiro C₄₋₅heterocycle.

In other examples, m is 1 and X⁷ and X³ are each independently C(R³⁵)₂, preferably X⁷ is CH₂. For instance, X⁸ is C(R³⁵)₂, wherein one of the two R³⁵ is fluorine, C(O)R⁹, C(O)OR⁹, C(O)N(R¹⁰)₂, N(R¹¹)C(O)R⁹, or optionally substituted C₁₋₆alkyl, and the other of the two R³⁵ is hydrogen, fluorine, CN, OH, OR⁹, N(R¹⁰)₂, or optionally substituted C₁₋₆alkyl. Alternatively, X⁸ is C(R³⁵)₂ and the two R³⁵ are taken together with their adjacent carbon atom to form a spiro C₄₋₅heterocycle.

According to further examples, m is 2 and X⁷ and X³ are each independently C(R³⁵)₂. For instance, X⁸ is CH₂ and X⁷ is C(R³⁵)₂, wherein one of the two R³⁵ is fluorine, C(O)R⁹, C(O)OR⁹, C(O)N(R¹⁰)₂, N(R¹¹)C(O)R⁹, or optionally substituted C₁₋₆alkyl, and the other of the two R³⁵ is hydrogen, fluorine, CN, OH, OR⁹, N(R¹⁰)₂, or optionally substituted C₁₋₆alkyl. In some preferred examples, R³⁵ is a C(O)N(R¹⁰)₂, or N(R¹¹)C(O)R⁹, and the other of the two R³⁵ is OR⁹ or an optionally substituted C₁₋₄alkyl, for instance, R³⁵ is OR⁹ and R⁹ is a C₁₋₆alkyl, preferably C₂₋₄alkyl, e.g. methyl, ethyl, n-propyl, isopropyl, i-butyl, or sec-butyl, preferably ethyl or isopropyl, most preferably isopropyl.

In other examples, m is 2 and X⁷ and X⁸ are each independently C(R³⁵)₂, for instance, X³ is CH₂ and X⁷ is C(R³⁵)₂ and the two R³⁵ are taken together with their adjacent carbon atom to form a spiro C₄₋₅heterocycle. Alternatively, X⁷ is CH₂ and X³ is C(R³⁵)₂, wherein one of the two R³⁵ is fluorine, C(O)R⁹, C(O)OR⁹, C(O)N(R¹⁰)₂, N(R¹¹)C(O)R⁹, or optionally substituted C₁₋₆alkyl, and the other of the two R³⁵ is hydrogen, fluorine, CN, OH, OR⁹, N(R¹⁰)₂, or optionally substituted C₁₋₆alkyl. For instance, one of the two R³⁵ is a C(O)N(R¹⁰)₂, or N(R¹¹)C(O)R⁹, and the other of the two R³⁵ is OR⁹ or an optionally substituted C₁₋₄alkyl, preferably R³⁵ is OR⁹ and R⁹ is a C₁₋₆alkyl, preferably C₂₋₄alkyl, for instance, methyl, ethyl, n-propyl, isopropyl, i-butyl, or sec-butyl, preferably ethyl or isopropyl, most preferably isopropyl.

In other examples, m is 2 and X⁷ and X⁸ are each independently C(R³⁵)₂, for instance, X⁷ is CH₂ and X³ is C(R³⁵)₂ and the two R³⁵ are taken together with their adjacent carbon atom to form a spiro C₄₋₅heterocycle.

In yet other examples of the compounds of Formula II, m is 2 or 3, X⁷ is O, S, SO₂, or NR³⁶ preferably O, SO₂, or NR³⁶, and X³ is C(R³⁵)₂. For instance, X⁷ is NR³⁶ and R³⁶ is selected from hydrogen, C(O)R⁹, C(O)OR⁹, C(O)N(R¹⁰)₂, and optionally substituted C₁₋₆alkyl. In one embodiment, one of the two R³⁵ in X⁸ is hydrogen, fluorine, C(O)R⁹, C(O)OR⁹, C(O)N(R¹⁰)₂, N(R¹¹)C(O)R⁹, or optionally substituted C₁₋₆alkyl, and the other of the two R³⁵ is hydrogen, fluorine, OH, OR⁹, N(R¹⁰)₂, or optionally substituted C₁₋₆alkyl, or one of the R³⁵ is taken together with R³³ and their adjacent atoms to form a bridged cycle. For instance, both R³⁵ in X⁸ are hydrogen.

Alternatively, one of the two R³⁵ is fluorine, C(O)R⁹, C(O)OR⁹, C(O)N(R¹⁰)₂, N(R¹¹)C(O)R⁹, or optionally substituted C₁₋₆alkyl, and the other of the two R³⁵ is hydrogen. In another alternative, R³⁵ together with R³³ form a C₁₋₃alkylene group.

In some examples of the compounds of Formula II, R³³ is independently in each occurrence selected from hydrogen, fluorine and optionally substituted C₁₋₆alkyl, and/or R³⁴ is independently in each occurrence selected from hydrogen, fluorine and optionally substituted C₁₋₆alkyl, and/or R³¹ and R³² are each hydrogen atoms.

Within the context of Formula II, examples of the R³ group present in Formula I include C1-C7, C10, C15, C16, C18-C22, C24-C28, C32-C40, and C47-C69 groups as defined above, preferably C1, C16 or C18, more preferably C18.

In a further embodiment, the compounds are of Formula III:

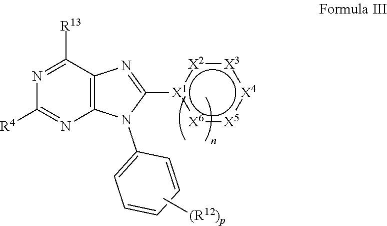

wherein,
R¹³ is an optionally substituted group selected from R⁷O—, N(R³)₂—, and C₅₋₆heteroaryl;
R⁴, R⁷ and R³ are as defined with respect to Formula I; and
R¹², X¹ to X⁶, n and p are as defined with respect to Formula II;
or an isomer or a tautomer thereof, or a pharmaceutically acceptable salt thereof.

According to some examples, R¹³ is R⁷O—. For instance, R⁷ may be an optionally substituted C₁-C₄alkyl.

In other examples, R¹³ is N(R⁸)₂—, preferably wherein one R³ is an optionally substituted C₄-C₁₀heterocycloalkyl and the other R³ is hydrogen or an optionally substituted C₁-C₆alkyl, or wherein one R³ is an optionally substituted C₄-C₇heterocycloalkyl and the other R³ is hydrogen or an optionally substituted C₁-C₄alkyl, or R³ is an optionally substituted C₁-C₆alkyl, and the other R³ is hydrogen or an optionally substituted C₁-C₆alkyl.

Within Formula I or III, examples of the R³ or R¹³ group include C8, C9, C11-C14, C17, C23, C29-C31, and C41-C46 groups as defined above.

In some compounds of Formulae II and III, R¹² is Cl and p is 1, preferably forming a 4-chlorophenyl group. In other compounds, p is zero and R¹² is absent, thereby forming an unsubstituted phenyl group.

In some examples of compounds of Formulae II and III, n is 1, X¹ is C and X² to X⁶ are each independently CR²¹, or n is 1, X¹ is C, one of X² to X⁶ is N and the others are CR²¹. Alternatively, n is zero, X⁶ is absent, and X¹ is C, preferably one or two of X² to X⁵ is N or NR¹¹ and the others are CR²¹, preferably R¹¹ is a C₁-C₆alkyl, or one of X² to X⁵ is S and the others are CR²¹. In these examples, all CR²¹ groups may be CH. Alternatively, one CR²¹ is other than CH, preferably R²¹ being selected from halogen, CN, N(C₁₋₆alkyl)₂, and C₁₋₆alkyl, more preferably halogen or CN, most preferably halogen (e.g. Cl), or one CR²¹ is other than CH, preferably R²¹ being selected from halogen, CN, N(C₁₋₆alkyl)₂, and C₁₋₆alkyl, more preferably halogen, CN, and C₁₋₆alkyl, most preferably CN, or one CR²¹ is other than CH, preferably R²¹ being selected from halogen, CN, N(C₁₋₆alkyl)₂, and C₁₋₆alkyl, more preferably CN and C₁₋₆alkyl, most preferably C₁₋₆alkyl.

Examples of R² in Formulae I, II or III include groups 1 to B23 as defined herein. For instance, R² may be selected from groups B1 to B4, for example R² is 1. Alternatively, R² is selected from groups B5 to B8, B12 to B19, and B21 to B23, for example R² is B14. In yet other examples, R² may be selected from groups B9 to B11 and B20.

In some examples, the compounds are of Formula I, II or III, wherein R⁴ is an R⁵O— group, wherein R⁵ is as defined above and is substituted with at least one hydroxyl group or hydroxy-substituted C₁-C₄alkyl group. For example, R⁵ may be a C₂-C₆alkyl, C₄-C₁₀heterocycloalkyl, or C₄-C₁₀heterocycloalkylC₁-C₃alkyl group substituted with a hydroxy group and optionally other substituents. In some examples, R⁵ is selected from 2-hydroxyethyl, 3-hydroxy-1-propyl, 2-hydroxy-1-propyl, 1-hydroxy-2-propyl, 2-hydroxy-2-methyl-1-propyl, 3-hydroxy-2-methyl-1-propyl, 2-hydroxy-1-methyl-1-propyl, 3-hydroxy-1-methyl-1-propyl, and 2-hydroxy-1,1-dimethyl-1-ethyl. In other examples, R⁵ is C₄-C₆heterocycloalkyl, or C₄-C₆heterocycloalkylC₁-C₃alkyl group substituted with a hydroxy group and optionally other substituents, preferably the C₄-C₆heterocycloalkyl is selected from pyrrolidinyl, imidazolidinyl, piperidinyl, and piperazinyl groups.

In other examples, the compounds are of Formula I, II or III, and R⁴ is an N(R⁶)₂-group, wherein R⁶ is as defined herein and at least one R⁶ is substituted with at least one hydroxyl group or hydroxy-substituted C₁-C₄alkyl group and optionally substituted with one or more other substituents. In one embodiment of these examples, one R⁶ is a C₂-C₆alkyl, C₃-C₇cycloalkyl, C₄-C₁₀heterocycloalkyl, C₃-C₇cycloalkylC₁-C₃alkyl, or C₄-C₁₀heterocycloalkylC₁-C₃alkyl group substituted with at least one hydroxyl or hydroxy-substituted C₁-C₄alkyl group, and the other R⁶ is a hydrogen or C₁-C₆alkyl and optionally substituted with one or more other substituents, preferably R⁶ is a C₂-C₆alkyl or C₃-C₇cycloalkylC₁-C₃alkyl group substituted with a hydroxyl or hydroxy-substituted C₁-C₄alkyl group, and the other R⁶ is a hydrogen or C₁-C₆alkyl. In another embodiment of these examples, the two R⁶ groups are taken together with their adjacent nitrogen atom to form a C₄-C₁₀heterocycloalkyl or C₅heteroaryl group substituted with at least one hydroxyl or hydroxy-substituted C₁-C₄alkyl group and optionally substituted with one or more other substituents. For instance, the two R⁶ groups are taken together with their adjacent nitrogen atom to form a C₄-C₇heterocycloalkyl substituted with at least one hydroxyl or hydroxy-substituted C₁-C₄alkyl group and optionally substituted with one or more other substituents, preferably wherein the C₄-C₇heterocycloalkyl group is selected from pyrrolidinyl, imidazolidinyl, piperidinyl, and piperazinyl groups substituted with at least one hydroxyl or hydroxy-substituted C₁-C₄alkyl group and optionally substituted with one or more other substituents; or the two R⁶ groups are taken together with their adjacent nitrogen atom to form a C₅heteroaryl group substituted with at least one hydroxyl or hydroxy-substituted C₁-C₄alkyl group and optionally substituted with one or more other substituents, preferably the C₅heteroaryl group being selected from an imidazole and a pyrrole group substituted with at least one hydroxyl or hydroxy-substituted C₁-C₄alkyl group and optionally substituted with one or more other substituents.

Examples of R⁴ in Formulae I, II or III include groups D5-D9, D12, D13, D19-D21, D23, D25, D27, D28, D30-D32, D36, D37, D43-D49, D52, D53, D67, D71, D75, D76, and D78-D81 as defined herein. For instance, R⁴ may be selected from groups D6-D9, D43-D47, D75 and D76, preferably D6-D9 and D43-D47, preferably D9, or from groups D12, D19, D21, D23, D30-D32, D36, D37, D48, D49, and D78-D81, preferably D19 or D21, preferably D5, D13, D20, D52, D53, D67, and D71, preferably D20.

Examples of compounds of Formula I, II or III include, but are not limited to, Compounds 5-9, 12, 13, 19, 20, 23-26, 29, 35-38, 40, 41, 43, 46-52, 54-59, 63-67, 72-76, 78-88, 90, 93-101, 103-116, 118-124, 128-133, 135-143, 148, 153-155, 157, 158, 161-163, 165-204, 206-227, 231, 233-238, and 240, or an isomer or a tautomer thereof, or a pharmaceutically acceptable salt thereof. Preferably, the compound is selected from Compounds 5-9, 12, 19, 20, 23-26, 29, 35-38, 40, 41, 43, 46-52, 54-59, 63-67, 72-76, 78-88, 90, 93-97, 99-101, 104-115, 118-124, 128-131, 133, 135-137, 139-143, 148, 153-155, 157, 158, 161-163, 165-175, 177-204, 206-227, 231, 233-238, and 240, or an isomer or a tautomer thereof, or a pharmaceutically acceptable salt thereof. More preferably, the compound is selected from Compounds 5-9, 12, 19, 20, 23-26, 35, 36, 38, 40, 41, 43, 46-50, 52, 54-56, 64-67, 72-76, 78-88, 90, 93, 94, 96, 99, 101, 108, 110, 113, 114, 119-121, 123, 124, 128, 130, 131, 133, 135-137,140-143,148,153-155,157,158, 161-163, 165-174, 177-204, 206, 207, 209-216, 220-227, 231, 233-237, and 240, or an isomer or a tautomer thereof, or a pharmaceutically acceptable salt thereof. Most preferably, the compound is selected from Compounds 6-9, 12, 19, 20, 23-26, 35, 36, 38, 41, 43, 46-48, 54-56, 65-67, 72-76, 78-83, 85, 86, 88, 90, 93, 94, 96, 123, 130, 131, 133, 136, 140-143, 148, 153, 154, 157, 158, 161-163, 165-167, 169-171, 173, 174, 177-186, 188, 189, 191, 192, 194-197, 199, 203, 206, 207, 211, 213-216, 222-226, 231, 234, and 237, or an isomer or a tautomer thereof, or a pharmaceutically acceptable salt thereof.

In yet another embodiment, the compounds are of Formula IV:

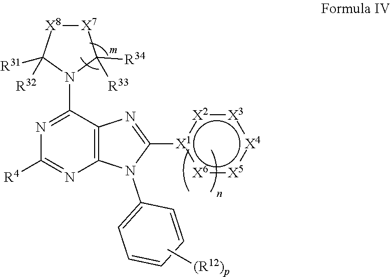

wherein X¹ to X⁸, R¹², R³¹ to R³⁴, m, n and p are as defined herein; and
R¹⁴ is N(R¹⁶)₂— or optionally substituted C₅-C₆heteroaryl; and
R¹⁶ is selected from H or a group selected from optionally substituted C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl, or two R¹⁶ groups are taken together with their adjacent nitrogen atom to form an optionally substituted C₄-C₁₀heterocycloalkyl or C₅-C₆heteroaryl;
or an isomer or a tautomer thereof, or a pharmaceutically acceptable salt thereof.

In some examples, R¹⁴ is N(R¹⁶)₂—. In one embodiment, one R¹⁶ is an optionally substituted C₁-C₆alkyl, C₃-C₇cycloalkyl, C₄-C₁₀heterocycloalkyl, C₃-C₇cycloalkylC₁-C₃alkyl, or C₄-C₁₀heterocycloalkylC₁-C₃alkyl group, and the other R¹⁶ is a hydrogen or C₁-C₆alkyl. For example, one R¹⁶ is an C₂-C₆alkyl group substituted with one or more substituents, and the other R¹⁶ is a hydrogen or C₁-C₆alkyl, the substituent preferably being selected from F, OH, CN, alkoxy, alkylcarbonylamino, akoxycarbonylamino, alkylsulfonamido, benzyl amino, aminocarbonyl, dialkylphosphino, phosphonato, dialkylamino, cycloalkyl, heterocycloalkyl, aryl, and heteroaryl. In another embodiment, two R¹⁶ groups are taken together with their adjacent nitrogen atom to form an optionally substituted C₄-C₁₀heterocycloalkyl or C₅heteroaryl group connected through the nitrogen. For example, two R¹⁶ groups are taken together with their adjacent nitrogen atom to form an optionally substituted C₄-C₇heterocycloalkyl connected through the nitrogen, preferably the C₄-C₇heterocycloalkyl group is selected from optionally substituted pyrrolidinyl, imidazolidinyl, piperidinyl, and piperazinyl groups. Alternatively, the two R¹⁶ groups are taken together with their adjacent nitrogen atom to form an optionally substituted C₅heteroaryl group connected through the nitrogen atom, preferably the C₅heteroaryl group is selected from an optionally substituted imidazole and pyrrole groups.

In other examples, R¹⁴ is an optionally substituted C₅-C₆heteroaryl.

Examples of R¹⁴ groups include D3-D5, D11-D16, D19-D28, D30-D32, D36, D37, D39-D41, D48-D54, D56, D57, D63-D67, D71, and D78-D81 as defined herein.

In yet another embodiment, the present compounds are of Formula V:

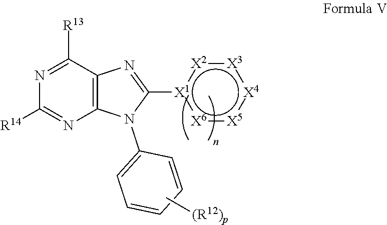

wherein X¹ to X⁶, R¹², R¹³, R¹⁴, n and p are as previously defined;
or an isomer or a tautomer thereof, or a pharmaceutically acceptable salt thereof.

In a further embodiment, the present compounds are of Formula VI:

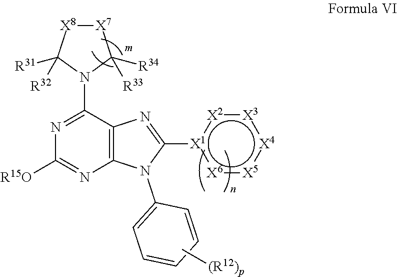

wherein X¹ to X⁸, R¹², R³¹ to R³⁴, m, n and p are as previously defined; and
R¹⁵ is a group selected from C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl;
or an isomer or a tautomer thereof, or a pharmaceutically acceptable salt thereof.

According to some examples, R¹⁵ is an optionally substituted C₁-C₆alkyl, C₄-C₁₀heterocycloalkyl, or C₄-C₁₀heterocycloalkylC₁-C₃alkyl group. For example, R¹⁵ is a C₁-C₆alkyl group substituted with one or more substituents, for instance selected from F, OH, CN, alkoxy, alkylcarbonylamino, akoxycarbonylamino, alkylsulfonamido, benzyl amino, aminocarbonyl, dialkylphosphino, phosphonato, dialkylamino, cycloalkyl, heterocycloalkyl, aryl, and heteroaryl; or R¹⁵ is an optionally substituted C₄-C₆heterocycloalkyl or C₄-C₆heterocycloalkylC₁-C₃alkyl group, preferably the C₄-C₆heterocycloalkyl is selected from pyrrolidinyl, imidazolidinyl, morpholinyl, piperidinyl, and piperazinyl groups.

Examples of OR¹⁵ groups include D6-D10, D17, D29, D33-D35, D43-D47, D55, D68-D70, and D72-D77.

In a further embodiment, the present compounds are of Formula VII:

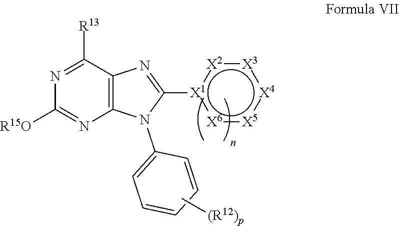

wherein X¹ to X, R¹², R¹³, R¹⁵, n and p are as previously defined;
or an isomer or a tautomer thereof, or a pharmaceutically acceptable salt thereof.

In another embodiment, the compounds are of Formula VIII:

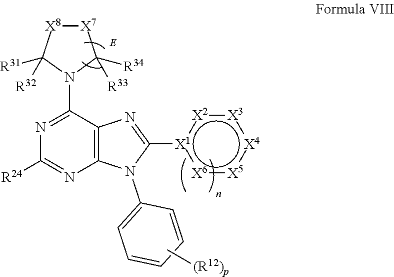

wherein X¹ to X⁸, R¹², R³¹ to R³⁴, m, n and p are as previously defined; and
R²⁴ is selected from Cl, CN, C(O)OH, R⁹C(O)N(R¹¹)—, R⁹C(O)NHC(NH)NH—, R⁹S(O)₂—, N(R¹⁰)₂C(O)—, and an optionally substituted C₁-C₆alkyl group, wherein R⁹ to R¹¹ are as previously defined;
or an isomer or a tautomer thereof, or a pharmaceutically acceptable salt thereof.

In a further embodiment, the compounds are of Formula IX:

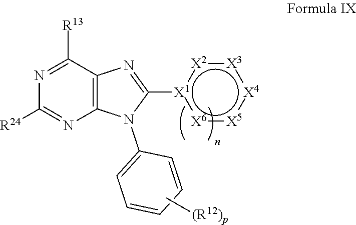

wherein X¹ to X⁶, R¹², R¹³, R²⁴, n and p are as previously defined;
or an isomer or a tautomer thereof, or a pharmaceutically acceptable salt thereof.

In some examples of the compounds of Formula VIII and IX, R²⁴ is selected from groups D1, D2, D18, D38, D42, and D58-D62.

In one embodiment, the present compound is selected from compounds 1 to 240 as defined herein, or an isomer or a tautomer thereof, or a pharmaceutically acceptable salt thereof.

In another embodiment, the compound is selected from Compounds 1-12, 14-28, 30-69, 71-97, 99-102, 104-115, 117-131, 133-137, 139-148, 150-158, 160-175, and 177-240, or an isomer or a tautomer thereof, or a pharmaceutically acceptable salt thereof.

In a further embodiment, the compound is selected from Compounds 1-12, 14-28, 31-36, 38-50, 52-56, 60-62, 64-68, 72-96, 99, 101, 102, 108, 110, 113, 114, 117, 119-121, 123-128, 130, 131, 133, 135-137, 140-146, 148, 150-155, 157, 158, 160-174, 177-204, 206, 207, 209-216, 220-237, and 240, or an isomer or a tautomer thereof, or a pharmaceutically acceptable salt thereof.

In yet another embodiment, the compound is selected from Compounds 6-10, 12, 15-17, 19, 20, 23-26, 32, 33, 35, 36, 38, 41-44, 46-48, 54-56, 62, 65-67, 72-76, 78-83, 85, 86, 88-94, 96, 117, 123, 130, 131, 133, 136, 140-146, 148, 150, 152-154, 157, 158, 161-163, 165-167, 169-171, 173, 174, 177-186, 188, 189, 191, 192, 194-197, 199, 203, 206, 207, 211, 213-216, 222-226, 228, 230-232, 234, and 237, or an isomer or a tautomer thereof, or a pharmaceutically acceptable salt thereof.

The present compounds may be prepared by conventional chemical synthesis such as those described in the Examples section below. As can be appreciated by the skilled artisan, further methods of synthesizing the compounds of the present formulae will be evident to those of ordinary skill in the art. Additionally, the various synthetic steps may be performed in an alternate sequence or order to give the desired compounds.

The compound as defined herein can be formulated in a pharmaceutical composition for administration to a subject, the compound being usually admixed with a at least one pharmaceutically acceptable carrier, diluent, or excipient.

The expression “pharmaceutically acceptable carrier, diluent, or excipient” and equivalent expressions, refer to a non-toxic carrier, diluent, or excipient that does not destroy the pharmacological activity of the compound with which it is formulated.

Compositions described herein may be administered orally, parenterally, by inhalation spray, topically, rectally, nasally, buccally, or via an implanted reservoir. The term “parenteral” as used herein includes subcutaneous, intravenous, intramuscular, intraarticular, intrasynovial, intrasternal, intrathecal, intrahepatic, and intralesional injection or infusion techniques. Other modes of administration also include intradermal or transdermal administration.

For instance, solid dosage forms for oral administration include capsules, tablets, pills, and granules. In a preferred alternative, the composition is a solid dosage form which comprises the compound as described herein and at least one binder as defined in the preceding paragraph, the binder preferably comprising microcrystalline cellulose.

Pharmaceutically acceptable carriers, diluents or excipients that may be used in oral compositions of this disclosure include, but are not limited to, binders, sweeteners, disintegrating agents, diluents, flavorings, coating agents, preservatives, lubricants, and/or polymers. Examples of binders include cellulose-based substances such as microcrystalline cellulose and carboxymethylcellulose, and other binders like gum acacia, gelatin, corn starch, gum tragacanth, sodium alginate, or polyethylene glycol (PEG). Examples of sweeteners include sucrose, lactose, glucose, aspartame or saccharine. Disintegrating agents include corn starch, methylcellulose, polyvinylpyrrolidone, xanthan gum, bentonite, alginic acid or agar. Examples of diluents include lactose, sorbitol, mannitol, dextrose, kaolin, cellulose, calcium carbonate, calcium silicate or dicalcium phosphate. Flavoring agents include peppermint oil, oil of wintergreen, cherry, orange, or raspberry flavoring. Coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Examples of excipients may further include a polymer selected from the group consisting of polyvinylpyrrolidone (PVP), polyvinylpyrrolidone-vinylacetate copolymer (PVP-VA), hydroxypropylmethylcellulose (HPMC), hypromellose-acetate-succinate (HPMCAS), and mixtures thereof.

The present compositions may also be employed as fillers in soft and hard-filled capsules. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that release the active ingredient(s) only, or preferentially, in a certain part of the intestinal tract, optionally, in a delayed manner. Examples of embedding compositions that can be used include polymeric substances and waxes. The composition may also be in micro-encapsulated form with one or more excipients as noted above.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, these oral compositions can also include adjuvants such as wetting agents, emulsifying and suspending agents, surfactants, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a provided compound, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled.

Examples of other biodegradable polymers include poly(orthoesters) and poly(anhydrides). Depot injectable formulations are also prepared by entrapping the compound in liposomes or microemulsions that are compatible with body tissues.

Dosage forms for topical or transdermal administration of a compound of the present description include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of the present description. Additionally, the description contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Pharmaceutically acceptable compositions provided herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promotors to enhance bioavailability, fluorocarbons, and/or other conventional solubilizing or dispersing agents.

The amount of compound that may be combined with carrier materials to produce a composition in a single dosage form will vary depending upon the patient to be treated and the particular mode of administration.

As used herein, the term “effective amount” means that amount of a compound that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in treatment, healing, prevention, or amelioration of a disease, disorder, or symptom thereof, or a decrease in the rate of advancement of a disease or disorder. The term also includes within its scope amounts effective to enhance normal physiological function.

As used herein, the terms “treatment”, “treat”, and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The term “patient” or “subject” as used herein refers to an animal such as a mammal. A subject may therefore refer to, for example, mice, rats, dogs, cats, horses, cows, pigs, guinea pigs, primates including humans and the like. Preferably the subject is a human.

The present compounds are useful for the treatment of diseases and disorders where inhibition of the cannabinoid receptor CB₁ is indicated. Accordingly, here are contemplated a use of the present compounds for the treatment of a disease or disorder as defined herein, a use of the present compounds in the manufacture of a medicament for the treatment of a disease or disorder as defined herein, a compound as herein defined for use in the treatment of the present diseases or disorders, as well as a method for treating a disease or disorder as defined herein comprising the administration of one of the present compounds to a subject in need thereof. Such diseases and disorders may generally be related to diabetes and metabolic disorders (e.g. metabolic syndrome). Preferably, the compound selectively targets the CB₁ receptor in peripheral tissue (e.g. adipose tissue, liver, muscle, lung, kidney, macrophages, pancreatic beta cells and gastrointestinal tract), while not or mainly not interacting with CB₁ receptors in brain tissue, thereby avoiding or reducing CNS-related side effects.

The effect of the present compounds may include reduced food intake, reduced body weight, reversed insulin and leptin resistance, reverse hepatic steatosis (fatty liver) and improved dyslipidemia. Examples of diseases and disorders to be treated include obesity, diabetes (type I or II), non-alcoholic and alcoholic fatty liver disease (a risk factor for insulin resistance), a co-morbidity of obesity, a co-morbidity of diabetes, Prader-Willi Syndrome (PWS), Pro-opiomelanocortin (POMC) deficiency obesity, leptin receptor (LepR) deficiency obesity, POMC heterozygous deficiency obesity, POMC epigenetic disorders, Bardet-Biedl (BB) syndrome, Alström syndrome, dyslipidemia predisposing to arteriosclerotic heart disease, diabetic nephropathy, fibrosis and fibrotic diseases of the skin, liver, lung or kidney such as Idiopathic Pulmonary Fibrosis (IPF), Progressing Fibrosis Interstitial Lung Diseases, Hermansky-Pudlak Syndrome pulmonary fibrosis (HPS-PF), cirrhosis, renal fibrosis, scleroderma, and gout. In addition, disorders of the skin include reducing scar formation (cicatrix, keloid) and alopecia, particularly that associated with male pattern baldness and metabolic syndrome. For instance, the co-morbidity of obesity is selected from metabolic syndrome, dementia, heart disease, hypertension, gallbladder disease, gastrointestinal disorders, menstrual irregularities, degenerative arthritis, venous statis ulcer, pulmonary hypoventilation syndrome, sleep apnea, snoring, asthma, obese asthma, coronary artery disease, arterial sclerotic disease, pseudotumor cerebri, osteoarthritis, high cholesterol, and increased incidence of malignancies of the liver, ovaries, cervix, uterus, breasts, prostate, or gallbladder. In preferred examples, the disease or disorder include diabetes (type I or II), obesity, and non-alcoholic fatty liver disease (e.g. non-alcoholic steatohepatitis). Examples of co-morbidities of diabetes (e.g. type I) include diabetic nephropathy, chronic kidney disease, diabetic retinopathy, and peripheral and autonomic neuropathy.

Diseases, disorders and conditions to be treated, including those above, may be separated into various categories, while some of the conditions may also coexist in one given subject. Examples of categories include disorders related to appetite and their complications, disorders related to glucose regulation and their complications, fibrosis related disorders and their complications, disorders related to metabolism and their complications, disorders related to skin and hair growth and healing, disorders related to the GI tract, and disorders related to obesity and their complications.

Examples of disorders related to appetite and their complications include, without limitation, Prader-Willi Syndrome (PWS), hypothalamic obesity, pro-opiomelanocortin (POMC) deficiency (including POMC obesity, heterozygous POMC deficiency obesity, POMC epigenetic disorders), Leptin receptor (LepR) deficiency, Bardet-Biedl (BB) syndrome, and Alström syndrome.

Examples of disorders related to glucose regulation and their complications include, without limitation, diabetes Type I, diabetes Type II, insulin resistance, pre-diabetes, pancreatic diseases (by β-cell protection and/or increased insulin production), and associated nephropathies, neuropathies and retinopathies.

Examples of fibrosis related disorders and their complications include, without limitation, progressive fibrosis associated with interstitial lung disease, idiopathic pulmonary fibrosis (IPF), Hermansky-Pudlak syndrome pulmonary fibrosis (HPS-PF), cirrhosis and other liver fibrosis disorders (such as nonalcoholic steatohepatitis (NASH), primary sclerosing cholangitis, primary biliary cholangitis), fibrotic renal diseases, skin fibrotic disorders (such as scleroderma), and chronic kidney diseases.

Examples of disorders related to metabolism and their complications include, without limitation, metabolic syndrome and hyperlipidemia (e.g. hyper-triglyceridemia, hyper-triglyceridemia in the setting of low HDL-cholesterol, elevation of LDL and/or total cholesterol and/or VLDL and/or elevated Apolipoprotein B, atherosclerotic cardiovascular disease, etc.).

Examples of disorders related to obesity and their complications include, without limitation, sleep apnea, snoring, asthma, pulmonary hypoventilation syndrome, dementia, heart disease, hypertension, gallbladder disease, gastrointestinal disorders, menstrual irregularities, degenerative arthritis, venous statis ulcer, coronary artery disease, arterial sclerotic disease, pseudotumor cerebri, osteoarthritis, high cholesterol, and increased incidence of malignancies of the liver, ovaries, cervix, uterus, breasts, prostate, or gallbladder.

Examples of disorders of the skin and hair include alopecia (male pattern baldness and alopecia associated with metabolic syndrome), excessive scar formation (cicatrix and keloid), scleroderma, among others.

Examples of disorders related to the GI tract include constipation, irritable bowel syndrome, inflammatory bowel syndrome, including ulcerative colitis and Crohn's disease, etc.

Other disorders may also benefit from the present compounds, including muscle wasting disorders including muscular dystrophy (such as Duchenne Muscular Dystrophy (DMD)), amyotrophic lateral sclerosis (ALS), multiple sclerosis (MS), spinal muscular atrophy, and others.

The present solid compounds and compositions may also be used in a method for preventing or reversing the deposition of adipose tissue in a subject, which is expected to contribute to a reduction of incidence or severity of obesity, which in turn would reduce the incidence or severity of associated co-morbidities.

The present description provides a method of treating a disorder (as described herein) in a subject, comprising administering to the subject identified as in need thereof, a compound or composition of the present description. The identification of those patients who are in need of treatment for the disorders described above is well within the ability and knowledge of one skilled in the art. Certain of the methods for identification of patients which are at risk of developing the above disorders which can be treated by the subject method are appreciated in the medical arts, such as family history, and the presence of risk factors associated with the development of that disease state in the subject patient. A clinician skilled in the art can readily identify such candidate patients, by the use of, for example, clinical tests, physical examination, medical/family history, and genetic determination.

A method of assessing the efficacy of a treatment in a subject includes determining the pre-treatment symptoms of a disorder by methods well known in the art and then administering a therapeutically effective amount of a compound of the present description, to the subject. After an appropriate period of time following the administration of the compound (e.g., 1 week, 2 weeks, one month, six months), the symptoms of the disorder are reevaluated. The modulation (e.g., decrease) of symptoms and/or of a biomarker of the disorder indicates efficacy of the treatment. The symptoms and/or biomarker of the disorder may be determined periodically throughout treatment. For example, the symptoms and/or biomarker of the disorder may be checked every few days, weeks or months to assess the further efficacy of the treatment. A decrease in symptoms and/or biomarker of the disorder indicates that the treatment is efficacious.

Pharmaceutical compositions provided herein are preferably adapted for oral administration. Such formulations may be administered with or without food. The compositions are formulated in unit dosage forms for ease of administration and uniformity of dosage. The expression “unit dosage form” as used herein refers to a physically discrete unit of agent appropriate for the patient to be treated. It will be understood, however, that the total daily usage of the solid dispersions and compositions of the present disclosure will be decided by the attending physician within the scope of sound medical judgment.

The amount of composition that may be included in a single dosage form will vary depending upon the patient to be treated (e.g. child vs adult, etc.) and the particular compound included in the composition. Provided compositions may be formulated such that a total daily dosage of, for instance, between 0.01 and 100 mg/kg body weight/day or between 0.01 and 20 mg/kg body weight/day of the compound can be administered to a patient receiving these compositions. Single dose compositions may contain such an amount, or the total daily dose may be divided in multiple dosage forms to be taken, for instance, one, two or three times a day. For instance, a single dose may include between 5 and 500 mg of the active ingredient, or between 20 and 200 mg. Treatment regimens may comprise administration to a patient a total amount of from about 10 mg to about 1000 mg of the compound(s) of the present description per day in a single dose or divided in multiple doses.

It will be understood that the total daily dose of the compound will be decided by the attending physician within the scope of sound medical judgment. For instance, a specific dosage or treatment regimen for any particular patient will depend upon a variety of factors, including age, body weight, general health, sex, diet, time of administration, rate of excretion, drug combination, the judgment of the treating physician, and the severity of the symptoms associated with the disease or disorder.

Depending upon the disease or disorder to be treated, additional therapeutic agents may also be present in the compositions of this disclosure or co-administered separately. Non-limiting examples of additional therapeutic agents which could be used in combination with the present solid dispersions and formulations include antidiabetic agents, cholesterol-lowering agents, anti-inflammatory agents, antimicrobial agents, matrix metalloproteinase inhibitors, lipoxygenase inhibitors, cytokine antagonists, immunosuppressants, anti-cancer agents, anti-viral agents, cytokines, growth factors, immunomodulators, prostaglandins, or anti-vascular hyperproliferation compound. The treatment may also be complemented with other treatments or interventions such as surgery, radiotherapy (e.g., gamma-radiation, neutron beam radiotherapy, electron beam radiotherapy, proton therapy, brachytherapy, and systemic radioactive isotopes), a biologic response modifier (e.g., an interferon, an interleukin, tumor necrosis factor (TNF)), and agents used to attenuate an adverse effect of the present compound or of a co-administered ingredient.

The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### EXAMPLES

The following non-limiting examples are illustrative embodiments and should not be construed as further limiting the scope of the present invention.

Unless otherwise indicated, all numbers expressing quantities of ingredients, reaction conditions, concentrations, properties, stabilities, and so forth used in the specification and claims are to be understood as being modified in all instances by the term “about.” At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contain certain errors resulting from variations in experiments, testing measurements, statistical analyses and such.

### ABBREVIATIONS

DIPEA: N,N-diisopropylethylamine
DMSO: Dimethylsulfoxide
NMP: N-methyl-2-pyrrolidone
DCM: Dichloromethane

### Example 1—Preparation of Starting Materials

### (i) Preparation of 6-chloro-N-(4-chlorophenyl)-2-methylsulfanyl-5-nitro-pyrimidin-4-amine (B-2)

To a solution of 4,6-dichloro-2-methylsulfanyl-5-nitro-pyrimidine (10 g, 41.7 mmol, 1 eq.) and potassium carbonate (17.2 g, 125 mmol, 3 eq.) in N,N-dimethylformamide (50 mL) was added 4-chloroaniline (5.6 g, 44.2 mmol, 1.06 eq.) at 0° C. with stirred. The mixture was stirred at 0° C. for 1 hour. The reaction mixture was stirred at 25° C. for 0.5 hour. The reaction mixture was added to the water (200 mL) with stirred to give a yellow solid. The residue was purified by flash silica gel chromatography (ISCO®; 120 g SepaFlash® Silica Flash Column, Eluent of 0˜50% Ethyl acetate/Petroleum ether gradient @100 mL/min) to give 6-chloro-N-(4-chlorophenyl)-2-methylsulfanyl-5-nitro-pyrimidin-4-amine (Intermediate B-2, 10 g, 30.2 mmol, 72.4% yield) as a yellow solid. ¹H NMR (400 MHz, CDCl₃-d) δ=9.66 (br s, 1H), 7.49 (d, J=8.8 Hz, 2H), 7.42-7.35 (m, 2H), 2.49 (s, 3H).

### (ii) Preparation of 1-hydroxy-N-methyl-cyclopropanecarboxamide

To a solution of 1-hydroxycyclopropanecarboxylic acid (4.50 g, 44.1 mmol, 1.0 eq.) in dichloromethane (50 mL) and N,N-dimethylformamide (10 mL) was added triethylamine (15.6 g, 154 mmol, 3.5 eq.), methanamine; hydrochloride (5.95 g, 88.2 mmol, 2.0 eq.), 1-(3-Dimethylaminopropyl)-3-ethylcarbodiimide hydrochloride (10.1 g, 52.9 mmol, 1.2 eq.) and 1-Hydroxybenzotriazole (7.15 g, 52.9 mmol, 1.2 eq.), the resulting mixture was stirred at 25° C. for 15 hr. The reaction mixture was concentrated in vacuum, triturated with ethyl acetate (100 mL), filtered and then concentrated in vacuum. The residue was purified by flash silica gel chromatography (ISCO®; 25 g SepaFlash® Silica Flash Column, Eluent of 0˜20% dichloromethane/methanol ether gradient @20 mL/min) to give 1-hydroxy-N-methyl-cyclopropanecarboxamide (2.4 g, 20.8 mmol, 47.2% yield) as a white solid. ¹H NMR (400 MHz, DMSO-d₆) δ=7.84 (br s, 1H), 6.14 (s, 1H), 2.63 (d, J=4.8 Hz, 3H), 1.05-0.92 (m, 2H), 0.85-0.61 (m, 2H).

### (iii) Preparation of 1-(methylaminomethyl)cyclopropanol

To a solution of 1-hydroxy-N-methyl-cyclopropanecarboxamide (500 mg, 4.34 mmol, 1.0 eq.) in tetrahydrofuran (5 mL) was added lithium aluminum hydride (494 mg, 13.0 mmol, 3 eq.) at 0° C., then the mixture was stirred at 60° C. for 1 hour. sodium sulfate decahydrate was added the reaction mixture until no bubble, filtered and concentrated under reduced pressure to give the crude product. Compound 1-(methylaminomethyl)cyclopropanol (400 mg, 3.95 mmol, 91.0% yield) was obtained as yellow oil.

### (iv) Preparation of 2-[(5-iodoimidazol-1-yl)methoxy]ethyl-trimethyl-silane

To a solution of 4-iodo-1H-imidazole (5.48 g, 28.2 mmol, 1.0 eq.) in N,N-dimethylformamide (50 mL) was added sodium hydride (1.36 g, 33.9 mmol, 60% purity, 1.2 eq.), the resulting mixture was stirred at 25° C. for 0.5 hour, then 2-(chloromethoxy)ethyl-trimethyl-silane (5.65 g, 33.9 mmol, 1.2 eq.) was added to reaction mixture dropwise, then stirred at 25° C. for 1 hour. The mixture was diluted with (200 mL) and extracted with ethyl acetate (100 mL*3), washed with brine (200 mL), dried over anhydrous sodium sulfate, filtered and concentrated to afford the residue. The residue was purified by flash silica gel chromatography (ISCO®; 50 g SepaFlash® Silica Flash Column, Eluent of 0˜60% Ethylacetate/Petroleum ether gradient @50 mL/min) to give 2-[(4-iodoimidazol-1-yl)methoxy]ethyl-trimethyl-silane (P1, 3.18 g, 9.81 mmol, 34.7% yield) was obtained as a colorless oil and 2-[(5-iodoimidazol-1-yl)methoxy]ethyl-trimethyl-silane (P2, 2.21 g, 6.82 mmol, 24.1% yield) was obtained as a colorless oil. P1: ¹H NMR (400 MHz, DMSO-d₆) δ=7.77 (d, J=1.2 Hz, 1H), 7.49 (d, J=1.2 Hz, 1H), 5.30 (s, 2H), 3.50-3.43 (m, 2H), 0.87-0.81 (m, 2H), 0.03 (s, 9H). P2: ¹H NMR (400 MHz, DMSO-d₆) δ=8.01 (d, J=0.8 Hz, 1H), 7.05 (d, J=0.8 Hz, 1H), 5.28 (s, 2H), 3.50-3.46 (m, 2H), 0.86-0.82 (m, 2H), 0.03 (s, 9H).

### (v) Preparation of trimethyl-[2-[(5-tributylstannylimidazol-1-yl)methoxy]ethyl]silane

To a solution of 2-[(5-iodoimidazol-1-yl)methoxy]ethyl-trimethyl-silane (2.90 g, 8.94 mmol, 1.0 eq.) in tetrahydrofuran (10 mL) was added chloro(isopropyl)magnesium (2.0 M, 6.70 mL, 1.5 eq.) at−10° C., the mixture was stirred at −10° C. for 1 hour, then tributyl(chloro)stannane (3.49 g, 10.7 mmol, 1.2 eq.) was added dropwise at −10° C., the resulting mixture was stirred at 25° C. for 15 hours. The mixture was quenched by saturated ammonium chloride (20 mL), diluted with ethyl acetate (100 mL), washed with water (50 mL), with brine (50 mL), filtered and concentrated in vacuum. The brown oil was purified by flash silica gel chromatography (ISCO®; 25 g SepaFlash® Silica Flash Column, Eluent of 0˜80% Ethyl acetate/Petroleum ether gradient @30 mL/min) to give trimethyl-[2-[(5-tributylstannylimidazol-1-yl)methoxy]ethyl]silane (2.1 g, 4.31 mmol, 48.1% yield) as a yellow solid. ¹H NMR (400 MHz, CDCl₃-d) δ=7.74 (s, 1H), 7.09 (d, J=0.6 Hz, 1H), 5.23 (s, 2H), 3.50-3.25 (m, 2H), 1.59-1.47 (m, 6H), 1.34 (s, 6H), 1.12 (s, 6H), 0.90 (t, J=7.3 Hz, 11H), −0.01 (s, 9H).

### (vi) Preparation of 2-(1H-imidazol-4-yl)ethylacetate

To a solution of 2-(1H-imidazol-4-yl)ethanol (1.2 g, 10.70 mmol, 1 eq.) and triethylamine (3.25 g, 32.11 mmol, 4.47 mL, 3 eq.) in dichloromethane (15 mL) was added dropwised acetyl chloride (1.26 g, 16.0 mmol, 1.15 mL, 1.5 eq.) at 0° C., the resulting mixture was stirred at 25° C. for 15 hours. The mixture was diluted with water (150 mL) and extracted with dichloromethane (60 mL*5). The combined organic layers were washed with a saturated aqueous sodium hydrogen carbonate solution (100 mL), dried over anhydrous sodium sulfate, filtered and concentrated under reduced pressure, the resulting residue was purified by flash silica gel chromatography (ISCO®; 20 g SepaFlash® Silica Flash Column, Eluent of 0˜10% Methanol/Dichloromethane @20 mL/min). The cut fraction was concentrated under reduced pressure to give compound 2-(1H-imidazol-4-yl)ethylacetate (0.36 g, 2.34 mmol, 21.8% yield) as a light yellow oil.

### (vii) Preparation of 4-methylpiperidine-4-carboxamide

A solution of tert-butyl 4-carbamoyl-4-methyl-piperidine-1-carboxylate (5 g, 20.63 mmol, 1 eq.) in dichloromethane (25 mL) was added hydrochloride/dioxane (4 M, 25 mL). The mixture was stirred at 25° C. for 1 hour. The mixture was concentrated under vacuum to give 4-methylpiperidine-4-carboxamide (3.6 g, 20.2 mmol, 97.6% yield, hydrochloride) as white solid.

### (viii) Preparation of (2R)-1-(methylamino)propan-2-ol

To a solution of tert-butyl N-[(2R)-2-hydroxypropyl]carbamate (5 g, 28.53 mmol, 1 eq.) in tetrahydrofuran (50 mL) was added lithium aluminum hydride (3.25 g, 85.60 mmol, 3 eq.) at 0° C., then the mixture was stirred at 60° C. for 1 hour. The reaction mixture was added to sodium sulfate decahydrate, filtered and concentrated under reduced pressure to give (2R)-1-(methylamino)propan-2-ol (2 g, crude) as colorless oil.

### (ix) Preparation of (2S)-1-(methylamino)propan-2-ol

To a solution of tert-butyl N-[(2S)-2-hydroxypropyl]carbamate (3 g, 17.1 mmol, 1 eq.) in tetrahydrofuran (50 mL) was added lithium aluminum hydride (1.95 g, 51.36 mmol, 3 eq.) at 0° C., then the mixture was stirred at 60° C. for 1 hour. The reaction mixture was added to sodium sulfate decahydrate, filtered and concentrated under reduced pressure to give (2S)-1-(methylamino)propan-2-ol (1 g, crude) as colorless oil.

### (x) Preparation of 1,4-dimethylimidazole-2-carbaldehyde and 1,5-dimethyl-1H-imidazole-2-carbaldehyde

To a solution of 4-methyl-1H-imidazole-2-carbaldehyde (500 mg, 4.54 mmol, 1 eq.) in N,N-dimethylformamide (5 mL) was added potassium carbonate (753 mg, 5.45 mmol, 1.2 eq.) and potassium iodide (773 mg, 5.45 mmol, 1.2 eq.). The mixture was stirred at 25° C. for 12 hours. The mixture was diluted with water (80 mL), extracted with ethyl acetate (60 mL*3). The organic layer was dried with anhydrous sodium sulfate, filtered and concentrated under vacuum to give 1,4-dimethylimidazole-2-carbaldehyde (200 mg, 1.61 mmol, 35.4% yield) and 1,5-dimethyl-1H-imidazole-2-carbaldehyde as a yellow oil. ¹H NMR (400 MHz, CDCl₃-d) δ=9.83-9.37 (m, 2H), 7.02 (s, 1H), 6.82 (s, 1H), 3.91 (s, 3H), 3.86 (s, 4H), 2.23 (s, 7H).

### (xi) Preparation of (2S)-2-(methylamino) propan-1-ol

To a solution of tert-butyl N-[(1S)-2-hydroxy-1-methyl-ethyl]carbamate (5 g, 28.5 mmol, 1 eq.) in tetrahydrofuran (50 mL) was added lithium aluminum hydride (3.25 g, 85.6 mmol, 3 eq.) at 0° C., then the mixture was stirred at 60° C. for 1 hour. The reaction mixture was added to sodium sulfate decahydrate, filtered and concentrated under reduced pressure to give (2S)-2-(methylamino) propan-1-ol (2 g, 22.4 mmol, 78.6% yield) as colorless oil. ¹H NMR (400 MHz, DMSO-d₆) δ=3.25-3.20 (m, 2H), 2.48-2.40 (m, 1H), 2.25 (s, 3H), 0.87 (d, J=6.4 Hz, 3H).

### (xii) Preparation of (2R)-2-(methylamino) propan-1-ol

To a solution of tert-butyl N-[(1R)-2-hydroxy-1-methyl-ethyl]carbamate (5 g, 28.5 mmol, 1 eq.) in tetrahydrofuran (50 mL) was added lithium aluminum hydride (3.25 g, 85.6 mmol, 3 eq.) at 0° C., then the mixture was stirred at 60° C. for 1 hour. The reaction mixture was added to sodium sulfate decahydrate, filtered and concentrated under reduced pressure to give (2R)-2-(methylamino) propan-1-ol (1 g, 11.2 mmol) as colorless oil. ¹H NMR (400 MHz, DMSO-d₆) δ=3.23-3.20 (m, 2H), 2.48-2.40 (m, 1H), 2.25 (s, 3H), 0.87 (d, J=6.4 Hz, 3H).

### (xiii) Preparation of dimethyl((methylamino)methyl)phosphine oxide

To a solution of 1,3,5-trimethyl-1,3,5-triazinane (5 g, 38.7 mmol, 5.44 mL, 1 eq.) in toluene (50 mL) was added methylphosphonoylmethane (3 g, 38.7 mmol, 1 eq.). The mixture was stirred at 120° C. for 15 hours. The reaction mixture was concentrated under vacuum. The residue was purified by flash silica gel chromatography (ISCO®; 120 g SepaFlash® Silica Flash Column, Eluent of 0˜20% Methanol/Dichloromethane @100 mL/min) to give dimethyl((methylamino)methyl)phosphine oxide (2.5 g, 20.6 mmol, 53.3% yield) as a colorless oil. ¹H NMR (400 MHz, CDCl₃-d) δ=2.86-2.59 (m, 2H), 2.51-2.22 (m, 3H), 1.55-1.24 (m, 6H)

### (xiv) Preparation of 5-formyl-6-methyl-pyridine-2-carbonitrile

Step 1: To a suspension of 5-bromo-6-methyl-pyridine-2-carbonitrile (2.43 g, 12.3 mmol, 1.0 eq.), 4,4,5,5-tetramethyl-2-vinyl-1,3,2-dioxaborolane (5.70 g, 37.0 mmol, 6.28 mL, 3.0 eq.), palladium triphenylphosphane (713 mg, 0.62 mmol, 0.05 eq.) and sodium carbonate (4.44 g, 41.9 mmol, 3.4 eq.) in toluene (10 mL), water (1.5 mL) and ethanol (5 mL) was stirred at 90° C. for 1 hour under nitrogen atmosphere. The reaction mixture was cooled at room temperature and filtered, the resulting filter cake was washed with ethanol (5 mL×3). The filter liquor was concentrated under reduced pressure to give the residue. The residue was purified by flash silica gel chromatography (ISCO®; 40 g SepaFlash® Silica Flash Column, Eluent of 0˜50% Ethyl acetate/Petroleum ether gradient @40 mL/min). The cut fraction was concentrated under reduced pressure to give compound 6-methyl-5-vinyl-pyridine-2-carbonitrile (460 mg, 3.19 mmol, 25.87% yield) as a white solid.

Step 2: A mixture of 6-methyl-5-vinyl-pyridine-2-carbonitrile (360 mg, 2.50 mmol, 1.0 eq.) and dipotassium; dioxido(dioxo)osmium; dihydrate (680.82 mg, 1.85 mmol, 0.74 eq.) and sodium periodate (2.67 g, 12.49 mmol, 5.0 eq.) in acetone (6 mL) and water (1 mL) was stirred at 25° C. for 2 hours. The reaction mixture was filtered and the filter cake was washed with acetone (3 mL×3). The filter liquor was diluted with water (50 mL) and extracted with ethyl acetate (20 mL×3). The combined organic layers were washed with brine (50 mL), dried over anhydrous sodium sulfate, filtered and concentrated under reduced pressure to give the residue. The residue was purified by flash silica gel chromatography (ISCO®; 20 g SepaFlash® Silica Flash Column, Eluent of 0˜50% Ethyl acetate/Petroleum ether gradient @30 mL/min). The cut fraction was concentrated under reduced pressure to give compound 5-formyl-6-methyl-pyridine-2-carbonitrile (142 mg, 0.97 mmol, 38.9% yield) as a light yellow solid. ¹H NMR (400 MHz, CDCl₃) δ=10.40 (s, 1H), 8.25 (d, J=7.8 Hz, 1H), 7.75 (d, J=7.8 Hz, 1H), 2.96 (s, 3H).

### (xv) Preparation of 5-formyl-4-methyl-pyridine-2-carbonitrile

Step 1: A suspension of 5-bromo-4-methyl-pyridine-2-carbonitrile (2.00 g, 10.2 mmol, 1.0 eq.), 4,4,5,5-tetramethyl-2-vinyl-1,3,2-dioxaborolane (4.70 g, 30.46 mmol, 5.16 mL, 3 eq.), palladium triphenylphosphane (586.48 mg, 507.54 umol, 0.05 eq.) and sodium carbonate (3.66 g, 34.52 mmol, 3.4 eq.) in toluene (15 mL), water (2 mL) and ethanol (7 mL) was stirred at 90° C. for 1 hour under N₂ atmosphere. The reaction mixture was cooled to room temperature and filtered, the resulting filter cake was washed with ethanol (5 mL*3). The filter liquor was concentrated under reduced pressure to give the residue. The residue was purified by flash silica gel chromatography (ISCO®; 40 g SepaFlash® Silica Flash Column, Eluent of 0˜10% Ethyl acetate/Petroleum ether gradient @40 mL/min). The cut fraction was concentrated under reduced pressure to give 4-methyl-5-vinyl-pyridine-2-carbonitrile (0.55 g, 3.74 mmol, 36.9% yield,) as a white solid.

Step 2: A mixture of 4-methyl-5-vinyl-pyridine-2-carbonitrile (550 mg, 3.81 mmol, 1 eq.) and dipotassium dioxido(dioxo)osmium dihydrate (1.04 g, 2.82 mmol, 0.74 eq.) and sodium periodate (2.45 g, 11.44 mmol, 3.0 eq.) in acetone (6 mL) and water (1 mL) was stirred at 25° C. for 3 hours. The reaction mixture was filtered and the filter cake was washed with acetone (3 mL*3). The resulting filter liquor was diluted with water (100 mL) and extracted with ethyl acetate (50 mL*2). The combined organic layers were washed with brine (100 mL), dried over anhydrous sodium sulfate, filtered and concentrated under reduced pressure, the resulting residue was purified by flash silica gel chromatography (ISCO®; 20 g SepaFlash® Silica Flash Column, Eluent of 0˜50% Ethyl acetate/Petroleum ether gradient @40 mL/min). The cut fraction was concentrated under reduced pressure to give 5-formyl-4-methyl-pyridine-2-carbonitrile (140 mg, 0.96 mmol, 25.1% yield) as a light yellow solid. ¹H NMR (400 MHz, DMSO-d₆) 5=10.34 (s, 1H), 9.03 (s, 1H), 8.12 (s, 1H), 2.69 (s, 3H).

### (xvi) Preparation of 4-ethoxypiperidine-4-carboxamide

A mixture of tert-butyl 4-carbamoyl-4-ethoxy-piperidine-1-carboxylate (650 mg, 2.39 mmol, 1.0 eq.) and hydrochloride/dioxane (4 M, 6 mL, 10.1 eq.) in dioxane (2 mL) was stirred at 25° C. for 1 hour. The reaction mixture was concentrated under reduced pressure to give 4-ethoxypiperidine-4-carboxamide (600 mg, crude) as a white solid. ¹H NMR (400 MHz, MeOD-d₄) δ=3.46-3.41 (m, 2H), 3.24-3.17 (m, 2H), 2.99 (s, 1H), 2.86 (s, 1H), 2.24-2.05 (m, 4H), 1.28 (t, J=7.2 Hz, 3H).

### (xvii) Preparation of 3-methylazetidin-3-ol

To a solution of tert-butyl 3-hydroxy-3-methyl-azetidine-1-carboxylate (2 g, 10.68 mmol, 1 eq.) in hydrochloride/dioxane (4 M, 20 mL) was stirred at 25° C. for 1 hour. The reaction mixture was concentrated under reduced pressure to the crude product 3-methylazetidin-3-ol (1 g, 8.09 mmol, 75.7% yield, hydrochloride) as a white solid. ¹H NMR (400 MHz, CDCl₃-d) δ=5.06 (br s, 2H), 3.71 (s, 2H).

### (xviii) Preparation of tert-butyl 3-methoxy-3-methyl-azetidine-1-carboxylate

Step 1: To a solution of tert-butyl 3-hydroxy-3-methyl-azetidine-1-carboxylate (2 g, 10.68 mmol, 1 eq.) in tetrahydrofuran (20 mL) was added sodium hydride (1 g, 32.05 mmol, 60% purity, 3 eq.) with stirred, then was added methyl iodide (9 g, 64.09 mmol, 4 mL, 6 eq.). The mixture was stirred at 25° C. for 1 hour. The mixture was diluted with water (50 mL), extracted with ethyl acetate (60 mL*3). The organic layer was dried with anhydrous sodium sulfate filtered and concentrated under vacuum. The residue was purified by flash silica gel chromatography (ISCO®; 40 g SepaFlash® Silica Flash Column, Eluent of 0˜30% Ethyl acetate/Petroleum ether gradient @100 mL/min) to give a spot (Rf=0.6) for tert-butyl 3-methoxy-3-methyl-azetidine-1-carboxylate (1.9 g, 9.44 mmol, 88.3% yield) as a white oil. ¹H NMR (400 MHz, CDCl3-d) δ=3.89 (d, J=8.9 Hz, 2H), 3.65 (d, J=9.0 Hz, 2H), 3.22 (s, 3H), 1.44 (br s, 3H), 1.43 (s, 9H).

Step 2: To a solution of tert-butyl 3-methoxy-3-methyl-azetidine-1-carboxylate (300 mg, 1.49 mmol, 1 eq.) in hydrochloride/dioxane (4 M, 3 mL) was stirred at 25° C. for 0.5 hour. The reaction mixture was concentrated under reduced pressure to give the crude product 3-methoxy-3-methyl-azetidine (195 mg, crude, hydrochloride) as a white solid was used into the next step without further purification.

### (xix) Preparation of 4-carbamoyl-4-isopropoxypiperidin-1-ium trifluoroacetate

Sodium hydride 60% in dispersion in mineral oil (3.9 mg, 98 mmol) was added portion wise over 20 min in a flask containing 2-propanol (150 mL) at room temperature. The heterogeneous solution was stirred at that temperature during 20 min and was then slowly transferred to a solution of 1-Boc-4-piperidone (5.00 g, 24.6 mmol) in bromoform (8.96 mL, 98.4 mmol) at 0° C. Full conversion was observed by LCMS after 10 minutes. Sat. NH₄Cl (10 mL) was added and the reaction mixture was concentrated to dryness. The residue was dissolved in EtOAc and washed with 2×NH₄Cl. the organic layer was dried with anhydrous Na₂SO₄ and concentrated. The material obtained was purified on normal phase flash chromatography (loading with DCM, 25 g) using 0-30% EtOAc in hexane affording desired intermediate ester compound (1.53 g, 19%).

Lithium hydroxide (742 mg, 30.4 mmol) was added to a vial containing a solution of the ester intermediate compound (2.00 g, 6.07 mmol) in EtOH (20 mL). The heterogeneous solution was vigorously stirred at 80° C. where full conversion was observed by LCMS overweekend. The reaction mixture was concentrated under reduced pressure and acidified to pH=1 with 3 N HCl. The aqueous solution was extracted with EtOAc, the organic layers were combined, dried with anhydrous Na₂SO₄ and concentrated affording the carboxylic acid (1.40 g, 80%) as a clear oil that was used as is in the next step. DMF (10 mL) was added to a flask containing the acid (1.40 g, 4.87 mmol) and HATU (2.1 g, 5.36 mmol). The solution was cooled to 0° C. and N,N-diisopropylethylamine (1.7 mL, 9.74 mmol) was added in one portion. The ice bath was removed, and the reaction mixture was stirred at room temperature during 45 minutes. The solution was cooled to 0° C. and ammonia (10 mL, 20 mmol) (2M in IPA) was added over 5 minutes. The ice bath was then removed, and the reaction mixture was stirred at room temperature overnight. EtOAc (100 mL) was added and the solution was washed 5 times with saturated NH₄Cl, dried with anhydrous Na₂SO₄ and concentrated. The residue was purified on normal phase flash chromatography (loading with DCM, 50 g) using 0˜100% ACN in DCM affording desired intermediate amide compound (350 mg, 25%).

Trifluoroacetic acid (4.21 mL, 54.5 mmol) was added to a solution of compound (780 mg, 2.72 mmol) in DCM (5.45 mL). the reaction mixture was stirred at room temperature 30 minutes where full conversion was observed by LCMS. The solvents were removed under reduced pressure and the residue lyophilized over weekend. 4-carbamoyl-4-isopropoxypiperidin-1-ium trifluoroacetate 880 mg (100%) was obtained.

### (xx) Preparation of ethyl 4-ethoxypiperidine-4-carboxylate

A solution of 1-boc-4-piperidinone (1.63 g, 8.04 mmol) in bromoform (10.5 mL, 116 mmol) was cooled to 0° C. Potassium hydroxide (4.25 g, 64.3 mmol) dissolved in anhydrous ethanol (25 mL) was added dropwise to the solution over 20 minutes. Then after stirring the mixture for 19 h at room temperature, it was concentrated in vacuo. The residue was partitioned between ethyl acetate (100 mL×2) and water (60 mL). The organic layer was dried over MgSO4 and concentrated in vacuo to a yellow oil residue. The residue was purified using 0% to 15% ethyl acetate in hexanes to afford the tert-butyl 4-carbamoyl-4-methylpiperidine-1-carboxylate (2.41 g, 99%) as an oil. To tert-butyl 4-carbamoyl-4-methylpiperidine-1-carboxylate (351 mg, 1.16 mmol) in DCM (8.5 mL) was added trifluoroacetic acid (2.2 mL, 28.4 mmol). The solution was stirred at room temperature for 1.5 hours and analyzed. The reaction mixture was evaporated and co-evaporated with diethyl ether and lyophilized to a yellow oil containing ethyl 4-ethoxypiperidine-4-carboxylate (148 mg, 40% yield), used without further purification and analysis.

### Example 2—Preparation of A-2 Intermediates

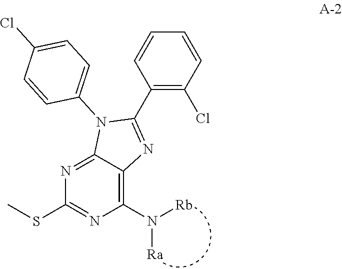

### Preparation of A-2 Intermediates

METHOD A: To a solution of 6-chloro-8-(2-chlorophenyl)-9-(4-chlorophenyl)-2-methylsulfanyl-purine (Intermediate A-1) (1 eq.) and nucleophile NHRaRb (1.5 eq) in N,N-dimethylformamide (5 mL) was added a base (3 eq.). The mixture was stirred at 60° C. for 12 hours. The reaction mixture was added to water (50 mL) under stirring. The mixture was filtered to give the filter residue as a yellow solid. The residue was purified by flash silica gel chromatography (ISCO®; 20 g SepaFlash® Silica Flash Column, Eluent of 0˜45% Ethyl acetate/Petroleum ether gradient @100 mL/min) to give Intermediate A-2 (70-89% yield) as an off-white solid.

Reaction Scale: 500 mg of Intermediate A-1.

METHOD B: A mixture of 6-chloro-8-(2-chlorophenyl)-9-(4-chlorophenyl)-2-methylsulfanyl-purine (Intermediate A-1) (1 eq.), nucleophile NHRaRb (5 eq.) and base (2 eq.) in N,N-dimethylformamide (8 mL) was stirred at 60° C. for 1-2 hours. The reaction mixture was cooled at room temperature and diluted with water (100 mL), then extracted with ethyl acetate (50 mL*3). The combined organic layers were washed with brine (100 mL), dried over anhydrous sodium sulfate, filtered and concentrated under reduced pressure to give Intermediate A-2 as a brown oil or a brown solid, which was used for the next step directly without further purification.

Reaction Scale: 500 mg of Intermediate A-1.

METHOD C: To a solution of 6-chloro-8-(2-chlorophenyl)-9-(4-chlorophenyl)-2-methylsulfanyl-purine (Intermediate A-1) (1.0 eq.) in N,N-dimethylformamide (3 mL) was added the base (2.0 eq.) and the nucleophile NHRaRb (5.0 eq.). The mixture was stirred at 25° C. for 1 hour. The reaction mixture was washed with water (10 mL) and extracted with ethyl acetate 30 mL (10 mL×3). The combined organic layers were washed with brine 20 mL (10 mL×2), dried over anhydrous sodium sulfate, filtered and concentrated under reduced pressure to give a residue. The residue was purified by flash silica gel chromatography (ISCO®; 20 g SepaFlash® Silica Flash Column, Eluent of 0˜100% Ethyl acetate/Petroleum ether gradient @30 mL/min) and the organic phase was concentrated under reduced pressure to give Intermediate A-2 (93-xx % yield) as a yellow solid.

Reaction Scale: 300 mg of Intermediate A-1.

METHOD D: To a solution of 6-chloro-8-(2-chlorophenyl)-9-(4-chlorophenyl)-2-methylsulfanyl-purine (Intermediate A-1) (1.0 eq.) in N,N-dimethylformamide (2 mL) was added a base (3.0 eq.) and a nucleophile NHRaRb (1.2 eq.). The mixture was stirred at 60° C. for 2 hours. The reaction mixture was washed with water (10 mL) and extracted with ethyl acetate 30 mL (10 mL×3). The combined organic layers were washed with brine 20 mL (10 mL×2), dried over anhydrous sodium sulfate, filtered and concentrated under reduced pressure to give a residue. The residue was purified by flash silica gel chromatography (ISCO®; 20 g SepaFlash® Silica Flash Column, Eluent of 0˜100% Ethyl acetate/Petroleum ether gradient @30 mL/min) and the organic phase was concentrated under reduced pressure to give Intermediate A-2 (xx-87% yield) as a yellow solid.

Reaction Scale: 200 mg of Intermediate A-1.

METHOD E: A base (2 eq.) was added to a vial containing a solution of 6-chloro-8-(2-chlorophenyl)-9-(4-chlorophenyl)-2-(methylthio)-9H-purine (Intermediate A-1) (1 eq.) and the nucleophile NHRaRb (1 eq.) in NMP (2.4 mL). The reaction mixture was stirred at room temperature until full conversion was observed by LCMS (1 hour). The reaction mixture was poured into ice water (100 mL). The solids were recovered by Buchner filtration and washing with cold water affording crude Intermediate A-2 (94% yield).

Reaction Scale: 400 mg of Intermediate A-1.

METHOD F: A nucleophile NHRaRb (1.7 eq.) was added to a 8 mL vial containing a solution of 6-chloro-8-(2-chlorophenyl)-9-(4-chlorophenyl)-2-(methylthio)-9H-purine (Intermediate A-1) (1 eq.) and a base (7 eq.) in NMP (1.1 mL). The reaction mixture was stirred at room temperature for 2.5 hours. Water was added and the precipitate that formed was filtered on a Buchner funnel. The aqueous filtrate was also extracted by DCM and the DCM extract was evaporated and combined with the filtered material, affording crude Intermediate A-2, which was used in the next step.

Reaction Scale: 100 mg of Intermediate A-1.

TABLE 2
Reaction conditions and data for Intermediates A-2

| Intermediates | | | Compound name | ¹H NMR |
|---|---|---|---|---|
| A-2 | Base | Nucleophile | and yield | (400 MHz) |
| A-2 (39) | DIPEA | 4- | 1-(8-(2-chlorophenyl)-9- | — |
| NRaRb = | | (trifluoromethyl)- | (4-chlorophenyl)-2- | |
| 4-(trifluoromethyl)- | | piperidine | (methylthio)-9H-purin-6- | |
| piperidine | | | yl)-4-(trifluoromethyl)- | |
| METHOD E | | | piperidine | |
| A-2 (44) | DIPEA | 4-methyl- | 1-(8-(2-chlorophenyl)-9- | — |
| NRaRb = | | piperidine-4- | (4-chlorophenyl)-2- | |
| 4-methyl-piperidine- | | carboxamide | (methylthio)-9H-purin-6- | |
| 4-carboxamide | | | yl)-4-methyl-piperidine- | |
| METHOD E | | | 4-carboxamide | |
| A-2(74): | DIPEA | 3-methylazetidin- | 1-[8-(2-chlorophenyl)-9- | DMSO-d₆, δ = 7.68 (dd, |
| NRaRb = | | 3-ol | (4-chlorophenyl)-2- | J = 1.3, 7.3 Hz, 1H), |
| 3-methylazetidin-3- | | | methylsulfanyl-purin-6- | 7.56-7.38 (m, 5H), |
| ol | | | yl]-3-methyl-azetidin-3-ol | 7.35-7.25 (m, 2H), |
| METHOD A | | | 70% yield | 4.68-4.05 (m, 4H), |
| | | | | 2.41 (s, 3H), 1.47 (s, |
| | | | | 3H) |
| A-2(75): | Cs₂CO₃ | 3-methylazetidin- | 1-[8-(2-chlorophenyl)-9- | DMSO-d₆, δ = 7.74- |
| NRaRb = | | 3-carbonitrile | (4-chlorophenyl)-2- | 7.64 (m, 1H), 7.59- |
| 3-methylazetidin-3- | | | methylsulfanyl-purin-6- | 7.40 (m, 5H), 7.36- |
| carbonitrile | | | yl]-3-methyl-azetidine-3- | 7.25 (m, 2H), 4.90- |
| METHOD A | | | carbonitrile | 4.16 (m, 4H), 2.43 |
| | | | 79% yield | (s, 3H), 1.71 (s, 3H) |
| A-2(76): | DIPEA | 3-methoxy-3- | 8-(2-chlorophenyl)-9-(4- | DMSO-d₆, δ = 7.69 (dd, |
| NRaRb = | | methylazetidin | chlorophenyl)-6-(3- | J = 1.3, 7.3 Hz, 1H), |
| 3-methoxy-3- | | | methoxy-3-methyl- | 7.56-7.39 (m, 5H), |
| methylazetidin | | | azetidin-1-yl)-2- | 7.31 (d, J = 8.8 Hz, 2H), |
| METHOD A | | | methylsulfanyl-purine | 4.69-4.04 (m, 4H), |
| | | | 89% yield | 3.24 (s, 3H), 2.42 (s, |
| | | | | 3H), 1.50 (s, 3H) |
| A-2(87): | DIPEA | propan-2-amine | 8-(2-chlorophenyl)-9-(4- | — |
| NRaRb = | | | chlorophenyl)-N- | |
| propan-2-amine | | | isopropyl-2- | |
| METHOD B | | | methylsulfanyl-purin-6- | |
| | | | amine | |
| | | | crude | |
| A-2(97): | DIPEA | 2-(methylamino)ethanol | 2-[[8-(2-chlorophenyl)-9- | DMSO-d₆, δ = 7.67 (d, |
| NRaRb = | | | (4-chlorophenyl)-2- | J = 7.2 Hz, 1H), 7.53- |
| 2-(methylamino) | | | methylsulfanyl-purin-6- | 7.41 (m, 5H), 7.31 (d, |
| ethanol | | | yl]-methyl-amino]ethanol | J = 8.8 Hz, 2H), 4.77 (s, |
| METHOD C | | | 93% yield | 1H), 4.29 (s, 1H), 3.71 |
| | | | | (d, J = 4.4 Hz, 4H), 3.41- |
| | | | | 3.20 (m, 2H), 2.44 (s, |
| | | | | 3H) |
| A-2(98): | DIPEA | 2-aminoethanol | 2-[[8-(2-chlorophenyl)-9- | — |
| NRaRb = | | | (4-chlorophenyl)-2- | |
| 2-aminoethanol | | | methylsulfanyl-purin-6- | |
| METHOD C | | | yl]amino]ethanol | |
| | | | crude | |
| A-2(100): | DIPEA | 2- | 2-[[8-(2-chlorophenyl)-9- | — |
| NRaRb = | | aminoacetamide | (4-chlorophenyl)-2- | |
| 2-aminoacetamide | | | methylsulfanyl-purin-6- | |
| METHOD B | | | yl]amino]acetamide | |
| | | | crude | |
| A-2(119): | DIPEA | (3R)-3- | (3R)-1-[8-(2- | MeOD-d₄, δ = 7.59 (d, |
| NRaRb = | | methylpyrrolidin- | chlorophenyl)-9-(4- | J = 7.6 Hz, 1H), 7.54- |
| (3R)-3- | | 3-ol | chlorophenyl)-2- | 7.33 (m, 5H), 7.32- |
| methylpyrrolidin-3- | | | methylsulfanyl-purin-6- | 7.22 (m, 2H), 4.44- |
| ol | | | yl]-3-methyl-pyrrolidin- | 4.19 (m, 1H), 4.04- |
| METHOD D | | | 3-ol | 3.33 (m, 3H), 2.48 (s, |
| | | | 87% yield | 3H), 2.16-2.02 (m, |
| | | | | 2H), 1.48 (s, 3H) |
| A-2 (141) | DIPEA | 4-(ethylamino) | 1-(8-(2-chlorophenyl)-9- | — |
| NRaRb = | | piperidine-4- | (4-chlorophenyl)-2- | |
| 4-(ethylamino) | | carboxamide | (methylthio)-9H-purin-6- | |
| piperidine-4- | | | yl)-4- | |
| carboxamide | | | (ethylamino)piperidine- | |
| METHOD E | | | 4-carboxamide | |
| | | | 94% yield | |
| A-2 (154) | DIPEA | piperidine-4- | 1-(8-(2-chlorophenyl)-9- | — |
| NRaRb = | | carboxamide | (4-chlorophenyl)-2- | |
| piperidine-4- | | | (methylthio)-9H-purin-6- | |
| carboxamide | | | yl)-piperidine-4- | |
| METHOD E | | | carboxamide | |
| A-2 (164) | DIPEA | 1,1- | 1-(8-(2-chlorophenyl)-9- | — |
| NRaRb = | | Dioxidotetrahydro- | (4-chlorophenyl)-2- | |
| 1,1- | | 2H-thiopyran-4- | (methylthio)-9H-purin-6- | |
| Dioxidotetrahydro- | | ylamine | yl)-1,1- | |
| 2H-thiopyran-4- | | | Dioxidotetrahydro-2H- | |
| ylamine | | | thiopyran-4-ylamine | |
| METHOD E | | | | |
| A-2 (169) | DIPEA | (1,4-oxazinan-2- | 1-(8-(2-chlorophenyl)-9- | — |
| NRaRb = | | yl)methanol | (4-chlorophenyl)-2- | |
| (1,4-oxazinan-2- | | | (methylthio)-9H-purin-6- | |
| yl)methanol | | | yl)-(1,4-oxazinan-2- | |
| METHOD E | | | yl)methanol | |
| A-2 (171) | DIPEA | (1,4-oxazinan-2- | 1-(8-(2-chlorophenyl)-9- | — |
| NRaRb = | | yl)methanol | (4-chlorophenyl)-2- | |
| (1,4-oxazinan-2- | | | (methylthio)-9H-purin-6- | |
| yl)methanol | | | yl)-(1,4-oxazinan-2- | |
| METHOD E | | | yl)methanol | |
| A-2 (174) | DIPEA | (pyrrolidine-3- | 1-(8-(2-chlorophenyl)-9- | — |
| NRaRb = | | yl)methanol | (4-chlorophenyl)-2- | |
| (pyrrolidine-3- | | | (methylthio)-9H-purin-6- | |
| yl)methanol | | | yl)-(pyrrolidine-3- | |
| METHOD E | | | yl)methanol | |
| A-2 (178) | DIPEA | 4-aminotetrahydropyran | 1-(8-(2-chlorophenyl)-9- | — |
| NRaRb = | | | (4-chlorophenyl)-2- | |
| 4-aminotetrahydropyran | | | (methylthio)-9H-purin-6- | |
| METHOD E | | | yl)-4- | |
| | | | aminotetrahydropyran | |
| A-2 (181) | DIPEA | 4-methyl-4- | 1-(8-(2-chlorophenyl)-9- | — |
| NRaRb = | | piperidinol | (4-chlorophenyl)-2- | |
| 4-methyl-4- | | | (methylthio)-9H-purin-6- | |
| piperidinol | | | yl)-4-methyl-4- | |
| METHOD E | | | piperidinol | |
| A-2 (183) | DIPEA | morpholine | 1-(8-(2-chlorophenyl)-9- | — |
| NRaRb = | | | (4-chlorophenyl)-2- | |
| morpholine | | | (methylthio)-9H-purin-6- | |
| METHOD E | | | yl)-morpholine | |
| A-2 (184) | DIPEA | 4,4- | 1-(8-(2-chlorophenyl)-9- | — |
| NRaRb = | | difluoropiperidine | (4-chlorophenyl)-2- | |
| 4,4- | | | (methylthio)-9H-purin-6- | |
| difluoropiperidine | | | yl)-4,4-difluoropiperidine | |
| METHOD E | | | | |
| A-2 (185) | DIPEA | 3-methoxy- | 1-(8-(2-chlorophenyl)-9- | — |
| NRaRb = | | pyrrolidine | (4-chlorophenyl)-2- | |
| 3-methoxy- | | | (methylthio)-9H-purin-6- | |
| pyrrolidine | | | yl)-3-methoxy- | |
| METHOD E | | | pyrrolidine | |
| A-2 (186) | DIPEA | piperidine | — | — |
| NRaRb = | | derivative | | |
| piperidine | | (R3 = C36) | | |
| derivative | | | | |
| (R3 = C36) | | | | |
| METHOD E | | | | |
| A-2 (187) | DIPEA | azetidine | — | — |
| NRaRb = | | derivative | | |
| azetidine derivative | | (R3 = C37) | | |
| (R3 = C37) | | | | |
| METHOD E | | | | |
| A-2 (188) | DIPEA | azetidine | — | — |
| NRaRb = | | derivative | | |
| azetidine derivative | | (R3 = C38) | | |
| (R3 = C38) | | | | |
| METHOD E | | | | |
| A-2 (190) | DIPEA | piperidine | — | — |
| NRaRb = | | derivative | | |
| piperidine | | (R3 = C39) | | |
| derivative | | | | |
| (R3 = C39) | | | | |
| METHOD E | | | | |
| A-2 (191) | DIPEA | piperidine | — | — |
| NRaRb = | | derivative | | |
| piperidine | | (R3 = C40) | | |
| derivative | | | | |
| (R3 = C40) | | | | |
| METHOD E | | | | |
| A-2 (192) | DIPEA | (1,1- | 1-(8-(2-chlorophenyl)-9- | — |
| NRaRb = | | dioxidotetrahydro- | (4-chlorophenyl)-2- | |
| METHOD E | | 3-thienyl)amine | (methylthio)-9H-purin-6- | |
| | | | yl)-(1,1- | |
| | | | dioxidotetrahydro-3- | |
| | | | thienyl)amine | |
| A-2 (193) | DIPEA | (1,1- | 1-(8-(2-chlorophenyl)-9- | — |
| NRaRb = | | dioxidotetrahydro- | (4-chlorophenyl)-2- | |
| METHOD E | | 3-thienyl)amine | (methylthio)-9H-purin-6- | |
| | | | yl)-(1,1- | |
| | | | dioxidotetrahydro-3- | |
| | | | thienyl)amine | |
| A-2 (194) | DIPEA | 3-aminotetrahydrofuran | 1-(8-(2-chlorophenyl)-9- | — |
| NRaRb = | | | (4-chlorophenyl)-2- | |
| 3-aminotetrahydrofuran | | | (methylthio)-9H-purin-6- | |
| METHOD E | | | yl)-3-aminotetrahydrofuran | |
| A-2 (195) | DIPEA | 3-aminotetrahydrofuran | 1-(8-(2-chlorophenyl)-9- | — |
| NRaRb = | | | (4-chlorophenyl)-2- | |
| 3-aminotetrahydrofuran | | | (methylthio)-9H-purin-6- | |
| METHOD E | | | yl)-3-aminotetrahydrofuran | |
| A-2 (201) | DIPEA | nipecotamide | 1-(8-(2-chlorophenyl)-9- | — |
| NRaRb = | | | (4-chlorophenyl)-2- | |
| nipecotamide | | | (methylthio)-9H-purin-6- | |
| METHOD F | | | yl)-nipecotamide | |
| A-2 (202) | DIPEA | nipecotamide | 1-(8-(2-chlorophenyl)-9- | — |
| NRaRb = | | | (4-chlorophenyl)-2- | |
| nipecotamide | | | (methylthio)-9H-purin-6- | |
| METHOD F | | | yl)-nipecotamide | |
| A-2 (203) | DIPEA | morpholine | — | — |
| NRaRb = | | derivative | | |
| morpholine | | (R3 = C49) | | |
| derivative | | | | |
| (R3 = C49) | | | | |
| METHOD F | | | | |
| A-2 (204) | DIPEA | morpholine | — | — |
| NRaRb = | | derivative | | |
| morpholine | | (R3 = C50) | | |
| derivative | | | | |
| (R3 = C50) | | | | |
| METHOD F | | | | |
| A-2 (206) | DIPEA | 2- | 1-(8-(2-chlorophenyl)-9- | — |
| NRaRb = | | methylmorpholine | (4-chlorophenyl)-2- | |
| 2-methylmorpholine | | | (methylthio)-9H-purin-6- | |
| METHOD F | | | yl)-2-methylmorpholine | |
| A-2 (207) | DIPEA | 4-ethoxy- | 1-(8-(2-chlorophenyl)-9- | — |
| NRaRb = | | piperidine-4- | (4-chlorophenyl)-2- | |
| 4-ethoxy-piperidine- | | carboxamide | (methylthio)-9H-purin-6- | |
| 4-carboxamide | | | yl-4-ethoxy-piperidine-4- | |
| METHOD F | | | carboxamide | |
| A-2 (210) | DIPEA | 2- | 1-(8-(2-chlorophenyl)-9- | — |
| NRaRb = | | methylmorpholine | (4-chlorophenyl)-2- | |
| 2-methylmorpholine | | | (methylthio)-9H-purin-6- | |
| METHOD F | | | yl)-2-methylmorpholine | |
| A-2 (211) | DIPEA | 3- | 1-(8-(2-chlorophenyl)-9- | — |
| NRaRb = | | methylmorpholine | (4-chlorophenyl)-2- | |
| 3-methylmorpholine | | | (methylthio)-9H-purin-6- | |
| METHOD F | | | yl)-3-methylmorpholine | |
| A-2 (212) | DIPEA | 3- | 1-(8-(2-chlorophenyl)-9- | — |
| NRaRb = | | methylmorpholine | (4-chlorophenyl)-2- | |
| 3-methylmorpholine | | | (methylthio)-9H-purin-6- | |
| METHOD F | | | yl)-3-methylmorpholine | |
| A-2 (213) | DIPEA | 4-methoxy- | 1-(8-(2-chlorophenyl)-9- | — |
| NRaRb = | | piperidine-4- | (4-chlorophenyl)-2- | |
| 4-methoxy- | | carboxamide | (methylthio)-9H-purin-6- | |
| piperidine-4- | | | yl)-4-methoxy- | |
| carboxamide | | | piperidine-4- | |
| METHOD F | | | carboxamide | |
| A-2 (214) | DIPEA | secondary amine | — | — |
| NRaRb = | | derivative | | |
| secondary amine | | (R3 = C56) | | |
| derivative | | | | |
| (R3 = C56) | | | | |
| METHOD F | | | | |
| A-2 (215) | DIPEA | 4-isopropoxy- | 1-(8-(2-chlorophenyl)-9- | — |
| NRaRb = | | piperidine-4- | (4-chlorophenyl)-2- | |
| 4-isopropoxy- | | carboxamide | (methylthio)-9H-purin-6- | |
| piperidine-4- | | | yl)-4-isopropoxy- | |
| carboxamide | | | piperidine-4- | |
| METHOD F | | | carboxamide | |
| A-2 (216) | DIPEA | 3-ethoxy- | 1-(8-(2-chlorophenyl)-9- | — |
| NRaRb = | | piperidine-3- | (4-chlorophenyl)-2- | |
| 3-ethoxy-piperidine- | | carboxamide | (methylthio)-9H-purin-6- | |
| 3-carboxamide | | | yl)-3-ethoxy-piperidine- | |
| METHOD F | | | 3-carboxamide | |
| A-2 (222) | DIPEA | 1-acetylpiperazine | 1-(8-(2-chlorophenyl)-9- | — |
| NRaRb = | | | (4-chlorophenyl)-2- | |
| 1-acetylpiperazine | | | (methylthio)-9H-purin-6- | |
| METHOD F | | | yl)-1-acetylpiperazine | |
| A-2 (224) | DIPEA | methyl(piperazine- | 1-(8-(2-chlorophenyl)-9- | — |
| NRaRb = | | 1-carboxylate) | (4-chlorophenyl)-2- | |
| methyl(piperazine- | | | (methylthio)-9H-purin-6- | |
| 1-carboxylate) | | | yl)-methyl(piperazine-1- | |
| METHOD F | | | carboxylate) | |
| A-2 (226) | DIPEA | 4-methyl- | 1-(8-(2-chlorophenyl)-9- | — |
| NRaRb = | | piperidine-4-N- | (4-chlorophenyl)-2- | |
| 4-methyl-piperidine- | | acetamide | (methylthio)-9H-purin-6- | |
| 4-N-acetamide | | | yl)-4-methyl-piperidine- | |
| METHOD F | | | 4-N-acetamide | |
| A-2 (227) | DIPEA | 1- | 1-(8-(2-chlorophenyl)-9- | — |
| NRaRb = | | methylpiperazine | (4-chlorophenyl)-2- | |
| 1-methyl | | | (methylthio)-9H-purin-6- | |
| piperazine | | | yl)-1-methylpiperazine | |
| METHOD F | | | | |
| A-2 (233) | DIPEA | secondary amine | — | — |
| NRaRb = | | derivative | | |
| secondary amine | | (R3 = C64) | | |
| derivative | | | | |
| (R3 = C64) | | | | |
| METHOD F | | | | |
| A-2 (237) | DIPEA | 3-carboxamide- | 1-(8-(2-chlorophenyl)-9- | — |
| NRaRb = | | azetidin | (4-chlorophenyl)-2- | |
| 3-carboxamide- | | | (methylthio)-9H-purin-6- | |
| azetidin | | | yl)-3-carboxamide- | |
| METHOD E | | | azetidin | |

### Example 3—Preparation of A3 Intermediates

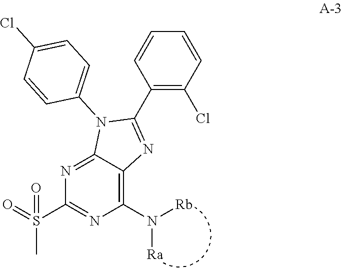

### Preparation of A-3 Intermediates

METHOD A: To a solution of Intermediate A-2 (1 eq.) and potassium peroxymonosulfate (10 eq.) in N-methyl pyrrolidone (3 mL) was added water (0.3 mL). The mixture was stirred at 60° C. for 12 hours. The reaction mixture was added to water (30 mL) under stirring. The mixture was filtered to give Intermediate A-3 (90-93% yield) as a yellow or brown solid.

Reaction Scale: 200 mg of Intermediate A-2.

METHOD B: To solution of Intermediate A-2 (1 eq.) in N-methy-2-pyrrolidone (10 mL) was added a solution of potassium peroxymonosulfate (6 eq.) in water (1 mL), the resulting mixture was stirred at 60° C. for 27 hours. The reaction mixture was added to water (100 mL) and filtered. The resulting filter cake was purified by flash silica gel chromatography (ISCO®; 20 g SepaFlash® Silica Flash Column, Eluent of 0˜80% Ethyl acetate/Petroleum ether gradient @40 mL/min). The cut fraction was concentrated under reduced pressure to give Intermediate A-3 (46% yield) as a white solid.

Reaction Scale: 500 mg of Intermediate A-2.

METHOD C: To a solution of Intermediate A-2 (1.0 eq.) in N-methyl-2-pyrrolidone (5 mL) and water (0.5 mL) was added potassium peroxymonosulfate (1.17 g, 6.95 mmol, 10 eq.). The mixture was stirred at 60° C. for 1 hour. The reaction mixture was added dropwise into water (40 mL), filtered and the filtration residue was concentrated under reduced pressure to give Intermediate A-3 (73-94% yield) as a light-yellow solid.

Reaction Scale: 300 mg of Intermediate A-2.

METHOD D: To solution of Intermediate A-2 (1.0 eq.) in N-methy-2-pyrrolidone (10 mL) was added a solution of potassium peroxymonosulfate (8.0 eq.) in water (2 mL), the resulting mixture was stirred at 60° C. for 18 hours. The reaction mixture was diluted with water (100 mL) and extracted with ethyl acetate (50 mL*2). The combined organic layers were washed with brine (100 mL), dried over anhydrous sodium sulfate, filtered and concentrated under reduced pressure to give Intermediate A-3 (crude) as a yellow solid.

Reaction Scale: 500 mg of Intermediate A-2.

METHOD E: A mixture of Intermediate A-2 (1.0 eq.) and potassium peroxymonosulfate (8.0 eq.) in N-methy-2-pyrrolidone (6 mL) was stirred at 60° C. for 4 hours. The reaction mixture was diluted with water (100 mL) and extracted with ethyl acetate (30 mL*3). The combined organic layers were washed with brine (100 mL), dried over anhydrous sodium sulfate, filtered and concentrated under reduced pressure to give Intermediate A-3 (crude) as a yellow solid, which was used for the next step directly without further purification.

### Reaction Scale: 500 mg of Intermediate A-2.

METHOD F: Crude Intermediate A-2 (1 eq.) was dissolved in NMP (5.5 mL) then water (0.55 mL) was added dropwise making sure that the solution stays clear. Potassium peroxymonosulfate (3 eq.) was then added in one portion and the reaction mixture was stirred at room temperature overnight. Water (50 mL) was slowly added and the precipitate was recovered by filtration on Buchner. The solids were washed with water and were then lyophilized affording Intermediate A-3 (90% yield).

METHOD G: Potassium peroxymonosulfate (3.5 eq.), NMP (1.6 m), and water (160 μL) were added in a 8 mL vial containing crude intermediate A-2 at 60° C. overnight. Water was added and the precipitate obtained was filtered on a Buchner funnel to afford Intermediate A-3 (97% yield) which was used in the next step without purification.

Reaction Scale: 100 mg of Intermediate A-2.

TABLE 3
Reaction conditions and data for Intermediates A-3

| | Starting | | |
|---|---|---|---|
| Intermediates A-3 | material | Compound name and yield | ¹H NMR (400 MHz) |
| A-3(39) | A-2(39) | 1-[8-(2-chlorophenyl)-9-(4- | — |
| NRaRb = | | chlorophenyl)-2-methylsulfonyl- | |
| 4-(trifluoromethyl)- | | purin-6-yl]-4-(trifluoromethyl)- | |
| piperidine | | piperidine | |
| METHOD F | | | |
| A-3(44) | A-2(44) | 1-[8-(2-chlorophenyl)-9-(4- | — |
| NRaRb = | | chlorophenyl)-2-methylsulfonyl- | |
| 4-methyl-piperidine-4- | | purin-6-yl]-4-methyl-piperidine- | |
| carboxamide | | 4-carboxamide | |
| METHOD F | | | |
| A-3(74): | A-2(74) | 1-[8-(2-chlorophenyl)-9-(4- | DMSO-d₆, δ = 7.71 (dd, J = |
| NRaRb = | | chlorophenyl)-2-methylsulfonyl- | 1.2, 7.5 Hz, 1H), 7.59-7.44 |
| 3-methylazetidin-3-ol | | purin-6-yl]-3-methyl-azetidin-3- | (m, 5H), 7.37 (d, J = 8.7 Hz, |
| METHOD A | | ol | 2H), 5.89 (s, 1H), 4.79-4.08 |
| | | 93% yield | (m, 4H), 3.28 (s, 3H), 1.49 (s, |
| | | | 3H) |
| A-3(75): | A-2(75) | 1-[8-(2-chlorophenyl)-9-(4- | DMSO-d₆, δ = 7.76-7.68 (m, |
| NRaRb = | | chlorophenyl)-2-methylsulfonyl- | 1H), 7.59-7.45 (m, 5H), 7.37 |
| 3-methylazetidin-3- | | purin-6-yl]-3-methyl-azetidine- | (d, J = 8.7 Hz, 2H), 5.18-4.21 |
| carbonitrile | | 3-carbonitrile | (m, 4H), 3.30(s, 3H), 1.74 (s, |
| METHOD A | | 92% yield | 3H) |
| A-3(76): | A-2(76) | 8-(2-chlorophenyl)-9-(4- | DMSO-d₆, δ = 7.78-7.65 (m, |
| NRaRb = | | chlorophenyl)-6-(3-methoxy-3- | 1H), 7.60-7.42 (m, 5H), 7.37 |
| 3-methoxy-3- | | methyl-azetidin-1-yl)-2- | (d, J = 8.7 Hz, 2H), 4.68-4.38 |
| methylazetidin | | methylsulfonyl-purine | (m, 2H), 4.34-4.07 (m, 2H), |
| METHOD A | | 90% yield | 3.27 (d, J = 11.9 Hz, 6H), 1.53 |
| | | | (s, 3H) |
| A-3(87): | A-2(87) | 8-(2-chlorophenyl)-9-(4- | — |
| NRaRb = | | chlorophenyl)-N-isopropyl-2- | |
| propan-2-amine | | methylsulfonyl-purin-6-amine | |
| METHOD B | | crude | |
| A-3(97): | A-2(97) | 2-[[8-(2-chlorophenyl)-9-(4- | DMSO-d₆, δ = 7.70 (d, J = 5.6 |
| NRaRb = | | chlorophenyl)-2-methylsulfonyl- | Hz, 1H), 7.59-7.44 (m, 5H), |
| 2-(methylamino)ethanol | | purin-6-yl]-methyl- | 7.38 (d, J = 8.8 Hz, 2H), 4.38 |
| METHOD C | | amino]ethanol | (s, 1H), 3.84 (s, 2H), 3.80- |
| | | 73% yield | 3.68 (m, 2H), 3.33-3.29 (m, 6H) |
| A-3(98): | A-2(98) | 2-[[8-(2-chlorophenyl)-9-(4- | — |
| NRaRb = | | chlorophenyl)-2-methylsulfonyl- | |
| 2-aminoethanol | | purin-6-yl]amino]ethanol | |
| METHOD D | | crude | |
| A-3(100): | A-2(100) | 2-[[8-(2-chlorophenyl)-9-(4- | — |
| NRaRb = | | chlorophenyl)-2-methylsulfonyl- | |
| 2-amino | | purin-6-yl]amino]acetamide | |
| acetamide | | crude | |
| METHOD E | | | |
| A-3(119): | A-2(119) | (3R)-1-[8-(2-chlorophenyl)-9- | CDCl₃-d, δ = 7.51 (t, J = 6.0 |
| NRaRb = | | (4-chlorophenyl)-2- | Hz, 1H), 7.45-7.31 (m, 5H), |
| (3R)-3- | | methylsulfonyl-purin-6-yl]-3- | 7.19 (d, J = 8.8 Hz, 2H), 3.39 |
| methylpyrrolidin-3-ol | | methyl-pyrrolidin-3-ol | (t, J = 7.2 Hz, 4H), 3.30 (d, J = |
| METHOD C | | 94% yield | 3.6 Hz, 2H), 2.85 (s, 3H), 1.56 |
| | | | (s, 3H) |
| A-3 (141) | A-2(141) | 1-(8-(2-chlorophenyl)-9-(4- | — |
| NRaRb = | | chlorophenyl)-2- | |
| 4-(ethylamino) | | (methylsulfonyl)-9H-purin-6-yl)- | |
| piperidine-4- | | 4-(ethylamino)piperidine-4- | |
| carboxamide | | carboxamide | |
| METHOD F | | | |
| A-3 (154) | A2(154) | 1-(8-(2-chlorophenyl)-9-(4- | — |
| NRaRb = | | chlorophenyl)-2- | |
| piperidine-4- | | (methylsulfonyl)-9H-purin-6-yl)- | |
| carboxamide | | piperidine-4-carboxamide | |
| METHOD F | | | |
| A-3 (164) | A2(164) | 1-(8-(2-chlorophenyl)-9-(4- | — |
| NRaRb = | | chlorophenyl)-2- | |
| 1,1-Dioxidotetrahydro- | | (methylsulfonyl)-9H-purin-6-yl)- | |
| 2H-thiopyran-4-ylamine | | 1,1-Dioxidotetrahydro-2H- | |
| METHOD F | | thiopyran-4-ylamine | |
| A-3 (169) | A2(169) | 1-(8-(2-chlorophenyl)-9-(4- | — |
| NRaRb = | | chlorophenyl)-2- | |
| (1,4-oxazinan-2- | | (methylsulfonyl)-9H-purin-6-yl)- | |
| yl)methanol | | (1,4-oxazinan-2-yl) methanol | |
| METHOD F | | | |
| A-3 (171) | A2(171) | 1-(8-(2-chlorophenyl)-9-(4- | — |
| NRaRb = | | chlorophenyl)-2- | |
| (1,4-oxazinan-2- | | (methylsulfonyl)-9H-purin-6-yl)- | |
| yl)methanol | | (1,4-oxazinan-2-yl)methanol | |
| METHOD F | | | |
| A-3 (174) | A2(174) | 1-(8-(2-chlorophenyl)-9-(4- | — |
| NRaRb = | | chlorophenyl)-2- | |
| (pyrrolidine-3- | | (methylsulfonyl)-9H-purin-6-yl)- | |
| yl)methanol | | (pyrrolidine-3-yl)methanol | |
| METHOD F | | | |
| A-3 (178) | A2(178) | 1-(8-(2-chlorophenyl)-9-(4- | — |
| NRaRb = | | chlorophenyl)-2- | |
| 4-aminotetrahydropyran | | (methylsulfonyl)-9H-purin-6-yl)- | |
| METHOD F | | 4-aminotetrahydropyran | |
| A-3(181) | A-2(181) | 1-(8-(2-chlorophenyl)-9-(4- | — |
| NRaRb = | | chlorophenyl)-2- | |
| 4-methyl | | (methylsulfonyl)-9H-purin-6-yl)- | |
| 4-piperidinol | | 4-methyl-4-piperidinol | |
| METHOD F | | | |
| A-3(183) | A-2(183) | 1-(8-(2-chlorophenyl)-9-(4- | — |
| NRaRb = | | chlorophenyl)-2- | |
| morpholine | | (methylsulfonyl)-9H-purin-6-yl)- | |
| METHOD F | | morpholine | |
| A-3(184) | A-2(184) | 1-(8-(2-chlorophenyl)-9-(4- | — |
| NRaRb = | | chlorophenyl)-2- | |
| 4,4-difluoropiperidine | | (methylsulfonyl)-9H-purin-6-yl)- | |
| METHOD F | | 4,4-difluoropiperidine | |
| A-3(185) | A-2(185) | 1-(8-(2-chlorophenyl)-9-(4- | — |
| NRaRb = | | chlorophenyl)-2- | |
| 3-methoxy-pyrrolidine | | (methylsulfonyl)-9H-purin-6-yl)- | |
| METHOD F | | 3-methoxy-pyrrolidine | |
| A-3(186) | A-2(186) | — | — |
| NRaRb = | | | |
| piperidine derivative | | | |
| (R3 = C36) | | | |
| METHOD F | | | |
| A-3(187) | A-2(187) | — | — |
| NRaRb = | | | |
| azetidine derivative | | | |
| (R3 = C37) | | | |
| METHOD F | | | |
| A-3(188) | A-2(188) | — | — |
| NRaRb = | | | |
| azetidine derivative | | | |
| (R3 = C38) | | | |
| METHOD F | | | |
| A-3(190) | A-2(190) | — | — |
| NRaRb = | | | |
| piperidine derivative | | | |
| (R3 = C39) | | | |
| METHOD F | | | |
| A-3(191) | A-2(191) | — | — |
| NRaRb = | | | |
| piperidine derivative | | | |
| (R3 = C40) | | | |
| METHOD F | | | |
| A-3(192) | A-2(192) | 1-(8-(2-chlorophenyl)-9-(4- | — |
| NRaRb = | | chlorophenyl)-2- | |
| (1,1-dioxidotetrahydro- | | (methylsulfonyl)-9H-purin-6-yl)- | |
| 3-thienyl)amine | | (1,1-dioxidotetrahydro-3- | |
| METHOD F | | thienyl)amine | |
| A-3(193) | A-2(193) | 1-(8-(2-chlorophenyl)-9-(4- | — |
| NRaRb = | | chlorophenyl)-2- | |
| (1,1-dioxidotetrahydro- | | (methylsulfonyl)-9H-purin-6-yl)- | |
| 3-thienyl)amine | | (1,1-dioxidotetrahydro-3- | |
| METHOD F | | thienyl)amine | |
| A-3(194) | A-2(194) | 1-(8-(2-chlorophenyl)-9-(4- | — |
| NRaRb = | | chlorophenyl)-2- | |
| 3-aminotetrahydrofuran | | (methylsulfonyl)-9H-purin-6-yl)- | |
| METHOD F | | 3-aminotetrahydrofuran | |
| A-3(195) | A-2(195) | 1-(8-(2-chlorophenyl)-9-(4- | — |
| NRaRb = | | chlorophenyl)-2- | |
| 3-aminotetrahydrofuran | | (methylsulfonyl)-9H-purin-6-yl)- | |
| METHOD F | | 3-aminotetrahydrofuran | |
| A-3(201) | A-2(201) | 1-(8-(2-chlorophenyl)-9-(4- | — |
| NRaRb = | | chlorophenyl)-2- | |
| nipecotamide | | (methylsulfonyl)-9H-purin-6-yl)- | |
| METHOD G | | nipecotamide | |
| A-3(202) | A-2(202) | 1-(8-(2-chlorophenyl)-9-(4- | — |
| NRaRb = | | chlorophenyl)-2- | |
| nipecotamide | | (methylsulfonyl)-9H-purin-6-yl)- | |
| METHOD G | | nipecotamide | |
| A-3(203) | A-2(203) | — | — |
| NRaRb = | | | |
| morpholine derivative | | | |
| (R3 = C49) | | | |
| METHOD G | | | |
| A-3(204) | A-2(204) | — | — |
| NRaRb = | | | |
| morpholine derivative | | | |
| (R3 = C50) | | | |
| METHOD G | | | |
| A-3(206) | A-2(206) | 1-(8-(2-chlorophenyl)-9-(4- | — |
| NRaRb = | | chlorophenyl)-2- | |
| 2-methylmorpholine | | (methylsulfonyl)-9H-purin-6-yl)- | |
| METHOD G | | 2-methylmorpholine | |
| A-3(207) | A-2(207) | 1-(8-(2-chlorophenyl)-9-(4- | — |
| NRaRb = | | chlorophenyl)-2- | |
| 4-ethoxy-piperidine-4- | | (methylsulfonyl)-9H-purin-6-yl)- | |
| carboxamide | | 4-ethoxy-piperidine-4- | |
| METHOD G | | carboxamide | |
| A-3(210) | A-2(210) | 1-(8-(2-chlorophenyl)-9-(4- | — |
| NRaRb = | | chlorophenyl)-2- | |
| 2-methylmorpholine | | (methylsulfonyl)-9H-purin-6-yl)- | |
| METHOD G | | 2-methylmorpholine | |
| A-3(211) | A-2(211) | 1-(8-(2-chlorophenyl)-9-(4- | — |
| NRaRb = | | chlorophenyl)-2- | |
| 3-methylmorpholine | | (methylsulfonyl)-9H-purin-6-yl)- | |
| METHOD G | | 3-methylmorpholine | |
| A-3(212) | A-2(212) | 1-(8-(2-chlorophenyl)-9-(4- | — |
| NRaRb = | | chlorophenyl)-2- | |
| 3-methylmorpholine | | (methylsulfonyl)-9H-purin-6-yl)- | |
| METHOD G | | 3-methylmorpholine | |
| A-3(213) | A-2(213) | 1-(8-(2-chlorophenyl)-9-(4- | — |
| NRaRb = | | chlorophenyl)-2- | |
| 4-methoxy-piperidine- | | (methylsulfonyl)-9H-purin-6-yl)- | |
| 4-carboxamide | | 4-methoxy-piperidine-4- | |
| METHOD G | | carboxamide | |
| A-3(214) | A-2(214) | — | — |
| NRaRb = | | | |
| Secondary amine | | | |
| derivative (R3 = C56) | | | |
| METHOD G | | | |
| A-3(215) | A-2(215) | 1-(8-(2-chlorophenyl)-9-(4- | — |
| NRaRb = | | chlorophenyl)-2- | |
| 4-isopropoxy- | | (methylsulfonyl)-9H-purin-6-yl)- | |
| piperidine-4- | | 4-isopropoxy-piperidine-4- | |
| carboxamide | | carboxamide | |
| METHOD G | | | |
| A-3(216) | A-2(216) | 1-(8-(2-chlorophenyl)-9-(4- | — |
| NRaRb = | | chlorophenyl)-2- | |
| 3-ethoxy-piperidine-3- | | (methylsulfonyl)-9H-purin-6-yl)- | |
| carboxamide | | 3-ethoxy-piperidine-3- | |
| METHOD G | | carboxamide | |
| A-3(222) | A-2(222) | 1-(8-(2-chlorophenyl)-9-(4- | — |
| NRaRb = | | chlorophenyl)-2- | |
| 1-acetylpiperazine | | (methylsulfonyl)-9H-purin-6-yl)- | |
| METHOD G | | 1-acetylpiperazine | |
| A-3(224) | A-2(224) | 1-(8-(2-chlorophenyl)-9-(4- | — |
| NRaRb = | | chlorophenyl)-2- | |
| methyl(piperazine-1- | | (methylsulfonyl)-9H-purin-6-yl)- | |
| carboxylate) | | methyl(piperazine-1- | |
| METHOD G | | carboxylate) | |
| A-3(226) | A-2(226) | 1-(8-(2-chlorophenyl)-9-(4- | — |
| NRaRb = | | chlorophenyl)-2- | |
| 4-methyl-piperidine-4- | | (methylsulfonyl)-9H-purin-6-yl)- | |
| N-acetamide | | 4-methyl-piperidine-4-N- | |
| METHOD G | | acetamide | |
| A-3(227) | A-2(227) | 1-(8-(2-chlorophenyl)-9-(4- | — |
| NRaRb = | | chlorophenyl)-2- | |
| 1-methylpiperazine | | (methylsulfonyl)-9H-purin-6-yl)- | |
| METHOD G | | 1-methylpiperazine | |
| A-3(233) | A-2(233) | — | — |
| NRaRb = | | | |
| secondary amine | | | |
| derivative (R3 = C64) | | | |
| METHOD G | | | |
| A-3(237) | A-2(237) | 1-(8-(2-chlorophenyl)-9-(4- | — |
| NRaRb = | | chlorophenyl)-2- | |
| 3-carboxamide-azetidin | | (methylsulfonyl)-9H-purin-6-yl)- | |
| METHOD F | | 3-carboxamide-azetidin | |

### Example 4—Preparation of B-3 Intermediates

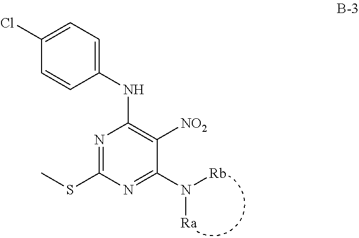

### (i) Preparation of Intermediate B-3(36) (NRaRb: 4-methyl-piperidine-4-carboxamide)

To a solution of 6-chloro-N-(4-chlorophenyl)-2-methylsulfanyl-5-nitro-pyrimidin-4-amine (Intermediate B-2, Example 2i) (1 g, 3.02 mmol, 1 eq.) and 4-methylpiperidine-4-carboxamide (539 mg, 3.02 mmol, 1 eq., hydrochloride) in N,N-dimethylformamide (5 mL) was added potassium carbonate (417.32 mg, 3.02 mmol, 1 eq.). The mixture was stirred at 25° C. for 12 hours. The reaction mixture was then poured in water (100 mL) under stirring. The mixture was filtered to give the filter residue as a yellow solid. The yellow solid was purified by flash silica gel chromatography (ISCO®; 40 g SepaFlash® Silica Flash Column, Eluent of 0˜30% Ethyl acetate/Petroleum ether gradient @100 mL/min) to give 1-[6-(4-chloroanilino)-2-methylsulfanyl-5-nitro-pyrimidin-4-yl]-4-methyl-piperidine-4-carboxamide (Intermediate B-3(36), 1 g, 2.21 mmol, 73.0% yield) as a yellow solid (R_{f}=0.5). ¹H NMR (400 MHz, DMSO-d₆) δ=10.28 (s, 1H), 7.63 (d, J=8.8 Hz, 2H), 7.41 (d, J=8.9 Hz, 2H), 7.29 (s, 1H), 7.04-6.96 (m, 1H), 3.62 (br d, J=13.6 Hz, 2H), 3.28-3.14 (m, 2H), 2.40 (s, 3H), 2.09 (br d, J=14.2 Hz, 2H), 1.61-1.29 (m, 2H).

### (ii) Preparation of Intermediate B-3(112) (NRaRb: 3-methyl-azetidin-3-ol)

To a solution of 6-chloro-N-(4-chlorophenyl)-2-methylsulfanyl-5-nitro-pyrimidin-4-amine (Intermediate B-2, Example 2i) (250 mg, 0.75 mmol, 1.0 eq.) and 3-methylazetidin-3-ol (140 mg, 1.13 mmol, 1.5 eq., hydrochloride) in N,N-dimethylformamide (4 mL) was added cesium carbonate (738 mg, 2.26 mmol, 3.0 eq.). The mixture was stirred at 25° C. for 1 hour. The reaction mixture was added to the water (20 mL), and the mixture was filtered to give a yellow solid. The residue was purified by flash silica gel chromatography (ISCO®; 20 g SepaFlash® Silica Flash Column, Eluent of 0˜40% Ethyl acetate/Petroleum ether gradient @80 mL/min) to give a spot (Rf=0.4) 1-[6-(4-chloroanilino)-2-methylsulfanyl-5-nitro-pyrimidin-4-yl]-3-methyl-azetidin-3-ol (Intermediate B-3(112), 170 mg, 0.44 mmol, 58% yield, 98.6% purity) as a yellow solid. ¹H NMR (400 MHz, DMSO-d₆) δ=10.32 (s, 1H), 7.62 (br d, J=8.7 Hz, 2H), 7.42 (br d, J=8.7 Hz, 2H), 5.75 (s, 1H), 4.09-3.88 (m, 4H), 3.30 (br s, 3H), 1.38 (s, 3H).

### (iii) Preparation of Intermediate B-3(c) (NRaRb: 3-methyl-azetidine-3-carbonitrile)

To a solution of 6-chloro-N-(4-chlorophenyl)-2-methylsulfanyl-5-nitro-pyrimidin-4-amine (Intermediate B-2, Example 2i) (1 g, 3.02 mmol, 1.0 eq.) and 3-methyl-3-azetidinecarbonitrile hydrochloride (480 mg, 3.62 mmol, 1.2 eq.) in N,N-dimethylformamide (10 mL) was added cesium carbonate (2.95 g, 9.06 mmol, 3.0 eq.). The mixture was stirred at 25° C. for 1 hour. The reaction mixture was added dropwise into water 150 mL, filtered and the filtration residue was concentrated under reduced pressure to give 1-[6-(4-chloroanilino)-2-methylsulfanyl-5-nitro-pyrimidin-4-yl]-3-methyl-azetidine-3-carbonitrile (Intermediate B-3(113), 1.17 g, 2.99 mmol, 99.1% yield) as a yellow solid. ¹H NMR (400 MHz, DMSO-d₆) δ=10.39 (s, 1H), 7.66-7.57 (m, 2H), 7.46-7.39 (m, 2H), 4.47 (d, J=10.4 Hz, 2H), 4.11 (d, J=10.4 Hz, 2H), 2.39 (s, 3H), 1.64 (s, 3H).

### (iv) Preparation of Intermediate B-3(114) (NRaRb: 3-methoxy-3-methyl-azetidin-1-yl)

A mixture of 6-chloro-N-(4-chlorophenyl)-2-methylsulfanyl-5-nitro-pyrimidin-4-amine (Intermediate B-2, 600 mg, 1.81 mmol, 1.0 eq.), 3-methoxy-3-methyl-azetidine (274 mg, 1.99 mmol, 1.1 eq.) and potassium carbonate (751 mg, 5.44 mmol, 3 eq.) in N,N-dimethylformamide (6 mL) was stirred at 25° C. for 1 hour. The reaction mixture was washed with water (20 mL) and extracted with ethyl acetate 60 mL (20 mL×3). The combined organic layers were washed with brine 40 mL (20 mL×2), dried over anhydrous sodium sulfate, filtered and concentrated under reduced pressure to give a residue. The residue was purified by flash silica gel chromatography (ISCO®; 40 g SepaFlash® Silica Flash Column, Eluent of 0˜100% Ethyl acetate/Petroleum ether gradient @60 mL/min) and the organic phase was concentrated under reduced pressure to give N-(4-chlorophenyl)-6-(3-methoxy-3-methyl-azetidin-1-yl)-2-methylsulfanyl-5-nitro-pyrimidin-4-amine (Intermediate B-3(114), 250 mg, 625 μmol, 34.5% yield) as a yellow solid.

### (v) Preparation of Intermediate B-3(118) (NRaRb: 4-methyl-piperidine-4-carboxylate)

To a solution of 6-chloro-N-(4-chlorophenyl)-2-methylsulfanyl-5-nitro-pyrimidin-4-amine (Intermediate B-2, 500 mg, 1.51 mmol, 1.0 eq.) and ethyl 4-methylpiperidine-4-carboxylate (345 mg, 1.66 mmol, 1.1 eq.) in N,N-dimethylformamide (5 mL) was added cesium carbonate (1.48 g, 4.53 mmol, 3.0 eq.). The mixture was stirred at 25° C. for 1 hour. The reaction mixture was added to water (50 mL) with stirred, the mixture was filtered, the residue was washed with ethyl alcohol (20 mL) and concentrated under reduced pressure to give ethyl 1-[6-(4-chloroanilino)-2-methylsulfanyl-5-nitro-pyrimidin-4-yl]-4-methyl-piperidine-4-carboxylate (Intermediate B-3(118), 600 mg, 1.20 mmol, 79.3% yield) as a yellow solid. ¹H NMR (400 MHz, DMSO-d₆) δ=10.28 (s, 1H), 7.63 (d, J=8.8 Hz, 2H), 7.42 (d, J=8.8 Hz, 2H), 4.21-4.07 (m, 2H), 3.67 (d, J=14.0 Hz, 2H), 3.30-3.18 (m, 2H), 2.40 (s, 3H), 2.12-2.02 (m, 2H), 1.59-1.49 (m, 2H), 1.25-1.16 (m, 6H).

### (vi) Preparation of Intermediate B-3(120) (NRaRb: 4-ethoxy-piperidine-4-carboxamide)

A mixture of 6-chloro-N-(4-chlorophenyl)-2-methylsulfanyl-5-nitro-pyrimidin-4-amine (Intermediate B-2, 550 mg, 1.66 mmol, 1.0 eq.), 4-ethoxypiperidine-4-carboxamide (416 mg, 1.99 mmol, 1.2 eq.) and cesium carbonate (1.62 g, 4.98 mmol, 3.0 eq.) in N,N-dimethylformamide (5.5 mL) was stirred at 25° C. for 1 hour. The reaction mixture was washed with water (20 mL) and extracted with ethyl acetate 60 mL (20 mL×3). The combined organic layers were washed with brine 40 mL (20 mL×2), dried over anhydrous sodium sulfate, filtered and concentrated under reduced pressure to give 1-[6-(4-chloroanilino)-2-methylsulfanyl-5-nitro-pyrimidin-4-yl]-4-ethoxy-piperidine-4-carboxamide (Intermediate B-3(120), 590 mg, 1.12 mmol, 67.6% yield) as a yellow solid. ¹H NMR (400 MHz, DMSO-d₆) δ=10.29 (s, 1H), 7.68-7.58 (m, 2H), 7.42 (d, J=8.8 Hz, 2H), 7.35-7.19 (m, 2H), 3.77-3.62 (m, 2H), 3.45-3.41 (m, 2H), 3.35 (d, J=7.2 Hz, 2H), 2.41 (s, 3H), 1.98-1.90 (m, 2H), 1.19 (s, 2H), 1.05 (t, J=7.2 Hz, 3H).

### (vii) Preparation of Intermediate B-3(135) (NRaRb: 4-isopropoxy-piperidine-4-carboxamide)

A mixture of 6-chloro-N-(4-chlorophenyl)-2-methylsulfanyl-5-nitro-pyrimidin-4-amine (Intermediate B-2, 1.00 g, 3.02 mmol, 1.0 eq.), 4-isopropoxypiperidine-4-carboxamide (740 mg, 3.32 mmol, 1.1 eq. hydrochloride) and cesium carbonate (2.95 g, 9.06 mmol, 3.0 eq.) in N,N-dimethylformamide (12 mL) was stirred at 25° C. for 4 hours. The reaction mixture diluted with water (200 mL) extracted with dichloromethane (50 mL*3). The combined organic layer was washed with brine (200 mL), dried over anhydrous sodium sulfate, filtered and concentrated under reduced pressure and purified by flash silica gel chromatography (ISCO®; 40 g SepaFlash® Silica Flash Column, Eluent of 0˜50% Ethyl acetate/Petroleum ether gradient @50 mL/min) to give 1-[6-(4-chloroanilino)-2-methylsulfanyl-5-nitro-pyrimidin-4-yl]-4-isopropoxy-piperidine-4-carboxamide (Intermediate B-3(135), 1.08 g, 2.20 mmol, 73% yield) as a yellow solid. ¹H NMR (400 MHz, DMSO-d₆) δ=10.30 (s, 1H), 7.67-7.60 (m, 2H), 7.46-7.39 (m, 2H), 7.36-7.22 (m, 2H), 3.78-3.69 (m, 1H), 3.67-3.57 (m, 2H), 3.45-3.35 (m, 2H), 2.41 (s, 3H), 1.99-1.85 (m, 4H), 1.14 (d, J=6.0 Hz, 6H).

### Example 5—Preparation of B-5 Intermediates

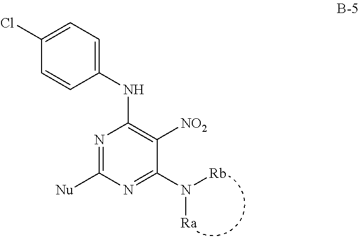

### (i) Preparation of Intermediate B-5(36) (NRaRb: 4-methyl-piperidine-4-carboxamide/Nu: (2S)-2-(hydroxymethyl)pyrrolidin-1-yl)

Step 1: To a solution of 1-[6-(4-chloroanilino)-2-methylsulfanyl-5-nitro-pyrimidin-4-yl]-4-methyl-piperidine-4-carboxamide (Intermediate B-3(36), Example 3i) (950 mg, 2.17 mmol, 1 eq.) in N-methylpyrrolidone (10 mL) was added potassium oxidooxy hydrogen sulfate (3.66 g, 21.74 mmol, 10 eq.) and water (1 mL). The mixture was stirred at 60° C. for 1 hour. The reaction mixture was added to the water (100 mL) with stirred. The mixture was filtered to give the filter residue for 1-[6-(4-chloroanilino)-2-methylsulfonyl-5-nitro-pyrimidin-4-yl]-4-methyl-piperidine-4-carboxamide (Intermediate B-4(36), 680 mg, 938 μmol, 43.1% yield) as a yellow solid. ¹H NMR (400 MHz, DMSO-d6) δ=10.36 (s, 1H), 7.66-7.56 (m, 2H), 7.45 (d, J=8.9 Hz, 2H), 7.32 (s, 1H), 7.04 (s, 1H), 3.79-3.60 (m, 2H), 3.29-3.28 (m, 2H), 3.24-3.18 (m, 3H), 2.19-1.99 (m, 2H), 1.48 (ddd, J=3.4, 10.4, 13.7 Hz, 2H), 1.20-1.10 (m, 3H).

Step 2: To a solution of 1-[6-(4-chloroanilino)-2-methylsulfonyl-5-nitro-pyrimidin-4-yl]-4-methyl-piperidine-4-carboxamide (Intermediate B-4(36)) (680 mg, 1.45 mmol, 1 eq.) in [(2S)-pyrrolidin-2-yl]methanol (2.20 g, 21.7 mmol, 2.12 mL, 15 eq.) was stirred at 60° C. for 12 hours. The reaction mixture was concentrated under reduced pressure. The residue was purified by prep-HPLC (basic condition) to give the compound 1-[6-(4-chloroanilino)-2-[(2S)-2-(hydroxymethyl)pyrrolidin-1-yl]-5-nitro-pyrimidin-4-yl]-4-methyl-piperidine-4-carboxamide (Intermediate B-5(36), 500 mg, 1.02 mmol, 70.3% yield) as a yellow solid. ¹H NMR (400 MHz, CDCl₃-d) δ=10.92-10.56 (m, 1H), 7.64 (d, J=8.8 Hz, 1H), 7.47 (br d, J=8.8 Hz, 1H), 7.39-7.29 (m, 2H), 5.68 (br d, J=5.1 Hz, 1H), 5.41 (br s, 1H), 4.56-4.14 (m, 1H), 3.79-3.35 (m, 8H), 2.25-1.67 (m, 8H), 1.40-1.23 (m, 3H).

### (ii) Preparation of Intermediate B-5(48) (NRaRb: 4-methyl-piperidine-4-carboxamide/Nu: 2-hydroxyethyl(methyl)amino)

Step 1: Synthesis of 1-[6-(4-chloroanilino)-2-methylsulfonyl-5-nitro-pyrimidin-4-yl]-4-methyl-piperidine-4-carboxamide from 1-[6-(4-chloroanilino)-2-methylsulfanyl-5-nitro-pyrimidin-4-yl]-4-methyl-piperidine-4-carboxamide (Intermediate B-3(36), Example 3i) as described in Step 1 of Example 4i.

Step 2: The mixture of 1-[6-(4-chloroanilino)-2-methylsulfonyl-5-nitro-pyrimidin-4-yl]-4-methyl-piperidine-4-carboxamide (1 g, 2.13 mmol, 1 eq.) in 2-(methylamino)ethanol (2 g, 32.0 mmol, 2.57 mL, 15 eq.), the resulting mixture was stirred at 60° C. for 1 hour. The reaction mixture was concentrated under reduced pressure. The residue was purified by column chromatography (silicon dioxide, dichloromethane/methanol=1/0 to 0/1) and concentrated under vacuum to give 1-[6-(4-chloroanilino)-2-[2-hydroxyethyl(methyl)amino]-5-nitro-pyrimidin-4-yl]-4-methyl-piperidine-4-carboxamide (580 mg, 1.25 mmol, 58.6% yield) as yellow solid. The yellow solid was dissolved with methanol (1 mL) and dimethylsulfoxide (2 mL) to purity. The residue was purified by prep-HPLC (column: Waters Xbridge 150*25 mm*5 μm; mobile phase: [water (ammonia hydroxide v/v)-ACN]; B %: 28%-58%, 9 min) to give 1-[6-(4-chloroanilino)-2-[2-hydroxyethyl(methyl)amino]-5-nitro-pyrimidin-4-yl]-4-methyl-piperidine-4-carboxamide (Intermediate B-5(48), 105 mg, 182.07 μmol, 28.1% yield) as a yellow solid. ¹H NMR (400 MHz, CDCl₃-d) δ=11.00-10.52 (m, 1H), 7.61 (br d, J=8.6 Hz, 1H), 7.49 (br d, J=8.3 Hz, 1H), 7.33(br d, J=8.6 Hz, 2H), 5.80-5.10 (m, 2H), 3.97-3.56 (m, 6H), 3.51-3.35 (m, 2H), 3.22 (br s, 3H), 2.23-2.07 (m, 2H), 1.71-1.61 (m, 2H).

### (iii) Preparation of Intermediate B-5(103) (NRaRb: 4-methyl-piperidine-4-carboxamide/Nu: 2-hydroxy-2-methyl-propoxy)

Step 1: Synthesis of 1-[6-(4-chloroanilino)-2-methylsulfonyl-5-nitro-pyrimidin-4-yl]-4-methyl-piperidine-4-carboxamide from 1-[6-(4-chloroanilino)-2-methylsulfanyl-5-nitro-pyrimidin-4-yl]-4-methyl-piperidine-4-carboxamide (Intermediate B-3(36), Example 3i) as described in Step 1 of Example 4i.

Step 2: To a solution of 2-methylpropane-1,2-diol (288 mg, 3.20 mmol, 5.0 eq.) in tetrahydrofuran (3 mL) was added sodium hydride (77 mg, 1.92 mmol, 60% purity, 3.0 eq.). The mixture was stirred at 25° C. for 10 minutes under nitrogen. Then the 1-[6-(4-chloroanilino)-2-methylsulfonyl-5-nitro-pyrimidin-4-yl]-4-methyl-piperidine-4-carboxamide (300 mg, 640 μmol, 1.0 eq.) was added to the mixture. The mixture was stirred at 25° C. for 50 minutes under nitrogen. The mixture was diluted with water (10 mL), extracted with ethyl acetate (10 mL*3). The organic layer was dried with anhydrous sodium sulfate filtered and concentrated under vacuum to give a residue. The crude product was wash with dichloromethane (5 mL), the mixture was filtered to give the filter residue for 1-[6-(4-chloroanilino)-2-(2-hydroxy-2-methyl-propoxy)-5-nitro-pyrimidin-4-yl]-4-methyl-piperidine-4-carboxamide (Intermediate B-5(103), 130 mg, 0.26 mmol, 40.7% yield) as a yellow solid.

### (iv) Preparation of Intermediate B-5(110) (NRaRb: 4-methyl-piperidine-4-carboxamide/Nu: (2-hydroxy-2-methyl-propyl)-methyl-amino)

Step 1: Synthesis of 1-[6-(4-chloroanilino)-2-methylsulfonyl-5-nitro-pyrimidin-4-yl]-4-methyl-piperidine-4-carboxamide from 1-[6-(4-chloroanilino)-2-methylsulfanyl-5-nitro-pyrimidin-4-yl]-4-methyl-piperidine-4-carboxamide (Intermediate B-3(36), Example 3i) as described in Step 1 of Example 4i.

Step 2: To a solution of 1-[6-(4-chloroanilino)-2-methylsulfonyl-5-nitro-pyrimidin-4-yl]-4-methyl-piperidine-4-carboxamide (200 mg, 0.43 mmol, 1.0 eq.) in 2-methyl-1-(methylamino)propan-2-ol (660 mg, 6.40 mmol, 15 eq.). The mixture was stirred at 120° C. for 1 hour. The reaction mixture was added methanol (1 mL) to purity. The residue was purified by prep-HPLC (column: Phenomenex luna C18 150*25 mm*10 μm; mobile phase: [water(formic acid)-acetonitrile]; B %: 44%-74%, 10 min) to give 1-[6-(4-chloroanilino)-2-[(2-hydroxy-2-methyl-propyl)-methyl-amino]-5-nitro-pyrimidin-4-yl]-4-methyl-piperidine-4-carboxamide (Intermediate B-5(110), 100 mg, 0.20 mmol, 47.0% yield) as a yellow solid. ¹H NMR (400 MHz, DMSO-d₆) δ=7.77 (d, J=8.8 Hz, 1H), 7.66 (d, J=8.8 Hz, 1H), 7.40 (t, J=8.8 Hz, 2H), 7.26 (d, J=2.0 Hz, 1H), 6.98 (br s, 1H), 4.63-4.35 (m, 1H), 4.19-3.98 (m, 3H), 3.67-3.49 (m, 4H), 2.13-1.94 (m, 2H), 1.52-1.29 (m, 2H), 1.19-1.05 (m, 6H), 0.99 (s, 3H).

### (v) Preparation of Intermediate B-5(111) (NRaRb: 4-methyl-piperidine-4-carboxamide/Nu:(2-hydroxy-2-methyl-propyl)amino)

Step 1: Synthesis of 1-[6-(4-chloroanilino)-2-methylsulfonyl-5-nitro-pyrimidin-4-yl]-4-methyl-piperidine-4-carboxamide from 1-[6-(4-chloroanilino)-2-methylsulfanyl-5-nitro-pyrimidin-4-yl]-4-methyl-piperidine-4-carboxamide (Intermediate B-3(36), Example 3i) as described in Step 1 of Example 4i.

Step 2: A mixture of 1-[6-(4-chloroanilino)-2-methylsulfonyl-5-nitro-pyrimidin-4-yl]-4-methyl-piperidine-4-carboxamide (Cpd.1, 400 mg, 0.85 mmol, 1.0 eq.) and 1-amino-2-methyl-propan-2-ol (Cpd.2, 2.28 g, 25.6 mmol, 30 eq.) was stirred at 60° C. for 3 hrs. The reaction mixture was cooled at room temperature and diluted with water (30 mL), extracted with ethyl acetate (10 mL×3). The combined organic layers were washed with brine (30 mL), dried over anhydrous sodium sulfate, filtered and concentrated under reduced pressure, the resulting residue was purified by flash silica gel chromatography (ISCO®; 20 g SepaFlash® Silica Flash Column, Eluent of 0˜100% Ethyl acetate/Petroleum ethergradient @20 mL/min). The cut fraction was concentrated under reduced pressure to give compound 1-[6-(4-chloroanilino)-2-[(2-hydroxy-2-methyl-propyl)amino]-5-nitro-pyrimidin-4-yl]-4-methyl-piperidine-4-carboxamide (Intermediate B-5(111), 230 mg, 0.48 mmol, 56.4% yield) as a yellow solid.

### (vi) Preparation of Intermediate B-5(112) (NRaRb: 3-methyl-azetidin-3-ol/Nu: 2-hydroxy-2-methyl-propoxy)

Step 1: To a solution of 1-[6-(4-chloroanilino)-2-methylsulfanyl-5-nitro-pyrimidin-4-yl]-3-methyl-azetidin-3-ol (Intermediate B-3(112), Example 3ii) (170 mg, 0.45 mmol, 1.0 eq.) in N-methylpyrrolidone (5 mL) and water (0.5 mL) was added potassium; oxidooxy hydrogen sulfate (749 mg, 4.45 mmol, 10 eq.). The mixture was stirred at 60° C. for 12 hours. The reaction mixture was added to the water (50 mL) with stirred, and the mixture was filtered to give 1-[6-(4-chloroanilino)-2-methylsulfonyl-5-nitro-pyrimidin-4-yl]-3-methyl-azetidin-3-ol (150 mg, 344.41 μmol, 77.3% yield, 95.0% purity) as a yellow solid. ¹H NMR (400 MHz, DMSO-d₆) δ=10.35 (s, 1H), 7.62-7.57 (m, 2H), 7.47-7.43 (m, 2H), 5.88-5.82 (m, 1H), 5.78-5.71 (m, 2H), 4.04 (br s, 2H), 3.20 (s, 3H), 1.40 (s, 3H).

Step 2: To a solution of 2-methylpropane-1,2-diol (109 mg, 1.21 mmol, 5 eq) in tetrahydrofuran (3 mL) was added sodium hydride (39 mg, 0.97 mmol, 60% purity, 4.0 eq.). The mixture was stirred at 25° C. for 10 min. Then was added 1-[6-(4-chloroanilino)-2-methylsulfonyl-5-nitro-pyrimidin-4-yl]-3-methyl-azetidin-3-ol (100 mg, 0.24 mmol, 1.0 eq.). The mixture was stirred at 25° C. for 50 minutes. The mixture was diluted with water (20 mL) and extracted with ethyl acetate (20 mL*3). The organic layer was dried with anhydrous sodium sulfate filtered and concentrated under vacuum to give a residue. The residue was purified by prep-HPLC (column: Phenomenex luna C18 150*25 mm*10 μm; mobile phase: [water(formic acid)-ACN]; B %: 36%-66%, 10 min) to give the compound 1-[6-(4-chloroanilino)-2-(2-hydroxy-2-methyl-propoxy)-5-nitro-pyrimidin-4-yl]-3-methyl-azetidin-3-ol (Intermediate B-5(112), 100 mg, 0.23 mmol, 96.8% yield) as a yellow solid. ¹H NMR (400 MHz, DMSO-d₆) δ=10.41 (s, 1H), 7.72-7.59 (m, 2H), 7.42 (d, J=8.8 Hz, 2H), 5.74 (s, 1H), 4.64 (s, 1H), 3.99 (s, 4H), 1.38 (s, 3H), 1.13 (s, 6H).

### (vii) Preparation of Intermediate B-5(113) (NRaRb: 3-methyl-azetidine-3-carbonitrile/Nu: 2-hydroxy-2-methyl-propoxy)

Step 1: To a solution of 1-[6-(4-chloroanilino)-2-methylsulfanyl-5-nitro-pyrimidin-4-yl]-3-methyl-azetidine-3-carbonitrile (Intermediate B-3(113), Example 3iii) (1.1 g, 2.81 mmol, 1.0 eq.) in N-methyl-2-pyrrolidone (20 mL) and water (2 mL) was added potassium peroxymonosulfate (4.73 g, 28.14 mmol, 10 eq.). The mixture was stirred at 60° C. for 1 hour. The reaction mixture was added dropwise into water 80 mL, filtered and the filtration residue was concentrated under reduced pressure to give 1-[6-(4-chloroanilino)-2-methylsulfonyl-5-nitro-pyrimidin-4-yl]-3-methyl-azetidine-3-carbonitrile (1.1 g, 2.45 mmol, 87.0% yield) as a yellow solid. ¹H NMR (400 MHz, DMSO-d₆) δ=10.42 (s, 1H), 7.63-7.56 (m, 2H), 7.49-7.43 (m, 2H), 4.53 (d, J=10.4 Hz, 2H), 4.16 (d, J=10.4 Hz, 2H), 3.24-3.19 (m, 3H), 1.66 (s, 3H).

Step 2: To a solution of 1-[6-(4-chloroanilino)-2-methylsulfonyl-5-nitro-pyrimidin-4-yl]-3-methyl-azetidine-3-carbonitrile (1 g, 2.36 mmol, 1.0 eq.) in N-methyl-2-pyrrolidone (10 mL) was added 2-methylpropane-1,2-diol (1.07 g, 11.82 mmol, 5.0 eq.) and potassium tert-butoxide (796 mg, 7.09 mmol, 3.0 eq.). The mixture was stirred at 80° C. for 1 hour. The reaction mixture was washed with ammonium chloride saturated solution (30 mL) and extracted with ethyl acetate 90 mL (30 mL×3). The combined organic layers were washed with brine 60 mL (30 mL×2), dried over anhydrous sodium sulfate, filtered and concentrated under reduced pressure to give a residue. The residue was purified by flash silica gel chromatography (ISCO®; 40 g SepaFlash® Silica Flash Column, Eluent of 0˜100% Ethyl acetate/Petroleum ether gradient to 0˜100% Methanol/Ethyl acetate @30 mL/min) and the organic phase was concentrated under reduced pressure to give 1-[6-(4-chloroanilino)-2-(2-hydroxy-2-methyl-propoxy)-5-nitro-pyrimidin-4-yl]-3-methyl-azetidine-3-carbonitrile (Intermediate B-5(113), 230 mg, 0.50 mmol, 21% yield) as a yellow solid.

### (viii) Preparation of Intermediate B-5(114) (NRaRb: 3-methoxy-3-methyl-azetidin-1-yl/Nu: oxy-2-methyl-propan-2-ol)

Step 1: To a solution of N-(4-chlorophenyl)-6-(3-methoxy-3-methyl-azetidin-1-yl)-2-methylsulfanyl-5-nitro-pyrimidin-4-amine (Intermediate B-3(114), Example 3iv) (230 mg, 0.58 mmol, 1.0 eq.) in N-methyl-2-pyrrolidone (3 mL) and water (0.3 mL) was added potassium peroxymonosulfate (977 mg, 5.81 mmol, 10 eq.). The mixture was stirred at 60° C. for 1 hour. The reaction mixture was added dropwise into water 20 mL, filtered and the filtration residue was concentrated under reduced pressure to give N-(4-chlorophenyl)-6-(3-methoxy-3-methyl-azetidin-1-yl)-2-methylsulfonyl-5-nitro-pyrimidin-4-amine (210 mg, 0.49 mmol, 84.4% yield) as a white solid.

Step 2: A mixture of N-(4-chlorophenyl)-6-(3-methoxy-3-methyl-azetidin-1-yl)-2-methylsulfonyl-5-nitro-pyrimidin-4-amine (200 mg, 0.47 mmol, 1.0 eq.) in N-methyl-2-pyrrolidone (3 mL) was added 2-methylpropane-1,2-diol (211 mg, 2.34 mmol, 5.0 eq.) and potassium tert-butoxide (157 mg, 1.40 mmol, 3.0 eq.). The mixture was stirred at 80° C. for 1 hour. The reaction mixture was washed with ammonium chloride saturated solution (10 mL) and extracted with ethyl acetate 30 mL (10 mL×3). The combined organic layers were washed with brine 20 mL (10 mL×2), dried over anhydrous sodium sulfate, filtered and concentrated under reduced pressure to give a residue. The residue was purified by flash silica gel chromatography (ISCO®; 12 g SepaFlash® Silica Flash Column, Eluent of 0˜100% Ethyl acetate/Petroleum ether gradient @20 mL/min) and the organic phase was concentrated under reduced pressure to give 1-[4-(4-chloroanilino)-6-(3-methoxy-3-methyl-azetidin-1-yl)-5-nitro-pyrimidin-2-yl]oxy-2-methyl-propan-2-ol (Intermediate B-5(113), 50 mg, 0.11 mmol, 24.0% yield) as a yellow solid.

### (ix) Preparation of Intermediate B-5(118) (NRaRb: 4-methyl-piperidine-4-carboxylic acid/Nu: 2-hydroxy-2-methyl-propoxy)

Step 1: To a solution of ethyl 1-[6-(4-chloroanilino)-2-methylsulfanyl-5-nitro-pyrimidin-4-yl]-4-methyl-piperidine-4-carboxylate (Intermediate B-3(118), Example 3v) (600 mg, 1.29 mmol, 1.0 eq.) in N-methyl-2-pyrrolidone (10 mL) and water (1 mL) was added potassium peroxymonosulfate (2.17 g, 12.88 mmol, 10 eq.). The mixture was stirred at 60° C. for 1 hour. The reaction mixture was added dropwise into water 50 mL, filtered and the filtration residue was concentrated under reduced pressure to give ethyl 1-[6-(4-chloroanilino)-2-methylsulfonyl-5-nitro-pyrimidin-4-yl]-4-methyl-piperidine-4-carboxylate (590 mg, 1.14 mmol, 88.2% yield) as a yellow solid. ¹H NMR (400 MHz, DMSO-d₆) δ=10.37 (s, 1H), 7.64-7.58 (m, 2H), 7.45 (d, J=8.8 Hz, 2H), 4.26-4.07 (m, 2H), 3.71 (d, J=14.0 Hz, 2H), 3.30 (d, J=2.8 Hz, 2H), 3.22 (s, 3H), 2.10 (d, J=14.0 Hz, 2H), 1.64-1.53 (m, 2H), 1.25-1.19 (m, 6H).

Step 2: To a solution of ethyl 1-[6-(4-chloroanilino)-2-methylsulfonyl-5-nitro-pyrimidin-4-yl]-4-methyl-piperidine-4-carboxylate (490 mg, 0.98 mmol, 1.0 eq.) in N-methyl-2-pyrrolidone (5 mL) was added 2-methylpropane-1,2-diol (443 mg, 4.92 mmol, 5 eq.) and potassium tert-butoxide (331 mg, 2.95 mmol, 3.0 eq.). The mixture was stirred at 80° C. for 1 hour. The reaction mixture was added to aqueous citric acid solution (20 mL) (pH=3) and extracted with ethyl acetate 60 mL (20 mL×3). The combined organic layers were washed with brine 20 mL (10 mL×2), dried over anhydrous sodium sulfate, filtered and concentrated under reduced pressure to give a residue. The residue was purified by flash silica gel chromatography (ISCO®; 20 g SepaFlash® Silica Flash Column, Eluent of 0˜100% Petroleum ether gradient/Ethyl acetate@30 mL/min) and the organic phase was concentrated under reduced pressure to give 1-[6-(4-chloroanilino)-2-(2-hydroxy-2-methyl-propoxy)-5-nitro-pyrimidin-4-yl]-4-methyl-piperidine-4-carboxylic acid (Intermediate B-5(118), 191 mg, 0.40 mmol, 40.4% yield) as a yellow solid. ¹H NMR (400 MHz, DMSO-d₆) δ=10.36 (s, 1H), 7.70-7.63 (m, 2H), 7.46-7.39 (m, 2H), 4.00 (s, 2H), 3.71-3.59 (m, 2H), 3.26-3.17 (m, 2H), 2.10-2.00 (m, 2H), 1.56-1.44 (m, 2H), 1.19 (s, 3H), 1.14 (s, 6H).

### (x) Preparation of Intermediate B-5(120) (NRaRb: 4-ethoxy-piperidine-4-carboxamide/Nu: 2-hydroxy-2-methyl-propoxy)

Step 1: To a solution of 1-[6-(4-chloroanilino)-2-methylsulfanyl-5-nitro-pyrimidin-4-yl]-4-ethoxy-piperidine-4-carboxamide (Intermediate B-3(120), Example 3vi) (590 mg, 1.26 mmol, 1.0 eq.) in N-methyl-2-pyrrolidone (6 mL) and water (0.6 mL) was added potassium peroxymonosulfate (2.12 g, 12.64 mmol, 10 eq.). The mixture was stirred at 60° C. for 1 hour. The reaction mixture was added dropwise into water 80 mL, filtered and the filtration residue was concentrated under reduced pressure to give 1-[6-(4-chloroanilino)-2-methylsulfonyl-5-nitro-pyrimidin-4-yl]-4-ethoxy-piperidine-4-carboxamide (300 mg, 0.52 mmol, 40.9% yield) as a light-yellow solid. ¹H NMR (400 MHz, DMSO-d₆) δ=10.37 (s, 1H), 7.66-7.57 (m, 2H), 7.45 (d, J=8.8 Hz, 2H), 7.38-7.23 (m, 2H), 3.79-3.65 (m, 2H), 3.36 (d, J=6.4 Hz, 4H), 3.22 (s, 3H), 2.02-1.89 (m, 4H), 1.23-1.17 (m, 3H). LCMS: (ES+) m/z=499.1 (M+H).

Step 2: To a solution of 1-[6-(4-chloroanilino)-2-methylsulfonyl-5-nitro-pyrimidin-4-yl]-4-ethoxy-piperidine-4-carboxamide (250 mg, 0.50 mmol, 1.0 eq.) in N-methyl-2-pyrrolidone (5 mL) was added 2-methylpropane-1,2-diol (226 mg, 2.51 mmol, 5 eq.) and potassium tert-butoxide (169 mg, 1.50 mmol, 3 eq.). The mixture was stirred at 80° C. for 1 hour. The reaction mixture was washed with water (20 mL) and extracted with ethyl acetate 60 mL (20 mL×3). The combined organic layers were washed with brine 60 mL (30 mL×2), dried over anhydrous sodium sulfate, filtered and concentrated under reduced pressure to give a residue. The residue was purified by flash silica gel chromatography (ISCO®; 20 g SepaFlash® Silica Flash Column, Eluent of 0˜100% Ethyl acetate/Methanol @30 mL/min) and the organic phase was concentrated under reduced pressure to give 1-[6-(4-chloroanilino)-2-(2-hydroxy-2-methyl-propoxy)-5-nitro-pyrimidin-4-yl]-4-ethoxy-piperidine-4-carboxamide (Intermediate B-5(120), 200 mg, 0.33 mmol, 66.3% yield) as a yellow solid.

### (xi) Preparation of Intermediate B-5(131) (NRaRb: 4-methyl-piperidine-4-carboxamide/Nu: (3R)-3-hydroxypyrrolidin-1-yl)

Step 1: Synthesis of 1-[6-(4-chloroanilino)-2-methylsulfonyl-5-nitro-pyrimidin-4-yl]-4-methyl-piperidine-4-carboxamide from 1-[6-(4-chloroanilino)-2-methylsulfanyl-5-nitro-pyrimidin-4-yl]-4-methyl-piperidine-4-carboxamide (Intermediate B-3(36), Example 3i) as described in Step 1 of Example 4i.

Step 2: A mixture of 1-[6-(4-chloroanilino)-2-methylsulfonyl-5-nitro-pyrimidin-4-yl]-4-methyl-piperidine-4-carboxamide (1.00 g, 2.13 mmol, 1.0 eq.) and (3R)-pyrrolidin-3-ol (1.05 g, 12.05 mmol, 1 mL, 5.7 eq.) was stirred at 100° C. for 1 hour. The reaction mixture was cooled at room temperature and concentrated under reduced pressure to give a residue. The residue was purified by triturated methanol (8 mL) at 25° C. Then filter cake was collected and concentrated under reduced pressure to give compound 1-[6-(4-chloroanilino)-2-[(3R)-3-hydroxypyrrolidin-1-yl]-5-nitro-pyrimidin-4-yl]-4-methyl-piperidine-4-carboxamide (Intermediate B-5(131), 850 mg, 1.79 mmol, 83.7% yield) as a yellow solid. ¹H NMR (400 MHz, DMSO-d₆) δ=10.63 (d, J=5.6 Hz, 1H), 7.86-7.76 (m, 2H), 7.41 (d, J=8.9 Hz, 2H), 7.30-6.94 (m, 2H), 4.99 (br s, 1H), 4.35 (br s, 1H), 3.67-3.45 (m, 6H), 3.26-3.14 (m, 2H), 2.10-1.83 (m, 4H), 1.48-1.36 (m, 2H), 1.14 (s, 3H).

### (xii) Preparation of Intermediate B-5(132) (NRaRb: 4-ethoxy-piperidine-4-carboxamide/Nu: (2-hydroxy-2-methyl-propyl)amino)

Step 1: Synthesis of 1-[6-(4-chloroanilino)-2-methylsulfonyl-5-nitro-pyrimidin-4-yl]-4-ethoxy-piperidine-4-carboxamide from 1-[6-(4-chloroanilino)-2-methylsulfanyl-5-nitro-pyrimidin-4-yl]-4-ethoxy-piperidine-4-carboxamide (Intermediate B-3(120), Example 3vi) as described in Step 1 of Example 4x.

Step 2: To a solution of 1-[6-(4-chloroanilino)-2-methylsulfonyl-5-nitro-pyrimidin-4-yl]-4-ethoxy-piperidine-4-carboxamide (180 mg, 0.36 mmol, 1.0 eq.) in 1-amino-2-methyl-propan-2-ol (322 mg, 3.61 mmol, 10 eq.) was stirred at 120° C. for 1 hour. The reaction mixture was added with water (5 mL), filtered and the filtration residue was concentrated under reduced pressure to give the residue. The residue was purified by flash silica gel chromatography (ISCO®; 12 g SepaFlash® Silica Flash Column, Eluent of 0˜100% Dichloromethane/Methanol @20 mL/min) and the organic phase was concentrated under reduced pressure to give 1-[6-(4-chloroanilino)-2-[(2-hydroxy-2-methyl-propyl)amino]-5-nitro-pyrimidin-4-yl]-4-ethoxy-piperidine-4-carboxamide (Intermediate B-5(132), 110 mg, 0.21 mmol, 58.7% yield) as a yellow solid.

### (xiii) Preparation of Intermediate B-5(133) (NRaRb: 4-ethoxy-piperidine-4-carboxamide/Nu: (2S)-2-(hydroxymethyl)pyrrolidin-1-yl)

Step 1: Synthesis of 1-[6-(4-chloroanilino)-2-methylsulfonyl-5-nitro-pyrimidin-4-yl]-4-ethoxy-piperidine-4-carboxamide from 1-[6-(4-chloroanilino)-2-methylsulfanyl-5-nitro-pyrimidin-4-yl]-4-ethoxy-piperidine-4-carboxamide (Intermediate B-3(120)) as described in Step 1 of Example 4x.

Step 2: To a solution of 1-[6-(4-chloroanilino)-2-methylsulfonyl-5-nitro-pyrimidin-4-yl]-4-ethoxy-piperidine-4-carboxamide (180 mg, 0.36 mmol, 1 eq.) in [(2S)-pyrrolidin-2-yl]methanol (365 mg, 3.61 mmol, 0.35 mL, 10 eq.) was stirred at 120° C. for 1 hour. The residue was purified by flash silica gel chromatography (ISCO®; 12 g SepaFlash® Silica Flash Column, Eluent of 0˜100% Dichloromethane/Methanol @20 mL/min) and the organic phase was concentrated under reduced pressure to give 1-[6-(4-chloroanilino)-2-[(2S)-2-(hydroxymethyl)pyrrolidin-1-yl]-5-nitro-pyrimidin-4-yl]-4-ethoxy-piperidine-4-carboxamide (Intermediate B-5(133), 180 mg, 0.34 mmol, 94.5% yield) as a yellow solid.

### (xiv) Preparation of Intermediate B-5(135) (NRaRb: 4-isopropoxy-piperidine-4-carboxamide/Nu: 2-hydroxy-2-methyl-propoxy)

Step 1: A mixture of 1-[6-(4-chloroanilino)-2-methylsulfanyl-5-nitro-pyrimidin-4-yl]-4-isopropoxy-piperidine-4-carboxamide (Intermediate B-3(135)) (1.00 g, 2.08 mmol, 1 eq.) and potassium oxidooxy hydrogen sulfate (3.50 g, 20.79 mmol, 10 eq.) in N-methylpyrrolidone (16 mL) and water (4 mL) was stirred at 60° C. for 3 hours. The reaction mixture was cooled at room temperature and diluted with water (200 mL), extracted with ethyl acetate (50 mL*3). The combined organic layer were washed with brine (200 mL), dried over anhydrous sodium sulfate, filtered and concentrated under reduced pressure to give a residue. The residue was purified by flash silica gel chromatography (ISCO®; 40 g SepaFlash® Silica Flash Column, Eluent of 50˜100% Ethyl acetate/Petroleum ether gradient @40 mL/min). The cut fraction was concentrated under reduced pressure to give 1-[6-(4-chloroanilino)-2-methylsulfonyl-5-nitro-pyrimidin-4-yl]-4-isopropoxy-piperidine-4-carboxamide (700 mg, 1.36 mmol, 66% yield) as a yellow solid. ¹H NMR (400 MHz, DMSO-d₆) δ=10.38 (s, 1H), 7.66-7.59 (m, 2H), 7.49-7.42 (m, 2H), 7.39-7.24 (m, 2H), 3.79-3.62 (m, 3H), 3.51-3.40 (m, 2H), 3.23 (s, 3H), 1.99-1.88 (m, 4H), 1.16-1.15 (m, 6H).

Step 2: A mixture of 1-[6-(4-chloroanilino)-2-methylsulfonyl-5-nitro-pyrimidin-4-yl]-4-isopropoxy-piperidine-4-carboxamide (350 mg, 0.69 mmol, 1.0 eq.) and the nucleophile (10.3 mmol, 1 mL, 15.1 eq.) was heated at 120° C. for 1 hour. The mixture was triturated with ethyl acetate (10 mL), filtered to give Intermediate B-5(135) (200 mg, 374.5 μmol, 55% yield) as a yellow solid. ¹H NMR (400 MHz, DMSO-d₆) δ=10.71-10.59 (m, 1H), 7.85-7.74 (m, 2H), 7.45-7.36 (m, 2H), 7.34-7.29 (m, 1H), 7.27-7.12 (m, 1H), 4.82-4.70 (m, 1H), 4.23-4.03 (m, 1H), 3.86-3.66 (m, 1H), 3.66-3.48 (m, 5H), 3.47-3.35 (m, 2H), 2.09-1.80 (m, 8H), 1.14 (d, J=6.0 Hz, 6H).

### (xv) Preparation of Intermediate B-5(136) (NRaRb: 4-isopropoxy-piperidine-4-carboxamide/Nu: (2S)-2-(hydroxymethyl)pyrrolidin-1-yl)

Step 1: Synthesis of 1-[6-(4-chloroanilino)-2-methylsulfonyl-5-nitro-pyrimidin-4-yl]-4-isopropoxy-piperidine-4-carboxamide from 1-[6-(4-chloroanilino)-2-methylsulfanyl-5-nitro-pyrimidin-4-yl]-4-isopropoxy-piperidine-4-carboxamide (Intermediate B-3(135)) as described in Step 1 of Example 4xiv.

Step 2: A mixture of 1-[6-(4-chloroanilino)-2-methylsulfonyl-5-nitro-pyrimidin-4-yl]-4-isopropoxy-piperidine-4-carboxamide (350 mg, 0.68 mmol, 1 eq.) and [(2S)-pyrrolidin-2-yl]methanol (10.3 mmol, 1 mL, 15 eq.) was heated at 120° C. for 1 hour. The mixture was triturated with ethyl acetate (10 mL), filtered to give 1-[6-(4-chloroanilino)-2-[(2S)-2-(hydroxymethyl)pyrrolidin-1-yl]-5-nitro-pyrimidin-4-yl]-4-isopropoxy-piperidine-4-carboxamide (Intermediate B-5(136), 200 mg, 0.37 mmol, 54.8% yield) as a yellow solid. ¹H NMR (400 MHz, DMSO-d₆) δ=10.71-10.59 (m, 1H), 7.85-7.74 (m, 2H), 7.45-7.36 (m, 2H), 7.34-7.29 (m, 1H), 7.27-7.12 (m, 1H), 4.82-4.70 (m, 1H), 4.23-4.03 (m, 1H), 3.86-3.66 (m, 1H), 3.66-3.48 (m, 5H), 3.47-3.35 (m, 2H), 2.09-1.80 (m, 8H), 1.14 (d, J=6.0 Hz, 6H).

### Example 6—Preparation of Intermediates from General Synthetic Procedure C

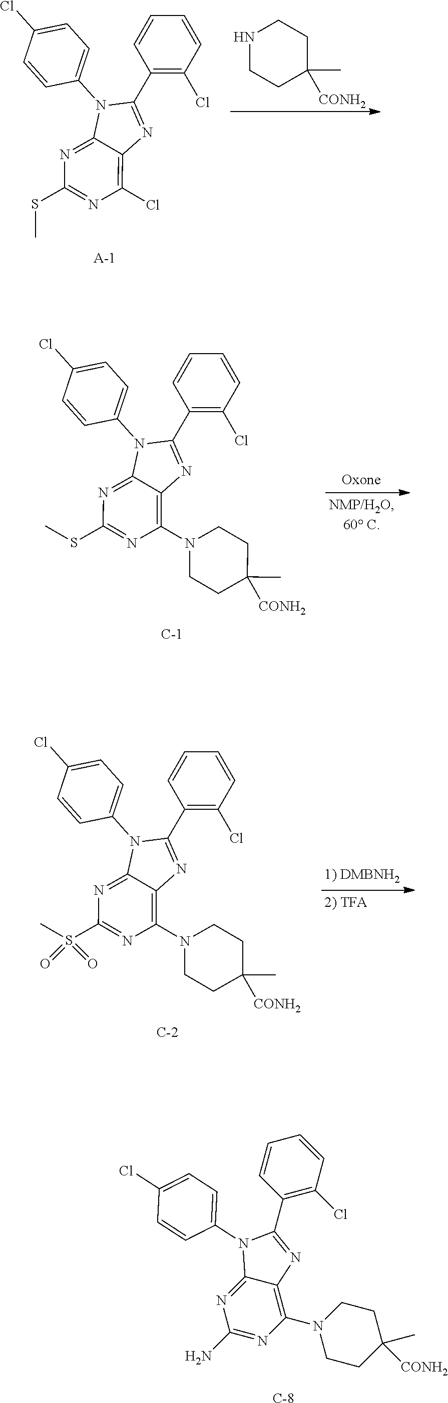

### (i) Preparation of Intermediate C-1

To a solution of 6-chloro-8-(2-chlorophenyl)-9-(4-chlorophenyl)-2-methylsulfanyl-purine (850 mg, 2.02 mmol, 1.0 eq.) and N,N-diisopropylethylamine (783 mg, 6.06 mmol, 3.0 eq.) in N,N-dimethylformamide (5 mL) was added 4-methylpiperidine-4-carboxamide (433 mg, 2.42 mmol, 1.2 eq., HCl salt), the resulting mixture was stirred at 60° C. for 1 hour. The reaction mixture was added to water (50 mL) and filtered to give a residue. The residue was triturated with acetonitrile (20 mL) to give 1-[8-(2-chlorophenyl)-9-(4-chlorophenyl)-2-methylsulfanyl-purin-6-yl]-4-methyl-piperidine-4-carboxamide (Intermediate C-1, 850 mg, 1.61 mmol, 79.7% yield) as an off-white solid.

### (ii) Preparation of Intermediate C-2

See Example 9, synthesis of Compound 68.

### (iii) Preparation of Intermediate C-8

Step 1: A mixture of 1-[8-(2-chlorophenyl)-9-(4-chlorophenyl)-2-methylsulfonyl-purin-6-yl]-4-methyl-piperidine-4-carboxamide (Compound 68, Intermediate C-2) (300 mg, 535 μmol, 1 eq) and (2,4-dimethoxyphenyl)methanamine (555 mg, 3.32 mmol, 6.20 eq) was stirred at 140° C. for 1 hour. After cooling to room temperature, the reaction mixture was purified by flash silica gel chromatography (ISCO®; 25 SepaFlash® Silica Flash Column, Eluent of 10-100% Ethyl acetate/Petroleum ether gradient @50 mL/min) to give 1-[8-(2-chlorophenyl)-9-(4-chlorophenyl)-2-[(2,4-dimethoxyphenyl)methylamino]purin-6-yl]-4-methyl-piperidine-4-carboxamide (290 mg, 448 μmol, 83.8% yield) as a yellow solid. ¹H NMR (400 MHz, DMSO-d6) δ=7.64-7.59 (m, 1H), 7.50-7.37 (m, 5H), 7.30-7.18 (m, 3H), 7.13-7.06 (m, 1H), 6.96-6.90 (m, 1H), 6.77-6.68 (m, 1H), 6.53-6.48 (m, 1H), 6.44-6.37 (m, 1H), 5.01-4.39 (m, 2H), 4.36-4.24 (m, 2H), 3.77 (s, 3H), 3.71 (s, 3H), 3.68-3.41 (m, 2H), 2.08-2.00 (m, 2H), 1.43-1.26 (m, 2H), 1.12 (s, 3H).

Step 2: A mixture of 1-[8-(2-chlorophenyl)-9-(4-chlorophenyl)-2-[(2,4-dimethoxyphenyl)methylamino]purin-6-yl]-4-methyl-piperidine-4-carboxamide (290 mg, 448.52 μmol, 1 eq) in trifluoroacetic acid (3 mL, 90 eq) was stirred at 65° C. for 1 hour. The mixture was concentrated in vacuum to give a residue. The residue was purified by flash silica gel chromatography (ISCO®; 25 g SepaFlash® Silica Flash Column, Eluent of 0˜50% MeOH/DCM ether gradient @30 mL/min) to give 1-[2-amino-8-(2-chlorophenyl)-9-(4-chlorophenyl)purin-6-yl]-4-methyl-piperidine-4-carboxamide (Intermediate C-8, 180 mg, 362.62 μmol, 80.8% yield) as a yellow solid. ¹H NMR (400 MHz, DMSO-d6) δ=7.63 (d, J=7.1 Hz, 1H), 7.57-7.25 (m, 7H), 6.98 (br s, 1H), 3.86 (br s, 4H), 2.20-2.05 (m, 2H), 1.55-1.38 (m, 2H), 1.17 (s, 3H).

### Example 7—Preparation of Intermediates from General Synthetic Procedure D

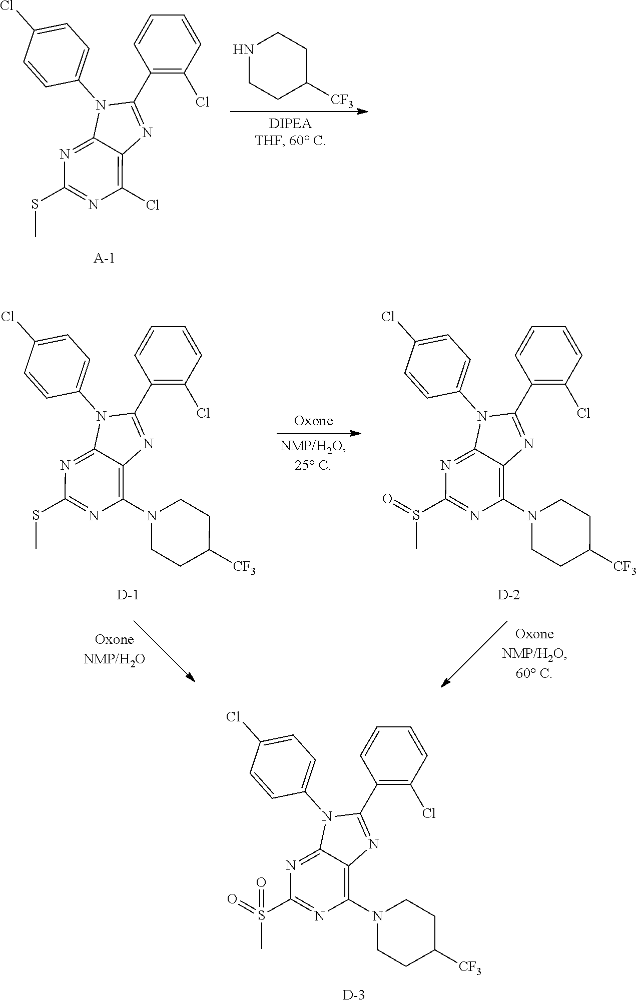

### (i) Preparation of Intermediate D-1

To a solution of 6-chloro-8-(2-chlorophenyl)-9-(4-chlorophenyl)-2-methylsulfanyl-purine (Intermediate A-1) (1.00 g, 2.37 mmol, 1.0 eq.) and N,N-diisopropylethylamine (368 mg, 2.85 mmol, 1.2 eq.) in tetrahydrofuran (10 mL) was added 4-(trifluoromethyl)piperidine (436 mg, 2.85 mmol, 1.2 eq.), the resulting mixture was stirred at 60° C. for 15 hours. The reaction mixture was concentrated under reduced pressure. The residue was triturated with ethanol (15 mL), and filtered to give 8-(2-chlorophenyl)-9-(4-chlorophenyl)-2-methylsulfanyl-6-[4-(trifluoromethyl)-1-piperidyl]purine (Intermediate D-1, 1.20 g, 2.23 mmol, 93.9% yield) as a red solid. ¹H NMR (400 MHz, DMSO-d₆) δ=7.69 (s, 1H), 7.50 (, J=8.4 Hz, 5H), 7.32 (d, J=8.4 Hz, 2H), 6.06-4.89 (m, 2H), 3.25-3.02 (m, 2H), 2.86-2.66 (m, 1H), 2.46-2.41 (m, 3H), 1.97 (br s, 2H), 1.61-1.39 (m, 2H).

### (ii) Preparation of Intermediate D-2

To a solution of 8-(2-chlorophenyl)-9-(4-chlorophenyl)-2-methylsulfanyl-6-[4-(trifluoromethyl)-1-piperidyl]purine (Intermediate D-1) (1.15 g, 2.14 mmol, 1.0 eq.) in N-methylpyrrolidone (15 mL) was added a solution of potassium oxidooxy hydrogen sulfate (1.08 g, 6.42 mmol, 3.0 eq.) in water (1.5 mL), the mixture was stirred at 25° C. for 15 hours. The reaction mixture was added to water (50 mL), filtered and concentrated in vacuum to give 8-(2-chlorophenyl)-9-(4-chlorophenyl)-2-methylsulfinyl-6-[4-(trifluoromethyl)-1-piperidyl]purine (Intermediate D-2, 1.10 g, 1.98 mmol, 92.7% yield) as a red solid. ¹H NMR (400 MHz, DMSO-d₆) δ=7.94-7.71 (m, 1H), 7.63-7.24 (m, 6H), 3.36-3.22 (m, 4H), 2.82 (s, 3H), 2.24-1.85 (m, 3H), 1.69-1.41 (m, 2H).

### (iii) Preparation of Intermediate D-3

### From D-2:

To a solution of 8-(2-chlorophenyl)-9-(4-chlorophenyl)-2-methylsulfinyl-6-[4-(trifluoromethyl)-1-piperidyl]purine (Intermediate D-2) (400 mg, 0.72 mmol, 1.0 eq.) in N-methylpyrrolidone (3.5 mL) and water (0.35 mL) was added oxone (121 mg, 0.72 mmol, 1.0 eq.), the reaction mixture was stirred at 60° C. for 1 hour. The reaction mixture was added to water (15 mL), filtered to give a solid. The solid was triturated with ethanol (15 mL), filtered to give solid. 8-(2-chlorophenyl)-9-(4-chlorophenyl)-2-methylsulfonyl-6-[4-(trifluoromethyl)-1-piperidyl]purine (Intermediate D-3, 300 mg, 0.53 mmol, 72.9% yield) as an off-white. ¹H NMR (400 MHz, DMSO-d₆) δ=7.74 (br d, J=7.0 Hz, 1H), 7.61-7.47 (m, 4H), 7.47-7.46 (m, 1H), 7.45-7.34 (m, 2H), 3.34-2.99 (m, 7H), 2.95-2.73 (m, 1H), 2.05 (br d, J=11.4 Hz, 2H), 1.67-1.45 (m, 2H).

### Example 8—Preparation of Other Intermediates

### (i) Preparation of Intermediates J

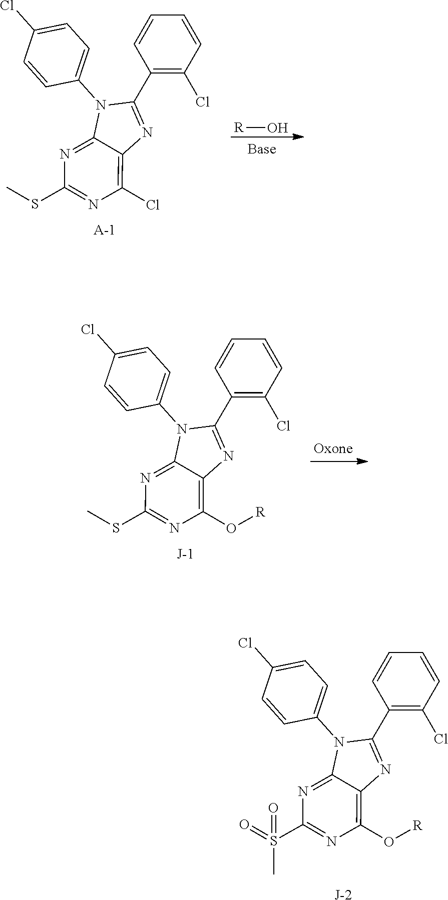

### Preparation of Intermediates J-1

Method A: a mixture of propan-2-ol (171 mg, 2.85 mmol, 1.2 eq.) and sodium hydride (332 mg, 8.30 mmol, 60% purity, 3.5 eq.) in N,N-dimethylformamide (10 mL) was stirred at 25° C. for 30 minutes, then 6-chloro-8-(2-chlorophenyl)-9-(4-chlorophenyl)-2-methylsulfanyl-purine (Intermediate A-1) (1 g, 2.37 mmol, 1 eq.) was added to the mixture and the mixture was stirred at 25° C. for 1.5 hours. The reaction mixture was added to water (60 mL) and filtered; the resulting filter cake was washed with water (3 mL*3) to give the crude intermediate J-1(86) (0.8 g) as a yellow solid.

Method B: a mixture of ethanol (109.24 mg, 2.37 mmol, 2.0 eq.) and sodium hydride (142.27 mg, 3.56 mmol, 60% purity, 3 eq.) in N,N-dimethylformamide (8 mL) was stirred at 25° C. for 30 minutes, then 6-chloro-8-(2-chlorophenyl)-9-(4-chlorophenyl)-2-methylsulfanyl-purine (Intermediate A-1) (500 mg, 1.19 mmol, 1.0 eq.) was added to the mixture and the mixture was stirred at 25° C. for 1.5 hours. The mixture was diluted with water (100 mL) and extracted with ethyl acetate (30 mL*3). The combined organic layers were washed with brine (100 mL), dried over anhydrous sodium sulfate, filtered, and concentrated under reduced pressure to give the crude intermediate J-1(99) (0.59 g) as a brown solid, which was used for the next step directly without further purification.

TABLE 4
Reaction conditions and data for Intermediates J-1

| Intermediates | | | | |
|---|---|---|---|---|
| J-1 | Base | Reagent | Compound name | ¹H NMR |
| J-1(86) | NaH | Propan-2-ol | 8-(2-chlorophenyl)-9-(4-chlorophenyl)- | — |
| Method A | | | 6-isopropoxy-2-methylsulfanyl-purine | |
| R = isopropyl | | | | |
| J-1(99) | NaH | Ethanol | 8-(2-chlorophenyl)-9-(4-chlorophenyl)- | — |
| Method B | | | 6-ethoxy-2-methylsulfanyl-purine | |
| R = ethyl | | | | |

### Preparation of Intermediate J-2

Method A: to solution of 8-(2-chlorophenyl)-9-(4-chlorophenyl)-6-isopropoxy-2-methylsulfanyl-purine (Intermediate J-1(86)) (0.8 g, 1.80 mmol, 1 eq.) in N-methy-2-pyrrolidone (12 mL) was added a solution of potassium oxidooxy hydrogen sulfate (2.82 g, 16.7 mmol, 9.3 eq.) in water (3 mL), the resulting mixture was stirred at 60° C. for 40 hours. The reaction mixture was added to water (120 mL) and filtered. The resulting filter cake was purified by flash silica gel chromatography (ISCO®; 40 g SepaFlash® Silica Flash Column, Eluent of 0˜80% Ethyl acetate/Petroleum ether gradient @50 mL/min). The cut fraction was concentrated under reduced pressure to give intermediate J-2(86) (0.56 g, 1.16 mmol, 65.1% yield) as a white solid.

Method B: to solution of 8-(2-chlorophenyl)-9-(4-chlorophenyl)-6-ethoxy-2-methylsulfanyl-purine (0.59 g, 1.37 mmol, 1.0 eq.) in N-methy-2-pyrrolidone (10 mL) was added a solution of potassium oxidooxy hydrogen sulfate (1.84 g, 10.94 mmol, 8.0 eq.) in water (2 mL), the resulting mixture was stirred at 60° C. for 18 hours. The reaction mixture was diluted with water (20 mL) and filtered; the resulting filter cake was purified by triturated with acetonitrile (2 mL) at 25° C. to give intermediate J-2(99) (378.5 mg, crude) as an off-white solid.

TABLE 5
Reaction conditions and data for Intermediates J-2

| Intermediates | Starting | | |
|---|---|---|---|
| J-2 | material | Compound name | ¹H NMR |
| J-2(86) | J-1(86) | 8-(2-chlorophenyl)-9-(4- | — |
| Method A | | chlorophenyl)-6-isopropoxy-2- | |
| R = isopropyl | | methylsulfonyl-purine | |
| J-2(99) | J-1(99) | 8-(2-chlorophenyl)-9-(4- | — |
| Method B | | chlorophenyl)-6-ethoxy-2- | |
| R = ethyl | | methylsulfonyl-purine | |

### (ii) Preparation of Intermediate L-1

To a solution of 4,6-dichloro-2-methylsulfanyl-5-nitro-pyrimidine (Intermediate B-1) (5 g, 20.8 mmol, 1 eq.) in N,N-dimethylformamide (20 mL) was added potassium carbonate (8.64 g, 62.5 mmol, 3 eq.) and aniline (2.06 g, 22.1 mmol, 2.02 mL, 1.06 eq.) at 0° C., the mixture was stirred at 0° C. for 1 hour. Then the mixture was stirred at 25° C. for 0.5 hour. The reaction mixture was washed with water (50 mL) and extracted with ethyl acetate 300 mL (100 mL×2). The combined organic layers were washed with brine 100 mL, dried over anhydrous sodium sulfate, filtered and concentrated under reduced pressure to give a residue. The residue was purified by flash silica gel chromatography (ISCO®; 80 g SepaFlash® Silica Flash Column, Eluent of 0˜100% Ethyl acetate/Petroleum ether gradient @60 mL/min) and the organic phase was concentrated under reduced pressure to give 6-chloro-2-methylsulfanyl-5-nitro-N-phenyl-pyrimidin-4-amine (Intermediate L-1, 7 g, 17.9 mmol, 86.0% yield, 76% purity) as a yellow solid. ¹H NMR (400 MHz, DMSO-d₆) δ=7.52 (d, J=7.6 Hz, 2H), 7.47-7.30 (m, 3H), 7.25-7.19 (m, 1H), 2.40 (s, 3H).

### (iii) Preparation of Intermediates K-2 and K-3

### Preparation of Intermediate K-2

To s solution of 4,6-dichloro-2-methyl-5-nitro-pyrimidine (Intermediate K-1) (5 g, 24.04 mmol, 1.0 eq.) in N, N-dimethylformamide (20 mL) was added potassium carbonate (9.97 g, 72.11 mmol, 3.0 eq.) and 4-chloroaniline (3.07 g, 24.04 mmol, 1.0 eq.) at 0° C., the mixture was stirred at 0° C. for 1 hour. Then the mixture was stirred at 25° C. for 0.5 hour. The reaction mixture was added dropwise into water 150 mL, filtered and the filtration residue was concentrated under reduced pressure to give the residue. The residue was added to ethyl alcohol 50 mL, filtered and the filtration residue was concentrated under reduced pressure to give the residue. The residue was added to ethyl acetate 50 mL, filtered and the filtrate was concentrated under reduced pressure to give 6-chloro-N-(4-chlorophenyl)-2-methyl-5-nitro-pyrimidin-4-amine (Intermediate K-2, 5 g, 16.16 mmol, 67.2% yield) as a yellow solid. ¹H NMR (400 MHz, DMSO-d₆) δ=10.00 (s, 1H), 7.63-7.55 (m, 2H), 7.47-7.40 (m, 2H), 2.46-2.38 (m, 3H).

### Preparation of Intermediates K-3(127) and K-3 (134)

K-3(127): To a solution of 6-chloro-N-(4-chlorophenyl)-2-methyl-5-nitro-pyrimidin-4-amine (Intermediate K-2) (200 mg, 0.67 mmol, 1 eq.) in N,N-dimethylformamide (2 mL) was added N,N-diisopropylethylamine (259 mg, 2.01 mmol, 0.35 mL, 3 eq.) and 4-methylpiperidine-4-carboxamide (143 mg, 0.80 mmol, 1.2 eq.). The mixture was stirred at 60° C. for 1 hour. The reaction mixture was added dropwise into water 20 mL, filtered and the filtration residue was concentrated under reduced pressure to give 1-[6-(4-chloroanilino)-2-methyl-5-nitro-pyrimidin-4-yl]-4-methyl-piperidine-4-carboxamide (Intermediate K-3(127), 260 mg, 0.61 mmol, 90.8% yield) as a light-yellow solid. ¹H NMR (400 MHz, DMSO-d₆) δ=10.11 (s, 1H), 7.75-7.66 (m, 2H), 7.43-7.37 (m, 2H), 7.29 (s, 1H), 7.00 (s, 1H), 3.63 (d, J=14.0 Hz, 2H), 3.26-3.18 (m, 2H), 2.28 (s, 3H), 2.11-2.05 (m, 2H), 1.46-1.37 (m, 2H), 1.14 (s, 3H).

K-3(134): To a solution of 6-chloro-N-(4-chlorophenyl)-2-methyl-5-nitro-pyrimidin-4-amine (Intermediate K-2) (200 mg, 0.67 mmol, 1 eq.) in N,N-dimethylformamide (2 mL) was added N,N-diisopropylethylamine (259 mg, 2.01 mmol, 0.35 mL, 3 eq.) and 4-ethoxypiperidine-4-carboxamide (167 mg, 0.80 mmol, 1.2 eq.). The mixture was stirred at 60° C. for 1 hour. The reaction mixture was added dropwise into water 20 mL, filtered and the filtration residue was concentrated under reduced pressure to give 1-[6-(4-chloroanilino)-2-methyl-5-nitro-pyrimidin-4-yl]-4-ethoxy-piperidine-4-carboxamide (Intermediate K-3(134), 280 mg, 0.62 mmol, 92.1% yield) as a light-yellow solid. ¹H NMR (400 MHz, DMSO-d₆) δ=10.12 (s, 1H), 7.74-7.66 (m, 2H), 7.44-7.37 (m, 2H), 7.30 (s, 1H), 7.24 (s, 1H), 3.77-3.64 (m, 2H), 3.38-3.32 (m, 2H), 3.30-3.24 (m, 2H), 2.29 (s, 3H), 1.88 (s, 4H), 1.19 (s, 3H).

### Example 9—Preparation of Compounds 1 to 240

### (i) General Synthetic Method A:

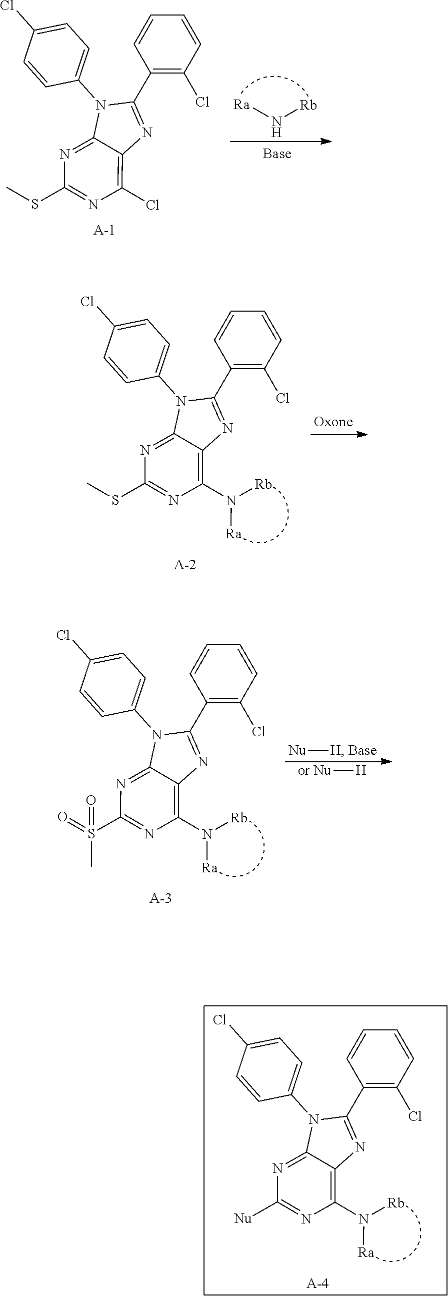

### Preparation of A-4 Compounds:

METHOD A: Crude Intermediate A-3 was dissolved in the nucleophile Nu-H (1 mL) and the reaction mixture was stirred at 100° C. overnight. EtOAc (15 mL) was added and the solution was washed 5 times with saturated NH₄Cl, dried with anhydrous Na₂SO₄ and concentrated. Crude residue was purified (injection with DMSO) on Buchi semi-prep (C18 column) using 20-60% ACN in AmF. The fractions were lyophilized affording compound A-4 (57% yield).

### Reaction Scale: 50 mg of Intermediate A-3

METHOD B: Sodium hydride 60% in dispersion in mineral oil (2 eq.) was added to a vial containing ethylene glycol (600 μL) in NMP (200 μL). The solution was stirred et 40° C. during 10 minutes then a solution of Intermediate A-3 (1 eq.) in NMP (200 μL) was added in one portion. The reaction mixture was stirred at 80° C. overnight where full conversion was observed by LCMS. 1 drop of water was added and the reaction mixture was purified (direct injection of reaction mixture) on Buchi semi-prep (C18 column) using 20-60% ACN in AmF. The desired fractions were lyophilized compound A-4 (57% yield) as an off-white powder.

### Reaction Scale: 40 mg of Intermediate A-3

METHOD C: To the Intermediate A-3 in a 8 mL vial was added potassium carbonate (2 eq.), a nucleophile Nu-H (11 eq.), and DMF (1.2 mL) and the mixture was stirred at 110° C. for 19 hours. The reaction mixture was purified by reverse phase flash chromatography using 10%-100% MeCN in 10 mM AmF (product elution at 72% MeCN), affording compound A-4 (73% yield).

### Reaction Scale: 120 mg of Intermediate A-3

METHOD D: To a solution of Intermediate A-3(74) (1 eq.) in nucleophile (Nu-H) (15 eq.) was stirred at 140° C. for 12 hours. The reaction mixture was concentrated under reduced pressure. The residue was purified by prep-HPLC (column: Unisil 3-100 C18 Ultra 150*50 mm*3 μm; mobile phase: [water(formic acid)-ACN]) to give compound A-4 as a white solid.

### Reaction Scale: 100 mg of Intermediate A-3

METHOD E: A mixture of intermediate A-3 (1 eq.), nucleophile (Nu-H) (110 mg, 1.22 mmol, 5 eq.) and potassium carbonate (67 mg, 0.48 mmol, 3 eq.) in N,N-dimethylformamide (1 mL) was stirred at 100° C. for 2-14 hours. The reaction mixture was filtered and concentrated under reduced pressure to give a residue. The residue was purified by prep-HPLC (column: C18 150*25 mm*10 μm; mobile phase: [water-ACN]) and the organic phase was concentrated under reduced pressure to remove acetonitrile, and the liquid was lyophilized to give compound A-4 as a white solid.

### Reaction Scale: 120 mg of Intermediate A-3

TABLE 6
Reaction conditions and data for A-4 compounds

| Compounds | | | Starting | | Exact |
|---|---|---|---|---|---|
| A-4 | Base | Nu-H | material | ¹H NMR, 400 MHz | Mass |
| 74 | No | 2-(methylamino)ethanol | A-3(74) | CDCl₃, δ = 7.48 (d, J = 6.8 Hz, | 499.3 |
| METHOD D | base | | | 1H), 7.39-7.28 (m, 5H), 7.17 (d, | (M + H)⁺ |
| 14% yield | | | | J = 8.7 Hz, 2H), 4.40 (br s, 4H), | |
| | | | | 3.92-3.77 (m, 2H), 3.74-3.66 | |
| | | | | (m, 2H), 3.17 (s, 3H), 1.62 (s, | |
| | | | | 3H) | |
| 75 | No | 2-(methylamino)ethanol | A-3(75) | DMSO-d₆, δ = 7.65 (br d, J = 7.5 | 508.3 |
| METHOD D | base | | | Hz, 1H), 7.53-7.38 (m, 5H), | (M + H)⁺ |
| 21% yield | | | | 7.26 (d, J = 8.7 Hz, 2H), 4.59 (br | |
| | | | | d, J = 4.9 Hz, 3H), 4.41-4.14 (m, | |
| | | | | 2H), 3.57 (br s, 4H), 3.08 (s, 3H), | |
| | | | | 1.69 (s, 3H) | |
| 76 | No | 2-(methylamino)ethanol | A-3(76) | CDCl₃, δ = 7.56-7.44 (m, 1H), | 513.2 |
| METHOD D | base | | | 7.27 (s, 5H), 7.21-7.12 (m, 2H), | (M + H)⁺ |
| 28% yield | | | | 4.77-3.94 (m, 4H), 3.91-3.79 | |
| | | | | (m, 2H), 3.71 (br s, 2H), 3.32 (s, | |
| | | | | 3H), 3.17 (s, 3H), 1.57 (s, 3H) | |
| 87 | K₂CO₃ | 2-methylpropane- | A-3(87) | DMSO-d₆, δ = 7.96 (d, J = 8.4 | 486.2 |
| METHOD E | | 1,2-diol | | Hz, 1H), 7.71 (d, J = 7.2 Hz, 1H), | (M + H)⁺ |
| 64% yield | | | | 7.53-7.41 (m, 5H), 7.30 (d, J = | |
| | | | | 8.4 Hz, 2H), 4.64 (s, 1H), 4.50- | |
| | | | | 4.37 (m, 1H), 3.98 (s, 2H), 1.25 | |
| | | | | (d, J = 6.4 Hz, 6H), 1.16 (s, 6H) | |
| 97 | K₂CO₃ | 2-methylpropane- | A-3(97) | CDCl₃, δ = 7.45-7.28 (m, 6H), | 502.2 |
| METHOD E | | 1,2-diol | | 7.21-7.13 (m, 2H), 4.25 (s, 4H), | (M + H)⁺ |
| 51% yield | | | | 4.00 (t, J = 4.8 Hz, 3H), 3.82- | |
| | | | | 3.01 (m, 4H), 1.31 (s, 6H) | |
| 98 | K₂CO₃ | 2-methylpropane- | A-3(98) | DMSO-d6, δ = 8.00-7.89 (m, | 488.1 |
| METHOD E | | 1,2-diol | | 1H), 7.69 (d, J = 7.2 Hz, 1H), | (M + H)⁺ |
| 10% yield | | | | 7.55-7.42 (m, 5H), 7.35-7.26 | |
| | | | | (m, 2H), 4.82-4.73 (m, 1H), 4.61 | |
| | | | | (s, 1H), 3.99 (s, 2H), 3.68-3.51 | |
| | | | | (m, 4H), 1.17 (s, 6H) | |
| 100 | K₂CO₃ | 2-methylpropane- | A-3(100) | DMSO-d₆, δ = 8.09-7.96 (m, | 501.1 |
| METHOD E | | 1,2-diol | | 1H), 7.70 (d, J = 7.2 Hz, 1H), | (M + H)⁺ |
| 13% yield | | | | 7.56-7.41 (m, 6H), 7.36-7.26 | |
| | | | | (m, 2H), 7.07 (s, 1H), 4.59 (s, | |
| | | | | 1H), 4.08-4.00 (m, 2H), 3.99 | |
| | | | | (s, 2H), 1.16 (s, 6H) | |
| 119 | K₂CO₃ | 2-methylpropane- | A-3(119) | DMSO-d₆, δ = 7.70-7.64 (m, | 528.3 |
| METHOD E | | 1,2-diol | | 1H), 7.53-7.40 (m, 5H), 7.29 (d, | (M + H)⁺ |
| 66% yield | | | | J = 8.8 Hz, 2H), 4.88 (d, J = 7.2 | |
| | | | | Hz, 1H), 4.62 (s, 1H), 4.33-4.19 | |
| | | | | (m, 1H), 4.15-4.04 (m, 1H), | |
| | | | | 4.00 (d, J = 7.2 Hz, 2H), 3.84- | |
| | | | | 3.68 (m, 2H), 2.01-1.84 (m, | |
| | | | | 2H), 1.38 (d, J = 4.8 Hz, 3H), | |
| | | | | 1.15 (s, 6H) | |
| 141 | No | ethanolamine | A-3(141) | DMSO-d6, δ = 7.66-7.60 (m, | 569.3 |
| METHOD A | base | | | 1H), 7.50-7.38 (m, 5H), 7.32 | (M + H)⁺ |
| | | | | (br s, 1H), 7.29-7.23 (m, 2H), | |
| | | | | 7.01 (s, 1H), 6.38 (t, 1H), 4.90 | |
| | | | | (br s, 1H), 4.59 (br s, 2H), 4.34 | |
| | | | | (br s, 1H), 4.05-3.55 (m, 2H), | |
| | | | | 3.48 (t, 2H), 2.39 (q, 2H), 1.91- | |
| | | | | 1.81 (m, 2H), 1.61 (br d, 2H), | |
| | | | | 1.04 (t, 3H) | |
| 142 | NaH | 1,2-ethanediol | A-3(141) | DMSO-d6, δ = 8.28 (s, 1H), 7.69 | 570.3 |
| METHOD B | | | | (d, 1H), 7.53-7.41 (m, 5H), | (M + H)⁺ |
| | | | | 7.38-7.27 (m, 3H), 7.02 (s, 1H), | |
| | | | | 5.04 (br s, 1H), 4.82 (br s, 1H), | |
| | | | | 4.30 (br s, 1H), 4.19 (t, 2H), 4.06 | |
| | | | | (br s, 1H), 3.66 (t, 2H), 3.64 (br | |
| | | | | s, 1H), 2.39 (q, 2H), 1.88 (br t, | |
| | | | | 2H), 1.65 (br d, 2H), 1.04 (t, 3H) | |
| 143 | No | [(2S)-pyrrolidin-2- | A-3(141) | DMSO-d6, δ = 7.67-7.63 (m, | 609.3 |
| METHOD A | base | yl]methanol | | 1H), 7.50-7.40 (m, 5H), 7.35 | (M + H)⁺ |
| | | | | (br s, 1H), 7.29-7.25 (m, 2H), | |
| | | | | 7.02 (br s, 1H), 4.92 (br s, 1H), | |
| | | | | 4.72 (t, 1H), 4.28 (br s, 1H), 4.03 | |
| | | | | (br s, 2H), 3.64 (br s, 2H), 3.42 | |
| | | | | (br s, 2H), 2.42 (br s, 2H), 1.96- | |
| | | | | 1.73 (m, 6H), 1.63 (br d, 2H), | |
| | | | | 1.06 (t, 3H) | |
| 145 | NaH | Acetamide | A-3(141) | DMSO-d6, δ = 9.93 (s, 1H), 7.73- | 567.3 |
| METHOD B | | | | 7.66 (m, 1H), 7.53-7.41 (m, | (M + H)⁺ |
| | | | | 5H), 7.37-7.31 (m, 3H), 7.03 (s, | |
| | | | | 1H), 5.04 (br s, 1H), 4.36 (br s, | |
| | | | | 1H), 4.06 (br s, 1H), 3.63 (br s, | |
| | | | | 1H), 2.40 (q, 2H), 2.15 (s, 3H), | |
| | | | | 1.88 (br s, 2H), 1.65 (br d, 2H), | |
| | | | | 1.05 (t, 3H) | |
| 148 | No | 2-(methylamino)ethanol | A-3(141) | DMSO-d6, δ = 7.67-7.63 (m, | 583.4 |
| METHOD A | base | | | 1H), 7.49-7.42 (m, 5H), 7.32 (s, | (M + H)⁺ |
| | | | | 1H), 7.29-7.24 (m, 2H), 7.00 (s, | |
| | | | | 1H), 4.94 (br s, 1H), 4.59 (t, 1H), | |
| | | | | 4.29 (br s, 1H), 3.97 (br s, 1H), | |
| | | | | 3.69 (br s, 1H), 3.57 (s, 4H), 3.07 | |
| | | | | (s, 3H), 2.39 (dd, 2H), 1.91- | |
| | | | | 1.80 (m, 2H), 1.61 (d, 2H), 1.04 | |
| | | | | (t, 3H) | |
| 149 | NaH | — | A-3(141) | DMSO-d6, δ = 10.83 (br s, 1H), | 526.3 |
| METHOD B | | | | 7.66 (d, 1H), 7.51-7.46 (m, | (M + H)⁺ |
| (Hydrolysis by- | | | | 4H), 7.46-7.38 (m, 1H), 7.32- | |
| product) | | | | 7.30 (m, 3H), 7.03 (s, 1H), 5.01 | |
| | | | | (br s, 1H), 4.30 (br s, 1H), 4.02 | |
| | | | | (br s, 1H), 3.65 (br s, 1H), 2.44- | |
| | | | | 2.36 (m, 2H), 1.87 (t, 2H), 1.63 | |
| | | | | (d, 2H), 1.04 (t, 3H) | |
| 150 | No | Piperidine | A-3(141) | DMSO-d6, δ = 7.68-7.62 (m, | 593.3 |
| METHOD A | base | | | 1H), 7.54-7.39 (m, 5H), 7.32 (s, | (M + H)⁺ |
| | | | | 1H), 7.29-7.24 (m, 2H), 7.00 (s, | |
| | | | | 1H), 3.68-3.58 (m, 4H), 2.95 (s, | |
| | | | | 1H), 2.39 (q, 2H), 1.92-1.79 | |
| | | | | (m, 2H), 1.68-1.52 (m, 4H), | |
| | | | | 1.48 (d, 3H), 1.08-1.00 (m, 3H) | |
| 151 | No | Benzylamine | A-3(141) | DMSO-d6, δ = 7.66-7.59 (m, | 615.4 |
| METHOD A | base | | | 1H), 7.52-7.35 (m, 5H), 7.33- | (M + H)⁺ |
| | | | | 7.14 (m, 8H), 6.98 (s, 1H), 4.42- | |
| | | | | 4.30 (m, 2H), 2.37 (q, 2H), | |
| | | | | 1.80 (s, 2H), 1.63-1.48 (m, 2H), | |
| | | | | 1.03 (t, 3H) | |
| 152 | No | Imidazole | A-3(141) | DMSO-d6, δ = 7.81 (t, 1H), 7.73 | 576.4 |
| METHOD A | base | | | (dd, 1H), 7.58-7.42 (m, 5H), | (M + H)⁺ |
| | | | | 7.42-7.32 (m, 3H), 7.07-7.01 | |
| | | | | (m, 2H), 5.21-5.06 (m, 1H), | |
| | | | | 4.54-4.40 (m, 1H), 4.21-4.07 | |
| | | | | (m, 1H), 3.84-3.67 (m, 1H), | |
| | | | | 2.41 (q, 2H), 2.00-1.84 (m, | |
| | | | | 2H), 1.77-1.64 (m, 2H), 1.05 (t, | |
| | | | | 3H) | |
| 153 | No | (R)-(−)-3- | A-3(141) | MeOD, δ = 7.60-7.55 (m, 1H), | 609.5 |
| METHOD A | base | Pyrrolidinemethanol | | 7.48-7.37 (m, 3H), 7.37-7.32 | (M + H)⁺ |
| | | | | (m, 2H), 7.29-7.25 (m, 2H), | |
| | | | | 4.32 (br s, 4H), 3.74-3.44 (m, | |
| | | | | 5H), 2.69 (q, 2H), 2.45 (dt, 1H), | |
| | | | | 2.21 (ddd, 2H), 2.10-2.01 (m, | |
| | | | | 1H), 1.86-1.77 (m, 2H), 1.77- | |
| | | | | 1.70 (m, 1H), 1.19 (t, 3H) | |
| 154 | No | (S)-(+)-2- | A-3(154) | DMSO-d6, δ = 7.69-7.62 (m, | 566.5 |
| METHOD A | base | Pyrrolidinemethanol | | 1H), 7.52-7.38 (m, 5H), 7.34- | (M + H)⁺ |
| | | | | 7.24 (m, 3H), 6.79 (s, 1H), 5.29 | |
| | | | | (s, 1H), 4.72 (t, 1H), 4.04 (s, | |
| | | | | 1H), 3.63 (s, 1H), 3.42 (s, 3H), | |
| | | | | 3.08 (s, 2H), 2.47-2.39 (m, 1H), | |
| | | | | 2.01-1.73 (m, 6H), 1.62-1.49 | |
| | | | | (m, 2H) | |
| 155 | No | N- | A-3(154) | DMSO-d6, δ = 7.67-7.64 (m, | 540.5 |
| METHOD A | base | methylethanolamine | | 1H), 7.51-7.40 (m, 5H), 7.33- | (M + H)⁺ |
| | | | | 7.25 (m, 3H), 6.79 (s, 1H), 5.27 | |
| | | | | (s, 1H), 4.60 (s, 1H), 3.57 (s, | |
| | | | | 4H), 3.07 (s, 3H), 3.07-3.00 (m, | |
| | | | | 2H), 2.47-2.40 (m, 1H), 1.85- | |
| | | | | 1.74 (m, 2H), 1.56 (qd, 2H) | |
| 156 | No | N- | A-3(154) | MeOH-d4, δ = 7.63-7.55 (m, | 540.5 |
| METHOD A | base | methylethanolamine | | 1H), 7.49-7.36 (m, 5H), 7.33- | (M + H)⁺ |
| | | | | 7.24 (m, 2H), 5.48 (bs, 1H), 4.60- | |
| | | | | 4.53 (m, 2H), 3.44-3.37 (m, | |
| | | | | 2H), 3.29-3.14 (m, 3H), 2.76 (s, | |
| | | | | 3H), 2.72-2.59 (m, 1H), 2.00- | |
| | | | | 1.90 (m, 2H), 1.77 (qd, 2H) | |
| 164 | No | Imidazole | A-3(164) | DMSO-d6, δ = 8.75 (d, 1H), 8.64 | 554.4 |
| METHOD A | base | | | (s, 1H), 7.88 (s, 1H), 7.76 (d, | (M + H)⁺ |
| | | | | 1H), 7.56-7.45 (m, 5H), 7.41- | |
| | | | | 7.35 (m, 2H), 7.09-7.02 (m, | |
| | | | | 1H), 4.79-4.65 (m, 1H), 3.57- | |
| | | | | 3.42 (m, 2H), 3.15 (d, 2H), 2.28- | |
| | | | | 2.16 (m, 4H) | |
| 166 | No | (S)-(+)-2- | A-3(164) | DMSO-d6, δ = 7.89-7.80 (m, | 587.4 |
| METHOD A | base | Pyrrolidinemethanol | | 1H), 7.70-7.64 (m, 1H), 7.50- | (M + H)⁺ |
| | | | | 7.40 (m, 5H), 7.30-7.24 (m, | |
| | | | | 2H), 4.74 (s, 1H), 4.40 (s, 1H), | |
| | | | | 4.07 (s, 1H), 3.71 (s, 1H), 3.42 | |
| | | | | (s, 1H), 3.32-3.24 (m, 3H), 3.22- | |
| | | | | 3.11 (m, 2H), 2.30-2.09 (m, | |
| | | | | 4H), 2.01-1.72 (m, 4H) | |
| 167 | No | N- | A-3(164) | DMSO-d6, δ = 7.83 (bs, 1H), | 561.4 |
| METHOD A | base | methylethanolamine | | 7.71-7.65 (m, 1H), 7.51-7.40 | (M + H)⁺ |
| | | | | (m, 5H), 7.30-7.23 (m, 2H), | |
| | | | | 4.59 (s, 1H), 4.39 (s, 1H), 3.66- | |
| | | | | 3.47 (m, 4H), 3.28-3.03 (m, | |
| | | | | 6H), 2.34-2.08 (m, 4H) | |
| 169 | No | (S)-(+)-2- | A-3(169) | DMSO-d6, δ = 7.69-7.64 (m, | 555.3 |
| METHOD A | base | Pyrrolidinemethanol | | 1H), 7.51-7.37 (m, 5H), 7.30- | (M + H)⁺ |
| | | | | 7.24 (m, 2H), 5.18 (bs, 1H), 4.81 | |
| | | | | (t, 1H), 4.70 (s, 1H), 4.13-3.90 | |
| | | | | (m, 2H), 3.70-3.37 (m, 7H), | |
| | | | | 3.20 (bs, 1H), 2.93 (bs, 1H), | |
| | | | | 2.02-1.71 (m, 4H) | |
| 170 | No | N- | A-3(169) | DMSO-d6, δ = 7.72-7.62 (m, | 529.3 |
| METHOD A | base | methylethanolamine | | 1H), 7.52-7.38 (m, 5H), 7.31- | (M + H)⁺ |
| | | | | 7.22 (m, 2H), 5.16 (bs, 1H), 4.87- | |
| | | | | 4.77 (m, 1H), 4.59 (s, 1H), 3.96 | |
| | | | | (dd, 1H), 3.62-3.47 (m, 7H), | |
| | | | | 3.46-3.38 (m, 1H), 3.19 (bs, | |
| | | | | 1H), 3.08 (s, 3H), 2.93 (bs, 1H) | |
| 171 | No | (S)-(+)-2- | A-3(171) | DMSO-d6, δ = 7.69-7.64 (m, | 555.3 |
| METHOD A | base | Pyrrolidinemethanol | | 1H), 7.51-7.41 (m, 5H), 7.30- | (M + H)⁺ |
| | | | | 7.25 (m, 2H), 5.20 (bs, 1H), 4.81 | |
| | | | | (t, 1H), 4.68 (s, 1H), 4.11-3.91 | |
| | | | | (m, 2H), 3.65-3.34 (m, 7H), | |
| | | | | 3.18 (bs, 1H), 2.94 (s, 1H), 2.01- | |
| | | | | 1.73 (m, 4H) | |
| 172 | No | N- | A-3(171) | DMSO-d6, δ = 7.70-7.62 (m, | 529.3 |
| METHOD A | base | methylethanolamine | | 1H), 7.51-7.40 (m, 5H), 7.31- | (M + H)⁺ |
| | | | | 7.24 (m, 2H), 5.16 (bs, 1H), 4.82 | |
| | | | | (t, 1H), 4.58 (t, 1H), 3.96 (dd, | |
| | | | | 1H), 3.62-3.47 (m, 7H), 3.46- | |
| | | | | 3.38 (m, 1H), 3.19 (bs, 1H), 3.08 | |
| | | | | (s, 3H), 2.93 (bs, 1H) | |
| 174 | No | N- | A-3(174) | DMSO-d6, δ = 7.64 (d, 1H), 7.51- | 513.3 |
| METHOD A | base | methylethanolamine | | 7.41 (m, 5H), 7.29-7.22 (m, | (M + H)⁺ |
| | | | | 2H), 4.74 (t, 1H), 4.60 (t, 1H), | |
| | | | | 4.19 (br s, 1H), 3.95 (br s, 1H), | |
| | | | | 3.74 (br s, 2H), 3.60-3.55 (m, | |
| | | | | 4H), 3.52-3.38 (m, 2H), 3.08 (s, | |
| | | | | 3H), 2.45-2.36 (m, 1H), 2.01 | |
| | | | | (br s, 1H), 1.73 (br d, 1H) | |
| 178 | No | N- | A-3(178) | DMSO-d6, δ = 7.67 (d, 1H), 7.60- | 513.3 |
| METHOD A | base | methylethanolamine | | 7.52 (m, 1H), 7.50-7.40 (m, | (M + H)⁺ |
| | | | | 5H), 7.30-7.23 (m, 2H), 4.60 (s, | |
| | | | | 1H), 4.22 (s, 1H), 3.96-3.85 (m, | |
| | | | | 2H), 3.58 (s, 4H), 3.39 (t, 2H), | |
| | | | | 3.08 (s, 3H), 1.92-1.59 (m, 4H). | |
| 180 | No | N- | A-3(174) | DMSO, δ = 7.64 (d, 1H), 7.49- | 513.3 |
| METHOD A | base | methylethanolamine | | 7.40 (m, 5H), 7.30-7.22 (m, | (M + H)⁺ |
| | | | | 2H), 4.84-4.52 (m, 2H), 4.21 (s, | |
| | | | | 1H), 3.96 (s, 1H), 3.74 (s, 2H), | |
| | | | | 3.58 (s, 4H), 3.53-3.40 (m, 3H), | |
| | | | | 3.08 (s, 3H), 2.01 (s, 1H), 1.70 | |
| | | | | (s, 1H) | |
| 181 | No | N- | A-3(181) | DMSO, δ = 7.64 (dd, 1H), 7.50- | 527.4 |
| METHOD A | base | methylethanolamine | | 7.40 (m, 5H), 7.30-7.23 (m, | (M + H)⁺ |
| | | | | 2H), 4.58 (s, 1H), 4.41 (s, 1H), | |
| | | | | 3.75-3.51 (m, 6H), 3.06 (s, 3H), | |
| | | | | 1.61-1.46 (m, 4H), 1.16 (s, 3H) | |
| 183 | No | N- | A-3(183) | DMSO-d6, δ = 7.68-7.63 (m, | 499.3 |
| METHOD A | base | methylethanolamine | | 1H), 7.50-7.40 (m, 5H), 7.30- | (M + H)⁺ |
| | | | | 7.24 (m, 2H), 4.62-4.56 (m, | |
| | | | | 1H), 4.15 (bs, 3H), 3.75-3.68 | |
| | | | | (m, 4H), 3.57 (s, 4H), 3.07 (s, | |
| | | | | 3H) | |
| 184 | No | N- | A-3(184) | DMSO-d6, δ = 7.68-7.64 (m, | 533.3 |
| METHOD A | base | methylethanolamine | | 1H), 7.51-7.41 (m, 5H), 7.30- | (M + H)⁺ |
| | | | | 7.25 (m, 2H), 4.60 (s, 1H), 4.29 | |
| | | | | (s, 3H), 3.58 (s, 4H), 3.08 (s, | |
| | | | | 3H), 2.15-1.99 (m, 4H) | |
| 185 | No | N- | A-3(185) | DMSO-d6, δ = 7.67-7.61 (m, | 413.4 |
| METHOD A | base | methylethanolamine | | 1H), 7.51-7.40 (m, 5H), 7.30- | (M + H)⁺ |
| | | | | 7.23 (m, 2H), 4.60 (s, 1H), 4.38- | |
| | | | | 4.15 (m, 1H), 4.08 (s, 1H), 3.92 | |
| | | | | (s, 1H), 3.78 (s, 1H), 3.58 (s, | |
| | | | | 5H), 3.27 (s, 3H), 3.08 (s, 3H), | |
| | | | | 2.05 (s, 2H) | |
| 186 | No | N- | A-3(186) | DMSO-d6, δ = 7.65 (dd, 1H), | 566.5 |
| METHOD A | base | methylethanolamine | | 7.61 (s, 1H), 7.51-7.39 (m, 5H), | (M + H)⁺ |
| | | | | 7.31-7.25 (m, 2H), 5.08 (s, 1H), | |
| | | | | 4.62 (s, 1H), 3.57 (s, 5H), 3.21 | |
| | | | | (t, 4H), 3.07 (s, 3H), 2.08 (t, 2H), | |
| | | | | 1.69 (td, 2H), 1.46 (d, 2H) | |
| 187 | No | N- | A-3(187) | DMSO-d6, δ = 7.68-7.63 (m, | 570.4 |
| METHOD A | base | methylethanolamine | | 1H), 7.52-7.40 (m, 5H), 7.30- | (M + H)⁺ |
| using 3- | | | | 7.22 (m, 2H), 4.97-4.55 (m, | |
| cyanoazetidine | | | | 2H), 4.37 (bs, 3H), 4.06-3.97 | |
| | | | | and 3.96-3.86 (m, 1H), 3.57 (s, | |
| | | | | 4H), 3.53-3.46 (m, 2H), 3.33- | |
| | | | | 3.26 (m, 3H), 3.07 (s, 3H), 2.94 | |
| | | | | and 2.86 (s, 3H) | |
| 188 | No | N- | A-3(188) | DMSO-d6, δ = 7.66-7.61 (m, | 511.4 |
| METHOD A | base | methylethanolamine | | 1H), 7.50-7.41 (m, 5H), 7.28- | (M + H)⁺ |
| | | | | 7.22 (m, 2H), 4.75 (s, 4H), 4.60 | |
| | | | | (s, 1H), 4.48 (s, 3H), 3.57 (s, | |
| | | | | 4H), 3.07 (s, 3H) | |
| 190 | No | N- | A-3(190) | DMSO-d6, δ = 10.74 (s, 1H), | 581.3 |
| METHOD A | base | methylethanolamine | | 8.67 (s, 1H), 7.66 (d, 1H), 7.45 | (M + H)⁺ |
| | | | | (s, 5H), 7.28 (d, 2H), 4.60 (s, | |
| | | | | 1H), 3.99-3.41 (m, 6H), 3.32- | |
| | | | | 3.26 (m, 1H), 3.07 (s, 3H), 1.86 | |
| | | | | (t, 2H), 1.72-1.57 (m, 2H) | |
| 191 | No | N- | A-3(191) | DMSO-d6, δ = 7.67-7.61 (m, | 553.3 |
| METHOD A | base | methylethanolamine | | 1H), 7.51-7.39 (m, 5H), 7.31- | (M + H)⁺ |
| | | | | 7.24 (m, 2H), 4.59 (t, 1H), 4.28 | |
| | | | | (br s, 2H), 4.04 (br s, 2H), 3.77 | |
| | | | | (t, 2H), 3.57 (s, 4H), 3.51 (s, 2H), | |
| | | | | 3.07 (s, 3H), 1.79 (t, 2H), 1.59 (t, | |
| | | | | 4H) | |
| 192 | No | N- | A-3(192) | DMSO-d6, δ = 8.09 (s, 1H), 7.67 | 547.3 |
| METHOD A | base | methylethanolamine | | (d, 1H), 7.53-7.39 (m, 5H), | (M + H)⁺ |
| | | | | 7.32-7.23 (m, 2H), 4.88 (s, 1H), | |
| | | | | 4.62 (s, 1H), 3.68-3.49 (m, 5H), | |
| | | | | 3.46-3.39 (m, 2H), 3.26-3.13 | |
| | | | | (m, 2H), 3.09 (s, 3H), 2.41-2.28 | |
| | | | | (m, 1H) | |
| 193 | No | N- | A-3(193) | DMSO-d6, δ = 8.09 (s, 1H), 7.67 | 547.3 |
| METHOD A | base | methylethanolamine | | (d, 1H), 7.54-7.38 (m, 5H), | (M + H)⁺ |
| | | | | 7.32-7.23 (m, 2H), 4.88 (s, 1H), | |
| | | | | 4.61 (s, 1H), 3.69-3.50 (m, 5H), | |
| | | | | 3.47-3.39 (m, 2H), 3.25-3.15 | |
| | | | | (m, 2H), 3.09 (s, 3H), 2.42-2.26 | |
| | | | | (m, 1H) | |
| 194 | No | N- | A-3(194) | DMSO-d6, δ = 7.81 (br s, 1H), | 499.3 |
| METHOD A | base | methylethanolamine | | 7.69-7.65 (m, 1H), 7.51-7.40 | (M + H)⁺ |
| | | | | (m, 5H), 7.31-7.22 (m, 2H), | |
| | | | | 4.64-4.58 (m, 2H), 3.98 (dd, | |
| | | | | 1H), 3.86 (dd, 1H), 3.74 (td, 1H), | |
| | | | | 3.66-3.54 (m, 5H), 3.08 (s, 3H), | |
| | | | | 2.24-2.13 (m, 1H), 2.11-2.02 | |
| | | | | (m, 1H) | |
| 195 | No | N- | A-3(195) | DMSO-d6, δ = 7.81 (br s, 1H), | 499.3 |
| METHOD A | base | methylethanolamine | | 7.71-7.64 (m, 1H), 7.52-7.39 | (M + H)⁺ |
| | | | | (m, 5H), 7.32-7.23 (m, 2H), | |
| | | | | 4.63-4.57 (m, 2H), 3.98 (dd, | |
| | | | | 1H), 3.86 (dd, 1H), 3.74 (td, 1H), | |
| | | | | 3.66-3.55 (m, 4H), 3.08 (s, 3H), | |
| | | | | 2.24-2.13 (m, 1H), 2.13-2.02 | |
| | | | | (m, 1H) | |
| 198 | K₂CO₃ | 2-methylpropane- | A-3(154) | DMSO-d6, δ = 7.75-7.64 (m, | 555.3 |
| METHOD C | | 1,2-diol | | 1H), 7.57-7.39 (m, 5H), 7.36- | (M + H)⁺ |
| | | | | 7.27 (m, 3H), 6.82 (s, 1H), 5.33 | |
| | | | | (bs, 1H), 4.62 (s, 1H), 3.98 (s, | |
| | | | | 2H), 3.32-3.01 (m, 2H), 2.49- | |
| | | | | 2.42 (m, 2H), 1.19-1.13 (m, | |
| | | | | 2H), 1.58 (qd, 2H), 1.15 (s, 6H) | |
| 199 | K₂CO₃ | 2-methylpropane- | A-3(181) | DMSO-d6, δ = 7.75-7.64 (m, | 542.3 |
| METHOD C | | 1,2-diol | | 1H), 7.56-7.36 (m, 5H), 7.33- | (M + H)⁺ |
| | | | | 7.27 (m, 2H), 5.18 (bs, 1H), 4.61 | |
| | | | | (s, 1H), 4.48 (s, 1H), 3.97 (s, | |
| | | | | 2H), 3.89-3.35 (m, 2H), 1.67- | |
| | | | | 1.49 (m, 4H), 1.17 (s, 3H, 1.15 | |
| | | | | (s, 6H) | |
| 200 | No | N- | A-3(97) | DMSO-d6, δ = 7.63 (d, 1H), 7.52- | 487.3 |
| METHOD A | base | methylethanolamine | | 7.37 (m, 5H), 7.30-7.21 (m, | (M + H)⁺ |
| | | | | 2H), 4.93-4.45 (m, 2H), 4.21 | |
| | | | | (bs, 1H), 3.96-3.65 (m, 4H), | |
| | | | | 3.57 (s, 5H), 3.30-3.17 (m, 1H), | |
| | | | | 3.07 (s, 3H) | |
| 201 | K₂CO₃ | 2-methylpropane- | A-3(201) | DMSO-d6, δ = 7.67 (d, 1H), 7.53- | 555.3 |
| METHOD C | | 1,2-diol | | 7.47 (m, 4H), 7.47-7.41 (m, | (M + H)⁺ |
| | | | | 1H), 7.37 (bs, 1H), 7.32-7.26 | |
| | | | | (m, 2H), 6.91 (s, 1H), 5.95-4.71 | |
| | | | | (m, 1H), 4.63 (s, 1H), 3.97 (s, | |
| | | | | 2H), 3.33-2.82 (m, 2H), 2.39 (t, | |
| | | | | 1H), 2.00-1.89 (m, 1H), 1.87- | |
| | | | | 1.76 (m, 1H), 1.74-1.59 (m, | |
| | | | | 1H), 1.57-1.41 (m, 1H), 1.15 (s, | |
| | | | | 6H) | |
| 202 | K₂CO₃ | 2-methylpropane- | A-3(202) | DMSO-d6, δ = 7.67 (d, 1H), 7.53- | 555.3 |
| METHOD C | | 1,2-diol | | 7.47 (m, 4H), 7.47-7.41 (m, | (M + H)⁺ |
| | | | | 1H), 7.38 (bs, 1H), 7.33-7.25 | |
| | | | | (m, 2H), 6.91 (s, 1H), 5.91-4.74 | |
| | | | | (m, 1H), 4.63 (s, 1H), 3.97 (s, | |
| | | | | 2H), 3.32-2.81 (m, 2H), 2.39 (t, | |
| | | | | 1H), 2.01-1.91 (m, 1H), 1.87- | |
| | | | | 1.77 (m, 1H), 1.75-1.60 (m, | |
| | | | | 1H), 1.56-1.40 (m, 1H), 1.15 (s, | |
| | | | | 6H) | |
| 203 | K₂CO₃ | 2-methylpropane- | A-3(203) | DMSO-d6, δ = 7.73-7.63 (m, | 526.3 |
| METHOD C | | 1,2-diol | | 1H), 7.54-7.39 (m, 5H), 7.34- | (M + H)⁺ |
| | | | | 7.27 (m, 2H), 5.98 (s) and 5.22 | |
| | | | | (s) (1H), 4.74 (s) (1H), 4.61 (s, | |
| | | | | 1H), 4.00 (d, 3H), 3.88 (t, 1H), | |
| | | | | 3.78 (t, 1H), 3.61 (dd, 1H), 2.03- | |
| | | | | 1.88 (m, 2H), 1.15 (s, 6H) | |
| 204 | K₂CO₃ | 2-methylpropane- | A-3(204) | DMSO-d6, as a mixture of | 526.3 |
| METHOD C | | 1,2-diol | | rotamers δ = 7.73-7.63 (m, 1H), | (M + H)⁺ |
| | | | | 7.54-7.39 (m, 5H), 7.34-7.27 | |
| | | | | (m, 2H), 5.98 (s) and 5.22 (s) | |
| | | | | (1H), 4.74 (s, 1H), 4.61 (s, 1H), | |
| | | | | 4.02-3.93 (m, 3H), 3.88 (t, 1H), | |
| | | | | 3.78 (t, 1H), 3.65 (d) and 3.57 (d) | |
| | | | | (1H), 2.03-1.87 (m, 2H), 1.15 | |
| | | | | (s, 6H) | |
| 206 | K₂CO₃ | 2-methylpropane- | A-3(206) | DMSO-d6, δ = 7.69 (dd, 1H), | 528.3 |
| METHOD C | | 1,2-diol | | 7.53-7.41 (m, 5H), 7.34-7.27 | (M + H)⁺ |
| | | | | (m, 2H), 5.23 (bs, 1H), 4.61 (s, | |
| | | | | 1H), 3.97 (s, 2H), 3.95 (s, 1H), | |
| | | | | 3.70-3.52 (m, 2H), 3.31-3.11 | |
| | | | | (m, 1H), 2.90 (bs, 1H), 1.17 (d, | |
| | | | | 3H), 1.15 (s, 6H) | |
| 207 | K₂CO₃ | 2-methylpropane- | A-3(207) | DMSO-d6, δ = 7.69 (dd, 1H), | 599.3 |
| METHOD C | | 1,2-diol | | 7.54-7.39 (m, 5H), 7.35-7.28 | (M + H)⁺ |
| | | | | (m, 3H), 7.22 (s, 1H), 5.40 (bs, | |
| | | | | 1H), 4.61 (s, 2H), 3.98 (s, 2H), | |
| | | | | 3.65 (bs, 1H), 3.38 (dd, 3H), | |
| | | | | 1.89 (s, 4H), 1.21 (t, 3H), 1.15 (s, | |
| | | | | 6H) | |
| 208 | K₂CO₃ | 2-methylpropane- | A-3(141) | DMSO-d6, δ = 7.69 (dd 1H), 7.53- | 571.3 |
| METHOD C | | 1,2-diol | | 7.40 (m, 5H), 7.34-7.29 (m, | (M + H)⁺ |
| | | | | 3H), 7.16 (d, 1H), 5.58 (br s, 1H), | |
| | | | | 5.51 (s, 1H), 4.77 (br s, 1H), 4.62 | |
| | | | | (s, 1H), 3.98 (s, 2H), 3.60-3.26 | |
| | | | | (br m, 2H), 1.96 (td, 2H), 1.60 (d, | |
| | | | | 2H), 1.15 (s, 6H) | |
| 209 | K₂CO₃ | 2-methylpropane- | A-3(164) | MeOH-d4, δ = 7.62-7.56 (m, | 562.2 |
| METHOD C | | 1,2-diol | | 1H), 7.50-7.37 (m, 5H), 7.31- | (M + H)⁺ |
| | | | | 7.25 (m, 2H), 4.80 (s, 3H), 4.15 | |
| | | | | (s, 2H), 3.30-3.26 (m, 5H), 1.29 | |
| | | | | (s, 6H) | |
| 210 | K₂CO₃ | 2-methylpropane- | A-3(210) | DMSO-d6, δ = 7.69 (dd, 1H), | 528.3 |
| METHOD C | | 1,2-diol | | 7.54-7.40 (m, 5H), 7.34-7.27 | (M + H)⁺ |
| | | | | (m, 2H), 5.90-4.66 (m, 1H), | |
| | | | | 4.61 (s, 1H), 3.97 (s, 2H), 3.95 | |
| | | | | (s, 1H), 3.69-3.52 (m, 2H), 3.31- | |
| | | | | 3.12 (m, 1H), 2.91 (bs, 1H), | |
| | | | | 1.17 (d, 3H), 1.15 (s, 6H) | |
| 211 | K₂CO₃ | 2-methylpropane- | A-3(211) | DMSO-d6, δ = 7.68 (dd, 1H), | 528.2 |
| METHOD C | | 1,2-diol | | 7.54-7.38 (m, 5H), 7.34-7.27 | (M + H)⁺ |
| | | | | (m, 2H), 5.40 (bs, 1H), 4.61 (s, | |
| | | | | 1H), 4.03-3.93 (m, 3H), 3.82- | |
| | | | | 3.73 (m, 1H), 3.68 (dd, 1H), 3.61- | |
| | | | | 3.48 (m, 1H), 3.47-3.35 (m, | |
| | | | | 1H), 1.34 (d, 3H), 1.15 (s, 6H) | |
| 212 | K₂CO₃ | 2-methylpropane- | A-3(212) | DMSO-d6, δ = 7.68 (dd, 1H), | 528.2 |
| METHOD C | | 1,2-diol | | 7.57-7.39 (m, 5H), 7.35-7.26 | (M + H)⁺ |
| | | | | (m, 2H), 5.84-4.71 (m, 1H), | |
| | | | | 4.61 (s, 1H), 4.06-3.91 (m, 3H), | |
| | | | | 3.83-3.74 (m, 1H), 3.69 (dd, | |
| | | | | 1H), 3.60-3.49 (m, 1H), 3.42 | |
| | | | | (bs, 1H), 1.34 (d, 3H), 1.15 (s, | |
| | | | | 6H) | |
| 213 | K₂CO₃ | 2-methylpropane- | A-3(213) | DMSO-d6, δ = 7.76-7.65 (m, | 585.3 |
| METHOD C | | 1,2-diol | | 1H), 7.58-7.36 (m, 6H), 7.35- | (M + H)⁺ |
| | | | | 7.27 (m, 2H), 7.22 (s, 1H), 5.43 | |
| | | | | (bs, 1H), 4.96-4.53 (m, 2H), | |
| | | | | 3.98 (s, 2H), 3.58 (bs, 1H), 3.31 | |
| | | | | (s, 1H), 3.26-3.18 (m, 3H), 1.98- | |
| | | | | 1.82 (m, 4H), 1.15 (s, 6H) | |
| 214 | K₂CO₃ | 2-methylpropane- | A-3(214) | DMSO-d6, δ = 7.68 (d, 1H), 7.58- | 540.2 |
| METHOD C | | 1,2-diol | | 7.39 (m, 5H), 7.31 (d, 2H), | (M + H)⁺ |
| | | | | 5.42 (s, 1H), 4.61 (s, 1H), 4.47 | |
| | | | | (s, 2H), 3.97 (s, 2H), 3.64-3.39 | |
| | | | | (m, 1H), 3.27-3.07 (m, 1H), | |
| | | | | 1.92-1.79 (m, 2H), 1.78-1.64 | |
| | | | | (m, 2H), 1.15 (s, 6H) | |
| 215 | K₂CO₃ | 2-methylpropane- | A-3(215) | DMSO-d6, δ = 7.69 (dd1H), 7.53- | 613.3 |
| METHOD C | | 1,2-diol | | 7.40 (m, 5H), 7.35-7.27 (m, | (M + H)⁺ |
| | | | | 3H), 7.22 (s, 1H), 5.12 (bs, 1H), | |
| | | | | 4.61 (s, 1H), 4.40 (bs, 1H), 3.98 | |
| | | | | (s, 3H), 3.81-3.68 (m, 1H), 3.53 | |
| | | | | (bs, 1H), 1.96-1.84 (m, 4H), | |
| | | | | 1.20-1.11 (m, 12H) | |
| 216 | K₂CO₃ | 2-methylpropane- | A-3(216) | DMSO-d6, δ = 7.75-7.64 (m, | 599.4 |
| METHOD C | | 1,2-diol | | 1H), 7.63-7.36 (m, 6H), 7.35- | (M + H)⁺ |
| | | | | 7.25 (m, 3H), 6.05-5.58 (m, | |
| | | | | 1H), 5.14-4.74 (m, 1H), 4.64 (s, | |
| | | | | 1H), 4.02-3.86 (m, 2H), 3.84- | |
| | | | | 3.37 (m, 2H), 3.29-2.84 (s, 1H), | |
| | | | | 2.43 (s, 1H), 2.06-1.54 (m, 4H), | |
| | | | | 1.15 (s, 6H), 1.07-0.83 (m, 3H) | |
| 217 | K₂CO₃ | 2-methylpropane- | A-3(216) | DMSO-d6, δ = 7.75-7.64 (m, | 599.4 |
| METHOD C | | 1,2-diol | | 1H), 7.63-7.36 (m, 6H), 7.35- | (M + H)⁺ |
| | | | | 7.25 (m, 3H), 6.05-5.58 (m, | |
| | | | | 1H), 5.14-4.74 (m, 1H), 4.64 (s, | |
| | | | | 1H), 4.02-3.86 (m, 2H), 3.84- | |
| | | | | 3.37 (m, 2H), 3.29-2.84 (s, 1H), | |
| | | | | 2.43 (s, 1H), 2.06-1.54 (m, 4H), | |
| | | | | 1.15 (s, 6H), 1.07-0.83 (m, 3H). | |
| 222 | K₂CO₃ | 2-methylpropane- | A-3(222) | DMSO-d6, δ = 7.69 (d, 1H), 7.55- | 555.2 |
| METHOD C | | 1,2-diol | | 7.41 (m, 5H), 7.31 (d, 2H), | (M + H)⁺ |
| | | | | 4.63 (s, 1H), 4.22 (bs, 3H), 3.99 | |
| | | | | (s, 2H), 3.61 (s, 4H), 2.06 (s, | |
| | | | | 3H), 1.16 (s, 6H) | |
| 223 | No | 3-methyl-3- | A-3(44) | DMSO-d6, δ = 7.65 (d, 1H), 7.51- | 580.3 |
| METHOD A | base | pyrrolidinol | | 7.40 (m, 5H), 7.29-7.23 (m, | (M + H)⁺ |
| | | | | 3H), 6.95 (s, 1H), 4.68 (s, 1H), | |
| | | | | 4.59 (br s, 1H), 3.53 (br m, 5H), | |
| | | | | 3.25 (br s, 1H), 2.10-2.02 (m, | |
| | | | | 2H), 1.88-1.73 (m, 2H), 1.39 | |
| | | | | (ddd, 2H), 1.30 (s, 3H), 1.15 (s, | |
| | | | | 3H) | |
| 224 | K₂CO₃ | 2-methylpropane- | A-3(224) | DMSO-d6, δ = 7.70 (d, 1H), 7.55- | 571.3 |
| METHOD C | | 1,2-diol | | 7.41 (m, 5H), 7.35-7.27 (m, | (M + H)⁺ |
| | | | | 2H), 4.62 (s, 1H), 4.24 (bs, 3H), | |
| | | | | 3.97 (s, 2H), 3.64 (s, 3H), 3.59- | |
| | | | | 3.49 (m, 4H), 3.31 (s, 1H), 1.15 | |
| | | | | (s, 6H) | |
| 225 | K₂CO₃ | 3-methyl-3- | A-3(207) | DMSO-d6, δ = 7.67 (d, 1H), 7.51- | 610.3 |
| METHOD C | | pyrrolidinol | | 7.39 (m, 5H), 7.34-7.25 (m, | (M + H)⁺ |
| | | | | 3H), 7.19 (s, 1H), 5.27 (bs, 1H), | |
| | | | | 4.69 (s, 1H), 3.50 (bs, 4H), 3.41- | |
| | | | | 3.29 (m, 4H), 3.26 (bs, 1H), | |
| | | | | 1.93-1.73 (m, 6H), 1.30 (s, 3H), | |
| | | | | 1.21 (t, 3H) | |
| 226 | No | 2-methylpropane- | A-3(226) | DMSO-d6, δ = 7.67 (d, 1H), 7.56- | 583.3 |
| METHOD C | base | 1,2-diol | | 7.40 (m, 6H), 7.30 (d, 2H), | (M + H)⁺ |
| | | | | 5.06 (br s, 1H), 4.61 (s, 1H), 4.36 | |
| | | | | (br s, 1H), 3.97 (s, 2H), 3.87 (br | |
| | | | | s, 1H), 3.46 (br s, 1H), 2.30- | |
| | | | | 2.14 (m, 2H), 1.84 (s, 3H), 1.59- | |
| | | | | 1.45 (m, 2H), 1.32 (s, 3H), 1.15 | |
| | | | | (s, 6H) | |
| 227 | No | 2-methylpropane- | A-3(227) | DMSO-d6, δ = 7.69 (dd, 1H), | 527.3 |
| METHOD C | base | 1,2-diol | | 7.56-7.36 (m, 5H), 7.34-7.27 | (M + H)⁺ |
| | | | | (m, 2H), 4.61 (s, 1H), 4.21 (s, | |
| | | | | 3H), 3.97 (s, 2H), 3.30 (s, 1H), | |
| | | | | 2.48-2.40 (m, 4H), 2.22 (s, 3H), | |
| | | | | 1.15 (s, 6H) | |
| 231 | K₂CO₃ | (S)-(+)-2- | A-3(215) | DMSO-d6, δ = 7.66 (ddd, 1H), | 624.3 |
| METHOD A | | Pyrrolidinemethanol | | 7.51-7.41 (m, 5H), 7.30 (s, 1H), | (M + H)⁺ |
| | | | | 7.30-7.25 (m, 2H), 7.19 (s, 1H), | |
| | | | | 5.01 (br s, 1H), 4.72 (t, 1H), 4.38 | |
| | | | | (br s, 1H), 4.03 (br s, 1H), 3.92- | |
| | | | | 3.36 (br m, 6H), 1.97-1.74 (m, | |
| | | | | 8H), 1.15 (d, 6H) | |
| 233 | K₂CO₃ | 2-methylpropane- | A-3(233) | DMSO-d6, δ = 8.39 (s, 1H), 7.67 | 555.2 |
| METHOD C | | 1,2-diol | | (dd, 1H), 7.53-7.39 (m, 5H), | (M + H)⁺ |
| | | | | 7.32-7.26 (m, 2H), 4.62 (s, 1H), | |
| | | | | 4.60 (br s, 1H), 4.39-4.27 (m, | |
| | | | | 2H), 4.06 (s, 1H), 3.96 (s, 2H), | |
| | | | | 1.84 (s, 3H), 1.55 (s, 3H), 1.15 | |
| | | | | (s, 6H) | |
| 234 | No | (S)-(+)-2- | A-3(233) | DMSO-d6, δ = 8.38 (s, 1H), 7.67- | 566.2 |
| METHOD A | base | Pyrrolidinemethanol | | 7.61 (m, 1H), 7.51-7.40 (m, | (M + H)⁺ |
| | | | | 5H), 7.30-7.22 (m, 2H), 4.87 | |
| | | | | (br s, 1H), 4.51 (br s, 1H), 4.26 | |
| | | | | (br s, 2H), 4.03 (br s, 2H), 3.61 | |
| | | | | (br s, 1H), 3.44-3.35 (m, 3H), | |
| | | | | 1.95-1.85 (m, 3H), 1.83 (s, 3H), | |
| | | | | 1.80-1.72 (m, 1H), 1.54 (s, 3H) | |
| 237 | No | (S)-(+)-2- | A-3(237) | DMSO-d6, δ = 7.65 (dd, 1H), | 538.3 |
| METHOD A | base | Pyrrolidinemethanol | | 7.54-7.40 (m, 6H), 7.29-7.22 | (M + H)⁺ |
| | | | | (m, 2H), 7.06 (s, 1H), 4.86 (s, | |
| | | | | 1H), 4.35 (bs, 4H), 4.04 (s, 1H), | |
| | | | | 3.62 (s, 1H), 3.55-3.37 (m, 4H), | |
| | | | | 1.94-1.72 (m, 4H) | |
| 238 | K₂CO₃ | 2-methylpropane- | A-3(237) | DMSO-d6, δ = 7.67 (dd, 1H), | 527.2 |
| METHOD C | | 1,2-diol | | 7.55 (s, 1H), 7.52-7.39 (m, 5H), | (M + H)⁺ |
| | | | | 7.34-7.24 (m, 2H), 7.10 (s, 1H), | |
| | | | | 4.62 (s, 2H), 4.53 (s, 1H), 4.43- | |
| | | | | 4.11 (m, 2H), 3.96 (s, 2H), 3.64- | |
| | | | | 3.44 (m, 1H), 1.15 (s, 6H) | |

### (iv) General Synthetic Method B:

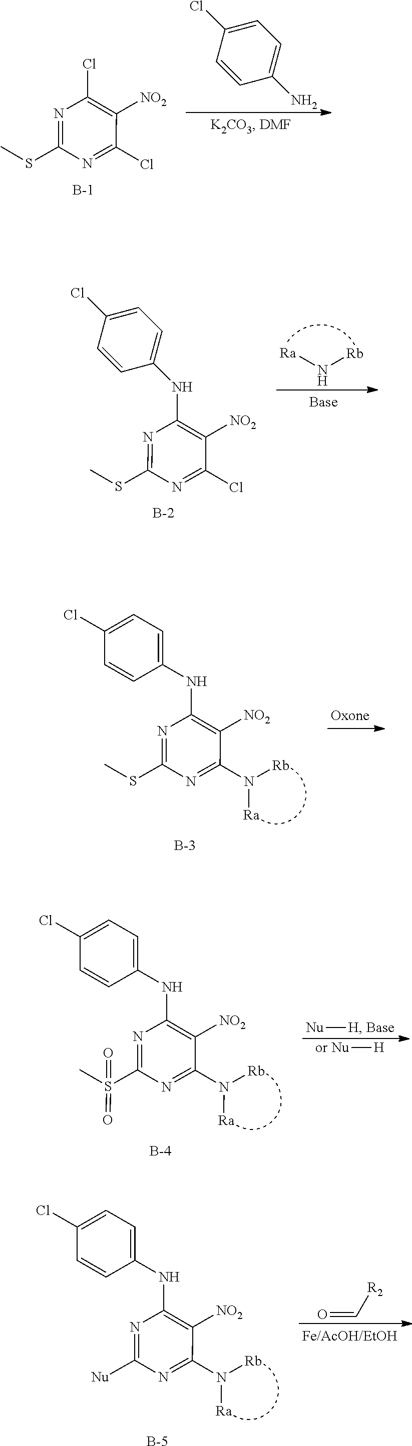

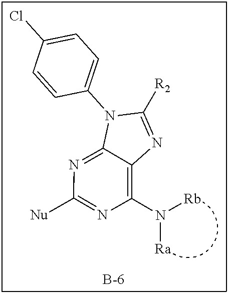

### Preparation of B8 Compounds can be Found in Following Detailed Protocols.

### (v) General Synthetic Method C:

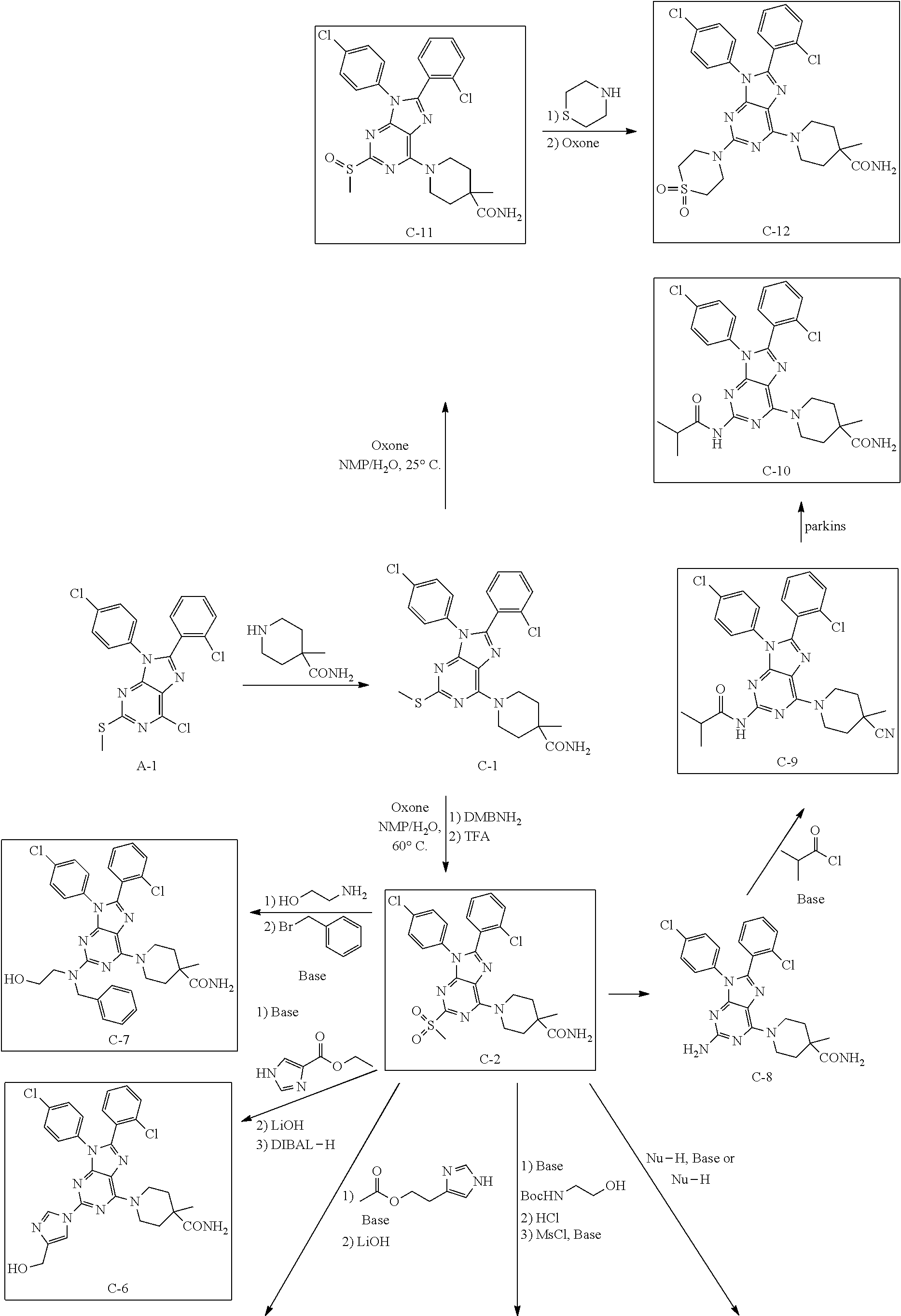

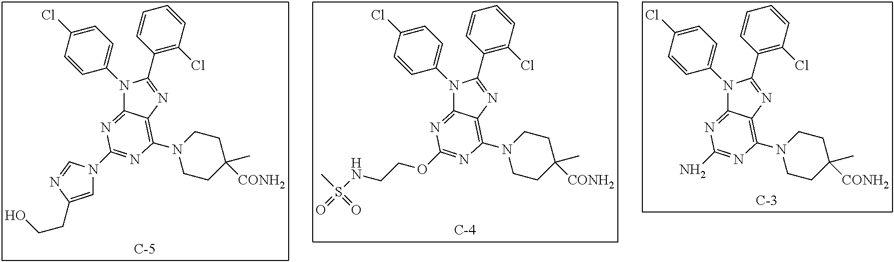

### Preparation of C Compounds can be Found in Following Detailed Protocols.

### (vi) General Synthetic Method D:

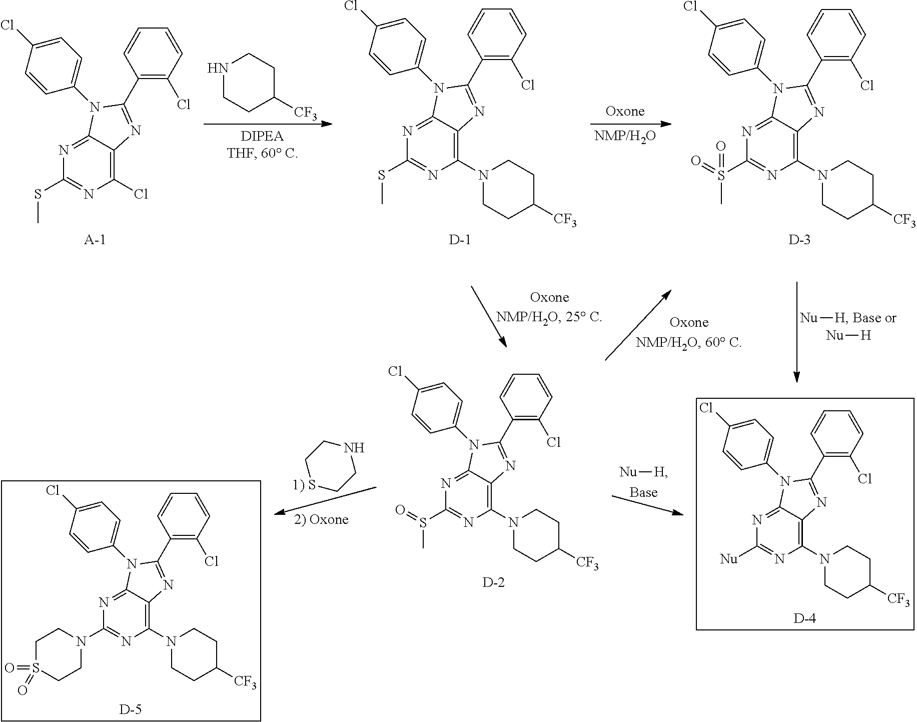

### Preparation of D Compounds can be Found in Following Detailed Protocols.

(vii) General Synthetic Method E:

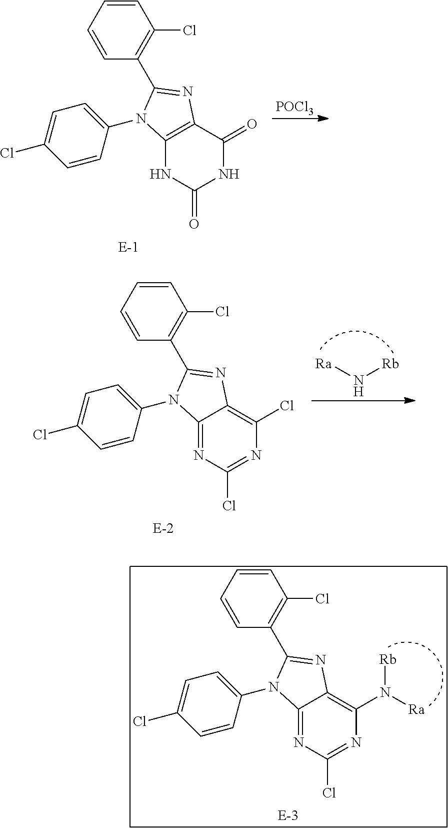

### Preparation of E-3 Compounds:

Method for compound 144: Step 1, a suspension of 8-(2-chlorophenyl)-9-(4-chlorophenyl)-1H-purine-2,6(3H,9H)-dione (400 mg, 1.07 mmol) in phosphorus oxychloride (4.4 mL, 47 mmol) was heated to 100° C. The solution was stirred at that temperature during 1 week. 20% SM was still remaining after 1 week, but the reaction was stopped there. The reaction mixture was concentrated under reduced pressure and the residue was dissolved in DCM before being transferred into a separation funnel containing sat. NaHCO₃. The solution was neutralized, dried with anhydrous Na₂SO₄ before being concentrated. The residue was purified on normal phase flash chromatography (loading with DCM, 25 g) using 50-100% DCM in hexane affording 2,6-dichloro-8-(2-chlorophenyl)-9-(4-chlorophenyl)-9H-purine (220 mg, 50%).

Step 2, N,N-Diisopropylethylamine (188 μL, 1.07 mmol) was added to a vial containing a solution of 2,6-dichloro-8-(2-chlorophenyl)-9-(4-chlorophenyl)-9H-purine (220 mg, 536 μmol) and 4-(ethylamino)piperidine-4-carboxamide (97 mg, 563 μmol) in NMP (1.3 mL). the reaction mixture was stirred at room temperature until full conversion was observed by LCMS (3 hours). Water (15 mL) was slowly added to the reaction mixture and the suspension was removed by filtration on Buchner. 15 mg of crude product was purified on a semi-prep system (injection of reaction mixture) using a gradient of 40-70% ACN in 10 mM AmB (pH 4) affording 1-(2-chloro-8-(2-chlorophenyl)-9-(4-chlorophenyl)-9H-purin-6-yl)-4-(ethylamino)piperidine-4-carboxamide (Compound 144, 10.5 mg) after lyophilizing pure fractions. ¹H NMR (400 MHz, DMSO-d₆) δ 7.70 (dd, 1H), 7.55-7.49 (m, 4H), 7.49-7.42 (m, 1H), 7.37-7.31 (m, 3H), 7.05 (s, 1H), 5.07 (br s, 1H), 4.28 (br s, 1H), 4.10 (br s, 1H), 3.63 (br s, 1H), 2.39 (q, 2H), 1.88 (br s, 2H), 1.69 (br d, 2H), 1.04 (t, 3H). LCMS: (ES⁺) m/z=544.2 (M+H).

(viii) General Synthetic Method F:

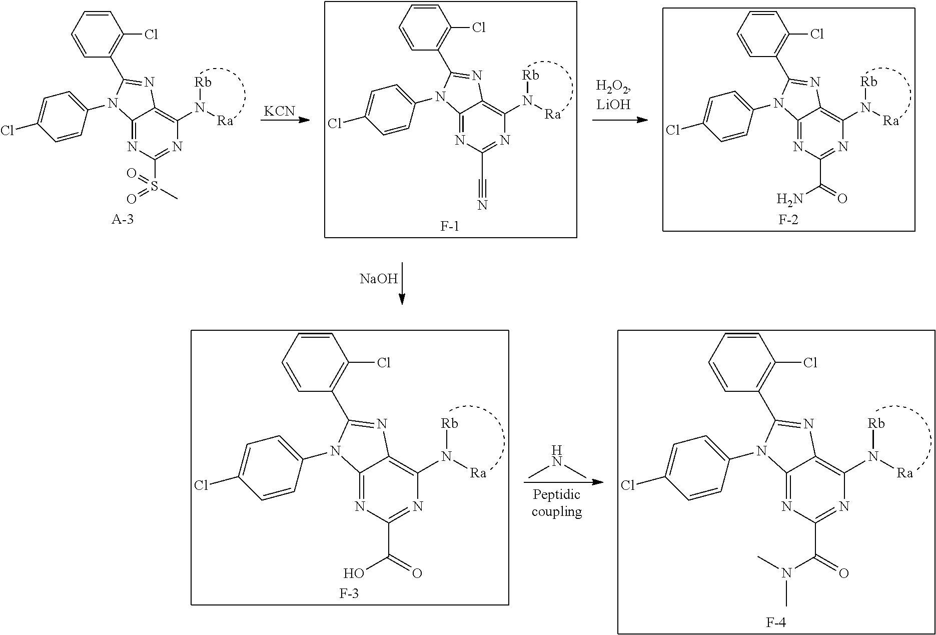

### Preparation of F Compounds:

Method A: To a solution of 1-[8-(2-chlorophenyl)-9-(4-chlorophenyl)-2-methylsulfonyl-purin-6-yl]-4-methyl-piperidine-4-carboxamide (Intermediate A-3) (500 mg, 0.89 mmol, 1 eq.) in dimethylsulfoxide (5 mL) was added potassium cyanide (87 mg, 1.34 mmol, 1.5 eq.), the resulting mixture was stirred at 100° C. for 15 hours. The mixture was diluted with ethyl acetate (50 mL), washed with brine (3*15 mL), dried over sodium sulfate, filtered and concentrated in vacuum to give the intermediate F-1 1-[8-(2-chlorophenyl)-9-(4-chlorophenyl)-2-cyano-purin-6-yl]-4-methyl-piperidine-4-carboxamide (Compound 146, 450 mg, crude) as a yellow solid.

Method B: A vial was charged with lithium hydroxide (0.4 mg, 18 μmol). water (250 μL) then hydrogen peroxide (2.5 μL, 23.9 μmol) (30 w % in water) were added in one portion. A solution of 1-(8-(2-chlorophenyl)-9-(4-chlorophenyl)-2-cyano-9H-purin-6-yl)-4-(ethylamino)piperidine-4-carboxamide (Compound 146, intermediate F-1) (8.0 mg, 14.9 μmol) in THF (250 μL) was then added and the reaction mixture was stirred at room temperature during 3 hours where full conversion was observed by LCMS. EtOAc (15 mL) was added and the solution was washed 5 times with saturated NH₄Cl, dried with anhydrous Na₂SO₄ and concentrated. The residue was purified on reverse phase flash chromatography (loading with DMSO, C18 12 g) using 10-60% ACN in 10 mM AmF (pH 4) affording intermediate F-2, 6-(4-carbamoyl-4-(ethylamino)piperidin-1-yl)-8-(2-chlorophenyl)-9-(4-chlorophenyl)-9H-purine-2-carboxamide (Compound 147, 3.5 mg, 42% yield) after lyophilisation.

Method C: To a solution of sodium hydroxide (178 mg, 4.44 mmol, 5.0 eq.) in water (3 mL) and ethanol (3 mL) was added 1-[8-(2-chlorophenyl)-9-(4-chlorophenyl)-2-cyano-purin-6-yl]-4-methyl-piperidine-4-carboxamide (Compound 146, intermediate F-1) (450 mg, 0.89 mmol, 1.0 eq.), the mixture was stirred at 60° C. for 3 hours. The mixture was diluted with water (10 mL), washed with ethyl acetate (2*10 mL), pH of the water phase was acidified by hydrochloric acid (1 N) to pH=3, then extracted with ethyl acetate (2*20 mL), the organic layers were washed with brine (5 mL), dried over sodium sulfate, filtered and concentrated in vacuum. The residue was purified by prep-HPLC (column: Phenomenex luna C18 150*40 mm*15 μm; mobile phase: [water (FA)-ACN]; B %: 42%-72%, 10 min) to give the crude product. The residue was purified by prep-HPLC (column: Phenomenex luna C18 150*25 mm*10 μm; mobile phase: [water (FA)-ACN]; B %: 40%-70%, 10 minutes) to give intermediate F-3, 6-(4-carbamoyl-4-methyl-1-piperidyl)-8-(2-chlorophenyl)-9-(4-chlorophenyl)purine-2-carboxylic acid (Compound 77, 8.06 mg, 15.3 μmol, 2.8% yield) as an off-white solid.

Method D: To a solution of 6-(4-carbamoyl-4-methyl-1-piperidyl)-8-(2-chlorophenyl)-9-(4-chlorophenyl) purine-2-carboxylic acid (Compound 77, intermediate F-3) (45 mg, 85.6 μmol, 1 eq.) in N,N-dimethylformamide (0.5 mL) was added N,N-diisopropylethylamine (44 mg, 0.34 mmol, 0.06 mL, 4 eq.), 1-hydroxybenzotriazole (14 mg, 0.10 mmol, 1.2 eq.) and 1-(3-dimethylaminopropyl)-3-ethylcarbodiimide hydrochloride (20 mg, 0.10 mmol, 1.2 eq.), the mixture was stirred at 25° C. for 0.5 hour. Then dimethylamine hydrochloride (14 mg, 0.17 mmol, 2 eq.) was added to the mixture, the mixture was stirred at 25° C. for 2 hours. The residue was dissolved in acetonitrile (2 mL) and purified by prep-HPLC (column: Waters Xbridge 150*25 mm*5 μm; mobile phase: [water (ammonium bicarbonate)-acetonitrile]; B %: 40%-70%, 8 min) and prep-HPLC (column: Phenomenex Luna C18 150*25 mm*10 μm; mobile phase: [water(methanoic acid)-acetonitrile]; B %: 40%-70%, 10 min) and the organic phase was concentrated under reduced pressure to remove acetonitrile, and the liquid was lyophilized to give intermediate F-4, 6-(4-carbamoyl-4-methyl-1-piperidyl)-8-(2-chlorophenyl)-9-(4-chlorophenyl)-N,N-dimethyl-purine-2-carboxamide (Compound 2, 16.52 mg, 30 μmol, 35% yield) as a yellow solid.

TABLE 7
Reaction conditions and data for F-1 to F-4 compounds

| Compounds F-1, | Starting | | Exact |
|---|---|---|---|
| F-2, F-3 and F-4 | material | ¹H NMR, 400 MHz | Mass |
| 2 | 77 | DMSO-d₆, δ = 7.74-7.66 (m, 1H), 7.56-7.43 (m, 5H), 7.36- | 552.3 |
| METHOD D | | 7.30 (m, 2H), 7.27 (s, 1H), 6.99-6.92 (m, 1H), 5.32-3.35 (m, | (M + H)⁺ |
| F-4 | | 2H), 3.29 (s, 2H), 2.94 (s, 3H), 2.82 (s, 3H), 2.16-2.08 (m, | |
| | | 2H), 1.50-1.38 (m, 2H), 1.17 (s, 3H) | |
| 77 | 146 | DMSO-d₆, δ = 7.74-7.68 (m, 1H), 7.55-7.43 (m, 5H), 7.39- | 525.2 |
| METHOD C | | 7.33 (m, 2H), 7.28 (s, 1H), 6.97 (s, 1H), 5.20-4.32 (m, 2H), | (M + H)⁺ |
| F-3 | | 4.03-3.57 (m, 2H), 2.15-2.07 (m, 2H), 1.45 (t, J = 9.6 Hz, | |
| | | 2H), 1.17 (s, 3H) | |
| 146 | A-3(141) | DMSO-d₆, = 7.71 (d, 1H), 7.59-7.42 (m, 5H), 7.43-7.31 (m, | 535.3 |
| METHOD A | | 3H), 7.05 (s, 1H), 5.11 (br s, 1H), 4.37 (br s, 1H), 4.12 (br s, | (M + H)⁺ |
| F-1 | | 1H), 3.67 (br s, 1H), 2.43-2.36 (m, 2H), 1.90 (br s, 2H), 1.71 | |
| | | (br d, 2H), 1.04 (t, 3H) | |
| 147 | 146 | CD₃CN, = 7.65 (s, 1H), 7.62-7.55 (m, 1H), 7.53-7.38 (m, | 553.4 |
| METHOD B | | 5H), 7.34-7.28 (m, 2H), 6.94 (s, 1H), 6.11 (s, 1H), 5.72 (s, | (M + H)⁺ |
| F-2 | | 1H), 5.11 (br s, 1H), 4.67 (br s, 1H), 3.97 (br s, 2H), 2.51 (q, | |
| | | 2H), 2.05 (ddd, 2H), 1.78-1.68 (m, 2H), 1.10 (t, 3H) | |

### (ix) General Synthetic Method G:

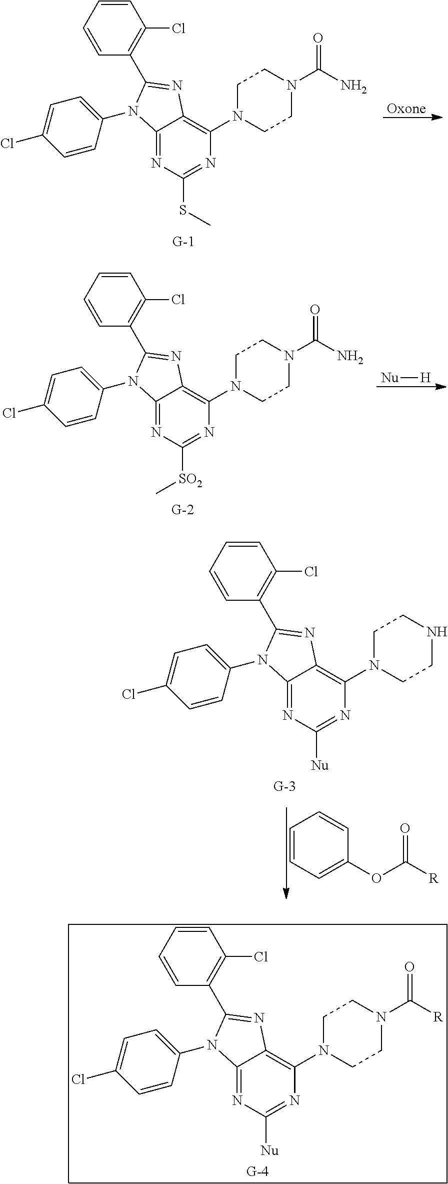

### Preparation of G-4 Compounds:

Method: Step 1, intermediate G-1 (515 μmol) was dissolved in NMP (3.1 mL) then water (300 μL) was added dropwise making sure that the solution stays clear. Oxone (950 mg, 1.55 mmol) was then added in one portion and the reaction mixture was stirred at room temperature overnight. Water (30 mL) was slowly added and the precipitate was recovered by filtration on Buchner. The solid was washed with water and it was then lyophilized affording intermediate G-2 (92% yield).

Step 2, cesium carbonate (494 μmol) was added to a solution of intermediate G-2 (165 μmol) in the nucleophile reagent (6.6 mmol). The solution was heated to 100° C. overnight. LCMS shows that the major product is the desired one. EtOAc (15 mL) was added and the solution was washed 5 times with saturated NH₄Cl, dried with anhydrous Na₂SO₄ and concentrated. The residue was purified on reverse phase flash chromatography (loading with DMSO, C18 12 g) using 10-60% ACN in 10 mM AmF (pH 4). The fractions were concentrated under reduced pressure. The residues were dissolved in DCM, dried with anhydrous Na₂SO₄, filtered and concentrated affording intermediate G-3 (78% yield).

Step 3, triethylamine (361 μmol) was added to a solution of intermediate G-3 (120 μmol) and phenolcarbamate (181 μmol) in DCM (1.2 mL) at room temperature. Full conversion was observed by LCMS after 90 minutes. The reaction mixture was concentrated to dryness and the residue was purified on reverse phase flash chromatography (loading with DMSO, C18 12 g) using 10-70% ACN in 10 mM AmF (pH 4) affording compound G-4 (11% yield) after lyophilization.

### Reaction Scale: 265 mg of Intermediate G-1.

TABLE 8
Reaction conditions and data for G-4 compounds

| Compounds | Starting | | Exact |
|---|---|---|---|
| G-4 | material | ¹H NMR, 400 MHz | Mass |
| 157 | G-1 | MeOD, δ = 7.62-7.55 (m, 1H), 7.48-7.33 (m, 5H), 7.30- | 541.4 |
| | | 7.24 (m, 2H), 4.27-4.22 (m, 4H), 3.77-3.67 (m, 4H), 3.58- | (M + H)⁺ |
| | | 3.54 (m, 4H), 3.18 (s, 3H) | |
| 173 | G-1 | DMSO-d6, δ = 7.69-7.65 (m, 1H), 7.52-7.41 (m, 5H), 7.30- | 556.3 |
| | | 7.25 (m, 2H), 4.59 (t, 1H), 4.17 (br s, 4H), 3.63 (s, 3H), 3.59- | (M + H)⁺ |
| | | 3.54 (m, 4H), 3.54-3.49 (m, 4H), 3.07 (s, 3H) | |
| 175 | G-1 | DMSO-d6, δ = 8.35-8.25 (m, 1H), 7.70-7.64 (m, 1H), 7.52- | 553.4 |
| | | 7.37 (m, 5H), 7.30-7.22 (m, 2H), 5.86 (s, 2H), 4.83-4.73 (m, | (M + H)⁺ |
| | | 1H), 4.67 (s, 1H), 4.16-3.98 (m, 3H), 3.88 (dd, 2H), 3.67 (s, | |
| | | 1H), 3.37 (s, 2H), 2.02-1.71 (m, 4H) | |
| 176 | G-1 | DMSO-d6, δ = 8.33-8.25 (m, 1H), 7.70-7.64 (m, 1H), 7.51- | 527.3 |
| | | 7.39 (m, 5H), 7.30-7.24 (m, 2H), 5.86 (s, 2H), 4.67-4.58 (m, | (M + H)⁺ |
| | | 1H), 4.09 (t, 2H), 3.94-3.84 (m, 2H), 3.58 (s, 4H), 3.08 (s, 3H) | |

### (x) General Synthetic Method H:

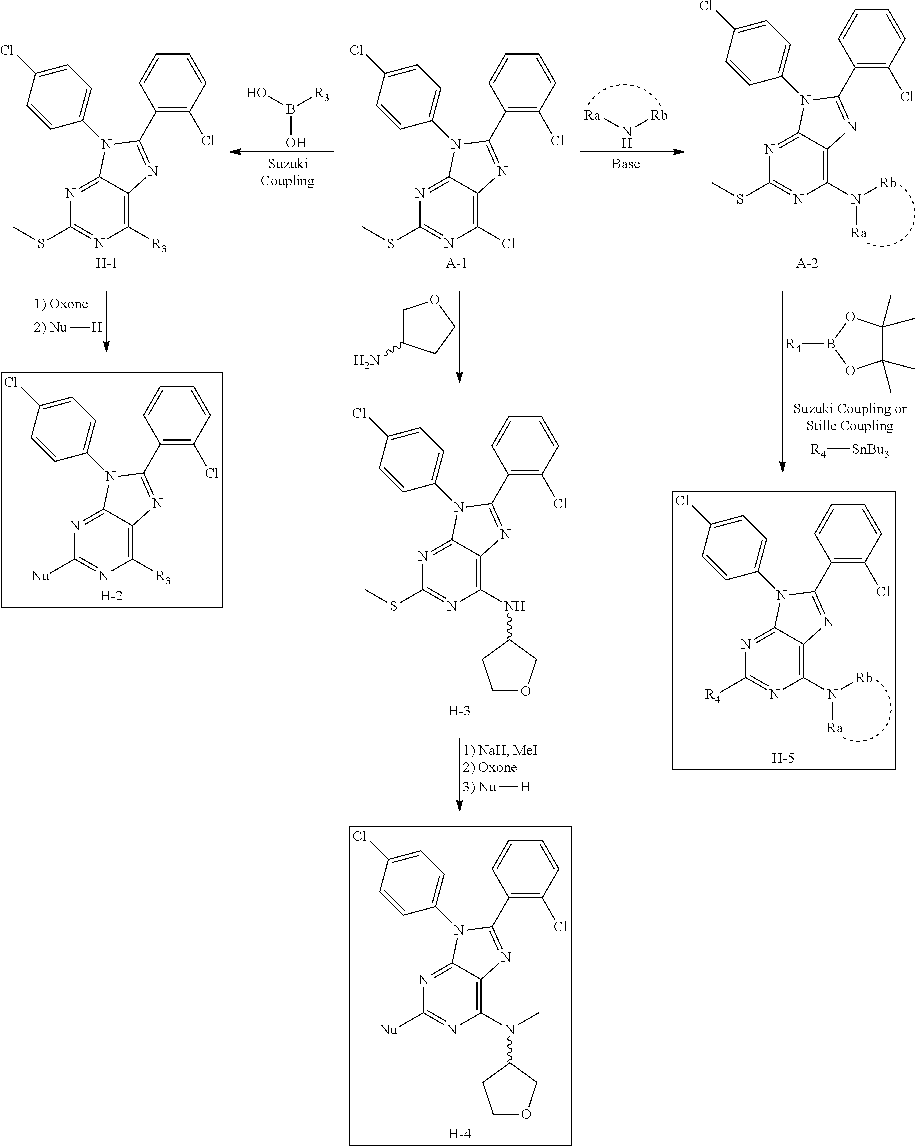

### Preparation of H-2 Compounds:

Method A: Step 1, 1,1′-Bis(diphenylphosphino)ferrocene dichloropalladium (II) (0.2 eq.) was added to a mixture of 6-chloro-8-(2-chlorophenyl)-9-(4-chlorophenyl)-2-(methylthio)-9H-purine (Intermediate A-1) (1 eq.), 2-(trifluoromethyl)pyridine-5-boronic acid (1.6 eq.) and sodium carbonate (4 eq.) in dioxane (10 mL) and water (1.6 mL). The reaction mixture was stirred at 70° C. for 4 hours. The reaction mixture was filtered over Celite (EtOAc and trace MeCN rinse). The filtrate was partially concentrated in vacuo, then diluted with water (15 mL) and extracted with ethyl acetate (70 mL×2). The organic phase was dried with magnesium sulfate and evaporated to afford crude 8-(2-chlorophenyl)-9-(4-chlorophenyl)-2-(methylthio)-6-(6-(trifluoromethyl)pyridin-3-yl)-9H-purine (Intermediate H-1, quantitative conversion).

Step 2, crude intermediate H-1 (1 eq.) was dissolved in NMP (8 mL), then water (600 μL) was added dropwise making sure that the solution stays clear. Oxone (3 eq.) was then added in one portion and the reaction mixture was stirred at room temperature for 16 hours and LCMS showed low conversion. More oxone (3 eq.) was added and the reaction was continued for further 24 hours, for a total 40 hours. Upon completion of the reaction, water (50 mL) was added, and the mixture was extracted with DCM (80 mL×2), dried over MgSO₄ and concentrated. To the residue (NMP solution) was added water (600 μL) and oxone (3.2 eq.) was added and the reaction was heated at 60° C. for 20 hours. Water (50 mL) was added and the mixture was extracted with DCM (90 mL×2), dried over MgSO₄ and evaporated to a yellow oil. Water (6 mL) was added to this oil (no precipitation). Then MeCN (3 mL) was added, and the resulting mixture was lyophilized and then subjected to flash chromatography using 0% to 40% ethyl acetate in hexane to afford intermediate 8-(2-chlorophenyl)-9-(4-chlorophenyl)-2-(methylsulfonyl)-6-(6-(trifluoromethyl)pyridin-3-yl)-9H-purine (81% yield).

Step 3, a solution of 8-(2-chlorophenyl)-9-(4-chlorophenyl)-2-(methylsulfonyl)-6-(6-(trifluoromethyl)pyridin-3-yl)-9H-purine (1 eq.) in the nucleophile Nu-H (30 eq.) was stirred at 100° C. for 21 hours. The compound precipitated by standing at room temperature. DMF, trace water, and trace MeCN were used to inject the compound onto a C-18 column for purification. Purification was done using 15% to 80% MeCN in AmF buffer and product came out impure. All fractions containing the product were evaporated to afford a yellow solid. MeCN (3 mL) and water (15 mL) were used to take up the solid which was then filtered, rinsing with water, and allowed to dry to afford the H-2 compound.

### Reaction Scale: 200 mg of Intermediate a-1

Method B: Step 1, to a solution of 6-chloro-8-(2-chlorophenyl)-9-(4-chlorophenyl)-2-methylsulfanyl-purine (Intermediate A-1) (1 eq.) in dioxane (10 mL) and water (2 mL) was added [6-(trifluoromethyl)-3-pyridyl]boronic acid (2.0 eq.), potassium phosphate (2.0 eq.) and tetrakis(triphenylphosphine)palladium (0.06 eq.). The mixture was stirred at 80° C. for 2 hours under nitrogen atmosphere. The reaction mixture was washed with water (50 mL) and extracted with ethyl acetate 150 mL (50 mL×3). The combined organic layers were washed with brine 200 mL (100 mL×2), dried over anhydrous sodium sulfate, filtered and concentrated under reduced pressure, the resulting residue was stirred with ethanol (100 mL), filtered and the filtration residue was concentrated under reduced pressure to give compound 8-(2-chlorophenyl)-9-(4-chlorophenyl)-2-methylsulfanyl-6-[6-(trifluoromethyl)-3-pyridyl]purine (Intermediate H-1, 87.1% yield) as a white solid. ¹H NMR (400 MHz, CDCl₃) δ=10.16 (s, 1H), 9.40-9.37 (m, 1H), 7.86 (d, J=8.0 Hz, 1H), 7.63-7.58 (m, 1H), 7.51-7.37 (m, 5H), 7.27-7.24 (m, 2H), 2.68 (s, 3H).

Step 2, to a solution of 8-(2-chlorophenyl)-9-(4-chlorophenyl)-2-methylsulfanyl-6-[6-(trifluoromethyl)-3-pyridyl]purine (Intermediate H-1) (1 eq.) in NMP (10 mL) and water (1 mL) was added potassium; oxidooxy hydrogen sulfate (3.16 g, 18.78 mmol, 10 eq.). The mixture was stirred at 60° C. for 1 hour. The reaction mixture was added dropwise into water 50 mL, filtered and the filtration residue was concentrated under reduced pressure to give compound 8-(2-chlorophenyl)-9-(4-chlorophenyl)-2-methylsulfonyl-6-[6-(trifluoromethyl)-3-pyridyl]purine (crude) as a light-yellow solid, which was used into the next step without further purification. ¹H NMR (400 MHz, CDCl₃) δ=10.26 (d, J=1.6 Hz, 1H), 9.43 (dd, J=1.7, 8.3 Hz, 1H), 7.91 (d, J=8.3 Hz, 1H), 7.64-7.59 (m, 1H), 7.56-7.50 (m, 1H), 7.50-7.40 (m, 4H), 7.31-7.27 (m, 2H), 3.48 (s, 3H).

Step 3, a mixture of 8-(2-chlorophenyl)-9-(4-chlorophenyl)-2-methylsulfonyl-6-[6-(trifluoromethyl)-3-pyridyl]purine (1 eq.), nucleophile Nu-H (5 eq.) and potassium carbonate (2 eq.) in N,N-dimethylformamide (1 mL) was stirred at 100° C. for 2 hours. The reaction mixture was diluted with N,N-dimethylformamide (1 mL) and filtered to afford the filter liquor. The filter liquor was purified by reversed-phase HPLC (column: Waters Xbridge 150*25 mm*5 μm; mobile phase: [water(ammonium bicarbonate)-ACN]; B %: 63%-93%, 8 minutes). The cut fraction was concentrated under reduced pressure to remove acetonitrile. The residue was lyophilized to give compound H-2 as a white solid.

### Reaction Scale: 1 g of Intermediate a-1

Method C: Step 1 and Step 2 can be achieved following Method A or Method B. Step 3, a mixture of 8-(2-chlorophenyl)-9-(4-chlorophenyl)-2-methylsulfonyl-6-[6-(trifluoromethyl)-3-pyridyl]purine (1 eq.) and nucleophile Nu-H (30 eq.) was stirred at 120° C. for 2 hours. The reaction mixture was cooled to room temperature and was purified by reversed-phase HPLC (column: Phenomenex luna C18 150*25 mm*10 μm; mobile phase: [water (formic acid)-ACN]; B %: 67%-97%, 10 minutes). The cut fraction was concentrated under reduced pressure to remove acetonitrile. The residue was lyophilized to give compound H-2 as a yellow solid.

Reaction Scale: 70 mg of 8-(2-chlorophenyl)-9-(4-chlorophenyl)-2-methylsulfonyl-6-[6-(trifluoromethyl)-3-pyridyl]purine

### Preparation of H-4 Compounds:

Method D: Step 1, sodium hydride 60% in dispersion in mineral oil (1.5 eq.) was added in one portion to a solution of intermediate H-3 (1 eq.) in NMP (1.6 mL) at room temperature. The reaction mixture was stirred during 5 minutes then iodomethane (2 eq.) was added. Full conversion was observed by LCMS after 5 minutes. The reaction mixture was transferred to a separation funnel containing saturated NH₄Cl. The aqueous solution was extracted with EtOAc, the organic layers were combined, dried with anhydrous Na₂SO₄ and concentrated affording crude intermediate (quantitative conversion).

Step 2, the solids were dissolved in NMP (3.0 mL). Water (300 uL) followed by oxone (3 eq.) were added. The reaction mixture was stirred at room temperature during 2 hours. Water (35 mL) was then added and the precipitate was recovered on Buchner.

Step 3, the resulting sulfone is a solid that was dissolved in 2-(methylamino)ethanol (Nu-H) (25 eq.) and heated to 100° C. overnight. The reaction mixture was injected on reverse phase column (C18 12 g) and was purified using 40-70% ACN in 10 mM AmF (pH 4) affording compound H-4 (33% yield).

### Reaction Scale: 150 mg of Intermediate H-3

### Preparation of H-5 Compounds:

Method E: To a solution of intermediate A-2 (1 eq.) in tetrahydrofuran (6 mL) was added tert-butyl 3-(4,4,5,5-tetramethyl-1,3,2-dioxaborolan-2-yl)pyrazole-1-carboxylate (2 eq.), palladium; triphenylphosphane (0.1 eq.) and cuprous 2-hydroxy-3-methyl-benzoate (3 eq.), the mixture was stirred at 60° C. for 12 hours. The mixture was diluted with water (10 mL), extracted with ethyl acetate (10 mL*3). The organic layer was dried with anhydrous anhydrous sodium sulfate, filtered and concentrated under vacuum. The residue was purified by column chromatography (silicon dioxide) and concentrated under vacuum. The residue was purified by Pre-HPLC (column: C18; mobile phase: [water(formic acid)-ACN]) and dried by lyophilization. Then the crude compound was purified by normal phase HPLC (column: Welch Ultimate XB-CN 250*50*10 μm; mobile phase: [Hexane-ethanol (0.1% ammonia hydroxide)]) and concentrated under vacuum to give compound H-5 as an off-white solid.

### Reaction Scale: 300 mg of Intermediate a-2

Method F: Step 1, to a solution of intermediate A-2 (1.0 eq.) in tetrahydrofuran (5 mL) was added trimethyl-[2-[(5-tributylstannylimidazol-1-yl)methoxy]ethyl]silane (2 eq.), palladium-triphenylphosphane (0.1 eq) and cuprous-2-hydroxy-3-methyl-benzoate (3.0 eq.), the mixture was stirred at 60° C. for 2 hours. The reaction mixture was filtered and concentrated in vacuum. black brown oil was purified by prep-HPLC (column: Phenomenex luna C18 150*25 mm*10 μm; mobile phase: [water(FA)-ACN]; B %: 42%-72%, 10 minutes) to give the intermediate (100 mg, 0.15 μmol, 15.5% yield) as a yellow oil.

Step 2, to a solution of the intermediate compound (50 mg, 73.8 μmol, 1.0 eq.) in dichloromethane (6 mL) and ethanol (0.2 mL) was added trifluoroacetic acid (2 mL), the mixture was stirred at 25 MC for 3 hours. The mixture was concentrated in vacuum, then tetrahydrofuran (10 mL) was added and sodium hydroxide (30 mg) was added, the resulting mixture was stirred at 80° C. for 1 hour then concentrated in vacuum, and the residue was triturated with N,N-dimethylformamide (5 mL, filtered and the filtrate was concentrated in vacuum. The residue was purified by prep-HPLC (column: Phenomenex Luna C18 150*25 mm*1 μm; mobile phase: [water(formic acid)-ACN]; B %: 16%-46%, 10 minutes) to give compound H-5 as an off-white solid.

### Reaction Scale: 500 mg of Intermediate a-2

TABLE 9
Reaction conditions and data for H-2, H-4 and H-5 compounds

| Compounds H-2, | Starting | | Exact |
|---|---|---|---|
| H-4 and H-5 | material | ¹H NMR, 400 MHz | Mass |
| 39 | A-2(39) | DMSO-d₆, δ = 13.44 (s, 1H), 7.74-7.73 (m, 1H), 7.56-7.43 | 558.2 |
| Method E | | (m, 5H), 7.39 (d, J = 8.8 Hz, 2H), 6.73 (s, 1H), 6.18-5.34 (m, | (M + H)⁺ |
| 4% yield | | 2H), 3.24-3.09 (m, 2H), 2.86-2.71 (m, 1H), 2.10-1.94 (m, | |
| | | 2H), 1.54-1.50 (m, 2H). | |
| | | ¹⁹F NMR (377 MHz, DMSO-d6) δ = −72.49 (s, 1F) | |
| 44 | A-2(44) | DMSO-d₆, δ = 13.67-13.11 (m, 1H), 7.73-7.71 (m, 1H), 7.54- | 547.2 |
| Method E | | 7.43 (m, 5H), 7.41-7.34 (m, 2H), 7.29 (s, 1H), 6.96 (s, 1H), | (M + H)⁺ |
| 42% yield | | 6.73 (br s, 1H), 5.26-4.37 (m, 2H), 4.10-3.47 (m, 2H), 2.17- | |
| | | 2.08 (m, 2H), 1.48-1.43 (m, 2H), 1.18 (s, 3H) | |
| 45 | A-2(44) | MeOD-d₄, δ = 8.17-7.86 (m, 1H), 7.80-7.42 (m, 7H), 7.40- | 547.2 |
| Method F | | 7.35 (m, 2H), 7.29 (s, 1H), 6.96 (s, 1H), 5.21-4.46 (m, 2H), | (M + H)⁺ |
| 15.5% yield | | 4.10-3.61 (m, 2H), 2.21-2.09 (m, 2H), 1.56-1.39 (m, 2H), | |
| (Step 1) | | 1.18 (s, 3H) | |
| 36% yield | | | |
| (Step 2) | | | |
| 123 | A-1 | DMSO-d₆, δ = 10.03 (s, 1H), 9.39 (d, J = 8.8 Hz, 1H), 8.22 (d, | 574.2 |
| Method B | | J = 8.4 Hz, 1H), 7.80 (d, J = 7.2 Hz, 1H), 7.62-7.55 (m, 4H), | (M + H)⁺ |
| 44.4% yield | | 7.55-7.50 (m, 1H), 7.46 (d, J = 8.8 Hz, 2H), 4.74 (s, 1H), | |
| (Step 3) | | 4.26 (s, 2H), 1.24 (s, 6H) | |
| 124 | A-1 | CDCl₃, δ = 10.04 (s, 1H), 9.39-9.27 (m, 1H), 7.85 (d, J = 8.4 | 587.2 |
| Method C | | Hz, 1H), 7.59 (d, J = 7.2 Hz, 1H), 7.48-7.36 (m, 5H), 7.24 | (M + H)⁺ |
| 56.6% yield | | (d, J = 8.0 Hz, 2H), 4.85-4.38 (m, 1H), 3.77 (s, 2H), 3.39 (s, | |
| (Step 3) | | 3H), 1.31 (s, 6H) | |
| 125 | A-1 | DMSO-d₆, δ = 9.98 (s, 1H), 9.40-9.34 (m, 1H), 8.18 (d, J = | 585.2 |
| Method C | | 8.4 Hz, 1H), 7.81-7.75 (m, 1H), 7.57-7.48 (m, 5H), 7.43- | (M + H)⁺ |
| 28.8% yield | | 7.39 (m, 2H), 4.66-4.59 (m, 2H), 4.45 (t, J = 6.0 Hz, 2H), | |
| (Step 3) | | 4.11-3.98 (m, 2H), 3.45-3.35 (m, 1H), 3.19 (s, 3H) | |
| 126 | A-1 | CDCl₃, δ = 10.01 (s, 1H), 9.40 (d, J = 8.4 Hz, 1H), 7.85 (d, | 599.1 |
| Method C | | J = 8.4 Hz, 1H), 7.63-7.57 (m, 1H), 7.46-7.36 (m, 5H), 7.27- | (M + H)⁺ |
| 35.8% yield | | 7.22 (m, 2H), 4.74 (d, J = 5.6 Hz, 2H), 4.34 (d, J = 4.8 Hz, | |
| (Step 3) | | 2H), 4.00 (s, 2H), 3.30 (s, 3H), 1.41 (s, 3H) | |
| 163 | A-1 | DMSO-d6, δ = 9.98 (s, 1H), 9.39 (s, 1H), 8.17 (d, 1H), 7.78 | 585.5 |
| Method A | | (d, 1H), 7.61-7.47 (m, 5H), 7.46-7.34 (m, 2H), 4.81 (bs, | (M + H)⁺ |
| 56% yield | | 1H), 4.50-4.03 (m, 1H), 3.88-3.43 (m, 3H), 2.20-1.79 | |
| (Step 3) | | (m, 5H) | |
| 165 | A-1 | DMSO-d6, δ = 9.97 (s, 1H), 9.35 (dd, 1H), 8.17 (d, 1H), 7.78 | 559.4 |
| Method A | | (dd, 1H), 7.59-7.46 (m, 5H), 7.44-7.35 (m, 2H), 4.71 (t, | (M + H)⁺ |
| 56% yield | | 1H), 3.79 (bs, 2H), 3.71-3.62 (m, 2H), 3.24 (s, 3H) | |
| (Step 3) | | | |
| 196 | H-3 | DMSO-d6, δ = 7.68-7.61 (m, 1H), 7.53-7.37 (m, 5H), 7.33- | 513.3 |
| Method D | | 7.19 (m, 2H), 6.09 (br s, 1H), 4.59 (t, 1H), 3.98 (td, 1H), | (M + H)⁺ |
| (Step 3) | | 3.86-3.76 (m, 2H), 3.65 (q, 1H), 3.59-3.54 (m, 4H), 3.30 | |
| | | (s, 3H), 3.08 (s, 3H), 2.31-2.21 (m, 1H), 2.05-1.95 (m, 1H) | |
| 197 | H-3 | DMSO-d6, δ = 7.67-7.62 (m, 1H), 7.51-7.40 (m, 5H), 7.29- | 513.3 |
| Method D | | 7.24 (m, 2H), 6.08 (br s, 1H), 4.59 (t, 1H), 3.98 (td, 1H), | (M + H)⁺ |
| (Step 3) | | 3.88-3.75 (m, 2H), 3.65 (q, 1H), 3.60-3.53 (m, 4H), 3.30 | |
| | | (br s, 3H), 3.08 (s, 3H), 2.32-2.22 (m, 1H), 2.07-1.94 (m, 1H) | |

### (xi) General Synthetic Method I:

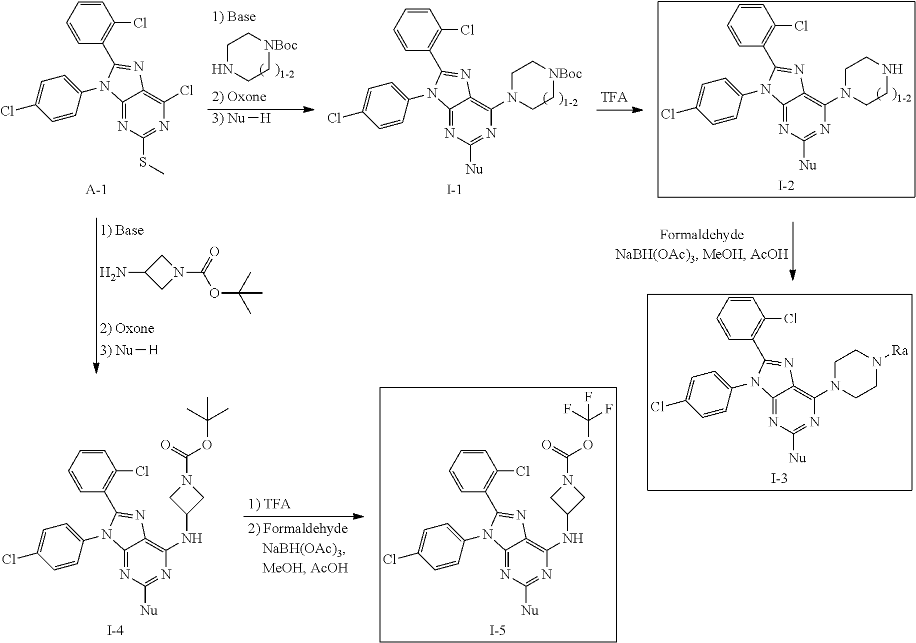

### Preparation of I-2 Compounds:

Method A: Step 1, the amine derivative (1.2 eq.) was added to a vial containing a solution of intermediate A-1 (1 eq.) and N,N-Diisopropylethylamine (2.2 eq.) in NMP (4.0 mL). The reaction mixture was stirred at room temperature until full conversion was observed overnight by LCMS. Water (50 mL) was slowly added to the reaction mixture and the precipitate was recovered by filtration on Buchner. The solids were washed with water and dried under vacuum. Crude intermediate material (89% yield) was obtained as a solid and was used in the next reaction without any purification.

The solids were dissolved in DCM (10.0 mL) and acetone (1 mL) and tetrabutylammonium bromide (0.05 eq.) followed by a solution of oxone (2.6 mmol) in water (5.0 mL) were added. The biphasic solution was vigorously stirred overnight at 40° C. and 70% conversion to the desired sulfone was observed by LCMS. DCM was added and the organic solution was washed with brine 3 times before being dried with anhydrous Na₂SO₄ and concentrated under pressure affording the crude intermediate material (59% yield).

The crude intermediate material was then dissolved in the nucleophile (Nu-H) (2.6 eq.) and NMP (200 μL). The reaction mixture was still cloudy after heating at 100° C. but became clearer as the product was formed. Full conversion was observed overnight by LCMS. EtOAc (30 mL) was added and the solution was washed 5 times with saturated NH₄Cl, dried with anhydrous Na₂SO₄ and concentrated. The residue was purified on normal phase flash chromatography (loading with DCM, 25 g) using 0˜30% EtOAc in DCM affording intermediate I-1 (20% yield).

Step 2, trifluoroacetic acid (14 eq.) was added to a solution of intermediate I-1 (1 eq.) in DCM (700 μL) at room temperature. The solution was stirred 1 hour and full conversion was observed by LCMS. The reaction mixture was concentrated to dryness, diluted in DCM and washed 3× with saturated NaHCO₃. The organic layer was dried with anhydrous Na₂SO₄, filtered and concentrated under reduced pressure to afford crude compound 1-2 (quantitative conversion).

### Reaction Scale: 1 g of Intermediate a-1

### Preparation of I-3 Compounds:

Method B: Sodium triacetoxyborohydride (3 eq.) was added portion wise to a solution of intermediate 1-2 (1 eq.) and formaldehyde (5 eq.) (37% in water) in MeOH (2.9 mL) and AcOH (600 μL) at room temperature. Full conversion was observed after 2.5 hours. The reaction mixture was concentrated under reduced pressure and the residue was dissolved in DCM. The organic layer was washed with saturated NaHCO₃ (3×) and 1M NaOH (2×). The residue obtained after concentration was purified on reverse phase flash chromatography (loading with DMSO, C18 12 g, 4 CV) using 10-100% ACN in 10 mM AmF (pH 4) affording compound 1-3 (58% yield) as a solid.

### Reaction Scale: 125 mg of Intermediate I-2

TABLE 10
Reaction conditions and data for I-2, I-3 and I-5 compounds

| Compounds I-2, | Starting | | Exact |
|---|---|---|---|
| I-3 and I-5 | material | ¹H NMR, 400 MHz | Mass |
| 96 | I-2 | DMSO-d6, δ = 7.69 (dd, 1H), 754-7.40 (m, 5H), 7.34- | 555.3 |
| Method B | | 7.26 (m, 2H), 4.60 (s, 1H), 4.15 (bs, 3H), 3.97 (s, 2H), 2.76- | (M + H)⁺ |
| Ra = isopropyl | | 2.63 (m, 1H), 2.60-2.53 (m, 4H), 1.15 (s, 6H), 0.99 (d, 6H) | |
| 168 | I-2 | DMSO-d6, δ = 7.68-7.64 (m, 1H), 7.51-7.41 (m, 5H), | 512.3 |
| Method B | | 7.30-7.24 (m, 2H), 4.61-4.57 (m, 1H), 4.16 (br s, 4H), | (M + H)⁺ |
| Ra = methyl | | 3.56 (s, 4H), 3.07 (s, 3H), 2.42 (t, 4H), 2.21 (s, 3H) | |
| 179 | A-1 | DMSO-d6, δ = 7.70-7.63 (m, 1H), 7.52-7.39 (m, 5H), | 580.3 |
| Method | | 7.31-7.23 (m, 2H), 4.90 (bs, 1H), 4.75 (t, 1H), 4.60 (t, | (M + H)⁺ |
| A + B | | 1H), 4.55-4.47 (m, 1H), 4.46-4.38 (m, 1H), 4.21 (dd, | |
| | | 1H), 3.64-3.51 (m, 4H), 3.08 (s, 3H) | |
| 219 | A-1 | DMSO-d6, δ = 7.69 (dd, 1H), 7.53-7.41 (m, 5H), 7.34- | 513.2 |
| Method A | | 7.27 (m, 2H), 4.61 (bs, 1H), 4.19 (bs, 3H), 3.97 (s, 2H), | (M + H)⁺ |
| | | 3.62-3.09 (m, 2H), 2.96-2.83 (m, 4H), 1.15 (s, 6H) | |
| 221 | A-1 | DMSO-d6, δ = 7.65 (d, 1H), 7.53-7.40 (m, 5H), 7.34- | 563.3 |
| Method A | | 7.27 (m, 2H), 5.04 (bs, 1H), 4.63 (s, 1H), 4.47 (bs, 2H), | (M + H)⁺ |
| | | 4.11-3.82 (m, 3H), 3.13-2.97 (m, 4H), 2.95-2.69 (m, | |
| | | 1H), 1.15 (s, 6H) | |
| 240 | I-2 | DMSO-d6, δ = 7.69 (d, 1H), 7.55-7.40 (m, 5H), 7.31 (d, | 541.3 |
| Method B | | 2H), 4.63 (s, 1H), 4.49-3.71 (m, 4H), 3.34 (s, 3H), 3.19- | (M + H)⁺ |
| Ra = ethyl | | 2.66 (m, 5H), 1.23-1.08 (m, 9H) | |

(xii) General Synthetic Method J:

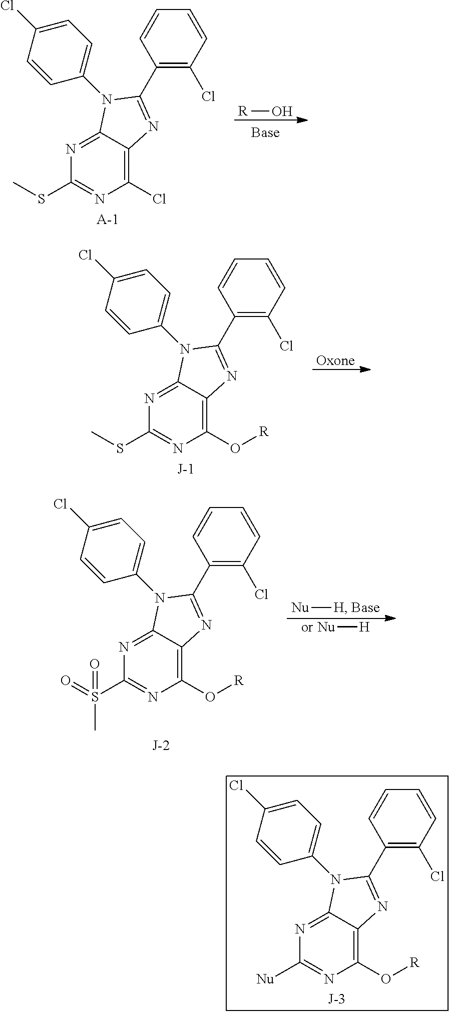

### Preparation of J-3 Compounds:

Method A: a mixture of intermediate J-2 (1 eq.), nucleophile (Nu-H) (5 eq.) and potassium carbonate (2 eq.) in N,N-dimethylformamide (1 mL) was stirred at 100° C. for 14 hours. The reaction mixture was cooled at room temperature and diluted with N,N-dimethylformamide (1 mL). The resulting mixture was filtered to afford the filter liquor. The filter liquor was purified by reversed-phase HPLC (column: Waters Xbridge 150*25 mm*5 μm; mobile phase: [water(ammonium bicarbonate)-ACN]; B %: 50%-80%) to give compound J-3 as a white solid.

### Reaction Scale: 80 mg of Intermediate J-2

Method B: a mixture of intermediate J-2 (1 eq.), nucleophile (Nu-H) (5 eq.) and potassium carbonate (2 eq.) in N,N-dimethylformamide (4 mL) was stirred at 100° C. for 6 hours. The reaction mixture was cooled at room temperature and filtered to afford the filter liquor. The filter liquor was purified by prep-HPLC (column: Waters Xbridge 150*25 mm*5 μm; mobile phase: [water(ammonium bicarbonate)-ACN]; B %: 51%-81%) to give desired compound as a white solid, which was further separated by SFC (column: DAICEL CHIRALPAK IC (250 mm*30 mm*10 μm); mobile phase: [0.1% ammonia hydroxide methanol] to give compound J-3 as a white solid.

### Reaction Scale: 300 mg of Intermediate J-2

Method C: a mixture of 8-(2-chlorophenyl)-9-(4-chlorophenyl)-6-ethoxy-2-methylsulfonyl-purine (1 eq.), nucleophile (Nu-H) (5 eq.) and potassium carbonate (2 eq.) in N,N-dimethylformamide (1.5 mL) was stirred at 100° C. for 6 hours. The reaction mixture was cooled at room temperature and diluted with N,N-dimethylformamide (2 mL). The resulting mixture was filtered to afford the filter liquor. The filter liquor was purified by reversed-phase HPLC (column: Unisil 3-100 C18 Ultra 150*50 mm*3 μm; mobile phase: [water(formic acid)-ACN]; B %: 45%-75%, 7 minutes). The cut fraction was concentrated under reduced pressure to remove acetonitrile. The residue was lyophilized to give compound J-3 as a white solid.

### Reaction Scale: 120 mg of Intermediate J-2

Method D: intermediate J-2 (1 eq.) was dissolved in the nucleophile (Nu-H) (1 mL) and the reaction mixture was stirred at 100° C. overnight. EtOAc (15 mL) was added and the solution was washed 5 times with saturated NH₄Cl, dried with anhydrous Na₂SO₄ and concentrated. Crude residue was purified (injection with DMSO) on Buchi semi-prep (C18 column) using 20-60% ACN in AmF. The fractions were lyophilized affording compound J-3.

### Reaction Scale: 50 mg of Intermediate J-2

Method E: to the intermediate J-2 (1 eq.) in a 8 mL vial was added potassium carbonate (2.2 eq.), nucleophile (Nu-H) (11 eq.), and DMF (1.2 mL) and the mixture was stirred at 110° C. for 19 hours. The reaction mixture was purified by reverse phase flash chromatography using 10%-100% MeCN in 10 mM AmF (product elution at 72% MeCN), affording compound J-3.

### Reaction Scale: 120 mg of Intermediate J-2

TABLE 11
Reaction conditions and data for J-3 compounds

| Compounds | Starting | | Exact |
|---|---|---|---|
| J-3 | material | ¹H NMR, 400 MHz | Mass |
| 86 | J-2(86) | DMSO-d6, δ = 7.74-7.68 (m, 1H), 7.56-7.43 (m, 5H), 7.38- | 487.2 |
| Method A | | 7.31 (m, 2H), 5.64-5.55 (m, 1H), 4.70 (s,1H), 4.04 (s, 2H), | (M + H)⁺ |
| R = isopropyl | | 1.43 (d, J = 6.4 Hz, 6H), 1.18 (s, 6H) | |
| 33% yield | | | |
| 88 | J-2(86) | DMSO-d6, δ = 7.74-7.68 (m, 1H), 7.56-7.44 (m, 5H), 7.37- | 473.1 |
| Method B | | 7.30 (m, 2H), 5.62-5.51 (m, 1H), 5.09-5.00 (m, 1H), 4.86 | (M + H)⁺ |
| R = isopropyl | | (t, J = 5.6 Hz, 1H), 3.61-3.48 (m, 2H), 1.43 (d, J = 6.0 Hz, | |
| 6.7% yield | | 6H), 1.25 (d, J = 6.4 Hz, 3H) | |
| 99 | J-2(99) | DMSO-d6, δ = 7.73-7.68 (m, 1H), 7.56-7.45 (m, 5H), 7.39- | 473.1 |
| Method C | | 7.32 (m, 2H), 4.67 (s, 1H), 4.66-4.58 (m, 2H), 4.05 (s, 2H), | (M + H)⁺ |
| R = ethyl | | 1.45 (t, J = 7.2 Hz, 3H), 1.18 (s, 6H) | |
| 43.6% yield | | | |
| 101 | J-2(86) | DMSO-d6, δ = 7.75-7.68 (m, 1H), 7.56-7.44 (m, 5H), 7.38- | 473.0 |
| Method B | | 7.30 (m, 2H), 5.63-5.53 (m, 1H), 4.89 (d, J= 4.8 Hz, 1H), | (M + H)⁺ |
| R = isopropyl | | 4.16-4.05 (m, 2H), 4.02-3.92 (m, 1H), 1.44 (d, J = 6.0 Hz, | |
| 21.7% yield | | 6H), 1.13 (d, J = 6.0 Hz, 3H) | |
| 189 | J-2(86) | DMSO-d6, δ = 7.70-7.64 (m, 1H), 7.52-7.42 (m, 5H), 7.32- | 472.3 |
| Method D | | 7.25 (m, 2H), 5.50 (hept, 1H), 4.65 (t, 1H), 3.67-3.55 (m, | (M + H)⁺ |
| R = isopropyl | | 4H), 3.11 (s, 3H), 1.41 (d, 6H) | |
| 205 | J-2(86) | DMSO-d6, δ = 7.70 (dd, 1H), 7.55-7.43 (m, 5H), 7.36-7.29 | 457.2 |
| Method E | | (m, 2H), 5.61-5.49 (m, 1H), 5.18-5.07 (m, 1H), 1.42 (d, | (M + H)⁺ |
| R = isopropyl | | 6H), 1.30 (d, 6H) | |

(xiii) General Synthetic Method K:

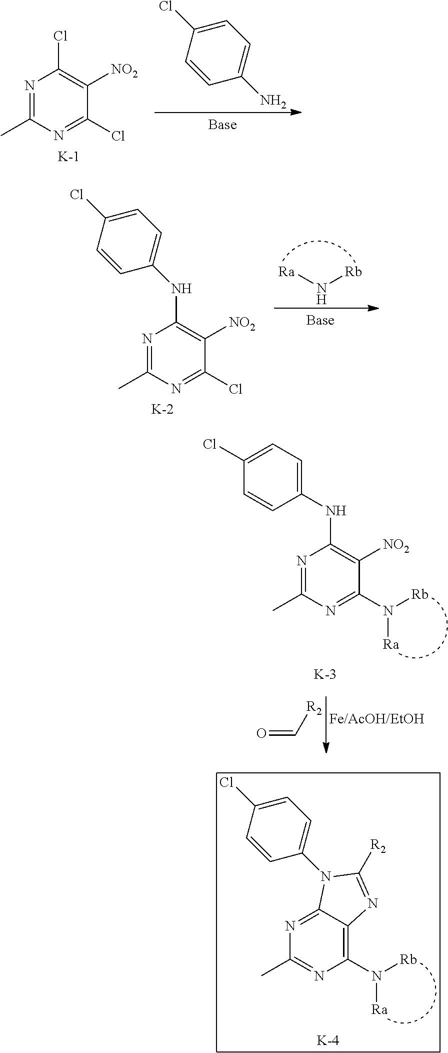

### Preparation of K-4 Compounds:

To a mixture of intermediate K-3 (1 eq.) and the aldehyde derivative (5 eq.) in ethyl alcohol (2.5 mL) was added iron (10 eq.) and acetic acid (1 mL), the mixture was stirred at 80° C. for 1 hour. The reaction mixture was filtered and concentrated under reduced pressure to give a residue. The residue was dissolved in acetonitrile (2 mL) and purified by prep-HPLC (column: Phenomenex luna C18 150*25 mm*10 μm; mobile phase: [water (formic acid)-ACN]; B %: 40%-70%, 10 minutes) and the organic phase was concentrated under reduced pressure to remove acetonitrile, and the liquid was lyophilized to give compound K-4 as a yellow solid.

### Reaction Scale: 220 mg of Intermediate K-3.

TABLE 12
Reaction conditions and data for K-4 compounds

| Compounds | Starting | | Exact |
|---|---|---|---|
| K-4 | material | ¹H NMR, 400 MHz | Mass |
| 127 | K-3(127) | DMSO-d₆, δ = 7.72-7.65 (m, 1H), 7.51-7.41 (m, 5H), 7.35- | 495.1 |
| 74.4% yield | | 7.28 (m, 2H), 7.26 (s, 1H), 6.95 (s, 1H), 5.11-4.31 (m, 2H), | (M + H)⁺ |
| | | 3.98-3.44 (m, 2H), 2.43-2.36 (m, 3H), 2.12-2.06 (m, 2H), | |
| | | 1.48-1.36 (m, 2H), 1.16 (s, 3H). | |
| 134 | K-3(134) | DMSO-d₆, δ = 8.85-8.76 (m, 1H), 8.09-8.02 (m, 1H), 7.99- | 517.3 |
| 38.7% yield | | 7.89 (m, 1H), 7.67-7.56 (m, 2H), 7.54-7.46 (m, 2H), 7.29 (s, | (M + H)⁺ |
| | | 1H), 7.21 (s, 1H), 5.80-4.26 (m, 2H), 4.03-3.45 (m, 2H), 3.44- | |
| | | 3.36 (m, 2H), 2.39 (s, 3H), 1.90 (s, 4H), 1.22 (t, J = 6.8 Hz, 3H) | |

(xiv) General Synthetic Method L:

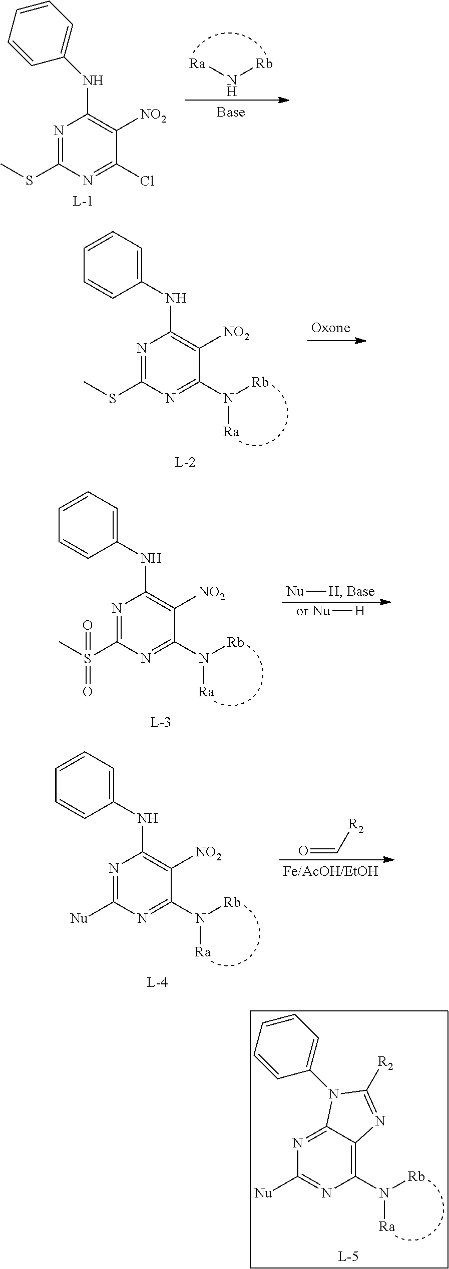

### Preparation of L-5 Compounds:

Method for compound 95: Step 1, to a solution of 6-chloro-2-methylsulfanyl-5-nitro-N-phenyl-pyrimidin-4-amine (Intermediate L-1) (700 mg, 1.79 mmol, 76% purity, 1 eq.) in N, N-dimethylformamide (5 mL) was added 4-methylpiperidine-4-carboxamide (384 mg, 2.15 mmol, 1.2 eq.) and potassium carbonate (743 mg, 5.38 mmol, 3 eq.), the mixture was stirred at 25° C. for 1 hour. The reaction mixture was added dropwise into water 40 mL, filtered and the filtration residue was washed with (petroleum ether:ethyl acetate=10:1) 50 mL, concentrated under reduced pressure to give 1-(6-anilino-2-methylsulfanyl-5-nitro-pyrimidin-4-yl)-4-methyl-piperidine-4-carboxamide (Intermediate L-2, 600 mg, 1.49 mmol) as a yellow solid. ¹H NMR (400 MHz, DMSO-d₆) δ=10.29 (s, 1H), 7.61 (d, J=7.6 Hz, 2H), 7.37 (t, J=8.0 Hz, 2H), 7.29 (s, 1H), 7.22-7.13 (m, 1H), 7.01 (s, 1H), 3.63 (d, J=13.2 Hz, 2H), 3.28-3.21 (m, 2H), 2.41 (s, 3H), 2.09 (d, J=14.0 Hz, 2H), 1.51-1.38 (m, 2H), 1.15 (s, 3H).

Step 2, to a solution of 1-(6-anilino-2-methylsulfanyl-5-nitro-pyrimidin-4-yl)-4-methyl-piperidine-4-carboxamide (Intermediate L-2) (400 mg, 0.99 mmol, 1 eq.) in N-methyl-2-pyrrolidone (8 mL) and water (0.8 mL) was added potassium peroxymonosulfate (1.67 g, 9.94 mmol, 10 eq.). The mixture was stirred at 60° C. for 1 hour. The reaction mixture was washed with water (30 mL) and extracted with ethyl acetate 90 mL (30 mL×3). The combined organic layers were washed with brine 100 mL (50 mL×2), dried over anhydrous sodium sulfate, filtered and concentrated under reduced pressure to give a residue. The residue was purified by flash silica gel chromatography (ISCO®; 40 g SepaFlash® Silica Flash Column, Eluent of 0˜100% Ethyl acetate/Petroleum ether gradient @30 mL/min) and the organic phase was concentrated under reduced pressure to give 1-(6-anilino-2-methylsulfonyl-5-nitro-pyrimidin-4-yl)-4-methyl-piperidine-4-carboxamide (Intermediate L-3, 400 mg, 0.91 mmol) as a yellow solid. ¹H NMR (400 MHz, CDCl₃-d) δ=10.25 (s, 1H), 7.54 (d, J=7.8 Hz, 2H), 7.40 (t, J=7.9 Hz, 2H), 7.26-7.21 (m, 1H), 5.92-5.38 (m, 2H), 3.54 (t, J=10.6 Hz, 2H), 3.17 (s, 3H), 2.25-2.14 (m, 2H), 2.03-2.01 (m, 2H), 1.70-1.62 (m, 2H), 1.33 (s, 3H).

Step 3, A mixture of 2-methylpropane-1,2-diol (236 mg, 2.62 mmol, 3 eq.) in tetrahydrofuran (5 mL) was added sodium hydride (105 mg, 2.62 mmol, 60% purity, 3.0 eq.) and stirred at 25° C. for minutes, then 1-(6-anilino-2-methylsulfonyl-5-nitro-pyrimidin-4-yl)-4-methyl-piperidine-4-carboxamide (Intermediate L-3) (380 mg, 0.87 mmol, 1 eq.) was added to the mixture, the mixture was stirred at 25° C. for 50 minutes. The reaction mixture was added dropwise into water (20 mL) and extracted with ethyl acetate 30 mL (10 mL×3). The combined organic layers were washed with brine 20 mL (10 mL×2), dried over anhydrous sodium sulfate, filtered and concentrated under reduced pressure to give a residue. The residue was purified by flash silica gel chromatography (ISCO®; 20 g SepaFlash® Silica Flash Column, Eluent of 0˜100% Ethyl acetate/Petroleum ether gradient @30 mL/min) and the organic phase was concentrated under reduced pressure to give the crude product. The residue was dissolved in acetonitrile (2 mL) and purified by prep-HPLC (column: Phenomenex luna C18 150*25 mm*10 μm; mobile phase: [water (formic acid)-ACN]; B %: 32%-62%, 10 minutes) and the organic phase was concentrated under reduced pressure to remove acetonitrile, and the liquid was lyophilized to give 1-[6-anilino-2-(2-hydroxy-2-methyl-propoxy)-5-nitro-pyrimidin-4-yl]-4-methyl-piperidine-4-carboxamide (Intermediate L-4, 300 mg, 675 μmol, 77.1% yield) as a yellow solid. ¹H NMR (400 MHz, CDCl₃-d) 6=10.48 (s, 1H), 7.59 (d, J=7.6 Hz, 2H), 7.40 (t, J=8.0 Hz, 2H), 7.24-7.19 (m, 1H), 5.73-5.28 (m, 2H), 4.17 (s, 2H), 3.69 (d, J=13.2 Hz, 2H), 3.54-3.40 (m, 2H), 2.65-2.37 (m, 1H), 2.22-2.09 (m, 2H), 1.70-1.63 (m, 2H), 1.33 (s, 3H), 1.29 (s, 6H).

Step 4, A mixture of 1-[6-anilino-2-(2-hydroxy-2-methyl-propoxy)-5-nitro-pyrimidin-4-yl]-4-methyl-piperidine-4-carboxamide (Intermediate L-4) (80 mg, 180 mmol, 1 eq.) and 2-chlorobenzaldehyde (253 mg, 1.80 mmol, 0.20 mL, 10 eq.) in ethyl alcohol (1 mL) was added iron (101 mg, 1.80 mmol, 10 eq.) and acetic acid (0.4 mL), the mixture was stirred at 80° C. for 1 hour. The reaction mixture was washed with water (10 mL) and extracted with ethyl acetate 30 mL (10 mL×3). The combined organic layers were washed with brine 20 mL (10 mL×2), dried over anhydrous sodium sulfate, filtered and concentrated under reduced pressure to give a residue. The residue was dissolved in acetonitrile (2 mL) and purified by prep-HPLC (column: Waters Xbridge 150*25 mm*5 μm; mobile phase: [water (ammonium bicarbonate)-ACN]; B %: 39%-69%, 8 minutes) and the organic phase was concentrated under reduced pressure to remove acetonitrile, and the liquid was lyophilized to give the crude product. The residue was dissolved in acetonitrile (2 mL) and purified by prep-HPLC (column: Phenomenex luna C18 150*25 mm*10 μm; mobile phase: [water (formic acid)-ACN]; B %: 45%-65%, 10 min) and the organic phase was concentrated under reduced pressure to remove acetonitrile, and the liquid was lyophilized to give 1-[8-(2-chlorophenyl)-2-(2-hydroxy-2-methyl-propoxy)-9-phenyl-purin-6-yl]-4-methyl-piperidine-4-carboxamide (Compound 95, 48.12 mg, 89.9 μmol, 50% yield) as an off-white solid. ¹H NMR (400 MHz, DMSO-d6) δ=7.68-7.61 (m, 1H), 7.51-7.32 (m, 6H), 7.31-7.17 (m, 3H), 6.97 (s, 1H), 5.30-4.67 (m, 1H), 4.61 (s, 1H), 4.57-4.10 (m, 1H), 3.96 (s, 2H), 3.91-3.36 (m, 2H), 2.14-2.06 (m, 2H), 1.43 (t, J=10.0 Hz, 2H), 1.16 (s, 3H), 1.14 (s, 6H). LCMS: (ES+) m/z=535.4 (M+H).

### (xv) General Synthetic Method M:

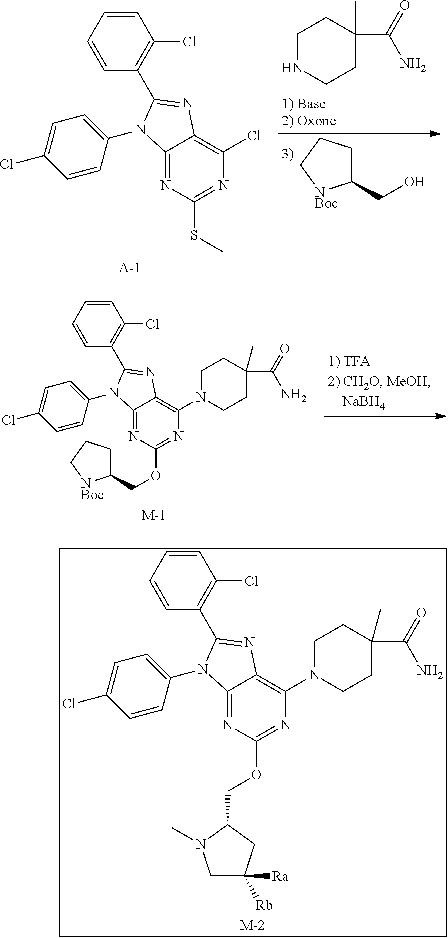

### Preparation of M-2 Compounds:

METHOD A: Step 1, 4-methyl-piperidine-4-carboxamide (1.7 eq.) was added to a 8 mL vial containing a solution of 6-chloro-8-(2-chlorophenyl)-9-(4-chlorophenyl)-2-(methylthio)-9H-purine (Intermediate A-1) (1 eq.) and N,N-diisopropylethylamine (7 eq.) in NMP (1.1 mL). The reaction mixture was stirred at room temperature for 2.5 hours. Water was added and the precipitate that formed was filtered on a Buchner funnel. The aqueous filtrate was also extracted by DCM and the DCM extract was evaporated and combined with the filtered material, affording crude internediate, which was used in the next step.

Step 2, oxone (3.5 eq.), NMP (1.6 mL), and water (160 uL) were added in a 8 mL vial containing crude intermediate at 60° C. overnight. Water was added and the precipitate obtained was filtered on a Buchner funnel to the intermediate (97% yield) which was used in the next step without purification.

Step 3, to the intermediate in a 8 mL vial was added potassium carbonate (2 eq.), 1-Boc-2-pyrrolidinemethanol derivative (11 eq.), and DMF (1.2 mL) and the mixture was stirred at 110° C. for 19 hours. The reaction mixture was purified by reverse phase flash chromatography using 10%-100% MeCN in 10 mM AmF (product elution at 72% MeCN), affording intermediate M-1 (73% yield).

### Reaction Scale: 100 mg of Intermediate a-1.

METHOD B: Step 1, to intermediate M-1 (1 eq.) was added DCM (1.1 mL) and trifluoroacetic acid (360 μL). The solution was first stirred for 1 hour at room temperature. Then additional trifluoroacetic acid (800 μL) was added and the reaction was continued for another 2.5 hours. The mixture was quenched with saturated NaHCO₃ (20 mL) and extracted with DCM (2×). The organic layers were dried (MgSO₄) and evaporated in vacuo to afford the intermediate deprotected pyrrolidine (quantitative yield).

Step 2, a solution of the intermediate deprotected pyrrolidine (1 eq.) and formaldehyde (6 eq.) (37% in water) in MeOH (3.7 mL) and AcOH (739 μL) was stirred for 3 minutes at room temperature, before being cooled on ice. Sodium triacetoxyborohydride (3 eq.) was added portion-wise, and the mixture was stirred at room temperature for 30 minutes. The reaction mixture was diluted with saturated NaHCO₃ (25 mL) and extracted with DCM (50 mL). The organic layer was separated and the aqueous was again extracted with DCM (50 mL), shaking the extraction funnel vigorously due to white emulsion tending to settle at the bottom. The combined DCM layers were dried with MgSO₄ and evaporated in vacuo. The residue was then purified on a 12 g C-18 column using 0%-80% MeCN in 10 mM aqueous ammonium formate (elution of product at 32% MeCN) to afford the compound M-2 (47% yield).

### Reaction Scale: 100 mg of Intermediate M-1

TABLE 13
Reaction conditions and data for M-2 compounds

| Compounds | Starting | | Exact |
|---|---|---|---|
| M-2 | material | ¹H NMR, 400 MHz | Mass |
| 228 | A-1 | DMSO-d6, δ = 7.68 (dd, 1H), 7.54-7.38 (m, 5H), 7.35-7.19 | 612.3 |
| Method A + | | (m, 3H), 6.97 (s, 1H), 5.27-4.94 (m, 1H), 4.34 (dd, 2H), 4.05 | (M + H)⁺ |
| Method B | | (dd, 1H), 3.98-3.46 (m, 2H), 3.13 (dd, 1H), 2.62-2.52 (m, | |
| | | 1H), 2.46-2.32 (m, 2H), 2.31-2.25 (m, 4H), 2.15-2.03 (m, | |
| | | 2H), 1.77 (ddd, 1H), 1.43 (t, 2H), 1.16 (s, 3H) | |
| 229 | A-1 | DMSO-d6, δ = 7.68 (dd, , 1H), 7.54-7.39 (m, 5H), 7.35-7.23 | 630.3 |
| Method A+ | | (m, 3H), 6.98 (s, 1H), 5.02 (bs, 1H), 4.34 (dd, 2H), 4.15 (dd | (M + H)⁺ |
| Method B | | 1H), 4.03-3.39 (m, 2H), 3.31-3.24 (m, 1H), 2.93-2.78 (m, | |
| | | 1H), 2.61 (ddd, 11.0 Hz, 1H), 2.48-2.36 (m, 1H), 2.29 (s, 3H), | |
| | | 2.25-2.03 (m, 3H), 1.42 (t, 2H), 1.16 (s, 3H) | |
| 230 | A-1 | DMSO-d6, δ = 7.68 (d, 1H), 7.56-7.39 (m, 5H), 7.37-7.20 | 594.3 |
| Method A + | | (m, 3H), 6.98 (s, 1H), 5.00 (bs, 1H), 4.27 (dd, 2H), 4.05-3.72 | (M + H)⁺ |
| Method B | | (m, 3H), 2.98-2.84 (m, 1H), 2.61-2.52 (m, 1H), 2.30 (s, 3H), | |
| | | 2.21-2.01 (m, 3H), 1.96-1.78 (m, 1H), 1.71-1.53 (m, 3H), | |
| | | 1.42 (t, 2H), 1.15 (s, 3H) | |
| 232 | A-1 | DMSO-d6, δ = 7.68 (d, 1H), 7.56-7.39 (m, 5H), 7.30 (d, 3H), | 612.3 |
| Method A + | | 6.98 (s, 1H), 5.29-4.81 (m, 2H), 4.32 (dd, 2H), 4.08 (dd, 1H), | (M + H)⁺ |
| Method B | | 3.88 (bs, 1H), 3.41 (ddd, 2H), 2.90-2.78 (m, 1H), 2.45-2.21 | |
| | | (m, 4H), 2.18-1.96 (m, 3H), 1.95-1.73 (m, 1H), 1.51-1.29 | |
| | | (m, 2H), 1.15 (s, 3H) | |
| 235 | M-1 | DMSO-d6, δ = 7.68 (d, 1H), 7.55-7.39 (m, 5H), 7.36-7.23 | 610.3 |
| Method B | | (m, 3H), 6.98 (s, 1H), 5.00 (bs, 1H), 4.26 (dd, 2H), 4.13 (dt 1H), | (M + H)⁺ |
| | | 3.98 (dd, 2H), 3.86 (bs, 2H), 3.16 (dd, 1H), 2.80-2.70 (m, 1H), | |
| | | 2.28 (s, 3H), 2.15-2.00 (m, 3H), 1.85-1.64 (m, 2H), 1.42 (t, | |
| | | 2H), 1.15 (s, 3H) | |
| 236 | M-1's | DMSO-d6, δ = 7.68 (dd, 1H), 7.53-7.41 (m, 5H), 7.34-7.25 | 610.3 |
| Method B | side- | (m, 3H), 6.98 (s, 1H), 5.17-5.07 (m, 1H), 5.00 (bs, 1H), 4.32 | (M + H)⁺ |
| | product | (s, 2H), 3.89 (s, 2H), 3.53-3.45 (m, 2H), 3.32 (dd, 1H), 2.48- | |
| | | 2.43 (m, 1H), 2.29 (s, 3H), 2.27-2.23 (m, 1H), 2.10 (d, 2H), | |
| | | 2.01-1.83 (m, 2H), 1.42 (t, 2H), 1.16 (s, 3H) | |

(xvi) General Synthetic Method N:

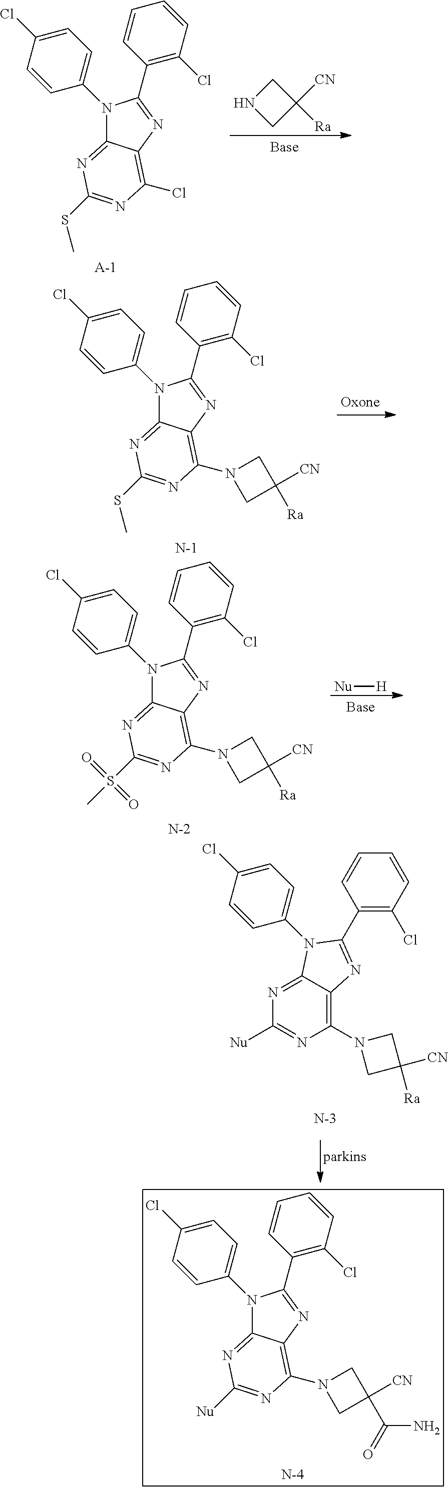

### Preparation of N-4 Compounds:

METHOD A: Step 1, to a solution of 6-chloro-8-(2-chlorophenyl)-9-(4-chlorophenyl)-2-methylsulfanyl-purine (Intermediate A-1) (1 eq.) in N,N-dimethylformamide (5 mL) was added N,N-diisopropylethylamine (3 eq.) and the secondary amine derivative (1.2 eq.). The mixture was stirred at 60° C. for 16 hours. The reaction mixture was washed with water (20 mL) and extracted with ethyl acetate 60 mL (20 mL×3). The combined organic layers were washed with brine 40 mL (20 mL×2), dried over anhydrous sodium sulfate, filtered and concentrated under reduced pressure to give a residue. The residue was purified by flash silica gel chromatography (ISCO®; 20 g SepaFlash® Silica Flash Column, Eluent of 0˜100% Ethyl acetate/Petroleum ether gradient @30 mL/min) and the organic phase was concentrated under reduced pressure to give intermediate N-1 (92% yield) as a light-yellow solid.

Step 2, a mixture of intermediate N-1 (1 eq.) in N-methyl-2-pyrrolidone (5 mL) and water (0.5 mL) was added potassium peroxymonosulfate (10 eq.). The mixture was stirred at 60° C. for 1 hour. The reaction mixture was added dropwise into water 40 mL, filtered and the filtration residue was concentrated under reduced pressure to give intermediate N-2 (crude) as a yellow solid.

Step 3, a mixture of intermediate N-2 (1 eq.), nucleophile (Nu-H) (5 eq.) and N,N-diisopropylethylamine (8.8 eq.) in N-methyl-2-pyrrolidone (5 mL) was stirred at 140° C. for 2 hours. The reaction mixture was washed with water (10 mL) and extracted with ethyl acetate 30 mL (10 mL×3). The combined organic layers were washed with brine 20 mL (10 mL×2), dried over anhydrous sodium sulfate, filtered and concentrated under reduced pressure to give a residue. The residue was purified by flash silica gel chromatography (ISCO®; 40 g SepaFlash® Silica Flash Column, Eluent of 0˜100% Ethyl acetate/Petroleum ether gradient @60 mL/min) and the organic phase was concentrated under reduced pressure to give intermediate N-3 (92% yield) as a white solid.

Step 4, a mixture of intermediate N-3 (1 eq.) in ethyl alcohol (1 mL) and water (0.1 mL) was added hydrido(dimethylphosphinousacid-kp)[hydrogenbis(dimethylphosphinito-kp)]platinum(II) (1 eq.). The mixture was stirred at 60° C. for 2 hours. The reaction mixture was filtered and concentrated under reduced pressure to give a residue. The residue was purified by prep-HPLC (column: Waters Xbridge 150*25 mm*5 μm; mobile phase: [water(ammonium hydroxide or ammonium bicarbonate)-ACN]) to give compound N-4 as a white solid.

### Reaction Scale: 500 mg of Intermediate a-1

METHOD B: Step 1, to a solution of 6-chloro-8-(2-chlorophenyl)-9-(4-chlorophenyl)-2-methylsulfanyl-purine (Intermediate A-1) (1.0 eq.) in N,N-dimethylformamide (5 mL) was added N,N-diisopropylethylamine (3.0 eq.) and the secondary amine derivative (1.2 eq.). The mixture was stirred at 60° C. for 2 hours. The reaction mixture was added dropwise into water 30 mL, filtered and the filtration residue was concentrated under reduced pressure to give intermediate N-1 (94% yield) as a light-yellow solid.

Step 2, a mixture of intermediate N-1 (1 eq.) in N-methyl-2-pyrrolidone (5 mL) and water (0.5 mL) was added potassium peroxymonosulfate (10 eq.). The mixture was stirred at 60° C. for 1 hour. The reaction mixture was added dropwise into water 40 mL, filtered and the filtration residue was concentrated under reduced pressure to give intermediate N-2 (crude) as a yellow solid.

Step 3, to a solution of intermediate N-2 (1.0 eq.) in the nucleophile (Nu-H) (10 eq.) was stirred at 140° C. for 12 hours. The residue was dissolved in acetonitrile (2 mL) and purified by prep-HPLC (column: Phenomenex luna C18 150*25 mm*10 μm; mobile phase: [water (formic acid)-ACN]; B %: 60%-90%, 10 minutes) and the organic phase was concentrated under reduced pressure to remove acetonitrile, and the liquid was lyophilized to give intermediate N-3 (54% yield) as a white solid.

Step 4, to a solution of intermediate N-3 (1.0 eq.) in ethyl alcohol (1 mL) and water (0.1 mL) was added hydrido(dimethylphosphinousacid-kp)[hydrogenbis(dimethylphosphinito-kp)]platinum(II) (1.0 eq.). The mixture was stirred at 60° C. for 1 hour. The reaction mixture was filtered and concentrated under reduced pressure to give a residue. The residue was dissolved in acetonitrile (2 mL) and purified by prep-HPLC (column: Phenomenex luna C18 150*25 mm*10 μm; mobile phase: [water(formic acid)-ACN]; B %: 39%-69%, 10 minutes) and the organic phase was concentrated under reduced pressure to remove acetonitrile, and the liquid was lyophilized to give compound N-4 as a white solid.

### Reaction Scale: 500 mg of Intermediate a-1

TABLE 14
Reaction conditions and data for N-4 compounds

| Compounds | Starting | | Exact |
|---|---|---|---|
| N-4 | material | ¹H NMR, 400 MHz | Mass |
| 72 | A-1 | CDCl₃-d, δ = 7.42-7.37 (m, 1H), 7.27-7.21 (m, 5H), 7.13- | 512.2 |
| Method A | | 7.05 (m, 2H), 5.93-5.76 (m, 1H), 5.44-5.28 (m, 1H), 4.73- | (M + H)⁺ |
| 38% yield | | 4.52 (m, 4H), 3.84-3.74 (m, 2H), 3.67-3.59 (m, 2H), 3.57- | |
| (Step 4) | | 3.46 (m, 1H), 3.09 (s, 3H) | |
| 73 | A-1 | CDCl₃-d, δ = 7.48 (br d, J = 6.6 Hz, 1H), 7.39-7.28 (m, 5H), | 526.3 |
| Method A | | 7.17 (d, J = 8.7 Hz, 2H), 5.91-5.23 (m, 2H), 5.18-4.91 (m, | (M + H)⁺ |
| 11.4% yield | | 1H), 4.73 (br d, J = 6.1 Hz, 2H), 4.38-4.09 (m, 2H), 3.88-3.79 | |
| (Step 4) | | (m, 2H), 3.76-3.66 (m, 2H), 3.18(s, 3H), 1.77-1.61 (m, 3H) | |
| 128 | A-1 | DMSO-d₆, δ = 7.67-7.62 (m, 1H), 7.51-7.42 (m, 6H), 7.26 (d, | 540.2 |
| Method B | | J = 8.4 Hz, 2H), 7.05 (s, 1H), 5.08-4.59 (m, 1H), 4.56-4.22 | (M + H)⁺ |
| 96.4% yield | | (m, 4H), 3.58-3.49 (m, 3H), 3.11 (s, 3H), 1.07 (s, 6H) | |
| (Step 4) | | | |

(xvii) General Synthetic Method O:

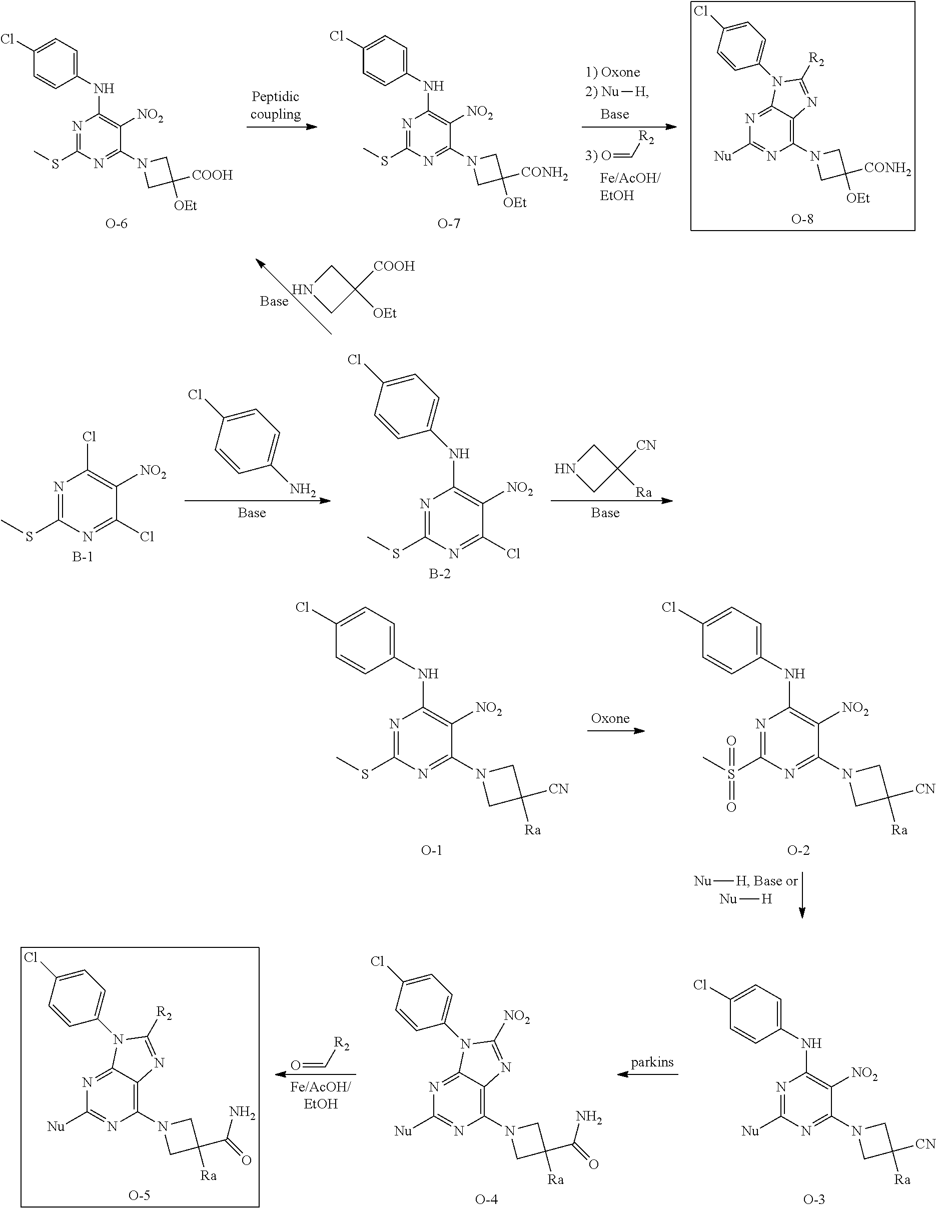

### Preparation of O-5 Compounds:

Method A: Step 1, to a solution of 6-chloro-N-(4-chlorophenyl)-2-methylsulfanyl-5-nitro-pyrimidin-4-amine (Intermediate B-2) (1 g, 3.02 mmol, 1 eq.) in N,N-dimethylformamide (10 mL) was added N,N-diisopropylethylamine (1.17 g, 9.06 mmol, 1.58 mL, 3 eq.) and azetidine-3-carbonitrile hydrochloride (430 mg, 3.62 mmol, 1.2 eq.). The mixture was stirred at 60° C. for 1 hour. The reaction mixture was added dropwise into water 50 mL, filtered and the filtration residue was concentrated under reduced pressure to give 1-[6-(4-chloroanilino)-2-methylsulfanyl-5-nitro-pyrimidin-4-yl]azetidine-3-carbonitrile (Intermediate 0˜1, crude) as a light-yellow solid. ¹H NMR (400 MHz, DMSO-d₆) δ=10.38 (s, 1H), 7.67-7.53 (m, 2H), 7.48-7.35 (m, 2H), 4.53-4.35 (m, 2H), 4.33 (d, J=6.0 Hz, 2H), 3.90-3.79 (m, 1H), 2.39 (s, 3H).

Step 2, to a solution of 1-[6-(4-chloroanilino)-2-methylsulfanyl-5-nitro-pyrimidin-4-yl]azetidine-3-carbonitrile (Intermediate 0˜1) (1.2 g, 3.18 mmol, 1 eq.) in N-methyl-2-pyrrolidone (12 mL) and water (1.2 mL) was added potassium peroxymonosulfate (5.36 g, 31.85 mmol, 10 eq.). The mixture was stirred at 60° C. for 1 hour. The reaction mixture was added dropwise into water 50 mL, filtered and the filtration residue was concentrated under reduced pressure to give 1-[6-(4-chloroanilino)-2-methylsulfonyl-5-nitro-pyrimidin-4-yl]azetidine-3-carbonitrile (Intermediate O-2, 74% yield) as a light-yellow solid. ¹H NMR (400 MHz, DMSO-d₆) δ=10.43 (s, 1H), 7.62-7.56 (m, 2H), 7.48-7.43 (m, 2H), 4.54-4.42 (m, 2H), 4.41-4.33 (m, 2H), 3.95-3.85 (m, 1H), 3.21 (s, 3H). LCMS: (ES⁺) m/z=409.0 (M+H).

Step 3, to a solution of 1-[6-(4-chloroanilino)-2-methylsulfonyl-5-nitro-pyrimidin-4-yl]azetidine-3-carbonitrile (Intermediate O-2) (1 eq.) in nucleophile (Nu-H) (5 eq.) was stirred at 140° C. for 1 hour.*,** The reaction mixture was filtered and concentrated under reduced pressure to give a residue. The residue was purified by flash silica gel chromatography and the organic phase was concentrated under reduced pressure to give intermediate O-3 (59% yield) as a yellow solid.

### Reaction Scale: 400 mg of Intermediate O-2

* for compound 137: 80° C., 1 hour in NMP (4 mL) with tBuOK (3 eq.). Extraction with EtOAc.

** for compound 140: 60° C., 1 hour.

Step 4, to a solution of intermediate O-3 (1 eq.) in ethyl alcohol (2.5 mL) and water (0.25 mL) was added hydrido (dimethylphosphinous acid-kp)[hydrogen bis (dimethylphosphinito-kp)]platinum(II) (1 eq.). The mixture was stirred at 60° C. for 1 hour. The reaction mixture was filtered and concentrated under reduced pressure to give a residue. The residue was purified by flash silica gel chromatography (ISCO®; 12 g SepaFlash® Silica Flash Column, Eluent of 0˜100% Ethyl acetate/Petroleum ether gradient @20 mL/min) and the organic phase was concentrated under reduced pressure to give intermediate O-4 (86% yield) as a yellow solid.

### Reaction Scale: 250 mg of Intermediate O-3

Step 5, a mixture of intermediate O-4 (1 eq.) and aldehyde derivative (5 eq.) in ethyl alcohol (1.5 mL) was added iron (10 eq.) and acetic acid (0.6 mL), the mixture was stirred at 80° C. for 1 hour. The reaction mixture was filtered and concentrated under reduced pressure to give a residue. The residue was purified by prep-HPLC (column: Phenomenex Luna C18 150*25 mm*10 μm; mobile phase: [water (formic acid)-ACN]) and the organic phase was concentrated under reduced pressure to remove acetonitrile, and the liquid was lyophilized to give compound O-5 as a yellow solid.

### Reaction Scale: 170 mg of Intermediate O-4

Method B: Step 1, a mixture of 6-chloro-N-(4-chlorophenyl)-2-methylsulfanyl-5-nitro-pyrimidin-4-amine (Intermediate B-2) (1 eq.), the secondary amine derivative (1.1 eq.) and cesium carbonate (3 eq.) in N, N-dimethylformamide (30 mL) was stirred at 25° C. for 2 hours. The reaction mixture was added dropwise into water 200 mL, filtered and the filter cake was concentrated under reduced pressure to give intermediate O-1 (crude) as a yellow solid, which was used for the next step directly without further purification.

### Reaction Scale: 2.3 g of Intermediate B-2

Step 2, a mixture of intermediate O-1 (1 eq.) and potassium; oxidooxy hydrogen sulfate (10 eq.) in N-methyl-2-pyrrolidone (30 mL) and water (5 mL) was stirred at 60° C. for 2 hours. The reaction mixture was added dropwise into water (150 mL), filtered and the filter cake was concentrated under reduced pressure, the resulting residue was purified by flash silica gel chromatography (ISCO®; 80 g SepaFlash® Silica Flash Column, Eluent of 30˜80% Ethyl acetate/Petroleum ether gradient @50 mL/min). The cut fraction was concentrated under reduced pressure to give intermediate O-2 (crude) as a yellow solid.

### Reaction Scale: 2.6 g of Intermediate O-1

Step 3, to a solution of nucleophile (Nu-H) (5 eq.) in tetrahydrofuran (5 mL) was added sodium hydride (60% purity, 5.0 eq.), the resulting mixture was stirred at 25° C. for 0.5 hour. Then intermediate O-2 (1 eq.) was added to the mixture and the mixture was stirred at 25° C. for 1.5 hour. The reaction mixture was diluted with water (50 mL) and extracted with ethyl acetate (20 mL×3). The combined organic layer was washed with brine (50 mL), dried over anhydrous sodium sulfate, filtered and concentrated under reduced pressure, the resulting residue was purified by flash silica gel chromatography (ISCO®; 20 g SepaFlash® Silica Flash Column, Eluent of 0˜50% Ethyl acetate/Petroleum ether gradient @30 mL/min). The cut fraction was concentrated under reduced pressure to give intermediate O-3 (89% yield) as a yellow solid.

### Reaction Scale: 0.5 g of Intermediate O-2

Step 4, a mixture of intermediate O-3 (1 eq.) and hydrido(dimethylphosphinous acid-kp)[hydrogen bis(dimethylphosphinito-kp)]platinum (II) (1 eq.) in ethanol (5 mL) and water (0.5 mL) was stirred at 80° C. for 1 hour. The reaction mixture was cooled at room temperature and filtered; the resulting filter liquor was concentrated under reduced pressure to give the residue. The residue was purified by flash silica gel chromatography (ISCO®; 20 g SepaFlash® Silica Flash Column, Eluent of 50-100% Ethyl acetate/Petroleum ether gradient @40 mL/min). The cut fraction was concentrated under reduced pressure to give intermediate O-4 (70% yield) as a yellow solid.

### Reaction Scale: 300 mg of Intermediate O-3

Step 5, a mixture of intermediate O-4 (1 eq.), aldehyde derivative (5 eq.) and iron (10 eq.) in ethanol (3 mL) and acetic acid (1.2 mL) was stirred at 80° C. for 2 hours. The reaction mixture was cooled at room temperature and filtered; the resulting filter liquor was concentrated under reduced pressure to give the residue. The residue was purified by flash silica gel chromatography (ISCO®; 20 g SepaFlash® Silica Flash Column, Eluent of 50-100% Ethyl acetate/Petroleum ether gradient @35 mL/min). The cut fraction was concentrated under reduced pressure, the resulting crude product was purified by reversed-phase HPLC (column: Phenomenex Synergi C18 150*25 mm*10 μm; mobile phase: [water (formic acid)-ACN]; B %: 33%-63%). The cut fraction was concentrated under reduced pressure to remove acetonitrile. The residue was lyophilized to give compound O-5 as a yellow solid.

### Reaction Scale: 200 mg of Intermediate O-4

### Preparation of O-8 Compounds:

Method C: Step 1, a mixture of 6-chloro-N-(4-chlorophenyl)-2-methylsulfanyl-5-nitro-pyrimidin-4-amine (912 mg, 2.75 mmol, 1.0 eq.), 3-ethoxyazetidine-3-carboxylic acid hydrochloride (500 mg, 2.75 mmol, 1.0 eq.) and cesium carbonate (2.69 g, 8.26 mmol, 3.0 eq.) in N,N-dimethylformamide (10 mL) was stirred at 25° C. for 1 hour. The reaction mixture was added dropwise into 1N hydrochloric acid 80 mL (pH=3), filtered and the filtration residue was concentrated under reduced pressure to give the residue. The residue was purified by flash silica gel chromatography (ISCO®; 20 g SepaFlash® Silica Flash Column, Eluent of 0˜100% Ethyl acetate/Petroleum ether gradient @30 mL/min) and the organic phase was concentrated under reduced pressure to give 1-[6-(4-chloroanilino)-2-methylsulfanyl-5-nitro-pyrimidin-4-yl]-3-ethoxy-azetidine-3-carboxylic acid (1 g, 2.27 mmol, 82.5% yield) as a yellow solid. ¹H NMR (400 MHz, DMSO-d₆) δ=10.39 (s, 1H), 7.62 (d, J=8.8 Hz, 2H), 7.43 (d, J=8.8 Hz, 2H), 4.67-4.25 (m, 2H), 4.18 (d, J=10.8 Hz, 2H), 3.51 (q, J=6.8 Hz, 2H), 2.39 (s, 3H), 1.19-1.13 (m, 3H).

Step 2, a mixture of 1-[6-(4-chloroanilino)-2-methylsulfanyl-5-nitro-pyrimidin-4-yl]-3-ethoxy-azetidine-3-carboxylic acid (400 mg, 0.91 mmol, 1.0 eq.), ammonium chloride (107 mg, 2.00 mmol, 2.2 eq.), 1-(3-dimethylaminopropyl)-3-ethylcarbodiimide hydrochloride (261 mg, 1.36 mmol, 1.5 eq.), 1-hydroxybenzotriazole (184 mg, 1.36 mmol, 1.5 eq.) and N,N-diisopropylethylamine (259 mg, 2.00 mmol, 0.35 mL, 2.2 eq.) in N,N-dimethylformamide (4 mL) was stirred at 25° C. for 2 hours. The mixture was concentrated in vacuum to remove N,N-dimethylformamide, then dissolved in 150 mL dichloromethane, washed with 150 mL 10% citric acid, washed with 80 mL saturated sodium bicarbonate, saturated with brine (80 mL), dryness with anhydrous sodium sulfate, filtered and concentrated in vacuum to give 1-[6-(4-chloroanilino)-2-methylsulfanyl-5-nitro-pyrimidin-4-yl]-3-ethoxy-azetidine-3-carboxamide (390 mg, 0.89 mmol, 97.7% yield) as a yellow solid. ¹H NMR (400 MHz, DMSO-d₆) δ=10.38 (s, 1H), 7.65-7.60 (m, 2H), 7.59-7.47 (m, 2H), 7.46-7.40 (m, 2H), 4.52-4.08 (m, 4H), 3.52-3.41 (m, 2H), 2.39 (s, 3H), 1.20 (t, J=6.8 Hz, 3H).

Step 3, to a solution of 1-[6-(4-chloroanilino)-2-methylsulfanyl-5-nitro-pyrimidin-4-yl]-3-ethoxy-azetidine-3-carboxamide (390 mg, 0.89 mmol, 1 eq.) in N-methyl-2-pyrrolidone (4 mL) and water (0.4 mL) was added potassium peroxymonosulfate (1.49 g, 8.89 mmol, 10 eq.). The mixture was stirred at 60° C. for 1 hour. The reaction mixture was added dropwise into water 100 mL, filtered and the filtration residue was washed with acetonitrile 100 mL and concentrated under reduced pressure to give 1-[6-(4-chloroanilino)-2-methylsulfonyl-5-nitro-pyrimidin-4-yl]-3-ethoxy-azetidine-3-carboxamide (380 mg, 0.81 mmol, 90.8% yield) as a yellow solid. ¹H NMR (400 MHz, DMSO-d₆) δ=10.43 (s, 1H), 7.59 (d, J=8.8 Hz, 4H), 7.45 (d, J=8.8 Hz, 2H), 4.23 (s, 4H), 3.49-3.44 (m, 2H), 3.22 (s, 3H), 1.21 (t, J=6.8 Hz, 3H). LCMS: (ES⁺) m/z=471.2 (M+H).

To a solution of 1-[6-(4-chloroanilino)-2-methylsulfonyl-5-nitro-pyrimidin-4-yl]-3-ethoxy-azetidine-3-carboxamide (320 mg, 0.68 mmol, 1.0 eq.) in N-methyl-2-pyrrolidone (3 mL) was added potassium tert-butoxide (229 mg, 2.04 mmol, 3.0 eq.) and 2-methylpropane-1,2-diol (306 mg, 3.40 mmol, 5.0 eq.). The mixture was stirred at 80° C. for 1 hour. The reaction mixture was washed with water (10 mL) and extracted with ethyl acetate 30 mL (10 mL×3). The combined organic layers were washed with brine 20 mL (10 mL×2), dried over anhydrous sodium sulfate, filtered and concentrated under reduced pressure to give a residue. The residue was purified by flash silica gel chromatography (ISCO®; 20 g SepaFlash® Silica Flash Column, Eluent of 0˜100% Ethyl acetate/Petroleum ether gradient @30 mL/min) and the organic phase was concentrated under reduced pressure to give 1-[6-(4-chloroanilino)-2-(2-hydroxy-2-methyl-propoxy)-5-nitro-pyrimidin-4-yl]-3-ethoxy-azetidine-3-carboxamide (200 mg, 0.37 mmol, 54% yield) as a yellow solid.

A mixture of 1-[6-(4-chloroanilino)-2-(2-hydroxy-2-methyl-propoxy)-5-nitro-pyrimidin-4-yl]-3-ethoxy-azetidine-3-carboxamide (170 mg, 0.35 mmol, 1.0 eq.) and 5-formylpyridine-2-carbonitrile (234 mg, 1.77 mmol, 5 eq.) in ethyl alcohol (1.7 mL) was added iron (197 mg, 3.54 mmol, 10 eq.) and acetic acid (0.34 mL), the mixture was stirred at 80° C. for 1 hour. The reaction mixture was filtered and concentrated under reduced pressure to give a residue. The residue was dissolved in N,N-dimethylformamide (2 mL) and purified by prep-HPLC (column: Phenomenex luna C18 150*25 mm*10 μm; mobile phase: [water (formic acid)-ACN]; B %: 44%-74%, 10 minutes) and the organic phase was concentrated under reduced pressure to remove acetonitrile, and the liquid was lyophilized to give compound J-3, the 1-[9-(4-chlorophenyl)-8-(6-cyano-3-pyridyl)-2-(2-hydroxy-2-methyl-propoxy)purin-6-yl]-3-ethoxy-azetidine-3-carboxamide (Compound 139, 48.34 mg, 81.57 μmol, 23% yield) as a yellow solid.

TABLE 15
Reaction conditions and data for O-5 and O-8 compounds

| Compounds | Starting | | Exact |
|---|---|---|---|
| O-5 and O-8 | material | ¹H NMR, 400 MHz | Mass |
| 129 | B-2 | DMSO-d₆, δ = 8.78-8.75 (m, 1H), 8.01 (d, J = 8.4 Hz, 1H), | 532.3 |
| Method A | | 7.86-7.79 (m, 1H), 7.62-7.58 (m, 2H), 7.55 (s, 1H), 7.44 (d, | (M + H)⁺ |
| 27% yield | | J = 8.4 Hz, 2H), 7.14-7.07 (m, 1H), 4.81-4.08 (m, 5H), 3.62- | |
| (Step 5) | | 3.45 (m, 3H), 3.15-2.98 (m, 3H), 1.05 (s, 6H) | |
| 137 | B-2 | DMSO-d₆, δ = 8.80 (s, 1H), 8.04 (d, J = 8.0 Hz, 1H), 7.92- | 519.1 |
| Method A | | 7.86 (m, 1H), 7.64 (d, J = 8.8 Hz, 2H), 7.57 (s, 1H), 7.49 (d, | (M + H)⁺ |
| 9% yield | | J = 8.8 Hz, 2H), 7.12 (s, 1H), 4.61 (s, 3H), 4.42-4.19 (m, | |
| (Step 5) | | 2H), 3.94 (s, 2H), 3.61-3.53 (m, 1H), 1.14 (s, 6H) | |
| 138 | B-2 | DMSO-d₆, δ = 8.80 (d, J = 1.6 Hz, 1H), 8.05 (d, J = 8.0 Hz, | 533.3 |
| Method B | | 1H), 7.92-7.87 (m, 1H), 7.67-7.62 (m, 2H), 7.56-7.46 (m, | (M + H)⁺ |
| 26% yield | | 3H), 7.16 (s, 1H), 4.91-4.74 (m, 1H), 4.61 (s, 1H), 4.49- | |
| (Step 5) | | 4.31 (m, 2H), 4.01-3.90 (m, 3H), 1.57 (s, 3H), 1.14 (s, 6H) | |
| 139 | B-2 | DMSO-d₆, δ = 8.81 (d, J = 1.6 Hz, 1H), 8.05 (d, J = 8.4 Hz, | 563.1 |
| Method C | | 1H), 7.92-7.90 (m, 1H), 7.67-7.62 (m, 2H), 7.58 (s, 1H), | (M + H)⁺ |
| | | 7.55-7.44 (m, 3H), 4.91-4.89 (m, 1H), 4.67-4.55 (m, 2H), | |
| | | 4.51-4.42 (m, 1H), 4.30-4.17 (m, 1H), 3.95 (d, J = 3.6 Hz, | |
| | | 2H), 3.55-3.50 (m, 2H), 1.23 (t, J = 6.8 Hz, 3H), 1.14 (s, 6H) | |
| 140 | B-2 | CDCl₃-d, δ = 8.82 (d, J = 1.6 Hz, 1H), 7.86-7.75 (m, 1H), | 530.1 |
| Method A | | 7.59 (d, J = 8.0 Hz, 1H), 7.51 (d, J = 8.4 Hz, 2H), 7.26-7.22 | (M + H)⁺ |
| 13% yield | | (m, 2H), 5.82-5.36 (m, 2H), 4.96-4.42 (m, 4H), 4.37-3.35 | |
| (Step 5) | | (m, 7H), 2.15-2.05 (m, 1H), 1.95-1.75 (m, 3H) | |

(xviii) General Synthetic Method P:

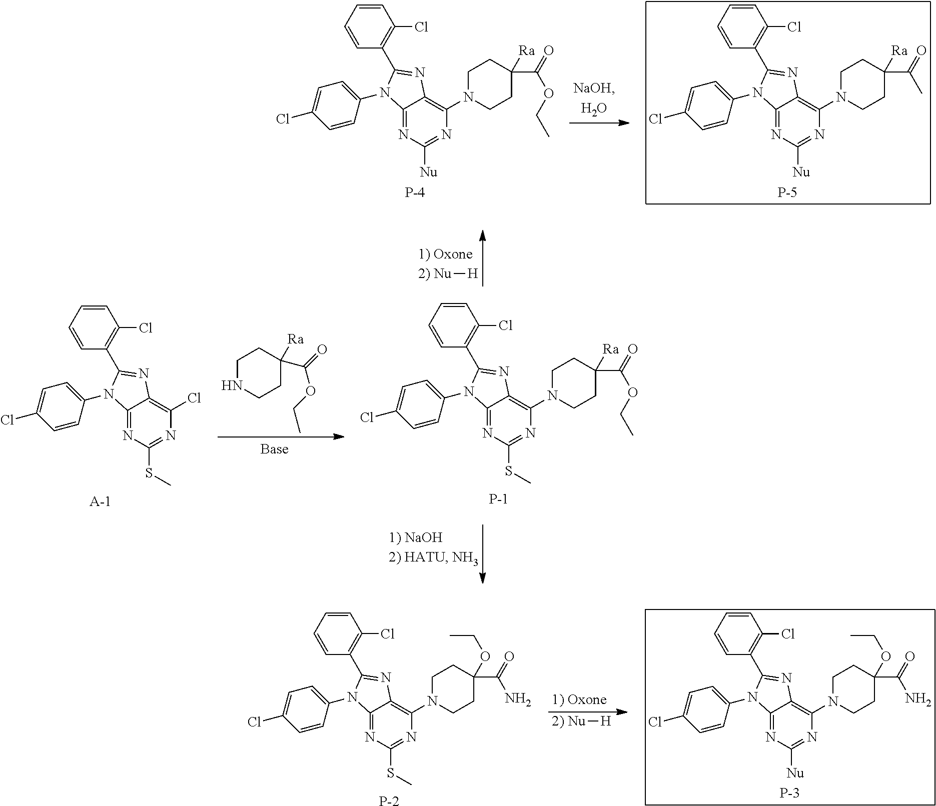

### Preparation of P-3 and P-5 Compounds:

Method A (for P-3 compounds): Step 1, to the secondary amine derivative (3 eq.) was added 6-chloro-8-(2-chlorophenyl)-9-(4-chlorophenyl)-2-(methylthio)-9H-purine (Intermediate A-1) (1 eq.) and N,N-diisopropylethylamine (30 eq.). Then NMP (2.5 mL) was added and the reaction mixture was stirred at room temperature overnight. With the addition of water, a precipitate and emulsion formed, which was filtered. The filtrate was extracted with ethyl acetate. The filtered precipitate was dissolved into the ethyl acetate extracts, which were combined, dried with magnesium sulfate, and evaporated to form intermediate P-1 (quantitative conversion) as an oil. The residue was used in the next step without any purification.

Step 2, intermediate P-1 (1 eq.) in THF (2.2 mL) was added 4 M sodium hydroxide (27 eq.). The mixture was stirred at 60° C. for 2.5 hours. After allowing the reaction flask to cool to room temperature, the mixture was concentrated. 6 M HCl was used to acidify the mixture to pH 4. The precipitate formed was filtered on a Buchner funnel to afford crude intermediate carboxylic acid which was used without further purification.

A mixture of the intermediate carboxylic acid (1 eq.), HATU (2.2 eq.), and N,N-Diisopropylethylamine (12 eq.) in EtOAc (5.5 mL) was stirred at room temperature for 15 minutes, after which 0.5 M Ammonia in dioxane (2 eq.) was added. The mixture was stirred at room temperature for 1.5 hours. The crude mixture was diluted with water (130 mL) and extracted with ethyl acetate (350 mL×2). The combined organic layers were dried with magnesium sulfate and evaporated in vacuo to afford intermediate P-2 (quantitative conversion) as a viscous oil which was used without further drying and purification.

Step 3, intermediate P-2 (1 eq.) was dissolved in NMP (3.5 mL), then water (300 μL) was added dropwise making sure that the solution stays clear. Oxone (3.2 eq.) was then added in one portion and the reaction mixture was stirred at room temperature overnight. Cold water was added and the precipitate obtained was filtered on a Buchner. The solid filtered was purified on a C-18 column (loading by minimal DMF and water) using 20% to 100% acetonitrile in aqueous AmF (product elution at 53% acetonitrile) to afford crude sulfone intermediate (43% yield).

A solution of the sulfone intermediate (1 eq.) in the nucleophile (Nu-H) (100 eq.) was stirred at 100° C. overnight. Upon cooling the solution, it was directly injected to a C-18 column and purified using 3% to 100% acetonitrile in 10 mM aqueous ammonium formate as eluent (product elution at 90% acetronitrile) to afford compound P-3 (27% yield).

### Reaction Scale: 185 mg of Intermediate A-1

Method B (for P-5 compounds): To a solution of intermediate P-4 (1 eq.) in THF (1.56 mL) was added 4 M sodium hydroxide (70 eq.). The mixture was stirred at 80° C. for 2 hours. The mixture was concentrated in vacuo and acidified using minimal aqueous HCl and lyophilized. The product was purified on a 12-g C-18 column using 15% to 100% MeCN in aqueous AmF buffer affording the P-5 compound (73% yield) after freeze drying.

### Reaction Scale: 140 mg of Intermediate P-4

TABLE 16
Reaction conditions and data for P-3 and P-5 compounds

| Compounds | Starting | | Exact |
|---|---|---|---|
| P-3 and P-5 | material | ¹H NMR, 400 MHz | Mass |
| 177 | A-1 | DMSO-d6, δ = 7.72-7.61 (m, 1H), 7.52-7.39 (m, 5H), 7.35- | 610.4 |
| Method A | | 7.25 (m, 3H), 7.20 (s, 1H), 4.72 (t, 1H), 4.03 (s, 1H), 3.63 (s, | (M + H)⁺ |
| | | 2H), 3.52-3.34 (m, 5H), 3.33-3.30 (m, 3H), 2.01-1.71 (m, | |
| | | 8H), 1.21 (t, 3H) | |
| 182 | A-1 | DMSO, δ = 7.66 (dd, 1H), 7.54-7.39 (m, 5H), 7.31-7.26 (m, | 584.4 |
| Method A | | 3H), 7.19 (s, 1H), 4.59 (s, 2H), 3.58 (s, 4H), 3.44-3.36 (m, | (M + H)⁺ |
| | | 5H), 3.08 (s, 3H), 1.91-1.80 (m, 4H), 1.21 (t, 3H) | |
| 218 | P-4 | DMSO-d6, δ = 7.69 (d, 1H), 7.54-7.39 (m, 5H), 7.35-7.27 | 600.3 |
| Method B | | (m, 2H), 5.28 (bs, 1H), 4.54 (bs, 2H), 3.98 (s, 2H), 3.89-3.55 | (M + H)⁺ |
| | | (m, 1H), 3.45 (q, 3H), 2.01-1.81 (m, 4H), 1.27-1.07 (m, 9H) | |
| 220 | P-4 | DMSO-d6, δ = 12.46 (br s, 1H), 7.68 (dd, 1H), 7.53-7.41 (m, | 570.2 |
| Method B | | 5H), 7.30 (d, 2H), 5.08 (br s, 1H), 4.61 (s, 1H), 4.44 (brs, 1H), | (M + H)⁺ |
| | | 3.97 (s, 2H), 3.91-3.40 (br m, 2H), 2.11-2.02 (m, 2H), 1.52- | |
| | | 1.42 (m, 2H), 1.20 (s, 3H), 1.15 (s, 6H) | |

(xix) General Synthetic Method Q:

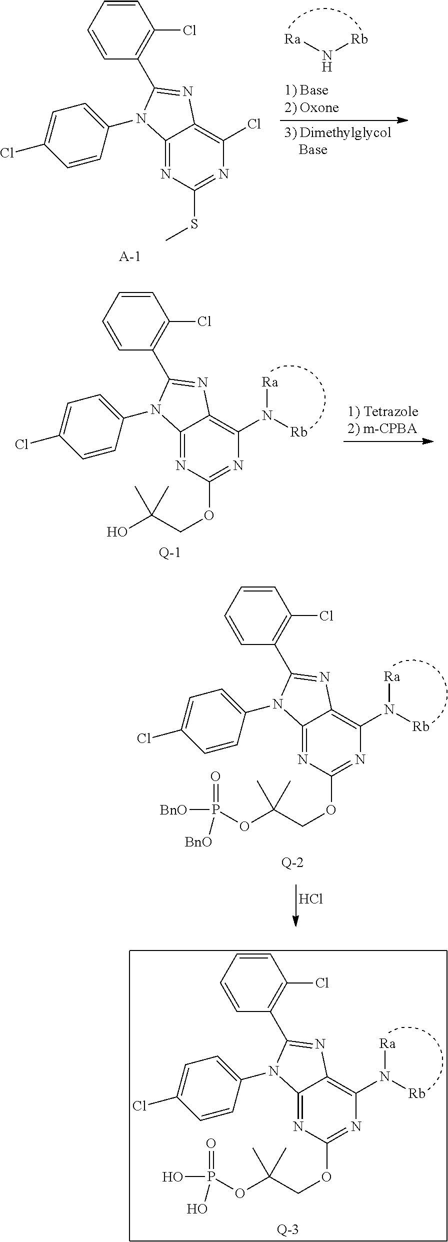

### Preparation of Q-3 Compounds:

Method for compound 239: Step 1, dibenzyl N,N-diisopropylphosphoramidite technical grade, 90% (84.1 μL, 238 μmol) followed by tetrazole solution (880 μL, 396 μmol) (0.45 M in MeCN) were added to a solution of intermediate Q-1 (44.0 mg, 79.2 μmol) in DCM (792 μL) at room temperature. The reaction mixture was stirred 5 minutes at that temperature.

Step 2, 3-chloroperbenzoic acid (28.4 mg, 127 μmol) was added in one portion to the reaction mixture and the solution was stirred at room temperature another 5 minutes where full conversion was observed by LCMS. Water (30 mL) and EtOAc (30 mL) were added and the organic layer was separated, and washed successively with saturated aqueous sodium bicarbonate (50 mL), 10% aqueous sodium bisulfite (50 mL), saturated aqueous sodium bicarbonate (50 mL), brine (50 mL). The organic layer was then dried (magnesium sulfate), filtered and concentrated. Crude material (intermediate Q-2) was used in the next step without purification.

Step 3, The residue was dissolved in DCM (500 μL) and hydrochloric acid (4M in dioxane) (198 μL, 792 μmol) was added. the reaction mixture was stirred overnight at room temperature. The volatiles were removed under reduced pressure and the residue was purified on reverse phase flash chromatography (loading with DMSO, C18 12 g) using 10-60% ACN in 10 mM AmB (pH 10) affording 1-((6-(3-acetamido-3-methylazetidin-1-yl)-8-(2-chlorophenyl)-9-(4-chlorophenyl)-9H-purin-2-yl)oxy)-2-methylpropan-2-yl dihydrogen phosphate (Compound 239, 27.6 mg, 55% yield). ¹H NMR (400 MHz, DMSO-d6): δ=8.45 (s, 1H), 7.67 (dd, 1H), 7.55-7.39 (m, 5H), 7.33-7.26 (m, 2H), 4.60 (br d, 1H), 4.42-4.24 (br m, 2H), 4.15 (br d, 2H), 4.07 (br s, 1H), 1.83 (s, 3H), 1.54 (s, 3H), 1.36 (s, 6H). LCMS: (ES⁺) m/z=635.2 (M+H)⁺.

### (xx) Compound 1:

To a solution of N-[8-(2-chlorophenyl)-9-(4-chlorophenyl)-6-(4-cyano-4-methyl-1-piperidyl)purin-2-yl]-2-methyl-propanamide (Compound 89) (22 mg, 40.1 μmol, 1.0 eq.) in ethanol (0.9 mL) and water (0.1 mL) was added dimethylphosphinite; dimethylphosphinous acid platinum (17 mg, 40.1 mol, 1.0 eq.), the mixture was stirred at 80° C. for 15 hours. The reaction was concentrated in vacuum. The residue was purified by prep-HPLC (column: Unisil 3-100 C18 Ultra 150*50 mm*3 um; mobile phase: [water(formic acid)-acetonitrile]; B %: 42%-72%, 7 min) to give 1-[8-(2-chlorophenyl)-9-(4-chlorophenyl)-2-(2-methylpropanoylamino)purin-6-yl]-4-methyl-piperidine-4-carboxamide (Compound 1, 16.84 mg, 11.5 μmol, 28.6% yield) as an off-white solid. ¹H NMR (400 MHz, MeOD-d₄) δ=7.60 (br d, J=7.6 Hz, 1H), 7.53-7.19 (m, 7H), 4.84-4.61 (m, 2H), 4.09-3.59 (m, 2H), 3.01-2.80 (m, 1H), 2.32-2.11 (m, 2H), 1.75-1.47 (m, 2H), 1.28 (s, 3H), 1.17 (d, J=6.8 Hz, 6H). LCMS: (ES⁺) m/z=566.3 (M+H).

(xxi) Compound 3: To a solution of 4-methyl-1H-pyrazole (37 mg, 447 μmol, 5 eq.) in N,N-dimethylformamide (0.5 mL) was added sodium hydride (5 mg, 107 μmol, 60% purity, 1.2 eq.) at 0° C., the resulting mixture was stirred at 25° C. for 0.5 hour. Then to the mixture was added 1-[8-(2-chlorophenyl)-9-(4-chlorophenyl)-2-methylsulfonyl-purin-6-yl]-4-methyl-piperidine-4-carboxamide (Compound 68) (50 mg, 89.4 μmol, 1 eq.), the resulting mixture was stirred at 25° C. for 2 hours. The mixture was diluted with ammonium chloride saturated solution (2 mL), extracted with ethyl acetate (2 mL*3). The organic layer was dried with anhydrous anhydrous sodium sulfate, filtered and concentrated under vacuum. The residue was purified by Pre-H PLC (column: Waters Xbridge 150*25 mm*5 μm; mobile phase: [water(ammonium bicarbonate)-ACN]; B %: 53%-83%, 10 min) and dried by lyophilization to give 1-[8-(2-chlorophenyl)-9-(4-chlorophenyl)-2-(4-methylpyrazol-1-yl)purin-6-yl]-4-methyl-piperidine-4-carboxamide (Compound 3, 16.05 mg, 28.6 μmol, 32% yield) as a white solid. ¹H NMR (400 MHz, DMSO-d₆) δ=8.33 (s, 1H), 7.75-7.67 (m, 1H), 7.57-7.42 (m, 6H), 7.40-7.33 (m, 2H), 7.29 (s, 1H), 6.97 (s, 1H), 5.25-4.31 (m, 2H), 4.12-3.46 (m, 2H), 2.14 (d, J=14.0 Hz, 2H), 2.08 (s, 3H), 1.50-1.45 (m, 2H), 1.18 (s, 3H). LCMS: (ES⁺) m/z=561.1 (M+H).
(xxii) Compound 4:

To a solution of 3-methyl-1H-pyrazole (73 mg, 894 μmol, 5 eq.) in N,N-dimethylformamide (1 mL) was added sodium hydride (9 mg, 214 μmol, 60% purity, 1.2 eq.) at 0° C., the resulting mixture was stirred at 25° C. for 0.5 hour. Then to the mixture was added 1-[8-(2-chlorophenyl)-9-(4-chlorophenyl)-2-methylsulfonyl-purin-6-yl]-4-methyl-piperidine-4-carboxamide (Compound 68) (0.1 g, 178.74 μmol, 1 eq.), the resulting mixture was stirred at 25° C. for 2 hours. The mixture was diluted with ammonium chloride saturated solution (2 mL), extracted with ethyl acetate (2 mL*3). The organic layer was dried with anhydrous anhydrous sodium sulfate, filtered and concentrated under vacuum. The residue was purified by Pre-HPLC (column: Waters Xbridge 150*25 mm*5 μm; mobile phase: [water(ammonium bicarbonate)-ACN]; B %: 53%-83%, 10 min) and dried by lyophilization to give the product 1-[8-(2-chlorophenyl)-9-(4-chlorophenyl)-2-(3-methylpyrazol-1-yl)purin-6-yl]-4-methyl-piperidine-4-carboxamide (Compound 4, 50.64 mg, 90.2 μmol, 50.4% yield) as white solid. ¹H NMR (400 MHz, DMSO-d₆) δ=8.46 (d, J=2.4 Hz, 1H), 7.74-7.68 (m, 1H), 7.57-7.42 (m, 5H), 7.41-7.34 (m, 2H), 7.29 (s, 1H), 6.97 (s, 1H), 6.29 (d, J=2.4 Hz, 1H), 5.39-4.24 (m, 2H), 4.14-3.43 (m, 2H), 2.24 (s, 3H), 2.14 (d, J=13.6 Hz, 2H), 1.50-1.48 (m, 2H), 1.18 (s, 3H). LCMS: (ES⁺) m/z=561.1 (M+H).

(xxiii) Compound 5:

To a solution of 1-[8-(2-chlorophenyl)-9-(4-chlorophenyl)-2-methylsulfonyl-purin-6-yl]- 4-methyl-piperidine-4-carboxamide (Compound 68) (50 mg, 0.09 mmol, 1 eq.) in N-methyl-2-pyrrolidone (0.5 mL) was added tert-butyl (3S)-3-(hydroxymethyl)piperazine-1-carboxylate (193 mg, 0.89 mmol, 10 eq.), the mixture was stirred at 200° C. for 2 hours under microwave. The reaction mixture was filtered and concentrated under reduced pressure to give a residue. The residue was purified by prep-HPLC (column: Waters Xbridge 150*25 mm*5 μm; mobile phase: [water (ammonium bicarbonate)-acetonitrile]; B %: 40%-70%, 8 min) to give 1-[8-(2-chlorophenyl)-9-(4-chlorophenyl)-2-[(2S)-2-(hydroxymethyl)piperazin-1-yl]purin-6-yl]-4-methyl-piperidine-4-carboxamide (Compound 5, 23.8 mg, 39.96 μmol, 44.7% yield) as a yellow solid. ¹H NMR (400 MHz, DMSO-d₆) δ=7.66-7.63 (m, 1H), 7.51-7.40 (m, 5H), 7.31-7.21 (m, 3H), 6.93 (s, 1H), 4.99-4.14 (m, 5H), 3.93-3.48 (m, 2H), 2.90 (d, J=11.6 Hz, 1H), 2.79-2.70 (m, 1H), 2.62-2.53 (m, 3H), 2.44 (d, J=12.4 Hz, 1H), 2.12-2.01 (m, 2H), 1.46-1.34 (m, 2H), 1.15 (s, 3H). LCMS: (ES⁺) m/z=595.2 (M+H).

(xxiv) Compound 6:

To a solution of 1-[8-(2-chlorophenyl)-9-(4-chlorophenyl)-2-methylsulfonyl-purin-6-yl]-4-methyl-piperidine-4-carboxamide (Compound 68) (80 mg, 143 μmol, 1 eq.) and propane-1,3-diol (54 mg, 714.97 μmol, 5 eq.) in N,N-dimethylformamide (1 mL) was added potassium carbonate (40 mg, 286 μmol, 2 eq.), the resulting mixture was stirred at 100° C. for 12 hours. The mixture was diluted with water (2 mL), extracted with ethyl acetate (2 mL*3). The organic layer was dried with anhydrous anhydrous sodium sulfate, filtered and concentrated under vacuum. The residue was purified by Pre-HPLC (column: Waters Xbridge 150*25 mm*5 μm; mobile phase: [water(ammonium bicarbonate)-ACN]; B %: 42%-72%, 9 min) and dried by lyophilization to give the product 1-[8-(2-chlorophenyl)-9-(4-chlorophenyl)-2-(3-hydroxypropoxy)purin-6-yl]-4-methyl-piperidine-4-carboxamide (Compound 6, 45.86 mg, 82.06 μmol, 57.3% yield) as yellow solid. ¹H NMR (400 MHz, MeOD-d₄) δ=7.61-7.55 (m, 1H), 7.48-7.35 (m, 5H), 7.29-7.23 (m, 2H), 4.75 (br s, 2H), 4.41-4.38 (m, 2H), 3.72-3.69 (m, 4H), 2.20 (d, J=14.2 Hz, 2H), 1.98-1.95 (m, 2H), 1.61-1.55 (m, 2H), 1.28 (s, 3H). LCMS: (ES⁺) m/z=555.2 (M+H).

(xxv) Compound 7:

To a solution of 1-[8-(2-chlorophenyl)-9-(4-chlorophenyl)-2-methylsulfonyl-purin-6-yl]-4-methyl-piperidine-4-carboxamide (Compound 68) (150 mg, 268 μmol, 1 eq.) and propane-1,2-diol (102 mg, 1.34 mmol, 5 eq.) in N,N-dimethylformamide (2 mL) was added potassium carbonate (74 mg, 536 μmol, 2 eq.), the resulting mixture was stirred at 100° C. for 1 hour. The mixture was diluted with water (2 mL), extracted with ethyl acetate (2 mL*3). The organic layer was dried with anhydrous anhydrous sodium sulfate, filtered and concentrated under vacuum. The residue was purified by Pre-HPLC (column: Waters Xbridge 150*25 mm*5 μm; mobile phase: [water(ammonium bicarbonate)-ACN]; B %: 43%-73%, 10 min) and dried by lyophilization to give a mixture (120 mg).

The mixture was separated by chiral SFC (column: DAICEL CHIRALPAK AD (250 mm*30 mm, 10 μm); mobile phase: [0.1% ammonium hydroxide IPA]; B %: 35%-35%, 5 min) to give two peaks.

One peak was purified by Pre-HPLC (column: Waters Xbridge 150*25 mm*5 μm; mobile phase: [water(NH4HCO3)-ACN]; B %: 43%-73%, 10 min) and dried by lyophilization to give 1-[8-(2-chlorophenyl)-9-(4-chlorophenyl)-2-(2-hydroxy-1-methyl-ethoxy)purin-6-yl]-4-methyl-piperidine-4-carboxamide (Compound 8, 19.08 mg, 33.0 μmol, 15.3% yield) as a white solid. ¹H NMR (400 MHz, MeOD-d₄) δ=7.61-7.56 (m, 1H), 7.49-7.35 (m, 5H), 7.29-7.23 (m, 2H), 5.21-5.09 (m, 1H), 4.85-4.66 (m, 2H), 3.98-3.55 (m, 4H), 2.20 (d, J=14.0 Hz, 2H), 1.66-1.51 (m, 2H), 1.32 (d, J=6.4 Hz, 3H), 1.28 (s, 3H). LCMS: (ES⁺) m/z=555.3 (M+H).

(xxvi) Compound 8:

The other peak, according to procedure from Compound 7, was purified by Pre-HPLC (column: Waters Xbridge 150*25 mm*5 μm; mobile phase: [water(ammonium bicarbonate)-ACN]; B %: 43%-73%, 10 min) and dried by lyophilization to give 1-[8-(2-chlorophenyl)-9-(4-chlorophenyl)-2-(2-hydroxypropoxy)purin-6-yl]-4-methyl-piperidine-4-carboxamide (Compound, 93.20 mg, 167.79 μmol, 77.6% yield) as white solid. ¹H NMR (400 MHz, MeOD-d₄) δ=7.61-7.56 (m, 1H), 7.49-7.34 (m, 5H), 7.30-7.23 (m, 2H), 4.84-4.67 (m, 2H), 4.24-4.15 (m, 2H), 4.13-4.04 (m, 1H), 3.95-3.65 (m, 2H), 2.20 (d, J=14.0 Hz, 2H), 1.62-1.56 (m, 2H), 1.28 (s, 3H), 1.23 (d, J=6.4 Hz, 3H). LCMS: (ES⁺) m/z=555.2 (M+H).

(xxvii) Compound 9:

To a solution of 1-[8-(2-chlorophenyl)-9-(4-chlorophenyl)-2-methylsulfonyl-purin-6-yl]-4-methyl-piperidine-4-carboxamide (Compound 68) (80 mg, 0.14 mmol, 1.0 eq.) and 2-methylpropane-1,2-diol (64 mg, 0.71 mmol, 5.0 eq.) in N,N-dimethylformamide (2 mL) was added potassium carbonate (40 mg, 0.29 mmol, 2.0 eq.), the resulting mixture was stirred at 100° C. for 12 hours. The mixture was diluted with water (2 mL), extracted with ethyl acetate (2 mL*3). The organic layer was dried with anhydrous anhydrous sodium sulfate, filtered and concentrated under vacuum. The residue was purified by Pre-HPLC (column: Waters Xbridge C18 150*50 mm*10 μm; mobile phase: [water(ammonium bicarbonate)-acetonitrile]; B %: 47%-77%, 10 min) and then lyophilized to give the product 1-[8-(2-chlorophenyl)-9-(4-chlorophenyl)-2-(2-hydroxy-2-methyl-propoxy)purin-6-yl]-4-methyl-piperidine-4-carboxamide (Compound 9, 37.57 mg, 65.09 μmol, 45.5% yield) as white solid. ¹H NMR (400 MHz, MeOD-d₄) δ=7.5-7.57 (m, 1H), 7.50-7.36 (m, 5H), 7.32-7.25 (m, 2H), 4.90 (br s, 2H), 4.16 (s, 2H), 4.00-3.71 (m, 2H), 2.22 (d, J=14.4 Hz, 2H), 1.70-1.52 (m, 2H), 1.32-1.26 (m, 9H). LCMS: (ES⁺) m/z=569.2 (M+H).

(xxviii) Compound 10:

To a solution of 1-[8-(2-chlorophenyl)-9-(4-chlorophenyl)-2-methylsulfonyl-purin-6-yl]-4-methyl-piperidine-4-carboxamide (Compound 68) (80 mg, 143 μmol, 1 eq.) and 2-methoxyethanol (54 mg, 715 μmol, 5 eq.) in N,N-dimethylformamide (1 mL) was added potassium carbonate (40 mg, 286 μmol, 2 eq.), the resulting mixture was stirred at 100° C. for 12 hours. The mixture was diluted with water (2 mL), extracted with ethyl acetate (2 mL*3). The organic layer was dried with anhydrous anhydrous sodium sulfate, filtered and concentrated under vacuum. The residue was purified by Pre-HPLC (column: Waters Xbridge 150*25 mm*5 μm; mobile phase: [water(NH₄HCO₃)-ACN]; B %: 47%-77%, 9 min) and dried by lyophilization to give 1-[8-(2-chlorophenyl)-9-(4-chlorophenyl)-2-(2-methoxyethoxy)purin-6-yl]-4-methyl-piperidine-4-carboxamide (Compound 10, 46.71 mg, 83.36 μmol, 58.3% yield) as white solid. ¹H NMR (400 MHz, MeOD-d₄) δ=7.61-7.55 (m, 1H), 7.49-7.35 (m, 5H), 7.30-7.22 (m, 2H), 4.82-4.80 (m, 1H), 4.44-4.41 (m, 2H), 3.98-3.65 (m, 4H), 3.38 (s, 3H), 2.25-2.15 (m, 2H), 1.61-1.54 (m, 2H), 1.28 (s, 3H). LCMS: (ES⁺) m/z=555.2 (M+H).

(xxix) Compound 11:

A mixture of 1-[8-(2-chlorophenyl)-9-(4-chlorophenyl)-2-methylsulfonyl-purin-6-yl]-4-methyl-piperidine-4-carboxamide (Compound 68) (0.2 g, 357 μmol, 1 eq.), 2-methylsulfonylethanamine (220.16 mg, 1.79 mmol, 5 eq.) and potassium carbonate (247 mg, 1.79 mmol, 5 eq.) in N-methylpyrrolidone (2 mL) was stirred at 140° C. for 15 hours. The reaction mixture was cooled at room temperature and filtered, the resulting filter liquor was purified by reversed-phase HPLC (column: Phenomenex Luna C18 150*25 mm*10 μm; mobile phase: [water(formic acid)-ACN]; B %: 36%-66%, 10 min). The cut fraction was concentrated under reduced pressure to remove acetonitrile. The residue was lyophilized to give 1-[8-(2-chlorophenyl)-9-(4-chlorophenyl)-2-(2-methylsulfonylethylamino)purin-6-yl]-4-methyl-piperidine-4-carboxamide (Compound 11, 21.43 mg, 35.57 μmol, 9.9% yield) as a white solid. ¹H NMR (400 MHz, CDCl₃-d) δ=7.49 (d, J=7.2 Hz, 1H), 7.38-7.29 (m, 5H), 7.17 (d, J=8.8 Hz, 2H), 5.73-5.57 (m, 2H), 5.44-5.32 (m, 1H), 4.62 (s, 1H), 4.02 (s, 2H), 3.90-3.85 (m, 2H), 3.38 (t, J=6.4 Hz, 2H), 2.89 (s, 3H), 2.21-2.10 (m, 2H), 1.70-1.59 (m, 2H), 1.33 (s, 3H). LCMS: (ES⁺) m/z=602.1 (M+H).

(xxx) Compound 12:

Step 1: A mixture of 1-[8-(2-chlorophenyl)-9-(4-chlorophenyl)-2-methylsulfonyl-purin-6-yl]-4-methyl-piperidine-4-carboxamide (Compound 68) (300 mg, 0.54 mmol, 1 eq.) and 2-aminoethanol (327 mg, 5.36 mmol, 0.32 mL, 10 eq.) was stirred at 140° C. for 12 hours. The reaction mixture was filtered and concentrated under reduced pressure to give the residue. The residue was purified by flash silica gel chromatography (ISCO®; 20 g SepaFlash® Silica Flash Column, Eluent of 0˜100% Ethyl acetate/Petroleum ethergradient @20 mL/min) and the organic phase was concentrated under reduced pressure to give 1-[8-(2-chlorophenyl)-9-(4-chlorophenyl)-2-(2-hydroxyethylamino)purin-6-yl]-4-methyl-piperidine-4-carboxamide (300 mg) as a yellow solid.

Step 2: A mixture of 1-[8-(2-chlorophenyl)-9-(4-chlorophenyl)-2-(2-hydroxyethylamino)purin-6-yl]-4-methyl-piperidine-4-carboxamide (100 mg, 0.18 mmol, 1 eq.), bromomethylbenzene (63 mg, 0.37 mmol, 2 eq.), potassium carbonate (51 mg, 0.37 mmol, 2 eq.) in acetonitrile (1 mL) was stirred at 80° C. for 16 hrs. The reaction mixture was filtered and concentrated under reduced pressure to give a residue. The residue was dissolved in acetonitrile (2 mL) and purified by prep-HPLC (column: Waters Xbridge 150*25 mm*5 μm; mobile phase: [water (ammonium bicarbonate)-acetonitrile]; B %: 61%-81%, 8 min) and by prep-HPLC (column: Phenomenex luna C18 150*25 mm*10 μm; mobile phase: [water (methanoic acid)-acetonitrile]; B %: 53%-83%, 10 min) to give 1-[2-[benzyl(2-hydroxyethyl)amino]-8-(2-chlorophenyl)-9-(4-chlorophenyl)purin-6-yl]-4-methyl-piperidine-4-carboxamide (Compound 12, 6.58 mg, 10.4 μmol, 5.6% yield) as an off-white solid. ¹H NMR (400 MHz, MeOD-d₄) δ=7.61-7.55 (m, 1H), 7.47-7.35 (m, 3H), 7.35-7.08 (m, 9H), 4.88-4.87 (m, 1H), 4.83-4.81 (m, 1H), 4.70-4.57 (m, 2H), 3.73-3.71 (m, 6H), 2.17-2.04 (m, 2H), 1.49 (t, J=10.0 Hz, 2H), 1.24 (s, 3H). LCMS: (ES⁺) m/z=630.2 (M+H).

(xxxi) Compound 13:

A mixture of 1-[8-(2-chlorophenyl)-9-(4-chlorophenyl)-2-methylsulfonyl-purin-6-yl]-4-methyl-piperidine-4-carboxamide (Compound 68) (100 mg, 179 μmol, 1 eq.) and [(2R)-pyrrolidin-2-yl]methanol (181 mg, 1.79 mmol, 0.17 mL, 10 eq.) was stirred at 140° C. for 12 hours. The reaction mixture was filtered and concentrated under reduced pressure to give a residue. The residue was purified by prep-HPLC (column: Waters Xbridge 150*25 mm*5 μm; mobile phase: [water (ammonium bicarbonate)-ACN]; B %: 53%-83%, 10 min) to give 1-[8-(2-chlorophenyl)-9-(4-chlorophenyl)-2-[(2R)-2-(hydroxymethyl)pyrrolidin-1-yl]purin-6-yl]-4-methyl-piperidine-4-carboxamide (Compound 13, 50.93 mg, 87.7 μmol, 49.0% yield) as a white solid. ¹H NMR (400 MHz, MeOD-d₄) δ=7.61-7.54 (m, 1H), 7.47-7.32 (m, 5H), 7.28-7.22 (m, 2H), 4.78-4.59 (m, 2H), 4.14 (d, J=4.0 Hz, 1H), 3.89-3.61 (m, 4H), 3.58-3.54 (m, 2H), 2.17 (d, J=14.4 Hz, 2H), 2.09-1.94 (m, 2H), 1.92-1.79 (m, 2H), 1.60-1.53 (m, 2H), 1.27 (s, 3H). LCMS: (ES⁺) m/z=580.3 (M+H).

(xxxii) Compound 14:

A mixture of 1-[8-(2-chlorophenyl)-9-(4-chlorophenyl)-2-methylsulfonyl-purin-6-yl]-4-methyl-piperidine-4-carboxamide (Compound 68) (0.05 g, 89.4 μmol, 1 eq.), 1,2,3,3a,4,5,6,6a-octahydrocyclopenta[c]pyrrole; hydrochloride (66 mg, 447 μmol, 5 eq.) and potassium carbonate (99 mg, 715 μmol, 8 eq.) in N-methylpyrrolidone (0.5 mL) was stirred at 100° C. for 12 hours. The reaction mixture was cooled at room temperature and diluted with N-methylpyrrolidone (1 mL). The resulting mixture was filtered to afford the filter liquor. The filter liquor was purified by Prep-HPLC (column: Waters Xbridge 150*25 mm*5 μm; mobile phase: [water(ammonium bicarbonate)-ACN]; B %: 66%-96%, 9 min). The cut fraction was concentrated under reduced pressure to remove acetonitrile. The residue was lyophilized to give 1-[2-[(3aS,6aR)-3,3a,4,5,6,6a-hexahydro-1H-cyclopenta[c]pyrrol-2-yl]-8-(2-chlorophenyl)-9-(4-chlorophenyl)purin-6-yl]-4-methyl-piperidine-4-carboxamide (Compound 14, 21 mg, 35.56 μmol, 39.7% yield) as a white solid. ¹H NMR (400 MHz, CDCl₃-d) δ=7.51 (d, J=6.8 Hz, 1H), 7.39-7.29 (m, 5H), 7.27-7.21 (m, 2H), 5.74-5.35 (m, 2H), 4.72-4.48 (m, 2H), 4.14-3.96 (m, 2H), 3.89-3.72 (m, 2H), 3.36 (d, J=10.0 Hz, 2H), 2.78-2.68 (m, 2H), 2.20-2.10 (m, 2H), 1.87-1.79 (m, 2H), 1.71-1.60 (m, 4H), 1.56-1.52 (m, 2H), 1.33 (s, 3H). LCMS: (ES⁺) m/z=590.2 (M+H).

(xxxiii) Compound 15:

A mixture of 1-[8-(2-chlorophenyl)-9-(4-chlorophenyl)-2-methylsulfonyl-purin-6-yl]-4-methyl-piperidine-4-carboxamide (Compound 68) (100 mg, 0.18 mmol, 1 eq.), 3-azabicyclo[3.1.0]hexane hydrochloride (107 mg, 0.89 mmol, 5 eq.) and cesium carbonate (291 mg, 0.89 mmol, 5 eq.) in N-methyl-2-pyrrolidone (1 mL) was stirred at 100° C. for 2 hours. The reaction mixture was filtered and concentrated under reduced pressure to give a residue. The residue was purified by prep-HPLC (column: Waters Xbridge 150*25 mm*5 μm; mobile phase: [water (trifluoroacetic acid)-acetonitrile]; B %: 68%-98%, 8 min) to give 1-[2-[(1R,5S)-3-azabicyclo[3.1.0]hexan-3-yl]-8-(2-chlorophenyl)-9-(4-chlorophenyl)purin-6-yl]-4-methyl-piperidine-4-carboxamide (Compound 15, 19.99 mg, 35.54 μmol, 19.9% yield) as a white solid. ¹H NMR (400 MHz, MeOD-d₄) δ=7.58-7.56 (m, 1H), 7.48-7.30 (m, 5H), 7.30-7.22 (m, 2H), 4.73-4.62 (m, 2H), 3.89-3.66 (m, 4H), 3.41 (d, J=11.2 Hz, 2H), 2.19-2.09 (m, 2H), 1.64-1.52 (m, 4H), 1.27 (s, 3H), 0.72-0.67 (m, 1H), 0.17-0.14 (m, 1H). LCMS: (ES⁺) m/z=562.1 (M+H).

(xxxiv) Compound 16:

Step 1: 1-[8-(2-chlorophenyl)-9-(4-chlorophenyl)-2-methylsulfinyl-purin-6-yl]-4-methyl-piperidine-4-carboxamide (Intermediate C-11) (0.3 g, 552 umol, 1 eq.) and thiomorpholine (569 mg, 5.52 mmol, 0.5 mL, 10 eq.) were taken up into a microwave tube in N-methylpyrrolidone (3 mL). The sealed tube was heated at 160° C. for 1 hour under microwave. The mixture was diluted with water (2 mL), extracted with ethyl acetate (2 mL*3). The organic layer was dried with anhydrous sodium sulfate, filtered and concentrated under vacuum. The residue was purified by Pre-HPLC (column: Waters Xbridge 150*25 mm*5 μm; mobile phase: [water(ammonium bicarbonate)-ACN]; B %: 60%-90%, 8 min) and concentrated under vacuum to give 1-[8-(2-chlorophenyl)-9-(4-chlorophenyl)-2-thiomorpholino-purin-6-yl]-4-methyl-piperidine-4-carboxamide (100 mg, 172 μmol, 31.1% yield) as yellow solid.

Step 2: To a solution of 1-[8-(2-chlorophenyl)-9-(4-chlorophenyl)-2-thiomorpholino-purin-6-yl]-4-methyl-piperidine-4-carboxamide (90 mg, 154 umol, 1 eq.) in N-methylpyrrolidone (1 mL) was added potassium xidooxy hydrogen sulfate (156 mg, 926.97 umol, 6 eq.) and water (0.2 mL). The mixture was stirred at 60° C. for 12 hr. The yellow solid was purified by prep-HPLC (column: Waters Xbridge 150*25 mm*5 um; mobile phase: [water(ammonium bicarbonate)-ACN]; B %: 48%-78%, 8 min) to give the compound 1-[8-(2-chlorophenyl)-9-(4-chlorophenyl)-2-(1,1-dioxo-1,4-thiazinan-4-yl)purin-6-yl]-4-methyl-piperidine-4-carboxamide (Compound 16, 47.74 mg, 77.68 μmol, 50.2% yield) as a white solid. ¹H NMR (400 MHz, CDCl₃-d) δ=7.49 (d, J=6.8 Hz, 1H), 7.40-7.28 (m, 5H), 7.14 (d, J=8.6 Hz, 2H), 5.81-5.12 (m, 2H), 4.86-4.18 (m, 6H), 4.12-3.73 (m, 2H), 3.19-2.87 (m, 4H), 2.26-2.05 (m, 2H), 1.69-1.59 (m, 2H), 1.33 (s, 3H) LCMS: (ES⁺) m/z=614.2 (M+H).

(xxxv) Compound 17:

To a solution of 1-[8-(2-chlorophenyl)-9-(4-chlorophenyl)-2-methylsulfonyl-purin-6-yl]-4-methyl-piperidine-4-carboxamide (Compound 68) (80 mg, 143 μmol, 1 eq.) and tetrahydropyran-3-ol (73 mg, 715 μmol, 5 eq.) in N,N-dimethylformamide (1 mL) was added potassium carbonate (40 mg, 286 μmol, 2 eq.), the resulting mixture was stirred at 100° C. for 12 hours. The mixture was diluted with water (2 mL), extracted with ethyl acetate (2 mL*3). The organic layer was dried with anhydrous anhydrous sodium sulfate, filtered and concentrated under vacuum. The residue was purified by Pre-HPLC (column: Waters Xbridge 150*25 mm*5 μm; mobile phase: [water(ammonium bicarbonate)-ACN]; B %: 53%-83%, 9 min) and dried by lyophilization to give the product 1-[8-(2-chlorophenyl)-9-(4-chlorophenyl)-2-tetrahydropyran-3-yloxy-purin-6-yl]-4-methyl-piperidine-4-carboxamide (Compound 17, 44.19 mg, 75.99 μmol, 53.1% yield) as an off-white solid. ¹H NMR (400 MHz, MeOD-d₄) δ=7.61-7.55 (m, 1H), 7.50-7.35 (m, 5H), 7.28-7.21 (m, 2H), 4.99-4.93 (m, 4H), 3.96-3.92 (m, 1H), 3.84-3.75 (m, 1H), 3.71-3.69 (m, 1H), 3.66-3.56 (m, 2H), 2.20 (d, J=14.4 Hz, 2H), 2.10-2.09 (m, 1H), 1.98-1.78 (m, 2H), 1.67-1.53 (m, 3H), 1.28 (s, 3H). LCMS: (ES⁺) m/z=581.1 (M+H).

(xxxvi) Compound 18:

A mixture of 1-[8-(2-chlorophenyl)-9-(4-chlorophenyl)-2-methylsulfonyl-purin-6-yl]-4-methyl-piperidine-4-carboxamide (Compound 68) (100 mg, 179 μmol, 1 eq.), N-carbamimidoylacetamide (90 mg, 894 μmol, 5 eq.) and potassium carbonate (123 mg, 894 μmol, 5 eq.) in dimethylsulfoxide (1 mL) was stirred at 100° C. for 12 hours. The reaction mixture was filtered and concentrated under reduced pressure to give a residue. The residue was purified by prep-HPLC (column: Waters Xbridge 150*25 mm*5 μm; mobile phase: [water (ammonium bicarbonate)-ACN]; B %: 43%-73%, 10 min) to give 1-[2-[(N-acetylcarbamimidoyl)amino]-8-(2-chlorophenyl)-9-(4-chlorophenyl)purin-6-yl]-4-methyl-piperidine-4-carboxamide (Compound 18, 22.95 mg, 39.5 μmol, 22.1% yield, 100% purity) as a white solid. ¹H NMR (400 MHz, MeOD-d₄) δ=7.64-7.57 (m, 1H), 7.49-7.40 (m, 5H), 7.33-7.26 (m, 2H), 4.81-4.78 (m, 2H), 3.90-3.69 (m, 2H), 2.25-2.18 (m, 2H), 2.06 (s, 3H), 1.64-1.55 (m, 2H), 1.29 (s, 3H). LCMS: (ES⁺) m/z=580.3 (M+H).

(xxxvii) Compound 19:

A mixture of 1-[8-(2-chlorophenyl)-9-(4-chlorophenyl)-2-methylsulfonyl-purin-6-yl]-4-methyl-piperidine-4-carboxamide (Compound 68) (130 mg, 232 μmol, 1 eq.), 2-(methylamino)ethanol (935 mg, 12.45 mmol, 1 mL, 54 eq.) was stirred at 100° C. for 18 hours. The reaction mixture was cooled at room temperature and diluted with N,N-dimethylformamide (2 mL). The resulting mixture was purified by reversed-phase HPLC (column: Waters Xbridge 150*25 mm*5 μm; mobile phase: [water(ammonium bicarbonate)-ACN]; B %: 46%-76%, 8 min). The cut fraction was concentrated under reduced pressure to remove acetonitrile. The residue was lyophilized to give 1-[8-(2-chlorophenyl)-9-(4-chlorophenyl)-2-[2-hydroxyethyl(methyl)amino]purin-6-yl]-4-methyl-piperidine-4-carboxamide (Compound 19, 48.31 mg, 87.13 μmol, 37.5% yield) as an off-white solid. ¹H NMR (400 MHz, DMSO-d₆) δ=7.64 (d, J=7.2 Hz, 1H), 7.54-7.39 (m, 5H), 7.27 (d, J=8.8 Hz, 3H), 6.94 (s, 1H), 4.98-4.26 (m, 3H), 3.70-3.52 (m, 6H), 3.07 (s, 3H), 2.12-2.02 (m, 2H), 1.48-1.34 (m, 2H), 1.16 (s, 3H). LCMS: (ES⁺) m/z=554.2.

(xxxviii) Compound 20:

A mixture of 1-[8-(2-chlorophenyl)-9-(4-chlorophenyl)-2-methylsulfinyl-purin-6-yl]-4-methyl-piperidine-4-carboxamide (Compound 68) (100 mg, 184 μmol, 1 eq.) and [(2S)-pyrrolidin-2-yl]methanol (186 mg, 1.84 mmol, 0.18 mL, 10 eq.) was stirred at 140° C. for 12 hours. The reaction mixture was filtered and concentrated under reduced pressure to give a residue. The residue was purified by prep-HPLC (column: Waters Xbridge 150*25 mm*5 μm; mobile phase: [water(ammonium bicarbonate)-ACN]; B %: 52%-82%, 10 min) to give 1-[8-(2-chlorophenyl)-9-(4-chlorophenyl)-2-[(2S)-2-(hydroxymethyl)pyrrolidin-1-yl]purin-6-yl]-4-methyl-piperidine-4-carboxamide (Compound 20, 53.2 mg, 91.09 μmol, 49.5% yield) as an off-white solid. ¹H NMR (400 MHz, MeOD-d₄) δ=7.59-7.52 (m, 1H), 7.47-7.33 (m, 5H), 7.24 (d, J=8.4 Hz, 2H), 4.75-4.62 (m, 2H), 4.14 (s, 1H), 3.81-3.63 (m, 4H), 3.58-3.54 (m, 2H), 2.16 (d, J=14.4 Hz, 2H), 2.08-1.93 (m, 2H), 1.90-1.80 (m, 2H), 1.63-1.49 (m, 2H), 1.27 (s, 3H). LCMS: (ES⁺) m/z=580.3 (M+H).

(xxxix) Compound 21:

To a solution of 4-methyl-1H-pyrazole (72 mg, 877 μmol, 5 eq.) in N,N-dimethylformamide (1 mL) was added sodium hydride (8 mg, 210 μmol, 60% purity, 1.2 eq.) at 0° C., the resulting mixture was stirred at 25° C. for 0.5 hour. Then to the mixture was added 8-(2-chlorophenyl)-9-(4-chlorophenyl)-2-methylsulfonyl-6-[4-(trifluoromethyl)-1-piperidyl]purine (Intermediate D-3) (0.1 g, 175 μmol, 1 eq.). The resulting mixture was stirred at 25° C. for 2 hours. The mixture was diluted with ammonium chloride saturated solution (2 mL), extracted with ethyl acetate (2 mL*3). The organic layer was dried with anhydrous anhydrous sodium sulfate, filtered and concentrated under vacuum. The residue was purified by Pre-HPLC (column: Waters Xbridge 150*25 mm*5 μm; mobile phase: [water(ammonium bicarbonate)-ACN]; B %: 70%-100%, 8 min) and dried by lyophilization to give the product 8-(2-chlorophenyl)-9-(4-chlorophenyl)-2-(4-methylpyrazol-1-yl)-6-[4-(trifluoromethyl)-1-piperidyl]purine (Compound 21, 78.63 mg, 137 μmol, 78.3% yield) as an off-white solid. ¹H NMR (400 MHz, CDCl₃-d) δ=8.27 (s, 1H), 7.60 (s, 1H), 7.53-7.52 (m, 1H), 7.44-7.32 (m, 5H), 7.26-7.21 (m, 2H), 6.10-5.38 (m, 2H), 3.17-3.12 (m, 2H), 2.44-2.41 (m, 1H), 2.15 (s, 3H), 2.08 (d, J=11.6 Hz, 2H), 1.80-1.73 (m, 2H). ¹⁹F NMR (377 MHz, CDCl₃-d) δ=−73.85 (s, 1F). LCMS: (ES⁺) m/z=572.2 (M+H).

### (xl) Compound 22:

To a solution of 3-methyl-1H-pyrazole (72 mg, 876.56 μmol, 5 eq.) in N,N-dimethylformamide (1 mL) was added sodium hydride (8 mg, 210.37 μmol, 60% purity, 1.2 eq.) at 0° C., the resulting mixture was stirred at 25° C. for 0.5 hour. Then to the mixture was added 8-(2-chlorophenyl)-9-(4-chlorophenyl)-2-methylsulfonyl-6-[4-(trifluoromethyl)-1-piperidyl]purine (Intermediate D-3) (0.1 g, 175 μmol, 1 eq.). The resulting mixture was stirred at 25° C. for 2 hours. The mixture was diluted with ammonium chloride saturated solution (2 mL), extracted with ethyl acetate (2 mL*3). The organic layer was dried with anhydrous anhydrous sodium sulfate, filtered and concentrated under vacuum. The residue was purified by Pre-HPLC (column: Waters Xbridge 150*25 mm*5 μm; mobile phase: [water(ammonium bicarbonate)-ACN]; B %: 67%-97%, 10 min) and dried by lyophilization to give the product 8-(2-chlorophenyl)-9-(4-chlorophenyl)-2-(3-methylpyrazol-1-yl)-6-[4-(trifluoromethyl)-1-piperidyl]purine (Compound 22, 23.98 mg, 41.89 μmol, 23.9% yield) as an off-white solid. ¹H NMR (400 MHz, DMSO-d₆) δ=8.48 (d, J=2.4 Hz, 1H), 7.73-7.71 (m, 1H), 7.57-7.43 (m, 5H), 7.42-7.36 (m, 2H), 6.30 (d, J=2.8 Hz, 1H), 6.15-4.98 (m, 2H), 3.30-3.05 (m, 2H), 2.87-2.70 (m, 1H), 2.24 (s, 3H), 2.03 (d, J=11.2 Hz, 2H), 1.58-1.50 (m, 2H). ¹⁹F NMR (377 MHz, DMSO-d₆) δ=−72.46 (s, 1F). LCMS: (ES⁺) m/z=572.1 (M+H).

(xli) Compound 23:

To a solution of 8-(2-chlorophenyl)-9-(4-chlorophenyl)-2-methylsulfinyl-6-[4-(trifluoromethyl)-1-piperidyl]purine (Intermediate D-3) (100 mg, 0.18 mol, 1 eq.) and propane-1,3-diol (68 mg, 0.90 mmol, 5 eq.) in N,N-dimethylformamide (1 mL) was added potassium carbonate (50 mg, 0.36 mmol, 2 eq.), the resulting mixture was stirred at 100° C. for 12 hours. The mixture was diluted with water (2 mL), extracted with ethyl acetate (4 mL*3). The organic layer was dried with anhydrous sodium sulfate, filtered and concentrated under vacuum. The residue was purified by Pre-HPLC (column: Waters Xbridge 150*25 mm*5 μm; mobile phase: [water(ammonium bicarbonate)-ACN]; B %: 59%-89%, 9 min) and dried by lyophilization. The residue was purified by Pre-HPLC (column: Waters Xbridge 150*25 mm*5 μm; mobile phase: [water (ammonia hydroxide v/v)-ACN]; B %: 54%-84%, 9 min) and dried by lyophilization to give 3-[8-(2-chlorophenyl)-9-(4-chlorophenyl)-6-[4-(trifluoromethyl)-1-piperidyl]purin-2-yl]oxypropan-1-ol (Compound 23, 57.46 mg, 101.45 μmol, 56.2% yield) as a white solid. ¹H NMR (400 MHz, MeOH-d₄) 5=7.59 (d, J=7.6 Hz, 1H), 7.46-7.36 (m, 5H), 7.27 (d, J=8.8 Hz, 2H), 5.71-5.45 (m, 2H), 4.42-4.39 (m, 2H), 3.73-3.69 (m, 2H), 3.22-3.08 (m, 2H), 2.73-2.51 (m, 1H), 2.08-1.93 (m, 4H), 1.68-1.54 (m, 2H). ¹⁹F NMR (377 MHz, MeOH-d₄) 5=−75.50 (s, 1F). LCMS: (ES⁺) m/z=566.2 (M+H).

(xlii) Compound 24:

To a solution of 8-(2-chlorophenyl)-9-(4-chlorophenyl)-2-methylsulfonyl-6-[4-(trifluoromethyl)-1-piperidyl]purine (Intermediate D-3) (150 mg, 263 μmol, 1 eq.) and propane-1,2-diol (100 mg, 1.31 mmol, 5 eq.) in N,N-dimethylformamide (2 mL) was added potassium carbonate (73 mg, 526 μmol, 2 eq.), the resulting mixture was stirred at 100° C. for 12 hours. The mixture was diluted with water (2 mL), extracted with ethyl acetate (2 mL*3). The organic layer was dried with anhydrous anhydrous sodium sulfate, filtered and concentrated under vacuum. The residue was purified by Pre-HPLC (column: Waters Xbridge 150*25 mm*5 μm; mobile phase: [water(ammonium bicarbonate)-ACN]; B %: 66%-96%, 9 min) and dried by lyophilization to give the mixture (60 mg). The mixture was separated by chiral SFC (column: Daicel ChiralPak IG (250*30 mm, 10 μm); mobile phase: [0.1% ammonium hydroxide ethanol]; B %: 30%-30%, 3.4 min) to give two peaks.

One peak was purified by Pre-HPLC (column: Waters Xbridge 150*25 mm*5 μm; mobile phase: [water(ammonium bicarbonate)-ACN]; B %: 62%-92%, 10 min) and dried by lyophilization to give 2-[8-(2-chlorophenyl)-9-(4-chlorophenyl)-6-[4-(trifluoromethyl)-1-piperidyl]purin-2-yl]oxypropan-1-01 (Compound 24, 11.07 mg, 19.54 μmol, 18.45% yield) as an off-white solid. ¹H NMR (400 MHz, MeOD-d₄) δ=7.63-7.57 (m, 1H), 7.49-7.35 (m, 5H), 7.30-7.23 (m, 2H), 5.76-5.39 (m, 2H), 5.24-5.11 (m, 1H), 3.76-3.58 (m, 2H), 3.18-3.11 (m, 2H), 2.61-2.59 (m, 1H), 2.02 (d, J=11.2 Hz, 2H), 1.71-1.55 (m, 2H), 1.32 (d, J=6.4 Hz, 3H). ¹⁹F NMR (376 MHz, MeOD-d₄) δ=−75.51 (br s, 1F). LCMS: (ES⁺) m/z=566.3 (M+H).

(xliii) Compound 25:

The other peak, according to procedure from Compound 24, was purified by Pre-HPLC (column: Waters Xbridge 150*25 mm*5 μm; mobile phase: [water(ammonium bicarbonate)-ACN]; B %: 62%-92%, 10 min) again and dried by lyophilization to give 1-[8-(2-chlorophenyl)-9-(4-chlorophenyl)-6-[4-(trifluoromethyl)-1-piperidyl]purin-2-yl]oxypropan-2-ol (Compound 25, 28.11 mg, 49.6 μmol, 46.8% yield) as a white solid. ¹H NMR (400 MHz, MeOD-d₄) δ=7.62-7.56 (m, 1H), 7.49-7.36 (m, 5H), 7.31-7.24 (m, 2H), 5.75-5.41 (m, 2H), 4.25-4.15 (m, 2H), 4.14-4.02 (m, 1H), 3.18-3.12 (m, 2H), 2.61-2.59 (m, 1H), 2.09-1.96 (m, 2H), 1.67-1.60 (m, 2H), 1.23 (d, J=6.4 Hz, 3H). ¹⁹F NMR (377 MHz, MeOD-d₄) δ=−75.51 (s, 1F). LCMS: (ES⁺) m/z=566.2 (M+H).

(xliv) Compound 26:

To a solution of 8-(2-chlorophenyl)-9-(4-chlorophenyl)-2-methylsulfonyl-6-[4-(trifluoromethyl)-1-piperidyl]purine (Intermediate D-3) (100 mg, 175 μmol, 1 eq.) and 2-methylpropane-1,2-diol (79 mg, 877 μmol, 5 eq.) in N,N-dimethylformamide (2 mL) was added potassium carbonate (48 mg, 351 μmol, 2 eq.), the resulting mixture was stirred at 100° C. for 12 hours. The mixture was diluted with water (2 mL), extracted with ethyl acetate (2 mL*3). The organic layer was dried with anhydrous sodium sulfate, filtered and concentrated under vacuum. The residue was purified by Pre-HPLC (column: Waters Xbridge 150*25 mm*5 μm; mobile phase: [water(ammonium bicarbonate)-ACN]; B %: 62%-92%, 9 min) and dried by lyophilization to give the product 1-[8-(2-chlorophenyl)-9-(4-chlorophenyl)-6-[4-(trifluoromethyl)-1-piperidyl]purin-2-yl]oxy-2-methyl-propan-2-ol (Compound 26, 50.93 mg, 87.7 μmol, 50.0% yield) as white solid. ¹H NMR (400 MHz, MeOD-d₄) δ=7.57 (d, J=8.8 Hz, 2H), 7.50-7.49 (m, 2H), 7.33-7.28 (m, 2H), 7.24 (d, J=7.2 Hz, 1H), 7.21-7.14 (m, 5H), 4.59 (s, 2H), 4.44-4.30 (m, 2H), 2.42-2.25 (m, 1H), 2.14 (s, 3H), 2.09-2.05 (m, 1H). ¹⁹F NMR (377 MHz, MeOD-d₄) δ=−110.05 (br s, 1 F). LCMS: (ES⁺) m/z=573.2 (M+H).

(xlv) Compound 27:

To a solution of 8-(2-chlorophenyl)-9-(4-chlorophenyl)-2-methylsulfonyl-6-[4-(trifluoro methyl)-1-piperidyl]purine (Intermediate D-3) (50 mg, 0.88 mmol, 1.0 eq.) and 2-methoxyethanol (20 mg, 0.26 mmol, 3.0 eq.) in tetrahydrofuran (1 mL) was added sodium hydride (4 mg, 0.10 mmol, 60% purity, 1.2 eq.), the resulting mixture was stirred at 80° C. for 1 hour. The mixture was diluted with ammonium chloride saturated solution (2 mL), extracted with ethyl acetate (2 mL*3). The organic layer was dried with anhydrous anhydrous sodium sulfate, filtered and concentrated under vacuum to give a crude product (60 mg).

To a solution of 8-(2-chlorophenyl)-9-(4-chlorophenyl)-2-methylsulfonyl-6-[4-(trifluoromethyl)-1-piperidyl]purine (20 mg, 0.04 mmol, 1.0 eq.) and 2-methoxyethanol (13 mg, 0.18 mmol, 5.0 eq.) in N,N-dimethylformamide (0.5 mL) was added potassium carbonate (10 mg, 0.07 mmol, 2.0 eq.), the resulting mixture was stirred at 100° C. for 12 hours. The mixture was diluted with water (1 mL), extracted with ethyl acetate (1 mL*3). The organic layer was dried with anhydrous sodium sulfate, filtered and concentrated under vacuum to give crude product (20 mg).

To a solution of potassium tert-butoxide (8 mg, 0.07 mmol, 2.0 eq.) in tetrahydrofuran (0.5 mL) was added 2-methoxyethanol (13 mg, 0.18 mmol, 5.0 eq.) and 8-(2-chlorophenyl)-9-(4-chlorophenyl)-2-methylsulfonyl-6-[4-(trifluoromethyl)-1-piperidyl]purine (20 mg, 0.04 mmol, 1.0 eq.) at 0° C., the resulting mixture was stirred at 60° C. for 12 hours. The mixture was diluted with water (1 mL), extracted with ethyl acetate (1 mL*3). The organic layer was dried with anhydrous anhydrous sodium sulfate, filtered and concentrated under vacuum to give crude product (20 mg).

Three batches were combined and purified by Pre-HPLC (column: Waters Xbridge 150*25 mm*5 μm; mobile phase: [water(ammonium bicarbonate)-ACN]; B %: 67%-97%, 10 min) and dried by lyophilization to give the product 8-(2-chlorophenyl)-9-(4-chlorophenyl)-2-(2-methoxyethoxy)-6-[4-(trifluoromethyl)-1-piperidyl]purine (Compound 27, 56.92 mg, 99.57 μmol, 63.1% yield, 99.0% purity) as off-white solid. ¹H NMR (400 MHz, MeOD-d₄) δ=7.59 (d, J=7.6 Hz, 1H), 7.50-7.34 (m, 5H), 7.27 (d, J=8.8 Hz, 2H), 5.57 (s, 2H), 4.49-4.40 (m, 2H), 3.76-3.68 (m, 2H), 3.38 (s, 3H), 3.17-3.11 (m, 2H), 2.67-2.55 (m, 1H), 2.02 (d, J=13.2 Hz, 2H), 1.70-1.57 (m, 2H). ¹⁹F NMR (376 MHz, MeOD-d₄) δ=75.514 (m, 1F). LCMS: (ES⁺) m/z=566.1 (M+H).

(xlvi) Compound 28:

A mixture of 8-(2-chlorophenyl)-9-(4-chlorophenyl)-2-methylsulfonyl-6-[4-(trifluoromethyl)-1-piperidyl]purine (Intermediate D-3) (0.2 g, 0.35 μmol, 1 eq.), 2-methylsulfonylethanamine (216 mg, 1.75 mmol, 5 eq.) and potassium carbonate (242.29 mg, 1.75 mmol, 5 eq.) in N-methy-2-pyrrolidone (2 mL) was stirred at 140° C. for 12 hours. The reaction mixture was cooled at room temperature and filtrated, the resulting filter liquor was purified by reversed-phase HPLC (column: Phenomenex Synergi C18 150*25 mm*10 μm; mobile phase: [water(formic acid)-ACN]; B %: 57%-87%, 10 min). The cut fraction was concentrated under reduced pressure to remove acetonitrile. The residue was lyophilized to give 8-(2-chlorophenyl)-9-(4-chlorophenyl)-N-(2-methylsulfonylethyl)-6-[4-(trifluoromethyl)-1-piperidyl]purin-2-amine (Compound 28, 13.62 mg, 22.2 μmol, 6.3% yield) as a white solid. ¹H NMR (400 MHz, CDCl₃-d) δ=7.53-7.47 (m, 1H), 7.40-7.30 (m, 5H), 7.21-7.15 (m, 2H), 5.76-5.53 (m, 2H), 5.27-5.13 (m, 1H), 3.93-3.85 (m, 2H), 3.38 (t, J=6.4 Hz, 2H), 3.04 (t, J=12.8 Hz, 2H), 2.88 (s, 3H), 2.43-2.38 (m, 1H), 2.03 (d, J=12.8 Hz, 2H), 1.76-1.69 (m, 2H). LCMS: (ES⁺) m/z=613.0 (M+H).

(xlvii) Compound 29:

A mixture of 8-(2-chlorophenyl)-9-(4-chlorophenyl)-2-methylsulfonyl-6-[4-(trifluoromethyl)-1-piperidyl]purine (Intermediate D-3) (50 mg, 87.7 μmol, 1 eq.) and [(2R)-pyrrolidin-2-yl]methanol (89 mg, 0.88 mmol, 0.09 mL, 10 eq.) was stirred at 140° C. for 5 hours. The reaction mixture was filtered and concentrated under reduced pressure to give a residue. The residue was purified by prep-HPLC (column: Waters Xbridge 150*25 mm*5 μm; mobile phase: [water(ammonium bicarbonate)-ACN]; B %: 80%-100%, 10 min) to give [(2R)-1-[8-(2-chlorophenyl)-9-(4-chlorophenyl)-6-[4-(trifluoromethyl)-1-piperidyl]purin-2-yl]pyrrolidin-2-yl]methanol (Compound 29, 29.97 mg, 50.7 μmol, 57.8% yield) as a white solid. ¹H NMR (400 MHz, MeOD-d₄) δ=7.61-7.55 (m, 1H), 7.50-7.32 (m, 5H), 7.29-7.21 (m, 2H), 5.52 (d, J=12.8 Hz, 2H), 4.14 (s, 1H), 3.77-3.49 (m, 4H), 3.08 (t, J=12.8 Hz, 2H), 2.65-2.49 (m, 1H), 2.12-1.80 (m, 6H), 1.66-1.55 (m, 2H). LCMS: (ES⁺) m/z=591.2 (M+H).

(xlviii) Compound 30:

A mixture of 8-(2-chlorophenyl)-9-(4-chlorophenyl)-2-methylsulfinyl-6-[4-(trifluoromethyl)-1-piperidyl]purine (Intermediate D-2) (50 mg, 90.2 μmol, 1 eq.), 1,2,3,3a,4,5,6,6a-octahydrocyclopenta[c]pyrrole; hydrochloride (27 mg, 180 μmol, 2 eq.) and potassium carbonate (37.39 mg, 271 μmol, 3 eq.). indimethylsulfoxide (0.5 mL) was stirred at 100° C. for 20 hours. The reaction mixture was cooled at room temperature and diluted with water (20 mL). The mixture was extracted with ethyl acetate (10 mL*3) and the combined organic layers were concentrated under reduced pressure, the resulting residue was purified by flash silica gel chromatography (ISCO®; 12 g SepaFlash® Silica Flash Column, Eluent of 0˜50% Ethyl acetate/Petroleum ethergradient @20 mL/min). The cut fraction was concentrated under reduced pressure and lyophilized to give compound 2-[(3aS,6aR)-3,3a,4,5,6,6a-hexahydro-1H-cyclopenta[c]pyrrol-2-yl]-8-(2-chlorophenyl)-9-(4-chlorophenyl)-6-[4-(trifluoromethyl)-1-piperidyl]purine (Compound 30, 0.02 g, 32.59 μmol, 36.1% yield) as a white solid. ¹H NMR (400 MHz, DMSO-d₆) δ=7.78-7.60 (m, 1H), 7.58-7.38 (m, 5H), 7.28 (d, J=8.8 Hz, 2H), 5.70-5.15 (m, 2H), 3.70-3.61 (m, 2H), 3.26-2.97 (m, 4H), 2.79-2.62 (m, 3H), 2.03-1.88 (m, 2H), 1.83-1.62 (m, 3H), 1.59-1.34 (m, 5H). LCMS: (ES⁺) m/z=602.9 (M+H).

(xlix) Compound 31:

A mixture of 8-(2-chlorophenyl)-9-(4-chlorophenyl)-2-methylsulfonyl-6-[4-(trifluoromethyl)-1-piperidyl]purine (Intermediate D-2) (0.1 g, 175 μmol, 1 eq.), 3-azabicyclo[3.1.0]hexane hydrochloride (210 mg, 1.75 mmol, 10 eq.) and cesium carbonate (856.80 mg, 2.63 mmol, 15 eq.) in N-methy-2-pyrrolidone (0.5 mL) was stirred at 100° C. for 12 hours. The reaction mixture was cooled at room temperature and diluted with N-methy-2-pyrrolidone (1 mL). The resulting mixture was filtered to afford the filter liquor. The filter liquor was purified by reversed-phase HPLC (column: Waters Xbridge 150*25 mm*5 μm; mobile phase: [water(ammonium bicarbonate)-ACN]; B %: 80%-100%, 9 min). The cut fraction was concentrated under reduced pressure to remove acetonitrile. The residue was lyophilized to give 2-[(1R,5S)-3-azabicyclo[3.1.0]hexan-3-yl]-8-(2-chlorophenyl)-9-(4-chlorophenyl)-6-[4-(trifluoromethyl)-1-piperidyl]purine (Compound 31, 20 mg, 34.9 μmol, 19.8% yield) as a white solid. ¹H NMR (400 MHz, CDCl₃-d) δ=7.31-7.26 (m, 1H), 7.14-7.04 (m, 5H), 7.01-6.96 (m, 2H), 5.52-5.27 (m, 2H), 3.64 (d, J=10.8 Hz, 2H), 3.22 (d, J=10.4 Hz, 2H), 2.76 (t, J=12.8 Hz, 2H), 2.19-2.05 (m, 1H), 1.79-1.70 (m, 2H), 1.58-1.38 (m, 2H), 1.31-1.26 (m, 2H), 0.52-0.38 (m, 1H), 0.09-0.03 (m, 1H). LCMS: (ES⁺) m/z=573.1 (M+H), Rt=1.215 min.

### (l) Compound 32:

Step 1: 8-(2-chlorophenyl)-9-(4-chlorophenyl)-2-methylsulfinyl-6-[4-(trifluoromethyl)-1-piperidyl]purine (Intermediate D-2) (300 mg, 541 μmol, 1 eq.) and thiomorpholine (558 mg, 5.41 mmol, 10 eq.) were taken up into a microwave tube in N-methylpyrrolidone (3 mL). The sealed tube was heated at 160° C. for 1 hour under microwave. The mixture was diluted with water (2 mL), extracted with ethyl acetate (2 mL*3). The organic layer was dried with anhydrous anhydrous sodium sulfate, filtered and concentrated under vacuum. The residue was purified by Pre-HPLC (column: Waters Xbridge C18 150*50 mm*10 μm; mobile phase: [water(ammonium bicarbonate)-ACN]; B %: 64%-94%, 10 min) and dried by lyophilization. The residue was purified by column chromatography (silicon dioxide, Petroleum ether/Ethyl acetate=1/0 to 10/1) and concentrated under vacuum to give the product 4-[8-(2-chlorophenyl)-9-(4-chlorophenyl)-6-[4-(trifluoromethyl)-1-piperidyl]purin-2-yl]thiomorpholine (60 mg, 101 μmol, 18.6% yield) as a white solid.

Step 2: To solution of 4-[8-(2-chlorophenyl)-9-(4-chlorophenyl)-6-[4-(trifluoromethyl)-1-piperidyl]purin-2-yl]thiomorpholine (50 mg, 84.2 μmol, 1 eq.) in N-methylpyrrolidone (0.5 mL) was added a solution of potassium oxidooxy hydrogen sulfate (85 mg, 505 μmol, 6 eq.) in water (0.1 mL), the mixture was stirred at 60° C. for 12 hours. The residue was purified by Pre-HPLC (column: Phenomenex Synergi C18 150*25 mm*10 μm; mobile phase: [water(formic acid)-ACN]; B %: 69%-99%, 10 min) and dried by lyophilization to give the product 4-[8-(2-chlorophenyl)-9-(4-chlorophenyl)-6-[4-(trifluoromethyl)-1-piperidyl]purin-2-yl]-1,4-thiazinane 1,1-dioxide (Compound 32, 16.17 mg, 25.8 μmol, 30.6% yield) as a white solid. ¹H NMR (400 MHz, CDCl₃-d) δ=7.50-7.48 (m, 1H), 7.38-7.29 (m, 5H), 7.18-7.13 (m, 2H), 5.84-5.39 (m, 2H), 4.30 (s, 4H), 3.13-2.93 (m, 6H), 2.52-2.29 (m, 1H), 2.02 (d, J=11.6 Hz, 2H), 1.72-1.63 (m, 2H). ¹⁹F NMR (376 MHz, CDCl₃-d) δ=−73.89 (s, 1F) LCMS: (ES⁺) m/z=626.9 (M+H).

### (li) Compound 33:

To a solution of 8-(2-chlorophenyl)-9-(4-chlorophenyl)-2-methylsulfonyl-6-[4-(trifluoromethyl)-1-piperidyl]purine (Intermediate D-3) (80 mg, 140 μmol, 1 eq.) and tetrahydropyran-3-ol (72 mg, 701 μmol, 5 eq.) in N,N-dimethylformamide (1 mL) was added potassium carbonate (39 mg, 280 μmol, 2 eq.), the resulting mixture was stirred at 100° C. for 12 hours. The mixture was diluted with water (2 mL), extracted with ethyl acetate (2 mL*3). The organic layer was dried with anhydrous Na₂SO₄, filtered and concentrated under vacuum. The residue was purified by Pre-HPLC (column: Waters Xbridge 150*25 mm*5 μm; mobile phase: [water(ammonium bicarbonate)-ACN]; B %: 70%-100%, 10 min) and dried by lyophilization to give the product 8-(2-chlorophenyl)-9-(4-chlorophenyl)-2-tetrahydropyran-3-yloxy-6-[4-(trifluoromethyl)-1-piperidyl]purine (Compound 33, 50.81 mg, 85.8 μmol, 61.1% yield) as a white solid. ¹H NMR (400 MHz, MeOD-d₄) δ=7.62-7.56 (m, 1H), 7.51-7.35 (m, 5H), 7.26 (d, J=8.8 Hz, 2H), 5.69-5.44 (m, 2H), 4.97-4.94 (m, 1H), 3.95-3.92 (m, 1H), 3.75-3.56 (m, 3H), 3.21-3.09 (m, 2H), 2.61-2.60 (m, 1H), 2.14-1.98 (m, 3H), 1.96-1.79 (m, 2H), 1.70-1.54 (m, 3H). ¹⁹F NMR (376 MHz, MeOD-d₄) δ=−75.51 (s, 1F). LCMS: (ES⁺) m/z=592.2 (M+H), Rt=1.160 min. (Method 4).

### (ii) Compound 34:

A mixture of 8-(2-chlorophenyl)-9-(4-chlorophenyl)-2-methylsulfinyl-6-[4-(trifluoromethyl)-1-piperidyl]purine (Intermediate D-2) (100 mg, 180 μmol, 1 eq.), N-carbamimidoylacetamide (36 mg, 360 μmol, 2 eq.) and potassium carbonate (75 mg, 541 μmol, 3 eq.) in dimethylsulfoxide (1 mL) was stirred at 100° C. for 12 hours. The reaction mixture was filtered and concentrated under reduced pressure to give a residue. The residue was dissolved in acetonitrile (2 mL) and purified by prep-HPLC (column: Waters Xbridge 150*25 mm*5 μm; mobile phase: [water(ammonium bicarbonate)-ACN]; B %: 61%-91%, 10 min) and the organic phase was concentrated under reduced pressure to remove acetonitrile, and the liquid was lyophilized to give N—[N-[8-(2-chlorophenyl)-9-(4-chlorophenyl)-6-[4-(trifluoromethyl)-1-piperidyl]purin-2-yl]carbamimidoyl]acetamide (Compound 34, 16.09 mg, 27.2 μmol, 15.0% yield) as an off-white solid. ¹H NMR (400 MHz, MeOD-d₄) δ=7.67-7.60 (m, 1H), 7.53-7.41 (m, 5H), 7.37-7.29 (m, 2H), 5.03-4.92 (m, 2H), 3.28-3.04 (m, 2H), 2.78-2.54 (m, 1H), 2.18 (s, 3H), 2.08 (d, J=11.6 Hz, 2H), 1.71-1.59 (m, 2H). LCMS: (ES⁺) m/z=591.3 (M+H).

(liii) Compound 35:

A mixture of 8-(2-chlorophenyl)-9-(4-chlorophenyl)-2-methylsulfinyl-6-[4-(trifluoromethyl)-1-piperidyl]purine (Intermediate D-2) (200 mg, 361 μmol, 1 eq.), [(2S)-pyrrolidin-2-yl]methanol (73 mg, 721 μmol, 0.07 mL, 2 eq.) and potassium carbonate (150 mg, 1.08 mmol, 3 eq.) in dimethylsulfoxide (2 mL) was stirred at 60° C. for 12 hours. The reaction mixture was filtered and concentrated under reduced pressure to give a residue. The residue was purified by prep-HPLC (column: Waters Xbridge 150*25 mm*5 μm; mobile phase: [water(ammonium bicarbonate)-ACN]; B %: 67%-97%, 9 min) to give [(2S)-1-[8-(2-chlorophenyl)-9-(4-chlorophenyl)-6-[4-(trifluoromethyl)-1-piperidyl]purin-2-yl]pyrrolidin-2-yl]methanol (Compound 35, 6.44 mg, 10.9 μmol, 3.02% yield) as a white solid. ¹H NMR (400 MHz, MeOD-d₄) δ=7.61-7.55 (m, 1H), 7.48-7.33 (m, 5H), 7.30-7.22 (m, 2H), 5.52 (d, J=12.0 Hz, 2H), 4.25-4.05 (m, 1H), 3.77-3.47 (m, 4H), 3.15-3.00 (m, 2H), 2.65-2.48 (m, 1H), 2.09-1.85 (m, 6H), 1.66-1.56 (m, 2H). LCMS: (ES⁺) m/z=591.3 (M+H).

(liv) Compound 36:

To a solution of 1-[6-(4-chloroanilino)-2-[(2S)-2-(hydroxymethyl)pyrrolidin-1-yl]-5-nitro-pyrimidin-4-yl]-4-methyl-piperidine-4-carboxamide (Intermediate B-5(36), Example 4i) (80 mg, 163 μmol, 1 eq.) and 4-formylbenzonitrile (107 mg, 816.40 μmol, 5 eq.) in ethanol (1 mL) and acetic acid (0.4 mL) was added iron (91 mg, 1.63 mmol, 10 eq.). The mixture was stirred at 80° C. for 12 hours. The reaction mixture was filtered and concentrated in vacuum. The residue was purified by prep-HPLC (column: Unisil 3-100 C18 Ultra 150*50 mm*3 μm; mobile phase: [water(formic acid)-ACN]; B %: 41%-71%, 7 min) to give a yellow solid. Then the yellow solid was purified by prep-HPLC (column: Waters Xbridge 150*25 mm*5 μm; mobile phase: [water(ammonium bicarbonate)-ACN]; B %: 51%-81%, 8 min) to give 1-[9-(4-chlorophenyl)-8-(4-cyanophenyl)-2-[(2S)-2-(hydroxymethyl)pyrrolidin-1-yl]purin-6-yl]-4-methyl-piperidine-4-carboxamide (Compound 36, 10.04 mg, 17.3 μmol, 10.6% yield) as a yellow solid. ¹H NMR (400 MHz, DMSO-d₆) δ=7.82 (d, J=8.4 Hz, 2H), 7.56 (m, 4H), 7.40 (d, J=8.7 Hz, 2H), 7.25 (s, 1H), 7.01-6.86 (m, 1H), 5.28-4.40 (m, 2H), 4.08-3.92 (m, 1H), 3.73-3.52 (m, 2H), 3.36-3.28 (m, 4H), 2.21-2.02 (m, 2H), 1.99-1.62 (m, 4H), 1.40 (m, 2H), 1.15 (s, 3H). LCMS: (ES⁺) m/z=571.4 (M+H).

### (lv) Compound 37:

To a solution of 1-[6-(4-chloroanilino)-2-[(2S)-2-(hydroxymethyl)pyrrolidin-1-yl]-5-nitro-pyrimidin-4-yl]-4-methyl-piperidine-4-carboxamide (Intermediate B-5(36), Example 4i) (80 mg, 163 μmol, 1 eq.) and 3-formylbenzonitrile (107 mg, 816 μmol, 5 eq.) in ethanol (1 mL) and acetic acid (0.4 mL) was added iron (91 mg, 1.63 mmol, 10 eq.). The mixture was stirred at 80° C. for 2 hours. The reaction mixture was filtered and concentrated in vacuum. The residue was purified by flash silica gel chromatography (ISCO®; X g SepaFlash® Silica Flash Column, Eluent of 0˜10% Methanol/Dichloromethane@80 mL/min) to give a yellow solid (R_{f}=0.3). The white solid was purified by prep-HPLC (column: Waters Xbridge 150*25 mm*5 μm; mobile phase: [water(ammonia hydroxide)-ACN]; B %: 50%-80%, 8 min) to give a yellow solid. Then the yellow solid was purified by prep-HPLC (column: Phenomenex luna C18 150*25 mm*10 μm; mobile phase: [water(formic acid)-ACN]; B %: 42%-72%, 10 min) to give the compound 1-[9-(4-chlorophenyl)-8-(3-cyanophenyl)-2-[(2S)-2-(hydroxymethyl)pyrrolidin-1-yl]purin-6-yl]-4-methyl-piperidine-4-carboxamide (Compound 37, 28.10 mg, 48.6 μmol, 29.7% yield) as a yellow solid. ¹H NMR (400 MHz, CDCl₃-d) δ=7.95 (s, 1H), 7.68-7.41 (m, 4H), 7.38-7.31 (m, 1H), 7.23 (d, J=8.6 Hz, 2H), 6.43-5.84 (m, 1H), 5.77-5.31 (m, 2H), 5.08-4.33 (m, 2H), 4.23-3.94 (m, 2H), 3.84-3.45 (m, 4H), 2.25-2.02 (m, 3H), 1.97-1.78 (m, 2H), 1.70-1.56 (m, 4H), 1.34 (s, 3H). LCMS: (ES⁺) m/z=571.3 (M+H).

(lvi) Compound 38:

A mixture of 8-(2-chlorophenyl)-9-(4-chlorophenyl)-2-methylsulfonyl-6-[4-(trifluoromethyl)-1-piperidyl]purine (Intermediate D-3) (50 mg, 87.7 μmol, 1 eq.) and 2-methyl-1-(methylamino)propan-2-ol (90 mg, 0.88 mmol, 10 eq.) was stirred at 140° C. for 12 hours. The reaction mixture was diluted with N,N-dimethylformamide 1 mL, the residue was purified by prep-HPLC (column: Waters Xbridge 150*25 mm*5 μm; mobile phase: [water(ammonia hydroxide)-ACN]; B %: 66%-96%, 11 min) to give 1-[[8-(2-chlorophenyl)-9-(4-chlorophenyl)-6-[4-(trifluoromethyl)-1-piperidyl]purin-2-yl]-methyl-amino]-2-methyl-propan-2-ol (Compound 38, 25.10 mg, 41.9 μmol, 47.8% yield) as a white solid. ¹H NMR (400 MHz, MeOD-d₄) δ=7.60-7.57 (m, 1H), 7.51-7.31 (m, 5H), 7.25 (d, J=8.8 Hz, 2H), 5.59-5.41 (m, 2H), 3.60 (s, 2H), 3.21 (s, 3H), 3.15-3.02 (m, 2H), 2.61-2.52 (m, 1H), 2.00 (d, J=11.6 Hz, 2H), 1.67-1.56 (m, 2H), 1.18 (s, 6H). LCMS: (ES⁺) m/z=593.2 (M+H).

(lvii) Compound 40:

A mixture of 1-[8-(2-chlorophenyl)-9-(4-chlorophenyl)-2-methylsulfonyl-purin-6-yl]-4-methyl-piperidine-4-carboxamide (Compound 68) (100 mg, 179 μmol, 1 eq.) and 2-methyl-1-(methylamino)propan-2-ol (184 mg, 1.79 mmol, 10 eq.) was stirred at 140° C. for 12 hours. The reaction mixture was filtered and concentrated under reduced pressure to give a residue. The residue was purified by prep-HPLC (column: Waters Xbridge 150*25 mm*5 μm; mobile phase: [water (ammonium bicarbonate)-ACN]; B %: 55%-85%, 10 min) to give 1-[8-(2-chlorophenyl)-9-(4-chlorophenyl)-2-[(2-hydroxy-2-methyl-propyl)-methyl-amino]purin-6-yl]-4-methyl-piperidine-4-carboxamide (Compound 40, 60.69 mg, 103 μmol, 57.5% yield) as an off-white solid. ¹H NMR (400 MHz, MeOD-d₄) δ=7.60-7.54 (m, 1H), 7.47-7.32 (m, 5H), 7.23 (d, J=8.8 Hz, 2H), 4.71-4.70 (m, 2H), 3.77 (t, J=10.4 Hz, 2H), 3.59 (s, 2H), 3.20 (s, 3H), 2.17 (d, J=14.4 Hz, 2H), 1.60-1.53 (m, 2H), 1.27 (s, 3H), 1.18 (s, 6H). LCMS: (ES⁺) m/z=582.5 (M+H).

(lviii) Compound 41:

A mixture of 1-[8-(2-chlorophenyl)-9-(4-chlorophenyl)-2-methylsulfonyl-purin-6-yl]-4-methyl-piperidine-4-carboxamide (Compound 68) (100 mg, 179 μmol, 1.0 eq.) and 1-(methylaminomethyl)cyclopropanol (181 mg, 1.79 mmol, 10 eq.) in N-methylpyrrolidone (0.3 mL) was stirred at 140° C. for 12 hours. The reaction mixture was filtered and concentrated under reduced pressure. The residue was purified flash silica gel chromatography (ISCO®; 25 g SepaFlash® Silica Flash Column, Eluent of 0˜50% methanol/dichloroethane ether gradient @30 mL/min) and prep-HPLC (column: Phenomenex luna C18 150*25 mm*10 μm; mobile phase: [water(FA)-ACN]; B %: 51%-81%, 10 min). 1-[8-(2-chlorophenyl)-9-(4-chlorophenyl)-2-[(1-hydroxycyclopropyl)methyl-methyl-amino]purin-6-yl]-4-methyl-piperidine-4-carboxamide (Compound 41, 7 mg, 11.9 μmol, 3.4% yield). ¹H NMR (400 MHz, MeOD-d₄) δ=7.69-7.15 (m, 8H), 4.77-4.63 (m, 2H), 4.25 (s, 2H), 3.86-3.58 (m, 2H), 3.24-3.17 (m, 3H), 2.50-2.33 (m, 2H), 2.28-2.01 (m, 2H), 1.71-1.46 (m, 2H), 1.28 (s, 3H), 0.98 (t, J=7.2 Hz, 2H). LCMS: (ES⁺) m/z=580.3 (M+H).

(lix) Compound 42:

A mixture of 1-[8-(2-chlorophenyl)-9-(4-chlorophenyl)-2-methylsulfonyl-purin-6-yl]-4-methyl-piperidine-4-carboxamide (Compound 68) (0.4 g, 0.71 mmol, 1 eq.), 2-methoxy-N-methyl-ethanamine (1.25 g, 14.0 mmol, 1.5 mL, 20 eq.) was stirred at 100° C. for 48 hours. The reaction mixture was cooled at room temperature and diluted with N,N-dimethylformamide (3 mL). The resulting mixture was purified by reversed-phase HPLC(column: Waters Xbridge 150*25 mm*5 μm; mobile phase: [water(ammonium bicarbonate)-ACN]; B %: 58%-88%, 8 min). The cut fraction was concentrated under reduced pressure to remove acetonitrile. The residue was lyophilized to give 1-[8-(2-chlorophenyl)-9-(4-chlorophenyl)-2-[2-methoxyethyl(methyl)amino]purin-6-yl]-4-methyl-piperidine-4-carboxamide (Compound 42, 103 mg, 181 μmol, 25.3% yield) as a white solid. ¹H NMR (400 MHz, CDCl₃-d) δ=7.53-7.49 (m, 1H), 7.39-7.29 (m, 5H), 7.24-7.19 (m, 2H), 5.82-5.31 (m, 2H), 4.75-4.43 (m, 2H), 4.12-3.90 (m, 2H), 3.83-3.76 (m, 2H), 3.65-3.59 (m, 2H), 3.36 (s, 3H), 3.21 (s, 3H), 2.21-2.12 (m, 2H), 1.70-1.65 (m, 2H), 1.33 (s, 3H). LCMS: (ES⁺) m/z=569.9 (M+H).

### (lx) Compound 43:

A mixture of 1H-imidazol-2-ylmethanol (140 mg, 1.43 mmol, 8 eq.) and sodium hydride (36 mg, 0.89 mmol, 60% purity, 5 eq.) in N,N-dimethylformamide (1.5 mL) was stirred at 25° C. for 30 min, then 1-[8-(2-chlorophenyl)-9-(4-chlorophenyl)-2-methylsulfonyl-purin-6-yl]-4-methyl-piperidine-4-carboxamide (Compound 68) (100 mg, 179 μmol, 1 eq.) was added to the mixture and the mixture was stirred at 60° C. for 1.5 hours. The reaction mixture was cooled at room temperature and diluted with N,N-dimethylformamide (1 mL). The resulting mixture was filtered to afford the filter liquor, which was purified by reversed-phase HPLC (column: Waters Xbridge 150*25 mm*5 μm; mobile phase: [water(ammonium bicarbonate)-ACN]; B %: 4%-70%, 8 min) to give 1-[8-(2-chlorophenyl)-9-(4-chlorophenyl)-2-[2-(hydroxymethyl)imidazol-1-yl]purin-6-yl]-4-methyl-piperidine-4-carboxamide (Compound 43, 8.7 mg, 14.76 μmol, 8.2% yield) as an off-white solid. ¹H NMR (400 MHz, DMSO-d₆) δ=7.81 (d, J=1.2 Hz, 1H), 7.75-7.71 (m, 1H), 7.57-7.46 (m, 5H), 7.43-7.39 (m, 2H), 7.31 (s, 1H), 6.99 (s, 1H), 6.92 (d, J=1.2 Hz, 1H), 5.30-4.96 (m, 1H), 4.87-4.80 (m, 1H), 4.79-4.72 (m, 2H), 4.61-4.29 (m, 1H), 4.21-3.85 (m, 1H), 3.70-3.44 (m, 1H), 2.21-2.12 (m, 2H), 1.56-1.44 (m, 2H), 1.19 (s, 3H). LCMS: (ES⁺) m/z=577.2 (M+H).

(lxi) Compound 46:

Step 1: A mixture of ethyl 1H-imidazole-5-carboxylate (200 mg, 1.43 mmol, 8 eq.) and sodium hydride (35.75 mg, 0.89 mmol, 60% purity, 5 eq.) in N,N-dimethylformamide (1 mL) was stirred at 25° C. for 30 min, then 1-[8-(2-chlorophenyl)-9-(4-chlorophenyl)-2-methylsulfonyl-purin-6-yl]-4-methyl-piperidine-4-carboxamide (Compound 68) (100 mg, 179 μmol, 1 eq.) was added to the mixture and the mixture was stirred at 60° C. for 5.5 hours. The reaction mixture was diluted with water (30 mL) and extracted with ethyl acetate (20 mL*3). The combined organic layers were washed with brine (30 mL*2), dried over sodium sulfate, filtered and concentrated under reduced pressure to give compound ethyl 1-[6-(4-carbamoyl-4-methyl-1-piperidyl)-8-(2-chlorophenyl)-9-(4-chlorophenyl)purin-2-yl]imidazole-4-carboxylate (132 mg, crude) as a white solid, which was used for the next step directly without further purification.

Step 2: To a solution of ethyl 1-[6-(4-carbamoyl-4-methyl-1-piperidyl)-8-(2-chlorophenyl)-9-(4-chlorophenyl)purin-2-yl]imidazole-4-carboxylate (105 mg, 0.17 mmol, 1 eq.) in tetrahydrofuran (1 mL) and methanol (2 mL) was added a solution of lithium hydroxide monohydrate (35.67 mg, 0.85 mmol, 5 eq.) in water (3 mL). The resulting mixture was stirred at 60° C. for 7 hours. The reaction mixture was concentrated under reduced pressure to remove methanol and tetrahydrofuran. The residue was diluted with water (5 mL), pH of the mixture was adjusted to 4 by hydrochloric acid (2 M) and then the mixture was filtered, the resulting solid was lyophilized to afford compound 1-[6-(4-carbamoyl-4-methyl-1-piperidyl)-8-(2-chlorophenyl)-9-(4-chlorophenyl)purin-2-yl]imidazole-4-carboxylic acid (90 mg, crude) as a white solid.

Step 3: To a solution of 1-[6-(4-carbamoyl-4-methyl-1-piperidyl)-8-(2-chlorophenyl)-9-(4-chlorophenyl)purin-2-yl]imidazole-4-carboxylic acid (90 mg, 152 μmol, 1 eq.) in tetrahydrofuran (1.5 mL) was added a solution of diisobutylaluminum hydride (1 M, 0.76 mL, 5 eq.) in toluene. The resulting mixture was stirred at 25° C. for 1 hour. The reaction mixture was poured into sodium hydroxide aqueous solution (1M, 3 mL) and then diluted with water (10 mL), extracted with ethyl acetate (8 mL*3). The combined organic layers were washed with brine (20 mL), dried over anhydrous sodium sulfate, filtered and concentrated under reduced pressure, the resulting residue was purified by reversed-phase HPLC (column: Phenomenex luna C18 150*25 mm*10 μm; mobile phase: [water(formic acid)-ACN]; B %: 32%-52%, 10 min). The cut fraction was concentrated under reduced pressure to remove acetonitrile. The residue was lyophilized to give compound 1-[8-(2-chlorophenyl)-9-(4-chlorophenyl)-2-[4-(hydroxymethyl)imidazol-1-yl]purin-6-yl]-4-methyl-piperidine-4-carboxamide (Compound 46.14 mg, 24.24 μmol, 15.9% yield) as a white solid. ¹H NMR (400 MHz, DMSO-d₆) δ=8.41 (s, 1H), 7.73 (d, J=7.2 Hz, 1H), 7.64 (s, 1H), 7.57-7.46 (m, 5H), 7.39 (d, J=8.8 Hz, 2H), 7.34-6.95 (m, 2H), 5.20-4.43 (m, 3H), 4.40 (d, J=5.6 Hz, 2H), 4.16-3.49 (m, 2H), 2.20-2.11 (m, 2H), 1.54-1.44 (m, 2H), 1.19 (s, 3H). LCMS: (ES⁺) m/z=577.3 (M+H).

(lxii) Compound 47:

Step 1: A mixture of 2-(1H-imidazol-4-yl)ethyl acetate (198 mg, 1.29 mmol, 9 eq.) and sodium hydride (29 mg, 0.71 mmol, 60% purity, 5 eq.) in N,N-dimethylformamide (2 mL) was stirred at 25° C. for 30 min, then 1-[8-(2-chlorophenyl)-9-(4-chlorophenyl)-2-methylsulfonyl-purin-6-yl]-4-methyl-piperidine-4-carboxamide (Compound 68) (80 mg, 143 μmol, 1 eq.) was added to the mixture and the mixture was stirred at 60° C. for 6.5 hours. The reaction mixture was diluted with water (20 mL) and extracted with ethyl acetate (15 mL*3). The combined organic layers were washed with brine (20 mL*2), dried over sodium sulfate, filtered and concentrated under reduced pressure, The resulting residue was purified by prep-HPLC (column: Waters Xbridge 150*25 mm*5 μm; mobile phase: [water(ammonium bicarbonate)-ACN]; B %: 45%-75%, 9 min). The cut fraction was concentrated under reduced pressure to remove acetonitrile. The residue was lyophilized to give 2-[1-[6-(4-carbamoyl-4-methyl-1-piperidyl)-8-(2-chlorophenyl)-9-(4-chlorophenyl)purin-2-yl]imidazol-4-yl]ethyl acetate (48 mg, 58.3 μmol, 40.8% yield) as a white solid.

Step 2: To a solution of 2-[1-[6-(4-carbamoyl-4-methyl-1-piperidyl)-8-(2-chlorophenyl)-9-(4-chlorophenyl)purin-2-yl]imidazol-4-yl]ethyl acetate (36 mg, 56.8 μmol, 1 eq.) in methanol (1 mL) and tetrahydrofuran (0.5 mL) was added a solution sodium hydroxide (11 mg, 284 μmol, 5 eq.) in water (0.5 mL). The resulting mixture was stirred at 50° C. for 4 hours. The reactant mixture was concentrated under reduced pressure to remove tetrahydrofuran and methanol. The residue was diluted with water (10 mL) and extracted with ethyl acetate (3*10 mL), the combined organic layers were washed with brine (10 mL), dried over sodium sulfate, filtered and concentrated under reduced pressure, the resulting residue was purified by reversed-phase HPLC (column: Waters Xbridge 150*25 mm*5 μm; mobile phase: [water(ammonium bicarbonate)-ACN]; B %: 38%-68%, 10 min). The cut fraction was concentrated under reduced pressure to remove acetonitrile. The residue was lyophilized to give 1-[8-(2-chlorophenyl)-9-(4-chlorophenyl)-2-[4-(2-hydroxyethyl)imidazol-1-yl]purin-6-yl]-4-methyl-piperidine-4-carboxamide (Compound 47, 18 mg, 30.4 μmol, 53.5% yield) as a white solid. ¹H NMR (400 MHz, CDCl₃-d) δ=8.46 (s, 1H), 7.60 (s, 1H), 7.56-7.52 (m, 1H), 7.46-7.35 (m, 5H), 7.25-7.18 (m, 2H), 5.73-5.35 (m, 2H), 5.11-3.35 (m, 6H), 2.86 (t, J=5.6 Hz, 2H), 2.26-2.18 (m, 2H), 1.73-1.67 (m, 2H), 1.36 (s, 3H). LCMS: (ES⁺) m/z=591.2 (M+H).

(lxiii) Compound 48:

To a solution of 1-[6-(4-chloroanilino)-2-[2-hydroxyethyl(methyl)amino]-5-nitro-pyrimidin-4-yl]-4-methyl-piperidine-4-carboxamide (Intermediate B-5(48), Example 4ii) (50 mg, 108 μmol, 1 eq.) and benzaldehyde (57 mg, 539 μmol, 5 eq.) in ethanol (1 mL) was added acetic acid (0.4 mL) and iron (30 mg, 539 μmol, 5 eq.). The mixture was stirred at 80° C. for 1 hour. The reaction mixture was filtered and concentrated in vacuum. The residue was purified by prep-HPLC (column: Waters Xbridge 150*25 mm*5 μm; mobile phase: [water(ammonium bicarbonate)-ACN]; B %: 49%-79%, 8 min) to give 1-[9-(4-chlorophenyl)-2-[2-hydroxyethyl(methyl)amino]-8-phenyl-purin-6-yl]-4-methyl-piperidine-4-carboxamide (Compound 48, 16.26 mg, 31.27 μmol, 29.0% yield) as an off-white solid. ¹H NMR (400 MHz, CDCl₃-d) δ=7.50-7.37 (m, 4H), 7.35-7.27 (m, 3H), 7.26-7.18 (m, 2H), 5.82-5.26 (m, 2H), 4.86-4.41 (m, 2H), 4.19-3.87 (m, 2H), 3.86-3.76 (m, 2H), 3.73-3.63 (m, 2H), 3.17 (s, 3H), 2.22-2.03 (m, 2H), 1.70-1.64 (m, 2H), 1.33 (s, 3H). LCMS: (ES⁺) m/z=520.2 (M+H).

(lxiv) Compound 49:

A mixture of 1-[6-(4-chloroanilino)-2-[2-hydroxyethyl(methyl)amino]-5-nitro-pyrimidin-4-yl]-4-methyl-piperidine-4-carboxamide (Intermediate B-5(48), Example 4ii) (100 mg, 0.22 mmol, 1 eq.) and 3-chloropyridine-2-carbaldehyde (152 mg, 1.08 mmol, 5 eq.) in ethyl alcohol (2 mL) was added iron (120 mg, 2.16 mmol, 10 eq.) and acetic acid (0.8 mL), the mixture was stirred at 80° C. for 1 hour. The reaction mixture was filtered and concentrated under reduced pressure to give a residue. The residue was purified by prep-HPLC (column: Shim-pack C18 150*25*10 μm; mobile phase: [water(methanoic acid)-acetonitrile]; B %: 35%-65%, 10 min) to give 1-[9-(4-chlorophenyl)-8-(3-chloro-2-pyridyl)-2-[2-hydroxyethyl(methyl)amino]purin-6-yl]-4-methyl-piperidine-4-carboxamide (Compound 49, 25.06 mg, 45.1 μmol, 20.9% yield) as a yellow solid. ¹H NMR (400 MHz, DMSO-d₆) δ=8.53 (d, J=3.6 Hz, 1H), 8.04 (d, J=8.0 Hz, 1H), 7.57-7.41 (m, 3H), 7.36-7.17 (m, 3H), 6.95 (s, 1H), 5.51-3.89 (m, 3H), 3.57 (s, 6H), 3.08 (s, 3H), 2.11-2.01 (m, 2H), 1.40 (t, J=9.6 Hz, 2H), 1.15 (s, 3H). LCMS: (ES⁺) m/z=555.4 (M+H).

(lxv) Compound 50:

To a solution of 1-[6-(4-chloroanilino)-2-[2-hydroxyethyl(methyl)amino]-5-nitro-pyrimidin-4-yl]-4-methyl-piperidine-4-carboxamide (Intermediate B-5(48), Example 4ii) (50 mg, 108 μmol, 1 eq.) and 3-fluoropyridine-2-carbaldehyde (67 mg, 539 μmol, 5 eq.) in ethanol (1 mL) was added acetic acid (0.4 mL) and iron (30 mg, 539 μmol, 5 eq.). The mixture was stirred at 80° C. for 1 hour. The reaction mixture was filtered and concentrated in vacuum. The residue was purified by prep-H PLC (column: Waters Xbridge 150*25 mm*5 μm; mobile phase: [water(ammonium bicarbonate)-ACN]; B %: 38%-68%, 8 min) to give the compound 1-[9-(4-chlorophenyl)-8-(3-fluoro-2-pyridyl)-2-[2-hydroxyethyl(methyl)amino]purin-6-yl]-4-methyl-piperidine-4-carboxamide (Compound 50, 12.10 mg, 22.2 μmol, 20.6% yield) as a yellow solid. ¹H NMR (400 MHz, CDCl₃-d) δ=8.41 (br d, J=4.3 Hz, 1H), 7.54-7.28 (m, 4H), 7.20 (br d, J=8.4 Hz, 2H), 5.70-5.31 (m, 2H), 4.98-4.37 (m, 3H), 4.21-3.88 (m, 2H), 3.87-3.59 (m, 4H), 3.19 (s, 3H), 2.24-2.03 (m, 2H), 1.67 (br s, 2H), 1.31 (s, 3H). LCMS: (ES⁺) m/z=539.2 (M+H).

(lxvi) Compound 51:

A mixture of 1-[6-(4-chloroanilino)-2-[2-hydroxyethyl(methyl)amino]-5-nitro-pyrimidin-4-yl]-4-methyl-piperidine-4-carboxamide (Intermediate B-5(48), Example 4ii) (50 mg, 0.11 mmol, 1 eq.) and 3-methylpyridine-2-carbaldehyde (65 mg, 0.54 mmol, 5 eq.) in ethyl alcohol (1 mL) was added iron (60 mg, 1.08 mmol, 10 eq.) and acetic acid (0.4 mL), the mixture was stirred at 80° C. for 1 hour. The reaction mixture was filtered and concentrated under reduced pressure to give a residue. The residue was purified by prep-HPLC (column: Waters Xbridge 150*25 mm*5 μm; mobile phase: [water (ammonium bicarbonate)-acetonitrile]; B %: 37%-67%, 10 min) to give the crude product. The crude product was purified by prep-HPLC (column: Phenomenex luna C18 150*25 mm*10 μm; mobile phase: [water (methanoic acid)-acetonitrile]; B %: 35%-65%, 10 min) to give 1-[9-(4-chlorophenyl)-2-[2-hydroxyethyl(methyl)amino]-8-(3-methyl-2-pyridyl)purin-6-yl]-4-methyl-piperidine-4-carboxamide (Compound 51, 6.11 mg, 11.4 μmol, 10.6% yield) as a yellow solid. ¹H NMR (400 MHz, DMSO-d₆) δ=8.30-8.20 (m, 1H), 7.75 (d, J=6.8 Hz, 1H), 7.45-7.40 (m, 2H), 7.30-7.27 (m, 1H), 7.25-7.23 (m, 2H), 7.23-7.21 (m, 1H), 6.94 (s, 1H), 4.58 (s, 2H), 3.57 (s, 6H), 3.07 (s, 3H), 2.42 (s, 3H), 2.13-2.03 (m, 2H), 1.43-1.37 (m, 2H), 1.15 (s, 3H). LCMS: (ES⁺) m/z=535.3 (M+H).

(lxvii) Compound 52:

To a solution of 1-[6-(4-chloroanilino)-2-[2-hydroxyethyl(methyl)amino]-5-nitro-pyrimidin-4-yl]-4-methyl-piperidine-4-carboxamide (Intermediate B-5(48), Example 4ii) (50 mg, 108 μmol, 1 eq.) and pyridine-4-carbaldehyde (58 mg, 539 μmol, 5 eq.) in ethanol (1 mL) and acetic acid (0.4 mL) was added iron (60 mg, 1.08 mmol, 10 eq.). The mixture was stirred at 80° C. for 1 hour. The reaction mixture was filtered and concentrated in vacuum. The residue was purified by prep-H PLC (column: Unisil 3-100 C18 Ultra 150*50 mm*3 μm; mobile phase: [water(formic acid)-ACN]; B %: 20%-40%, 10 min) to give 1-[9-(4-chlorophenyl)-2-[2-hydroxyethyl(methyl)amino]-8-(4-pyridyl)purin-6-yl]-4-methyl-piperidine-4-carboxamide (Compound 52, 12.41 mg, 23.6 μmol, 21.9% yield) as a yellow solid. ¹H NMR (400 MHz, DMSO-d₆) δ=8.53 (br d, J=4.8 Hz, 2H), 7.60 (d, J=8.6 Hz, 2H), 7.42 (d, J=8.6 Hz, 2H), 7.35-7.20 (m, 3H), 6.95 (s, 1H), 4.74-4.37 (m, 2H), 3.54 (s, 6H), 3.04 (s, 3H), 2.10 (br d, J=13.3 Hz, 2H), 1.48-1.35 (m, 2H), 1.16 (s, 3H). LCMS: (ES⁺) m/z=521.2 (M+H).

(lxviii) Compound 53:

To a solution of 8-(2-chlorophenyl)-9-(4-chlorophenyl)-2-methylsulfonyl-6-[4-(trifluoromethyl)-1-piperidyl]purine (Intermediate D-3) (80 mg, 140 μmol, 1 eq.) and potassium carbonate (19 mg, 140.25 μmol, 1 eq.) in N,N-dimethylformamide (1 mL) was added N-(2-hydroxyethyl)acetamide (72 mg, 701 μmol, 5 eq.), the resulting mixture was stirred at 100° C. for 12 hours. The reaction mixture was added in methanol (1 mL). The residue was purified by Pre-HPLC (column: Waters Xbridge 150*25 mm*5 μm; mobile phase: [water(ammonium bicarbonate)-ACN]; B %: 57%-87%, 10 min) and dried by lyophilization to give the product N-[2-[8-(2-chlorophenyl)-9-(4-chlorophenyl)-6-[4-(trifluoromethyl)-1-piperidyl]purin-2-yl]oxyethyl]acetamide (Compound 53, 49.60 mg, 83.6 μmol, 59.6% yield) as white solid. ¹H NMR (400 MHz, CDCl₃-d) δ=7.50-7.48 (m, 1H), 7.41-7.29 (m, 5H), 7.17 (d, J=8.8 Hz, 2H), 6.13 (br s, 1H), 5.97-5.33 (m, 2H), 4.49-4.32 (m, 2H), 3.76-3.57 (m, 2H), 3.10-3.05 (m, 2H), 2.42-2.39 (m, 1H), 2.04 (d, J=12.0 Hz, 2H), 1.93 (s, 3H), 1.76-1.64 (m, 2H). ¹⁹F NMR (376 MHz, CHLOROFORM-d) 5=−73.89 (s, 1F). LCMS: (ES⁺) m/z=593.2 (M+H).

(lxix) Compound 54:

A mixture of 1-[8-(2-chlorophenyl)-9-(4-chlorophenyl)-2-methylsulfonyl-purin-6-yl]-4-methyl-piperidine-4-carboxamide (Compound 68) (100 mg, 178.74 μmol, 1 eq.) and (2R)-1-(methylamino)propan-2-ol (159 mg, 1.79 mmol, 10 eq.) was stirred at 140° C. for 5 hours. The reaction mixture was filtered and concentrated under reduced pressure to give a residue. The residue was purified by prep-HPLC (column: Waters Xbridge 150*25 mm*5 μm; mobile phase: [water(ammonium bicarbonate)-ACN]; B %: 52%-82%, 8 min) to give 1-[8-(2-chlorophenyl)-9-(4-chlorophenyl)-2-[[(2R)-2-hydroxypropyl]-methyl-amino]purin-6-yl]-4-methyl-piperidine-4-carboxamide (Compound 54, 57.95 mg, 102 μmol, 57.03% yield) as a white solid. ¹H NMR (400 MHz, MeOD-d₄) δ=7.61-7.55 (m, 1H), 7.47-7.31 (m, 5H), 7.28-7.22 (m, 2H), 4.70-4.67 (m, 2H), 4.14-4.03 (m, 1H), 3.76 (t, J=10.4 Hz, 2H), 3.68-3.63 (m, 1H), 3.46-3.41 (m, 1H), 3.18 (s, 3H), 2.17 (d, J=14.4 Hz, 2H), 1.60-1.53 (m, 2H), 1.27 (s, 3H), 1.14 (d, J=6.4 Hz, 3H). LCMS: (ES⁺) m/z=570.1 (M+H).

(lxx) Compound 55:

A mixture of 1-[8-(2-chlorophenyl)-9-(4-chlorophenyl)-2-methylsulfonyl-purin-6-yl]-4-methyl-piperidine-4-carboxamide (Compound 68) (100 mg, 179 μmol, 1 eq.) and (2S)-1-(methylamino)propan-2-ol (159 mg, 1.79 mmol, 10 eq.) was stirred at 140° C. for 5 hours. The reaction mixture was filtered and concentrated under reduced pressure to give a residue. The residue was purified by prep-HPLC (column: Waters Xbridge 150*25 mm*5 μm; mobile phase: [water(ammonium bicarbonate)-ACN]; B %: 52%-82%, 8 min) to give 1-[8-(2-chlorophenyl)-9-(4-chlorophenyl)-2-[[(2S)-2-hydroxypropyl]-methyl-amino]purin-6-yl]-4-methyl-piperidine-4-carboxamide (Compound 55, 47.59 mg, 83.1 μmol, 46.5% yield) as a white solid. ¹H NMR (400 MHz, MeOD-d₄) δ=7.61-7.53 (m, 1H), 7.48-7.30 (m, 5H), 7.28-7.19 (m, 2H), 4.75-4.57 (m, 2H), 4.11-4.07 (m, 1H), 3.76 (t, J=10.4 Hz, 2H), 3.67-3.63 (m, 1H), 3.46-3.41 (m, 1H), 3.17 (s, 3H), 2.16 (d, J=14.4 Hz, 2H), 1.59-1.53 (m, 2H), 1.27 (s, 3H), 1.14 (d, J=6.4 Hz, 3H). LCMS: (ES⁺) m/z=568.1 (M+H).

(lxxi) Compound 56:

1-[8-(2-chlorophenyl)-9-(4-chlorophenyl)-2-methylsulfonyl-purin-6-yl]-4-methyl-piperidine-4-carboxamide (Compound 68) (150 mg, 0.27 mmol, 1.0 eq.) and 2-aminoethanol (1.01 g, 16.5 mmol, 10 eq.) were stirred at 140° C. for 1 hour. The mixture was diluted with ethyl acetate (30 mL), washed with water (10 mL), brine (10 mL), dried over sodium sulfate and concentrated in vacuum and purified by rep-HPLC (column: Phenomenex Luna C18 150*25 mm*10 μm; mobile phase: [water(formic acid)-ACN]; B %: 29%-59%, 10 min) to give 1-[8-(2-chlorophenyl)-9-(4-chlorophenyl)-2-(2-hydroxyethylamino)purin-6-yl]-4-methyl-piperidine-4-carboxamide (Compound 56, 30.16 mg, 55.8 μmol, 20.8% yield) as a white solid. ¹H NMR (400 MHz, DMSO-d₆) δ=7.70 (d, J=8.8 Hz, 2H), 7.48-7.09 (m, 7H), 4.82-4.77 (m, 2H), 4.49-4.26 (m, 1H), 4.01-3.88 (m, 1H). 7.64 (d, J=7.2 Hz, 1H), 7.52-7.37 (m, 5H), 7.32-7.17 (m, 3H), 6.94 (s, 1H), 6.38 (t, J=5.6 Hz, 1H), 5.29-4.25 (m, 3H), 3.74-3.47 (m, 4H), 3.29-3.25 (m, 2H), 2.13-2.01 (m, 2H), 1.54-1.33 (m, 2H), 1.16 (s, 3H). LCMS: (ES⁺) m/z=540.3 (M+H).

(lxxii) Compound 57:

To a solution of 1-[6-(4-chloroanilino)-2-[2-hydroxyethyl(methyl)amino]-5-nitro-pyrimidin-4-yl]-4-methyl-piperidine-4-carboxamide (Intermediate B-5(48), Example 4ii) (100 mg, 216 μmol, 1 eq.) and 1,4-dimethylimidazole-2-carbaldehyde (134 mg, 1.08 mmol, 5 eq.) in ethanol (2 mL) and acetic acid (0.8 mL) was added iron (120 mg, 2.16 mmol, 10 eq.). The mixture was stirred at 80° C. for 1 hour. The reaction mixture was filtered and concentrated under vacuum. The residue was purified by flash silica gel chromatography (ISCO®; 20 g SepaFlash® Silica Flash Column, Eluent of 0˜10% Methanol/Dichloromethane @80 mL/min) to give a yellow solid (Rf=0.4). The yellow solid was purified by prep-HPLC (column: Phenomenex Luna C18 150*25 mm*10 μm; mobile phase: [water(formic acid)-ACN]; B %: 16%-46%, 10 min) to give a yellow solid, which was further separated by SFC (column: REGIS(S,S)WHELK-01 (250 mm*25 mm, 10 μm); mobile phase: [0.1% ammonia hydroxide methanol]; B %: 60%-60%, 3.4 min) to give 1-[9-(4-chlorophenyl)-8-(1,4-dimethylimidazol-2-yl)-2-[2-hydroxyethyl(methyl)amino]purin-6-yl]-4-methyl-piperidine-4-carboxamide (Compound 57, 17.33 mg, 31.6 μmol, 14.6% yield) as a yellow solid and 1-(9-(4-chlorophenyl)-8-(1,5-dimethyl-1H-imidazol-2-yl)-2-((2-hydroxyethyl)(methyl)amino)-9H-purin-6-yl)-4-methylpiperidine-4-carboxamide (Compound 115, 20.29 mg, 36.8 μmol, 17.0% yield) as a yellow solid. ¹H NMR (400 MHz, CDCl₃-d) δ=7.28 (d, J=8.6 Hz, 2H), 7.21-7.09 (m, 3H), 6.51 (s, 1H), 5.76-5.16 (m, 2H), 4.63-4.36 (m, 2H), 3.96-3.74 (m, 2H), 3.72-3.65 (m, 2H), 3.62-3.59 (m, 3H), 3.59-3.55 (m, 2H), 3.05 (s, 3H), 2.06-1.99 (m, 2H), 1.99-1.92 (m, 3H), 1.52-1.47 (m, 2H), 1.20 (s, 3H). LCMS: (ES⁺) m/z=538.4 (M+H).

(xxiii) Compound 58:

To a solution of 1-[6-(4-chloroanilino)-2-[2-hydroxyethyl(methyl)amino]-5-nitro-pyrimidin-4-yl]-4-methyl-piperidine-4-carboxamide (Intermediate B-5(48), Example 4ii) (50 mg, 108 μmol, 1 eq.) and 3-methylimidazole-4-carbaldehyde (59.34 mg, 539 μmol, 5 eq) in ethanol (1 mL) and acetic acid (0.4 mL) was added iron (60 mg, 1.08 mmol, 10 eq.). The mixture was stirred at 80° C. for 1 hour. The reaction mixture was filtered and concentrated in vacuum. The residue was purified by prep-HPLC (column: Unisil 3-100 C18 Ultra 150*50 mm*3 μm; mobile phase: [water(formic acid)-ACN]; B %: 10%-40%, 10 min) to give 1-[9-(4-chlorophenyl)-2-[2-hydroxyethyl(methyl)amino]-8-(3-methylimidazol-4-yl)purin-6-yl]-4-methyl-piperidine-4-carboxamide (Compound 58, 24.60 mg, 46.9 μmol, 43.5% yield) as a brown solid. ¹H NMR (400 MHz, DMSO-d₆) δ=7.74 (s, 1H), 7.61 (d, J=8.4 Hz, 2H), 7.43 (d, J=8.6 Hz, 2H), 7.25 (br s, 1H), 6.94 (br s, 1H), 6.27 (s, 1H), 4.58 (br s, 1H), 3.86 (s, 3H), 3.52 (br s, 4H), 3.44-3.39 (m, 4H), 3.01 (s, 3H), 2.08 (br d, J=12.5 Hz, 2H), 1.50-1.31 (m, 2H), 1.15 (s, 3H). LCMS: (ES⁺) m/z=524.3 (M+H).

(lxxiv) Compound 59:

To a solution of 1-[6-(4-chloroanilino)-2-[2-hydroxyethyl(methyl)amino]-5-nitro-pyrimidin-4-yl]-4-methyl-piperidine-4-carboxamide (Intermediate B-5(48), Example 4ii) (50 mg, 108 μmol, 1 eq.) and 3-isopropylimidazole-4-carbaldehyde (74 mg, 539 μmol, 5 eq.) in ethanol (1 mL) and acetic acid (0.4 mL) was added iron (60 mg, 1.08 mmol, 10 eq.). The mixture was stirred at 80° C. for 1 hour. The reaction mixture was filtered and concentrated in vacuum. The residue was purified by prep-HPLC (column: Waters Xbridge 150*25 mm*5 μm; mobile phase: [water (ammonia hydroxide v/v)-ACN]; B %: 28%-56%, 9 min) to give 1-[9-(4-chlorophenyl)-2-[2-hydroxyethyl(methyl)amino]-8-(3-isopropylimidazol-4-yl)purin-6-yl]-4-methyl-piperidine-4-carboxamide (Compound 59, 19.67 mg, 35.5 μmol, 32.9% yield) as a yellow solid. ¹H NMR (400 MHz, CDCl₃-d) δ=7.68 (s, 1H), 7.47 (d, J=8.6 Hz, 2H), 7.22 (d, J=8.6 Hz, 2H), 6.49 (s, 1H), 5.84-5.37 (m, 2H), 5.24 (td, J=6.8, 13.5 Hz, 1H), 4.79-4.50 (m, 2H), 4.15-3.84 (m, 2H), 3.83-3.75 (m, 2H), 3.71-3.61 (m, 2H), 3.16 (s, 3H), 2.14 (br d, J=14.6 Hz, 2H), 1.66-1.59 (m, 2H), 1.53 (d, J=6.8 Hz, 6H), 1.32 (s, 3H). LCMS: (ES⁺) m/z=552.5 (M+H).

(lxxv) Compound 60:

A mixture of 1-[8-(2-chlorophenyl)-9-(4-chlorophenyl)-2-methylsulfonyl-purin-6-yl]-4-methyl-piperidine-4-carboxamide (Compound 68) (100 mg, 0.18 mmol, 1 eq.), tert-butyl N-(2-hydroxyethyl)carbamate (144 mg, 0.89 mmol, 5 eq.) and potassium carbonate (50 mg, 0.36 mmol, 2 eq.) in N,N-dimethylformamide (1 mL) was stirred at 100° C. for 12 hours. The reaction mixture was filtered and concentrated under reduced pressure to give a residue. The residue was dissolved in acetonitrile (2 mL) and purified by prep-HPLC (column: Waters Xbridge 150*25 mm*5 μm; mobile phase: [water (ammonium bicarbonate)-ACN]; B %: 54%-84%, 8 min) and the organic phase was concentrated under reduced pressure to remove acetonitrile, and the liquid was lyophilized to give tert-butyl N-[2-[6-(4-carbamoyl-4-methyl-1-piperidyl)-8-(2-chlorophenyl)-9-(4-chlorophenyl)purin-2-yl]oxyethyl]carbamate (Compound 60, 54.06 mg, 84.4 μmol, 47.2% yield) as an off-white solid. ¹H NMR (400 MHz, CDCl₃-d) δ=7.49 (d, J=7.2 Hz, 1H), 7.41-7.28 (m, 5H), 7.21-7.13 (m, 2H), 5.82-5.32 (m, 2H), 5.27-5.04 (m, 1H), 5.01-4.48 (m, 2H), 4.37 (t, J=5.2 Hz, 2H), 4.22-3.76 (m, 2H), 3.52 (d, J=4.4 Hz, 2H), 2.21-2.06 (m, 2H), 1.43 (s, 9H), 1.33 (s, 3H). LCMS: (ES⁺) m/z=640.2 (M+H).

(lxxvi) Compound 61:

Step 1: A mixture of 1-[8-(2-chlorophenyl)-9-(4-chlorophenyl)-2-methylsulfonyl-purin-6-yl]-4-methyl-piperidine-4-carboxamide (Compound 68) (100 mg, 0.18 mmol, 1 eq.), tert-butyl N-(2-hydroxyethyl)carbamate (144 mg, 0.89 mmol, 5 eq.) and potassium carbonate (50 mg, 0.36 mmol, 2 eq.) in N,N-dimethylformamide (1 mL) was stirred at 100° C. for 12 hours. The reaction mixture was filtered and concentrated under reduced pressure to give a residue. The residue was dissolved in acetonitrile (2 mL) and purified by prep-HPLC (column: Waters Xbridge 150*25 mm*5 μm; mobile phase: [water (ammonium bicarbonate)-ACN]; B %: 54%-84%, 8 min) and the organic phase was concentrated under reduced pressure to remove acetonitrile, and the liquid was lyophilized to give tert-butyl N-[2-[6-(4-carbamoyl-4-methyl-1-piperidyl)-8-(2-chlorophenyl)-9-(4-chlorophenyl)purin-2-yl]oxyethyl]carbamate (100 mg, 87.2 μmol) as a white solid. LCMS: (ES⁺) m/z=630.4 (M+H).

Step 2: To a solution of tert-butyl N-[2-[6-(4-carbamoyl-4-methyl-1-piperidyl)-8-(2-chlorophenyl)-9-(4-chlorophenyl)purin-2-yl]oxyethyl]carbamate (90 mg, 0.14 mmol, 1 eq.) in dioxane (1 mL) was added hydrochloric acid/dioxane (4 M, 0.04 mL, 1 eq.). The mixture was stirred at 25° C. for 1 hour. The reaction mixture was concentrated under reduced pressure to give 1-[2-(2-aminoethoxy)-8-(2-chlorophenyl)-9-(4-chlorophenyl)purin-6-yl]-4-methyl-piperidine-4-carboxamide (90 mg, crude) as a yellow solid.

Step 3: A mixture of 1-[2-(2-aminoethoxy)-8-(2-chlorophenyl)-9-(4-chlorophenyl)purin-6-yl]-4-methyl-piperidine-4-carboxamide (90 mg, 0.16 mmol, 1 eq.), triethylamine (24 mg, 0.23 mmol, 0.03 mL, 1.5 eq.) in dichloromethane (1 mL) was stirred at 0° C., then methanesulfonyl chloride (145 mg, 1.27 mmol, 0.1 mL, 8.11 eq.) was added to the mixture at 0° C., the mixture was stirred at 25° C. for 2 hours. The reaction mixture was washed with water (10 mL) and extracted with ethyl acetate 30 mL (10 mL×3). The combined organic layers were washed with brine 20 mL (10 mL×2), dried over anhydrous sodium sulfate, filtered and concentrated under reduced pressure to give a residue. The residue was dissolved in acetonitrile (2 mL) and purified by prep-HPLC (column: Waters Xbridge 150*25 mm*5 μm; mobile phase: [water (ammonium bicarbonate)-acetonitrile]; B %: 41%-71%, 8 min) and the organic phase was concentrated under reduced pressure to remove acetonitrile, and the liquid was lyophilized to give 1-[8-(2-chlorophenyl)-9-(4-chlorophenyl)-2-[2-(methanesulfonamido)ethoxy]purin-6-yl]-4-methyl-piperidine-4-carboxamide (Compound 61, 38.54 mg, 62.3 μmol, 40.0% yield) as a white solid. ¹H NMR (400 MHz, MeOD-d₄) δ=7.62-7.55 (m, 1H), 7.51-7.34 (m, 5H), 7.32-7.21 (m, 2H), 4.85-4.80 (m, 2H), 4.40 (t, J=5.6 Hz, 2H), 4.07-3.57 (m, 2H), 3.44 (t, J=5.6 Hz, 2H), 2.96 (s, 3H), 2.20 (d, J=14.4 Hz, 2H), 1.62-1.55 (m, 2H), 1.28 (s, 3H). LCMS: (ES⁺) m/z=618.2 (M+H).

(lxxvii) Compound 62:

A mixture of 8-(2-chlorophenyl)-9-(4-chlorophenyl)-2-methylsulfonyl-6-[4-(trifluoromethyl)-1-piperidyl]purine (Intermediate D-3) (50 mg, 87.7 μmol, 1 eq.), 2-(benzylamino)ethanol (535 mg, 3.54 mmol, 0.5 mL, 40 eq.) and potassium carbonate (61 mg, 438 μmol, 5 eq.) was stirred at 100° C. for 12 hours. The reaction mixture was cooled at room temperature and diluted with N,N-dimethylformamide (2 mL). The resulting mixture was filtered to afford the filter liquor. The filter liquor was purified by Prep-HPLC (column: Waters Xbridge 150*25 mm*5 μm; mobile phase: [water(ammonium bicarbonate)-ACN]; B %: 67%-97%, 9 min). The cut fraction was concentrated under reduced pressure to remove acetonitrile. The residue was lyophilized to give N-benzyl-2-[8-(2-chlorophenyl)-9-(4-chlorophenyl)-6-[4-(trifluoromethyl)-1-piperidyl]purin-2-yl]oxy-ethanamine (Compound 62, 21 mg, 32.7 μmol, 37.3% yield) as a white solid. ¹H NMR (400 MHz, MeOD-d₄) δ=7.61 (d, J=7.6 Hz, 1H), 7.50-7.37 (m, 5H), 7.36-7.22 (m, 7H), 5.75-5.42 (m, 2H), 4.44 (t, J=5.2 Hz, 2H), 3.81 (s, 2H), 3.22-3.10 (m, 2H), 2.96 (t, J=5.2 Hz, 2H), 2.68-2.54 (m, 1H), 2.03 (d, J=12.4 Hz, 2H), 1.70-1.55 (m, 2H). LCMS: (ES⁺) m/z=641.2(M+H).

(lxxviii) Compound 63:

To a solution of 1-[6-(4-chloroanilino)-2-[2-hydroxyethyl(methyl)amino]-5-nitro-pyrimidin-4-yl]-4-methyl-piperidine-4-carboxamide (Intermediate B-5(48), Example 4ii) (50 mg, 107.78 μmol, 1 eq.) and pyridine-2-carbaldehyde (57 mg, 539 μmol, 5 eq.) in ethanol (1 mL) was added acetic acid (0.4 mL) and iron (30 mg, 538.90 μmol, 5 eq.). The mixture was stirred at 80° C. for 1 hour. The reaction mixture was filtered and concentrated in vacuum. The residue was purified by prep-H PLC (column: Waters Xbridge 150*25 mm*5 μm; mobile phase: [water(ammonium bicarbonate)-ACN]; B %: 40%-70%, 8 min). to give 1-[9-(4-chlorophenyl)-2-[2-hydroxyethyl(methyl)amino]-8-(2-pyridyl)purin-6-yl]-4-methyl-piperidine-4-carboxamide (Compound 63, 25.87 mg, 48.7 μmol, 45.2% yield) was obtained as a pink solid. ¹H NMR (400 MHz, CDCl₃-d) δ=8.35 (d, J=4.2 Hz, 1H), 7.94 (d, J=7.9 Hz, 1H), 7.70 (br d, J=1.7 Hz, 1H), 7.46-7.35 (m, 2H), 7.26-7.20 (m, 2H), 7.19-7.14 (m, 1H), 5.78-5.20 (m, 2H), 4.92-4.35 (m, 3H), 4.16-3.88 (m, 2H), 3.86-3.75 (m, 2H), 3.72-3.63 (m, 2H), 3.17 (s, 3H), 2.26-2.05 (m, 2H), 1.66 (br s, 2H), 1.33 (s, 3H). LCMS: (ES⁺) m/z=521.4 (M+H).

(lxxix) Compound 64:

To a solution of 1-[6-(4-chloroanilino)-2-[2-hydroxyethyl(methyl)amino]-5-nitro-pyrimidin-4-yl]-4-methyl-piperidine-4-carboxamide (Intermediate B-5(48), Example 4ii) (50 mg, 108 μmol, 1 eq.) and 6-formylpyridine-3-carbonitrile (71 mg, 539 μmol, 5 eq.) in ethanol (1 mL) was added acetic acid (0.4 mL) and iron (30 mg, 539 μmol, 5 eq.). The mixture was stirred at 80° C. for 1 hour. The reaction mixture was filtered and concentrated in vacuum. The residue was purified by prep-H PLC (column: Waters Xbridge 150*25 mm*5 μm; mobile phase: [water(ammonium bicarbonate)-ACN]; B %: 44%-74%, 8 min) to give a yellow solid. The yellow solid was purified by prep-HPLC (column: Unisil 3-100 C18 Ultra 150*50 mm*3 μm; mobile phase: [water(formic acid)-ACN]; B %: 38%-68%, 10 min) to give 1-[9-(4-chlorophenyl)-8-(5-cyano-2-pyridyl)-2-[2-hydroxyethyl(methyl)amino]purin-6-yl]-4-methyl-piperidine-4-carboxamide (Compound 64, 14.34 mg, 3.77 μmol, 3.4% yield, 14.3% purity) as a yellow solid. ¹H NMR (400 MHz, CDCl₃-d) δ=8.50 (s, 1H), 8.24 (d, J=8.4 Hz, 1H), 7.94 (br d, J=1.5 Hz, 1H), 7.44 (br d, J=8.4 Hz, 2H), 7.21 (br d, J=8.6 Hz, 2H), 5.88-5.19 (m, 2H), 5.13-4.40 (m, 2H), 4.21-3.94 (m, 1H), 3.80 (br s, 2H), 3.67 (br s, 2H), 2.32-2.10 (m, 2H), 1.65 (br d, J=13.4 Hz, 4H), 1.34 (s, 3H). LCMS: (ES⁺) m/z=546.4 (M+H).

(lxxx) Compound 65:

To a solution of 1-[6-(4-chloroanilino)-2-[2-hydroxyethyl(methyl)amino]-5-nitro-pyrimidin-4-yl]-4-methyl-piperidine-4-carboxamide (Intermediate B-5(48), Example 4ii) (50 mg, 108 μmol, 1 eq.) and 5-formylpyridine-2-carbonitrile (71 mg, 539 μmol, 5 eq.) in ethanol (1 mL) was added acetic acid (0.4 mL) and iron (30 mg, 539 μmol, 5 eq.). The mixture was stirred at 80° C. for 1 hour. The reaction mixture was filtered and concentrated in vacuum. The residue was purified by prep-H PLC (column: Waters Xbridge 150*25 mm*5 μm; mobile phase: [water (ammonia hydroxide v/v)-acetonitrile]; B %: 33%-63%, 9 min) and prep-HPLC (column: Unisil 3-100 C18 Ultra 150*50 mm*3 μm; mobile phase: [water(formic acid)-acetonitrile]; B %: 37%-67%, 10 min) to give 1-[9-(4-chlorophenyl)-8-(6-cyano-3-pyridyl)-2-[2-hydroxyethyl(methyl)amino]purin-6-yl]-4-methyl-piperidine-4-carboxamide (Compound 65, 11.52 mg, 20.6 μmol, 19.1% yield) as a yellow solid. ¹H NMR (400 MHz, CDCl₃-d) δ=8.83 (d, J=2.0 Hz, 1H), 7.80 (m, J=2.2, 8.2 Hz, 1H), 7.58 (d, J=8.3 Hz, 1H), 7.53-7.48 (m, 2H), 7.27-7.22 (m, 2H), 5.83-5.25 (m, 2H), 4.40-3.90 (m, 2H), 3.83-3.77 (m, 2H), 3.73-3.64 (m, 2H), 3.17 (s, 3H), 2.24-2.10 (m, 2H), 1.75-1.52 (m, 4H), 1.34 (s, 3H). LCMS: (ES⁺) m/z=546.3 (M+H).

(lxxxi) Compound 66:

A mixture of 1-[8-(2-chlorophenyl)-9-(4-chlorophenyl)-2-methylsulfonyl-purin-6-yl]-4-methyl-piperidine-4-carboxamide (Compound 68) (100 mg, 0.18 mmol, 1 eq.) and (2S)-2-(methylamino) propan-1-ol (159 mg, 1.79 mmol, 10 eq.) was stirred at 140° C. for 5 hours. The reaction mixture was filtered and concentrated under reduced pressure to give a residue. The residue was purified by prep-HPLC (column: Waters Xbridge 150*25 mm*5 μm; mobile phase: [water (ammonium bicarbonate)-acetonitrile]; B %: 51%-81%, 8 min) to give 1-[8-(2-chlorophenyl)-9-(4-chlorophenyl)-2-[[(1S)-2-hydroxy-1-methyl-ethyl]-methyl-amino]purin-6-yl]-4-methyl-piperidine-4-carboxamide (Compound 66, 38.06 mg, 64.94 μmol, 36.3% yield) as an off-white solid. ¹H NMR (400 MHz, DMSO-d₆) δ=7.65-7.63 (m, 1H), 7.49-7.41 (m, 5H), 7.31-7.19 (m, 3H), 6.93 (s, 1H), 4.78-4.50 (m, 3H), 3.71-3.54 (m, 1H), 3.51-3.45 (m, 1H), 3.44-3.38 (m, 1H), 3.32-3.18 (m, 2H), 2.89 (s, 3H), 2.10-2.04 (m, 2H), 1.44-1.36 (m, 2H), 1.15 (s, 3H), 1.05 (d, J=6.8 Hz, 3H). LCMS: (ES⁺) m/z=568.2 (M+H).

(lxxxii) Compound 67:

A mixture of 1-[8-(2-chlorophenyl)-9-(4-chlorophenyl)-2-methylsulfonyl-purin-6-yl]-4-methyl-piperidine-4-carboxamide (Compound 68) (100 mg, 0.18 mmol, 1 eq.) and (2R)-2-(methylamino) propan-1-ol (159 mg, 1.79 mmol, 10 eq.) was stirred at 140° C. for 5 hours. The reaction mixture was filtered and concentrated under reduced pressure to give a residue. The residue was purified by prep-HPLC (column: Waters Xbridge 150*25 mm*5 μm; mobile phase: [water (ammonium bicarbonate)-acetonitrile]; B %: 48%-78%, 9 min) to give 1-[8-(2-chlorophenyl)-9-(4-chlorophenyl)-2-[[(1R)-2-hydroxy-1-methyl-ethyl]-methyl-amino]purin-6-yl]-4-methyl-piperidine-4-carboxamide (Compound 67, 31.97 mg, 54.5 μmol, 30.5% yield) as a white solid. ¹H NMR (400 MHz, DMSO-d₆) δ=7.65-7.63 (m, 1H), 7.54-7.36 (m, 5H), 7.31-7.21 (m, 3H), 6.93 (s, 1H), 4.81-4.49 (m, 3H), 3.78-3.56 (m, 1H), 3.52-3.44 (m, 1H), 3.44-3.38 (m, 1H), 3.31 (s, 2H), 2.89 (s, 3H), 2.13-2.03 (m, 2H), 1.46-1.34 (m, 2H), 1.15 (s, 3H), 1.05 (d, J=6.8 Hz, 3H). LCMS: (ES⁺) m/z=568.2 (M+H).

(lxxxiii) Compound 68:

To solution of 1-[8-(2-chlorophenyl)-9-(4-chlorophenyl)-2-methylsulfanyl-purin-6-yl]-4-methyl-piperidine-4-carboxamide (Intermediate C-1, Example 5i) (0.1 g, 0.19 mmol, 1 eq.) in N-methyl-2-pyrrolidone (1 mL) was added a solution of potassium peroxymonosulfate (0.19 g, 1.13 mmol, 6 eq.) in water (0.15 mL), the mixture was stirred at 60° C. for 12 hours. The reaction mixture was added to water (10 mL), filtered and the residue was purified by prep-HPLC (column: Waters Xbridge 150*25 mm*5 μm; mobile phase: [water (ammonium bicarbonate)-acetonitrile]; B %: 43%-73%, 8 min) to give 1-[8-(2-chlorophenyl)-9-(4-chlorophenyl)-2-methylsulfonyl-purin-6-yl]-4-methyl-piperidine-4-carboxamide (Compound 68, 55.96 mg, 100 μmol, 55.9% yield) as a white solid. ¹H NMR (400 MHz, DMSO-d₆) δ=7.73-7.71 (m, 1H), 7.61-7.51 (m, 4H), 7.50-7.45 (m, 1H), 7.41-7.35 (m, 2H), 7.31 (s, 1H), 7.01 (s, 1H), 5.21-4.98 (m, 1H), 4.50-4.27 (m, 1H), 4.13-3.92 (m, 1H), 3.65-3.47 (m, 1H), 3.30 (s, 3H), 2.15 (d, J=13.8 Hz, 2H), 1.49 (s, 2H), 1.18 (s, 3H). LCMS: (ES⁺) m/z=559.1 (M+H).

(lxxxiv) Compound 69:

A mixture of ethyl 1H-imidazole-5-carboxylate (400.8 mg, 2.86 mmol, 8.0 eq.) and sodium hydride (71.5 mg, 1.79 mmol, 60% purity, 5.0 eq.) in N,N-dimethylformamide (3 mL) was stirred at 25° C. for 30 min, then 1-[8-(2-chlorophenyl)-9-(4-chlorophenyl)-2-methylsulfonyl-purin-6-yl]-4-methyl-piperidine-4-carboxamide (Compound 68) (200 mg, 357.48 μmol, 1 eq.) was added to the mixture and the mixture was stirred at 60° C. for 5.5 hours. The reaction mixture was diluted with water (30 mL) and extracted with ethyl acetate (20 mL*3). The combined organic layers were washed with brine (30 mL*2), dried over sodium sulfate, filtered and concentrated under reduced pressure. The resulting residue was purified by prep-HPLC (column: Waters Xbridge 150*25 mm*5 μm; mobile phase: [water(ammonium bicarbonate)-ACN]; B %: 53%-83%, 8 min). The cut fraction was concentrated under reduced pressure to remove acetonitrile. The residue was lyophilized to give compound ethyl 1-[6-(4-carbamoyl-4-methyl-1-piperidyl)-8-(2-chlorophenyl)-9-(4-chlorophenyl)purin-2-yl]imidazole-4-carboxylate (Compound 69, 38 mg, 58.3 μmol, 16.3% yield) as a white solid. ¹H NMR (400 MHz, CDCl₃-d) δ=8.48 (s, 2H), 7.53 (m, 1H), 7.46-7.35 (m, 5H), 7.25-7.18 (m, 2H), 5.77-5.40 (m, 2H), 5.20-3.74 (m, 6H), 2.28-2.19 (m, 2H), 1.77-1.68 (m, 2H), 1.44 (t, J=7.2 Hz, 3H), 1.37 (s, 3H). LCMS: (ES⁺) m/z=619.2 (M+H).

(lxxxv) Compound 70:

A mixture of 1-[8-(2-chlorophenyl)-9-(4-chlorophenyl)-2-methylsulfonyl-purin-6-yl]-4-methyl-piperidine-4-carboxamide (Compound 68) (100 mg, 0.18 mmol, 1 eq.) and N-alpha-methyl-1-alanine hydrochloride (111 mg, 0.89 mmol, 5 eq.) and cesium carbonate (291 mg, 0.89 mmol, 5 eq.) in N-methyl-2-pyrrolidone (1 mL) was stirred at 140° C. for 5 hours. The reaction mixture was filtered and concentrated under reduced pressure to give a residue. The residue was purified by prep-HPLC (column: Phenomenex luna C18 150*25 mm*10 μm; mobile phase: [water (formic acid)-ACN]; B %: 46%-76%, 10 min) to give 1-[2-[(2-amino-2-oxo-ethyl)-methyl-amino]-8-(2-chlorophenyl)-9-(4-chlorophenyl)purin-6-yl]-4-methyl-piperidine-4-carboxamide (Compound 70, 13.46 mg, 23.7 μmol, 13.2% yield) as a white solid. ¹H NMR (400 MHz, DMSO-d₆) δ=7.70-7.62 (m, 1H), 7.55-7.36 (m, 5H), 7.34-7.17 (m, 3H), 6.93 (s, 1H), 5.22-3.38 (m, 4H), 3.28-3.19 (m, 2H), 3.08 (s, 3H), 2.08-2.04 (m, 2H), 1.45-1.33 (m, 2H), 1.14 (s, 3H). LCMS: (ES⁺) m/z=570.0 (M+H).

(lxxxvi) Compound 71:

To a solution of 1-[8-(2-chlorophenyl)-9-(4-chlorophenyl)-2-methylsulfonyl-purin-6-yl]-4-methyl-piperidine-4-carboxamide (Compound 68) (100 mg, 179 μmol, 1 eq.) in 1-dimethylphosphoryl-N-methyl-methanamine (325 mg, 2.68 mmol, 15 eq.) was stirred at 140° C. for 1 hour. The reaction mixture was concentrated under vacuum. The residue was purified by prep-HPLC (column: Waters Xbridge 150*25 mm*5 μm; mobile phase: [water(ammonium bicarbonate)-ACN]; B %: 39%-69%, 8 min) to give a white solid. The white solid was purified by prep-HPLC (column: Phenomenex luna C18 150*25 mm*10 μm; mobile phase: [water(formic acid)-ACN]; B %: 53%-73%, 10 min) to give the compound 1-[8-(2-chlorophenyl)-9-(4-chlorophenyl)-2-[dimethylphosphorylmethyl(methyl)amino]purin-6-yl]-4-methyl-piperidine-4-carboxamide (Compound 71, 10.13 mg, 16.9 μmol, 9.4% yield) as a white solid. ¹H NMR (400 MHz, CDCl₃-d) δ=7.53-7.44 (m, 1H), 7.38-7.26 (m, 5H), 7.15 (d, J=8.8 Hz, 2H), 5.82-5.13 (m, 2H), 4.94-4.28 (m, 2H), 4.23-3.77 (m, 4H), 3.26 (s, 3H), 2.23-2.02 (m, 2H), 1.68-1.61 (m, 2H), 1.44 (br d, J=12.6 Hz, 6H), 1.31 (s, 3H). LCMS: (ES⁺) m/z=600.2 (M+H).

(lxxxvii) Compound 78:

A mixture of 1-[8-(2-chlorophenyl)-9-(4-chlorophenyl)-2-methylsulfonyl-purin-6-yl]-4-methyl-piperidine-4-carboxamide (Compound 68) (80 mg, 143 μmol, 1 eq.), ethylene glycol (44 mg, 715 μmol, 5 eq.) and potassium carbonate (40 mg, 286 μmol, 2 eq.) in N,N-dimethylformamide (0.5 mL) was stirred at 100° C. for 8 hours. The reaction mixture was cooled at room temperature and diluted with N,N-dimethylformamide (2 mL). The resulting mixture was filtered to afford the filter liquor. The filter liquor was purified by reversed-phase HPLC (column: Waters Xbridge 150*25 mm*5 μm; mobile phase: [water(ammonium bicarbonate)-ACN]; B %: 39%-69%, 8 min). The cut fraction was concentrated under reduced pressure to remove acetonitrile. The residue was lyophilized to give compound 1-[8-(2-chlorophenyl)-9-(4-chlorophenyl)-2-(2-hydroxyethoxy)purin-6-yl]-4-methyl-piperidine-4-carboxamide (Compound 78, 35.4 mg, 63.4 μmol, 44.3% yield) as a white solid. ¹H NMR (400 MHz, DMSO-d₆) δ=7.71-7.67 (m, 1H), 7.54-7.43 (m, 5H), 7.34-7.24 (m, 3H), 6.96 (s, 1H), 5.18-4.85 (m, 1H), 4.81 (t, J=5.6 Hz, 1H), 4.61-4.24 (m, 1H), 4.20 (t, J=5.2 Hz, 2H), 4.04-3.74 (m, 1H), 3.70-3.64 (m, 2H), 3.57-3.38 (m, 1H), 2.14-2.07 (m, 2H), 1.49-1.37 (m, 2H), 1.17 (s, 3H). LCMS: (ES⁺) m/z=542.9 (M+H).

(lxxxviii) Compound 79:

A mixture of 1-[8-(2-chlorophenyl)-9-(4-chlorophenyl)-2-methylsulfonyl-purin-6-yl]-4-methyl-piperidine-4-carboxamide (Compound 68) (280 mg, 500 μmol, 1 eq.), (2R)-propane-1,2-diol (190 mg, 2.5 mmol, 5 eq.) and potassium carbonate (138 mg, 1.0 mmol, 2 eq.) in N,N-dimethylformamide (3 mL) was stirred at 100° C. for 7 hours. The reaction mixture was diluted with N,N-dimethylformamide(1 mL) and filtered to afford the filter liquor. The filter liquor was purified by prep-HPLC (column: Waters Xbridge 150*25 mm*5 um; mobile phase: [water(ammonium bicarbonate)-ACN]; B %: 39%-69%, 10 min) to give desired compound (100 mg) as a white solid, which was further separated by SFC (column: DAICEL CHIRALPAK AD (250 mm*30 mm*10 μm); mobile phase: [0.1% ammonia hydroxide IPA]; B %: 35%-35%, 3.25 min). The resulting solution was concentrated under reduced pressure to remove isopropanol, the residue was lyophilized to give 1-[8-(2-chlorophenyl)-9-(4-chlorophenyl)-2-[(2R)-2-hydroxypropoxy]purin-6-yl]-4-methyl-piperidine-4-carboxamide (Compound 79, 55.25 mg, 99.5 μmol, 39.7% yield) as a white solid and 1-[8-(2-chlorophenyl)-9-(4-chlorophenyl)-2-[(1R)-2-hydroxy-1-methyl-ethoxy]purin-6-yl]-4-methyl-piperidine-4-carboxamide (Compound 81, 9.3 mg, 16.7 μmol, 6.6% yield) as a white solid. ¹H NMR (400 MHz, DMSO-d₆) δ=7.71-7.66 (m, 1H), 7.54-7.42 (m, 5H), 7.35-7.24 (m, 3H), 6.97 (s, 1H), 5.25-4.87 (m, 1H), 4.82 (d, J=4.8 Hz, 1H), 4.56-4.18 (m, 1H), 4.11-3.86 (m, 4H), 3.65-3.42 (m, 1H), 2.16-2.05 (m, 2H), 1.49-1.37 (m, 2H), 1.17 (s, 3H), 1.10 (d, J=6.0 Hz, 3H). LCMS: (ES⁺) m/z=555.2 (M+H).

(lxxxix) Compound 80:

A mixture of 1-[8-(2-chlorophenyl)-9-(4-chlorophenyl)-2-methylsulfonyl-purin-6-yl]-4-methyl-piperidine-4-carboxamide (Compound 68) (300 mg, 536 μmol, 1 eq.), (2S)-propane-1,2-diol (204.02 mg, 2.68 mmol, 5 eq.) and potassium carbonate (148.22 mg, 1.07 mmol, 2 eq.) in N,N-dimethylformamide (3 mL) was stirred at 100° C. for 7 hours. Then the reaction mixture was diluted with N, N-dimethylformamide (1 mL) and filtered to afford the filter liquor. The resulting filter liquor was purified by reversed-phase HPLC(column: Waters Xbridge 150*25 mm*5 μm; mobile phase: [water(ammonium bicarbonate)-ACN]; B %: 39%-69%, 10 min) to give desired compound as a white solid, which was further separated by SFC (column: DAICEL CHIRALPAK AD (250 mm*30 mm*10 μm); mobile phase: [0.1% ammonia hydroxide IPA]; B %: 45%-45%, 2.6 min) to give 1-[8-(2-chlorophenyl)-9-(4-chlorophenyl)-2-[(2S)-2-hydroxypropoxy]purin-6-yl]-4-methyl-pi-peridine-4-carboxamide (Compound 80, 22.92 mg, 40.8 μmol, 15.2% yield) as a white solid and 1-[8-(2-chlorophenyl)-9-(4-chlorophenyl)-2-[(1S)-2-hydroxy-1-methyl-ethoxy]purin-6-yl]-4-methyl-piperidine-4-carboxamide (Compound 82, 4.79 mg, 8.62 μmol, 3.2% yield) as a white solid. ¹H NMR (400 MHz, CDCl₃-d) δ=7.52-7.44 (m, 1H), 7.42-7.29 (m, 5H), 7.17 (d, J=8.8 Hz, 2H), 5.74-5.29 (m, 2H), 4.98-4.40 (m, 2H), 4.38-4.29 (m, 1H), 4.25-4.17 (m, 2H), 4.17-3.83 (m, 2H), 3.25 (s, 1H), 2.25-2.10 (m, 2H), 1.72-1.61 (m, 2H), 1.33 (s, 3H), 1.25 (d, J=6.0 Hz, 3H). LCMS: (ES⁺) m/z=555.2 (M+H).

### (xc) Compound 81:

Compound 81, 1-[8-(2-chlorophenyl)-9-(4-chlorophenyl)-2-[(1R)-2-hydroxy-1-methyl-ethoxy]purin-6-yl]-4-methyl-piperidine-4-carboxamide was obtained as a by-product following the procedure described for Compound 79. ¹H NMR (400 MHz, DMSO-d₆) δ=7.71-7.65 (m, 1H), 7.54-7.42 (m, 5H), 7.34-7.25 (m, 3H), 6.97 (s, 1H), 5.21-4.73 (m, 3H), 4.64-4.18 (m, 1H), 4.05-3.76 (m, 1H), 3.66-3.39 (m, 3H), 2.15-2.06 (m, 2H), 1.50-1.37 (m, 2H), 1.21 (d, J=6.4 Hz, 3H), 1.17 (s, 3H). LCMS: (ES⁺) m/z=556.2 (M+H).

(xci) Compound 82:

Compound 82, 1-[8-(2-chlorophenyl)-9-(4-chlorophenyl)-2-[(1S)-2-hydroxy-1-methyl-ethoxy]purin-6-yl]-4-methyl-piperidine-4-carboxamide was obtained as a by-product following the procedure described for Compound 80. ¹H NMR (400 MHz, CDCl₃-d) δ=7.48 (d, J=6.8 Hz, 1H), 7.40-7.28 (m, 5H), 7.16 (d, J=8.8 Hz, 2H), 5.66-5.33 (m, 2H), 5.24-5.14 (m, 1H), 5.04-3.84 (m, 4H), 3.83-3.70 (m, 2H), 3.10-3.04 (m, 1H), 2.22-2.11 (m, 2H), 1.70-1.61 (m, 2H), 1.35 (d, J=6.4 Hz, 3H), 1.33 (s, 3H). LCMS: (ES⁺) m/z=555.2 (M+H).

(xcii) Compound 83:

A mixture of 1-[8-(2-chlorophenyl)-9-(4-chlorophenyl)-2-methylsulfonyl-purin-6-yl]-4-methyl-piperidine-4-carboxamide (Compound 68) (70 mg, 125.12 μmol, 1 eq.) and (2R)-1-aminopropan-2-ol (477 mg, 6.35 mmol, 0.5 mL, 50.8 eq.) was stirred at 140° C. for 6 hours. The reaction mixture was cooled at room temperature and diluted with N,N-dimethylformamide (2 mL). The resulting mixture was filtered to afford the filter liquor. The filter liquor was purified by reversed-phase HPLC (column: Waters Xbridge 150*25 mm*5 μm; mobile phase: [water(ammonium bicarbonate)-ACN]; B %: 42%-72%, 10 min). The cut fraction was concentrated under reduced pressure to remove acetonitrile. The residue was lyophilized to give 1-[8-(2-chlorophenyl)-9-(4-chlorophenyl)-2-[[(2R)-2-hydroxypropyl]amino]purin-6-yl]-4-methyl-piperidine-4-carboxamide (Compound 83, 44 mg, 79.4 μmol, 63.4% yield) as a white solid. ¹H NMR (400 MHz, DMSO-d₆) δ=7.64 (d, J=6.8 Hz, 1H), 7.50-7.39 (m, 5H), 7.26 (d, J=8.8 Hz, 3H), 6.97 (s, 1H), 6.43-6.35 (m, 1H), 4.93-4.25 (m, 3H), 3.81-3.72 (m, 1H), 3.69-3.42 (m, 2H), 3.21-3.11 (m, 2H), 2.11-2.02 (m, 2H), 1.45-1.33 (m, 2H), 1.15 (s, 3H), 1.06-1.00 (m, 3H). LCMS: (ES⁺) m/z=554.2 (M+H).

(xciii) Compound 84:

To a solution of 1-[8-(2-chlorophenyl)-9-(4-chlorophenyl)-2-methylsulfonyl-purin-6-yl]-4-methyl-piperidine-4-carboxamide (80 mg, 143 μmol, 1 eq.) in (2S)-1-aminopropan-2-ol (161 mg, 2.14 mmol, 15 eq.) was stirred at 140° C. for 1 hour. The reaction mixture was concentrated under reduced pressure. The residue was purified by prep-HPLC (column: Unisil 3-100 C18 Ultra 150*50 mm*3 μm; mobile phase: [water(formic acid)-ACN]; B %: 31%-61%, 7 min) to give the compound 1-[8-(2-chlorophenyl)-9-(4-chlorophenyl)-2-[[(2S)-2-hydroxypropyl]amino]purin-6-yl]-4-methyl-piperidine-4-carboxamide (42.97 mg, 76.0 μmol, 53.1% yield) as an off-white solid. ¹H NMR (400 MHz, CDCl₃-d) δ=7.47 (d, J=7.2 Hz, 1H), 7.38-7.27 (m, 5H), 7.20-7.09 (m, 2H), 5.87-5.32 (m, 2H), 5.19 (br t, J=6.0 Hz, 1H), 4.84-4.35 (m, 2H), 3.98 (m, J=2.3, 6.4 Hz, 3H), 3.65-3.43 (m, 1H), 3.36-3.18 (m, 1H), 2.27-2.06 (m, 2H), 1.63 (m, J=3.5, 9.4, 13.4 Hz, 2H), 1.30 (s, 3H), 1.18 (d, J=6.4 Hz, 3H). LCMS: (ES⁺) m/z=554.3 (M+H).

(xciv) Compound 85:

A mixture of 1-[8-(2-chlorophenyl)-9-(4-chlorophenyl)-2-methylsulfonyl-purin-6-yl]-4-methyl-piperidine-4-carboxamide (Compound 68) (80 mg, 143 μmol, 1 eq.) and 1-amino-2-methyl-propan-2-ol (637 mg, 7.15 mmol, 50 eq.) was stirred at 140° C. for 6 hours, The reaction mixture was cooled at room temperature and diluted with N,N-dimethylformamide (2 mL). The resulting mixture was purified by reversed-phase HPLC (column: Waters Xbridge 150*25 mm*5 μm; mobile phase: [water(ammonium bicarbonate)-ACN]; B %: 43%-73%, 10 min). The cut fraction was concentrated under reduced pressure to remove acetonitrile. The residue was lyophilized to give 1-[8-(2-chlorophenyl)-9-(4-chlorophenyl)-2-[(2-hydroxy-2-methyl-propyl)amino]purin-6-yl]-4-methyl-piperidine-4-carboxamide (Compound 85, 40 mg, 69.7 μmol, 48.7% yield) as a white solid. ¹H NMR (400 MHz, DMSO-d6) δ=7.67-7.61 (m, 1H), 7.51-7.39 (m, 5H), 7.31-7.23 (m, 3H), 6.96 (s, 1H), 6.27 (t, J=6.0 Hz, 1H), 5.17-4.20 (m, 3H), 4.04-3.43 (m, 2H), 3.26-3.17 (m, 2H), 2.12-2.02 (m, 2H), 1.45-1.34 (m, 2H), 1.15 (s, 3H), 1.07 (s, 6H). LCMS: (ES⁺) m/z=568.2 (M+H).

(xcv) Compound 89:

Step 1: A mixture of 1-[8-(2-chlorophenyl)-9-(4-chlorophenyl)-2-methylsulfonyl-purin-6-yl]-4-methyl-piperidine-4-carboxamide (Compound 68) (300 mg, 535 μmol, 1 eq.) and (2,4-dimethoxyphenyl)methanamine (555 mg, 3.32 mmol, 6.20 eq.) was stirred at 140° C. for 1 hour. After cooling to room temperature, the reaction mixture was purified by flash silica gel chromatography (ISCO®; 25 SepaFlash® Silica Flash Column, Eluent of 10-100% Ethyl acetate/Petroleum ether gradient @50 mL/min) to give 1-[8-(2-chlorophenyl)-9-(4-chlorophenyl)-2-[(2,4-dimethoxyphenyl)methylamino]purin-6-yl]-4-methyl-piperidine-4-carboxamide (290 mg, 448 μmol, 83.8% yield) as a yellow solid. ¹H NMR (400 MHz, DMSO-d6) δ=7.64-7.59 (m, 1H), 7.50-7.37 (m, 5H), 7.30-7.18 (m, 3H), 7.13-7.06 (m, 1H), 6.96-6.90 (m, 1H), 6.77-6.68 (m, 1H), 6.53-6.48 (m, 1H), 6.44-6.37 (m, 1H), 5.01-4.39 (m, 2H), 4.36-4.24 (m, 2H), 3.77 (s, 3H), 3.71 (s, 3H), 3.68-3.41 (m, 2H), 2.08-2.00 (m, 2H), 1.43-1.26 (m, 2H), 1.12 (s, 3H)

Step 2: A mixture of 1-[8-(2-chlorophenyl)-9-(4-chlorophenyl)-2-[(2,4-dimethoxyphenyl)methylamino]purin-6-yl]-4-methyl-piperidine-4-carboxamide (290 mg, 448.52 μmol, 1 eq.) in trifluoroacetic acid (3 mL, 90 eq.) was stirred at 65° C. for 1 hour. The mixture was concentrated in vacuum to give a residue. The residue was purified by flash silica gel chromatography (ISCO®; 25 g SepaFlash® Silica Flash Column, Eluent of 0˜50% MeOH/DCM ether gradient @30 mL/min) to give 1-[2-amino-8-(2-chlorophenyl)-9-(4-chlorophenyl)purin-6-yl]-4-methyl-piperidine-4-carboxamide (180 mg, 362.62 μmol, 80.8% yield) as a yellow solid. ¹H NMR (400 MHz, DMSO-d6) δ=7.63 (d, J=7.1 Hz, 1H), 7.57-7.25 (m, 7H), 6.98 (br s, 1H), 3.86 (br s, 4H), 2.20-2.05 (m, 2H), 1.55-1.38 (m, 2H), 1.17 (s, 3H).

Step 3: To a solution of 1-[2-amino-8-(2-chlorophenyl)-9-(4-chlorophenyl)purin-6-yl]-4-methyl-piperidine-4-carboxamide (60 mg, 120.87 μmol, 1 eq.) in DCM (1 mL) was added triethylamine (24 mg, 241.75 μmol, 2 eq.) and 2-methylpropanoyl chloride (15 mg, 145.05 μmol, 1.2 eq.), the resulting mixture was stirred at 25° C. for 15 minutes. The mixture was concentrated in vacuum to give N-[8-(2-chlorophenyl)-9-(4-chlorophenyl)-6-(4-cyano-4-methyl-1-piperidyl)purin-2-yl]-2-methyl-propanamide (65 mg, crude) as a brown solid. The brown solid was purified by prep-HPLC (column: Waters Xbridge 150*25 mm*5 μm; mobile phase: [water (ammonia hydroxide v/v)-ACN]; B %: 51%-81%, 9 min) to give a white solid. The white solid was further purified by prep-HPLC (column: Unisil 3-100 C18 Ultra 150*50 mm*3 μm; mobile phase: [water(FA)-ACN]; B %: 51%-81%, 7 min) to give N-[8-(2-chlorophenyl)-9-(4-chlorophenyl)-6-(4-cyano-4-methyl-1-piperidyl)purin-2-yl]-2-methyl-propanamide (Compound 89, 13.71 mg, 25.00 μmol, 22.8% yield) as a yellow solid. ¹H NMR (400 MHz, CDCl₃) δ=7.70 (s, 1H), 7.51 (br d, J=6.8 Hz, 1H), 7.44-7.30 (m, 5H), 7.19 (d, J=8.4 Hz, 2H), 6.08-4.93 (m, 2H), 3.63-3.29 (m, 2H), 3.23-3.05 (m, 1H), 2.09 (m, 2H), 1.70-1.62 (m, 2H), 1.46 (s, 3H), 1.23 (d, J=6.7 Hz, 6H). LCMS: (ES⁺) m/z=548.4 (M+H).

(xcvi) Compound 90:

A mixture of 1-[8-(2-chlorophenyl)-9-(4-chlorophenyl)-2-methylsulfonyl-purin-6-yl]-4-methyl-piperidine-4-carboxamide (Compound 68) (80 mg, 143 μmol, 1 eq.) and 2-(ethylamino)ethanol (127 mg, 1.43 mmol, 0.14 mL, 10 eq.) was stirred at 140° C. for 5 hours. The residue was purified by prep-HPLC (column: Waters Xbridge 150*25 mm*5 μm; mobile phase: [water (ammonium bicarbonate)-ACN]; B %: 52%-82%, 8 min) and the organic phase was concentrated under reduced pressure to remove acetonitrile, and the liquid was lyophilized to give 1-[8-(2-chlorophenyl)-9-(4-chlorophenyl)-2-[ethyl(2-hydroxyethyl)amino]purin-6-yl]-4-methyl-piperidine-4-carboxamide (Compound 90, 18.46 mg, 32.5 mol, 22.7% yield) as a white solid. ¹H NMR (400 MHz, DMSO-d₆) δ=7.64 (d, J=7.2 Hz, 1H), 7.50-7.41 (m, 5H), 7.26 (d, J=8.8 Hz, 3H), 6.94 (s, 1H), 4.77-4.42 (m, 3H), 3.53 (d, J=5.6 Hz, 8H), 2.10-2.02 (m, 2H), 1.43-1.35 (m, 2H), 1.14 (s, 3H), 1.08 (t, J=6.8 Hz, 3H). LCMS: (ES⁺) m/z=568.4 (M+H), Rt=0.905 min. (Method 1).

(xcvii) Compound 91:

To a solution of 1-[8-(2-chlorophenyl)-9-(4-chlorophenyl)-2-methylsulfonyl-purin-6-yl]-4-methyl-piperidine-4-carboxamide (Compound 68) (80 mg, 143 μmol, 1 eq.) in (3R)-3-methoxypyrrolidine (217 mg, 2.14 mmol, 15 eq.) was stirred at 140° C. for 1 hour. The reaction mixture was concentrated under reduced pressure. The residue was purified by prep-HPLC (column: Waters Xbridge 150*25 mm*5 μm; mobile phase: [water (ammonia hydroxide v/v)-ACN]; B %: 48%-78%, 8 min) to give an off-white solid. Then the off-white solid was purified by prep-HPLC (column: Phenomenex luna C18 150*25 mm*10 μm; mobile phase: [water(formic acid)-ACN]; B %: 53%-83%, 10 min) to give the compound 1-[8-(2-chlorophenyl)-9-(4-chlorophenyl)-2-[(3R)-3-methoxypyrrolidin-1-yl]purin-6-yl]-4-methyl-piperidine-4-carboxamide (Compound 91, 27.48 mg, 47.3 μmol, 33.1% yield) as an off-white solid. ¹H NMR (400 MHz, CDCl₃-d) δ=7.54-7.46 (m, 1H), 7.37-7.27 (m, 3H), 7.26-7.16 (m, 4H), 5.80-5.24 (m, 2H), 4.55 (br s, 2H), 4.01 (td, J=3.9, 7.8 Hz, 3H), 3.73-3.50 (m, 4H), 3.36 (s, 3H), 2.19-1.95 (m, 4H), 1.70-1.62 (m, 2H), 1.30 (s, 3H). LCMS: (ES⁺) m/z=580.2 (M+H).

(xcviii) Compound 92:

To a solution of 1-[8-(2-chlorophenyl)-9-(4-chlorophenyl)-2-methylsulfonyl-purin-6-yl]-4-methyl-piperidine-4-carboxamide (Compound 68) (80 mg, 143 μmol, 1 eq.) in (3S)-3-methoxypyrrolidine (217 mg, 2.14 mmol, 15 eq.) was stirred at 140° C. for 1 hour. The reaction mixture was concentrated under reduced pressure. The residue was purified by prep-HPLC (column: Waters Xbridge 150*25 mm*5 μm; mobile phase: [water (ammonia hydroxide v/v)-ACN]; B %: 50%-80%, 8 min) to give an off-white solid. Then the off-white solid was purified by prep-HPLC (column: Phenomenex luna C18 150*25 mm*10 μm; mobile phase: [water(formic acid)-ACN]; B %: 52%-82%, 10 min) to give the compound 1-[8-(2-chlorophenyl)-9-(4-chlorophenyl)-2-[(3S)-3-methoxypyrrolidin-1-yl]purin-6-yl]-4-methyl-piperidine-4-carboxamide (Compound 92, 22.08 mg, 38.0 μmol, 26.6% yield) as an off-white solid. ¹H NMR (400 MHz, CDCl₃-d) δ=7.58-7.45 (m, 1H), 7.37-7.26 (m, 4H), 7.25-7.18 (m, 3H), 5.92-5.13 (m, 2H), 4.54 (m, 2H), 4.14-3.89 (m, 3H), 3.76-3.51 (m, 4H), 3.36 (s, 3H), 2.23-1.93 (m, 4H), 1.69-1.62 (m, 2H), 1.30 (s, 3H). LCMS: (ES⁺) m/z=580.2 (M+H).

(xcix) Compound 93:

To a solution of 1-[8-(2-chlorophenyl)-9-(4-chlorophenyl)-2-methylsulfonyl-purin-6-yl]-4-methyl-piperidine-4-carboxamide (Compound 68) (80 mg, 0.14 mmol, 1 eq.) in (3R)-pyrrolidin-3-ol (125 mg, 1.43 mmol, 0.12 mL, 10 eq.) was stirred at 140° C. for 2 hours. The residue was dissolved in acetonitrile (2 mL) and purified by prep-HPLC (column: Waters Xbridge 150*25 mm*5 μm; mobile phase: [water (ammonium bicarbonate)-acetonitrile]; B %: 45%-75%, 8 min) and the organic phase was concentrated under reduced pressure to remove acetonitrile, and the liquid was lyophilized to give 1-[8-(2-chlorophenyl)-9-(4-chlorophenyl)-2-[(3R)-3-hydroxypyrrolidin-1-yl]purin-6-yl]-4-methyl-piperidine-4-carboxamide (Compound 93, 36.85 mg, 63.36 μmol, 44.3% yield) as a yellow solid. ¹H NMR (400 MHz, MeOD-d₄) δ=7.57 (d, J=6.4 Hz, 1H), 7.48-7.31 (m, 5H), 7.31-7.23 (m, 2H), 4.77-4.64 (m, 2H), 4.44 (s, 1H), 3.76 (t, J=10.8 Hz, 2H), 3.68-3.49 (m, 4H), 2.19-1.90 (m, 4H), 1.63-1.51 (m, 2H), 1.27 (s, 3H). LCMS: (ES⁺) m/z=566.2 (M+H).

### (c) Compound 94:

To a solution of 1-[8-(2-chlorophenyl)-9-(4-chlorophenyl)-2-methylsulfonyl-purin-6-yl]-4-methyl-piperidine-4-carboxamide (Compound 68) (80 mg, 0.14 mmol, 1 eq.) in (3S)-pyrrolidin-3-ol (125 mg, 1.43 mmol, 0.12 mL, 10 eq.) was stirred at 140° C. for 5 hours. The residue was dissolved in acetonitrile (2 mL) and purified by prep-HPLC (column: Waters Xbridge 150*25 mm*5 μm; mobile phase: [water(ammonium bicarbonate)-acetonitrile]; B %: 45%-75%, 8 min) and the organic phase was concentrated under reduced pressure to remove acetonitrile, and the liquid was lyophilized to give 1-[8-(2-chlorophenyl)-9-(4-chlorophenyl)-2-[(3S)-3-hydroxypyrrolidin-1-yl]purin-6-yl]-4-methyl-piperidine-4-carboxamide (Compound 94, 11.03 mg, 19.5 μmol, 13.6% yield) as a yellow solid. ¹H NMR (400 MHz, MeOD-d₄) δ=7.60-7.55 (m, 1H), 7.47-7.32 (m, 5H), 7.31-7.25 (m, 2H), 4.76-4.64 (m, 2H), 4.49-4.39 (m, 1H), 3.85-3.71 (m, 2H), 3.67-3.52 (m, 4H), 2.21-2.13 (m, 2H), 2.09-2.03 (m, 1H), 2.00-1.91 (m, 1H), 1.60-1.53 (m, 2H), 1.27 (s, 3H). LCMS: (ES⁺) m/z=566.3 (M+H).

### (ci) Compound 102:

To a solution of 1-[8-(2-chlorophenyl)-9-(4-chlorophenyl)-2-methylsulfonyl-purin-6-yl]-4-methyl-piperidine-4-carboxamide (Compound 68) (80 mg, 0.14 mmol, 1.0 eq.) in N′,N′-dimethylethane-1,2-diamine (126 mg, 1.43 mmol, 0.16 mL, 10 eq.) was stirred at 140° C. for 1 hour. The residue was dissolved in acetonitrile (2 mL) and purified by prep-HPLC (column: Phenomenex luna C18 150*25 mm*10 μm; mobile phase: [water (formic acid)-ACN]; B %: 16%-46%, 10 min) and the organic phase was concentrated under reduced pressure to remove acetonitrile, and the liquid was lyophilized to give 1-[8-(2-chlorophenyl)-9-(4-chlorophenyl)-2-[2-(dimethylamino)ethylamino]purin-6-yl]-4-methyl-piperidine-4-carboxamide (Compound 102, 15.48 mg, 27.3 μmol, 19.0% yield) as a white solid. ¹H NMR (400 MHz, MeOD-d₄) δ=8.55 (s, 1H), 7.60-7.53 (m, 1H), 7.49-7.34 (m, 5H), 7.32-7.23 (m, 2H), 4.72-4.71 (m, 2H), 3.77 (t, J=9.6 Hz, 2H), 3.56 (t, J=6.4 Hz, 2H), 2.85 (t, J=6.4 Hz, 2H), 2.47 (s, 6H), 2.16 (d, J=14.4 Hz, 2H), 1.59-1.52 (m, 2H), 1.27 (s, 3H). LCMS: (ES⁺) m/z=567.2 (M+H).

(cii) Compound 103:

To a solution of 1-[6-(4-chloroanilino)-2-(2-hydroxy-2-methyl-propoxy)-5-nitro-pyrimidin-4-yl]-4-methyl-piperidine-4-carboxamide (Intermediate B-5(103), Example 5iii) (50 mg, 104 μmol, 1.0 eq.) and pyridine-3-carbaldehyde (56 mg, 522 μmol, 5.0 eq.) in ethanol (1 mL) and acetic acid (0.4 mL) was added iron (58 mg, 1.04 mmol, 10 eq.). The mixture was stirred at 80° C. for 1 hour. The reaction mixture was filtered and concentrated under vacuum. The residue was purified by prep-HPLC (column: Phenomenex luna C18 150*25 mm*10 μm; mobile phase: [water(formic acid)-ACN]; B %: 41%-61%, 2 min) to give the compound 1-[9-(4-chlorophenyl)-2-(2-hydroxy-2-methyl-propoxy)-8-(3-pyridyl)purin-6-yl]-4-methyl-piperidine-4-carboxamide (Compound 103, 17.36 mg, 32.4 μmol, 31.0% yield) as a off-white solid. ¹HNMR (400 MHz, CDCl₃-d) δ=8.93-8.46 (m, 2H), 7.71 (br d, J=7.3 Hz, 1H), 7.47 (br d, J=8.2 Hz, 2H), 7.27-7.23 (m, 3H), 6.00-5.26 (m, 2H), 5.18-4.24 (m, 2H), 4.23-4.15 (m, 2H), 4.13-3.20 (m, 2H), 2.19 (br d, J=12.8 Hz, 2H), 1.58-1.42 (m, 2H), 1.38-1.32 (m, 3H), 1.32-1.27 (m, 6H). LCMS: (ES⁺) m/z=536.3 (M+H).

(ciii) Compound 104:

To a solution of 1-[6-(4-chloroanilino)-2-(2-hydroxy-2-methyl-propoxy)-5-nitro-pyrimidin-4-yl]-4-methyl-piperidine-4-carboxamide (Intermediate B-5(103), Example 4iii) (50 mg, 104 μmol, 1.0 eq.) and 2-methylpyridine-3-carbaldehyde (63 mg, 522 μmol, 5.0 eq.) in ethanol (1 mL) and acetic acid (0.4 mL) was added iron (58 mg, 1.04 mmol, 10 eq.). The mixture was stirred at 80° C. for 1 hour. The reaction mixture was filtered and concentrated under vacuum. The residue was purified by prep-HPLC (column: Unisil 3-100 C18 Ultra 150*50 mm*3 μm; mobile phase: [water(formic acid)-ACN]; B %: 27%-57%, 7 min) to give a white solid. The white solid was purified by prep-HPLC (column: Waters Xbridge 150*25 mm*5 μm; mobile phase: [water(ammonia hydroxide v/v)-ACN]; B %: 29%-59%, 9 min) to give the compound 1-[9-(4-chlorophenyl)-2-(2-hydroxy-2-methyl-propoxy)-8-(2-methyl-3-pyridyl)purin-6-yl]-4-methyl-piperidine-4-carboxamide (Compound 104, 10.57 mg, 19.2 μmol, 18.4% yield) as a white solid. ¹HNMR (400 MHz, CDCl₃-d) δ=8.55 (m, 1H), 7.49 (m, 1H), 7.35 (d, J=8.7 Hz, 2H), 7.18-7.07 (m, 3H), 5.79-5.26 (m, 2H), 5.21-4.33 (m, 2H), 4.24 (s, 2H), 4.13-3.33 (m, 2H), 2.47 (s, 3H), 2.25-2.11 (m, 2H), 1.65 (ddd, J=m, 2H), 1.34-1.29 (m, 9H). LCMS: (ES⁺) m/z=550.4 (M+H).

(civ) Compound 105:

To a solution of 1-[6-(4-chloroanilino)-2-(2-hydroxy-2-methyl-propoxy)-5-nitro-pyrimidin-4-yl]-4-methyl-piperidine-4-carboxamide (Intermediate B-5(103), Example 4iii) (50 mg, 104 μmol, 1.0 eq.) and 4-methylnicotinaldehyde (63 mg, 522 μmol, 5.0 eq.) in ethanol (1 mL) and acetic acid (0.4 mL) was added iron (58 mg, 1.04 mmol, 10 eq.). The mixture was stirred at 80° C. for 1 hour. The reaction mixture was filtered and concentrated under vacuum. The residue was purified by prep-HPLC (column: Phenomenex luna C18 150*25 mm*10 μm; mobile phase: [water(formic acid)-ACN]; B %:31%-61%, 10 min) to give the compound 1-[9-(4-chlorophenyl)-2-(2-hydroxy-2-methyl-propoxy)-8-(4-methyl-3-pyridyl)purin-6-yl]-4-methyl-piperidine-4-carboxamide (Compound 105, 25.50 mg, 46.36 μmol, 44.4% yield) as a pink solid. ¹HNMR (400 MHz, CDCl₃-d) δ=8.58-8.33 (m, 2H), 7.35 (d, J=8.6 Hz, 2H), 7.19-7.09 (m, 3H), 5.77-5.37 (m, 2H), 5.01-4.29 (m, 2H), 4.24 (s, 2H), 4.17-3.71 (m, 2H), 3.57-3.14 (m, 1H), 2.28-2.20 (m, 3H), 2.17 (br d, J=14.4 Hz, 2H), 1.71-1.62 (m, 2H), 1.41-1.20 (m, 9H). LCMS: (ES⁺) m/z=550.3 (M+H).

### (cv) Compound 106:

To a solution of 1-[6-(4-chloroanilino)-2-(2-hydroxy-2-methyl-propoxy)-5-nitro-pyrimidin-4-yl]-4-methyl-piperidine-4-carboxamide (Intermediate B-5(103), Example 4iii) (50 mg, 104 μmol, 1.0 eq.) and 4-formylbenzonitrile (137 mg, 1.04 mmol, 10 eq.) in ethanol (1 mL) and acetic acid (0.4 mL) was added iron (58 mg, 1.04 mmol, 10 eq.). The mixture was stirred at 80° C. for 1 hour. The reaction mixture was filtered and concentrated under vacuum. The residue was purified by prep-HPLC (column: Waters Xbridge 150*25 mm*5 μm; mobile phase: [water(ammonium bicarbonate)-ACN]; B %: 40%-70%, 8 min) to give a white solid. Then the white solid was purified by prep-HPLC (column: Phenomenex luna C18 150*25 mm*10 μm; mobile phase: [water(formic acid)-ACN]; B %: 42%-72%, 10 min) to give the compound 1-[9-(4-chlorophenyl)-8-(4-cyanophenyl)-2-(2-hydroxy-2-methyl-propoxy)purin-6-yl]-4-methyl-piperidine-4-carboxamide (Compound 106, 6.17 mg, 11.0 μmol, 10.5% yield) yellow solid. ¹H NMR (400 MHz, DMSO-d₆) δ=7.86 (d, J=8.6 Hz, 2H), 7.68-7.52 (m, 4H), 7.48-7.40 (m, 2H), 7.29 (s, 1H), 7.07-6.88 (m, 1H), 5.36-4.82 (m, 1H), 4.61 (br s, 1H), 4.51-4.14 (m, 1H), 3.95 (s, 3H), 3.61-3.43 (m, 1H), 2.19-2.09 (m, 2H), 1.49-1.37 (m, 2H), 1.18-1.11 (m, 9H). LCMS: (ES⁺) m/z=560.3 (M+H).

(cvi) Compound 107:

To a solution of 1-[6-(4-chloroanilino)-2-(2-hydroxy-2-methyl-propoxy)-5-nitro-pyrimidin-4-yl]-4-methyl-piperidine-4-carboxamide (Intermediate B-5(103), Example 4iii) (90 mg, 0.19 mmol, 1.0 eq.) and 5-formyl-6-methyl-pyridine-2-carbonitrile (37 mg, 0.94 mmol, 5.0 eq.) in ethanol (1 mL) and acetic acid (0.4 mL) was added iron (105 mg, 1.89 mmol, 10 eq.). The mixture was stirred at 80° C. for 1 hour. The reaction mixture was cooled at room temperature and filtered. The resulting filter liquor was diluted with N,N-dimethylformamide (2 mL) and purified by reversed-phase HPLC (column: Phenomenex Luna C18 150*25 mm*10 μm; mobile phase: [water (FA)-ACN]; B %: 41%-71%, 10 min). The cut fraction was concentrated under reduced pressure to remove acetonitrile. The residue was lyophilized to give compound 1-[9-(4-chlorophenyl)-8-(6-cyano-2-methyl-3-pyridyl)-2-(2-hydroxy-2-methyl-propoxy)purin-6-yl]-4-methyl-piperidine-4-carboxamide (Compound 107, 34.93 mg, 60.7 μmol, 31.5% yield) as a yellow solid. ¹H NMR (400 MHz, DMSO-d₆) 5=7.93-7.88 (m, 1H), 7.88-7.82 (m, 1H), 7.55 (d, J=8.8 Hz, 2H), 7.39 (d, J=8.8 Hz, 2H), 7.29 (s, 1H), 7.00 (s, 1H), 5.24-4.78 (m, 1H), 4.63 (s, 1H), 4.48-4.17 (m, 1H), 3.98 (s, 2H), 3.93-3.40 (m, 2H), 3.34 (s, 3H), 2.17-2.05 (m, 2H), 1.50-1.36 (m, 2H), 1.23-1.09 (m, 9H). LCMS: (ES⁺) m/z=575.3 (M+H).

(cvii) Compound 108:

To a solution of 1-[6-(4-chloroanilino)-2-(2-hydroxy-2-methyl-propoxy)-5-nitro-pyrimidin-4-yl]-4-methyl-piperidine-4-carboxamide (Intermediate B-5(103), Example 4iii) (50 mg, 104 μmol, 1.0 eq.) and 5-formyl-4-methyl-pyridine-2-carbonitrile (76 mg, 522 μmol, 5.0 eq.) in ethanol (1 mL) and AcOH (0.4 mL) was added Fe (58.30 mg, 1.04 mmol, 10 eq.). The resulting mixture was stirred at 80° C. for 1 hour. The reaction mixture was cooled to room temperature and filtered, the resulting filter liquor was concentrated under reduced pressure to remove ethanol. The residue was diluted with N,N-dimethylformamide (2 mL) and purified by reversed-phase HPLC (column: Phenomenex luna C18 150*25 mm*10 μm; mobile phase: [water(formic acid)-ACN]; B %: 41%-71%, 10 min). The cut fraction was concentrated under reduced pressure to remove acetonitrile and lyophilized to give 1-[9-(4-chlorophenyl)-8-(6-cyano-4-methyl-3-pyridyl)-2-(2-hydroxy-2-methyl-propoxy)purin-6-yl]-4-methyl-piperidine-4-carboxamide (Compound 108, 21.18 mg, 36.8 μmol, 35.2% yield) as a yellow solid. ¹H NMR (400 MHz, CDCl₃-d) δ=8.45 (s, 1H), 7.60 (s, 1H), 7.42 (d, J=8.4 Hz, 2H), 7.15 (d, J=8.4 Hz, 2H), 5.69-5.36 (m, 2H), 5.22-4.38 (m, 2H), 4.25 (s, 2H), 4.20-3.61 (m, 2H), 3.21 (s, 1H), 2.38 (s, 3H), 2.24-2.15 (m, 2H), 1.70-1.63 (m, 2H), 1.35 (s, 3H), 1.33 (s, 6H). LCMS: (ES⁺) m/z=575.3 (M+H).

(cviii) Compound 109:

To a solution of 1-[6-(4-chloroanilino)-2-(2-hydroxy-2-methyl-propoxy)-5-nitro-pyrimidin-4-yl]-4-methyl-piperidine-4-carboxamide (Intermediate B-5(103), Example 4iii) (50 mg, 104 μmol, 1.0 eq.) and 5-formylpyridine-2-carbonitrile (69 mg, 522 μmol, 5.0 eq.) in ethanol (1 mL) and acetic acid (0.4 mL) was added iron (58 mg, 1.04 mmol, 10 eq.). The mixture was stirred at 80° C. for 1 hour. The reaction mixture was filtered and concentrated under vacuum. The residue was purified by prep-HPLC (column: Phenomenex luna C18 150*25 mm*10 μm; mobile phase: [water(formic acid)-ACN]; B %: 40%-70%, 10 min) to give the compound 1-[9-(4-chlorophenyl)-8-(6-cyano-3-pyridyl)-2-(2-hydroxy-2-methyl-propoxy)purin-6-yl]-4-methyl-piperidine-4-carboxamide (Compound 109, 11.46 mg, 19.76 μmol, 18.9% yield, 96.7% purity) as a yellow solid. ¹H NMR (400 MHz, DMSO-d₆) δ=8.79 (d, J=2.1 Hz, 1H), 8.05 (d, J=8.3 Hz, 1H), 7.91 (m, 1H), 7.64 (d, J=8.7 Hz, 2H), 7.53-7.46 (m, 2H), 7.29 (s, 1H), 7.00 (s, 1H), 5.71-4.73 (m, 1H), 4.68-4.53 (m, 1H), 4.48-4.13 (m, 1H), 4.05-3.84 (m, 3H), 3.67-3.36 (m, 1H), 2.14 (br d, J=9.8 Hz, 2H), 1.48-1.39 (m, 2H), 1.18-1.13 (m, 9H). LCMS: (ES⁺) m/z=561.3 (M+H).

(cix) Compound 110:

To a solution of 1-[6-(4-chloroanilino)-2-[(2-hydroxy-2-methyl-propyl)-methyl-amino]-5-nitro-pyrimidin-4-yl]-4-methyl-piperidine-4-carboxamide (Intermediate B-5(110), Example 4iv) (90 mg, 0.18 mmol, 1.0 eq.) and 5-formylpyridine-2-carbonitrile (121 mg, 0.91 mmol, 5.0 eq.) in ethanol (2 mL) and acetic acid (0.8 mL) was added iron (102 mg, 1.83 mmol, 10 eq.). The mixture was stirred at 80° C. for 1 hour. The reaction mixture was filtered, the resulting filter liquor was concentrated under reduced pressure to give the residue. The residue was purified by flash silica gel chromatography (ISCO®; 20 g SepaFlash® Silica Flash Column, Eluent of 0˜10% methanol/dichloroethane@30 mL/min). The cut fraction was concentrated under reduced pressure to give the crude product. The crude product was purified by reversed-phase HPLC(column: Phenomenex luna C18 150*25 mm*10 μm; mobile phase: [water(formic acid)-acetonitrile]; B %: 44%-74%, 10 min) to give 1-[9-(4-chlorophenyl)-8-(6-cyano-3-pyridyl)-2-[(2-hydroxy-2-methyl-propyl)-methyl-amino]purin-6-yl]-4-methyl-piperidine-4-carboxamide (Compound 110, 23.43 mg, 39.2 μmol, 24.9% yield). ¹H NMR (400 MHz, CDCl₃-d) δ=8.85 (s, 1H), 7.81 (d, J=7.6 Hz, 1H), 7.60 (d, J=8.4 Hz, 1H), 7.51 (d, J=8.4 Hz, 2H), 7.25 (d, J=8.4 Hz, 2H), 5.80-5.34 (m, 3H), 5.11-4.25 (m, 2H), 4.22-3.74 (m, 2H), 3.53 (s, 2H), 3.20 (s, 3H), 2.24-2.13 (m, 2H), 1.72-1.67 (m, 2H), 1.35 (s, 3H), 1.22 (s, 6H). LCMS: (ES⁺) m/z=574.2 (M+H).

### (cx) Compound 111:

A mixture of 1-[6-(4-chloroanilino)-2-[(2-hydroxy-2-methyl-propyl)amino]-5-nitro-pyrimidin-4-yl]-4-methyl-piperidine-4-carboxamide (Intermediate B-5(111), Example 4v) (170 mg, 0.36 mmol, 1.0 eq.), 5-formylpyridine-2-carbonitrile (470 mg, 3.56 mmol, 10 eq.) and iron (199 mg, 3.56 mmol, 10 eq.) in acetic acid (0.8 mL) and ethanol (2 mL) was stirred at 80° C. for 1 hour. The reaction mixture was cooled at room temperature and filtered, the resulting filtrate was purified by flash silica gel chromatography (ISCO®; 12 g SepaFlash® Silica Flash Column, Eluent of 0˜10% Dichloromethane/Methanol @30 mL/min) and reversed-phase HPLC (column: Phenomenex luna C18 150*25 mm*10 μm; mobile phase: [water (formic acid)-ACN]; B %: 32%-62%, 10 min). The cut fraction was concentrated under reduced pressure to remove acetonitrile. The residue was lyophilized to give compound 1-[9-(4-chlorophenyl)-8-(6-cyano-3-pyridyl)-2-[(2-hydroxy-2-methyl-propyl)amino]purin-6-yl]-4-methyl-piperidine-4-carboxamide (Compound 111, 35.19 mg, 61.6 μmol, 17.3% yield) as a yellow solid. ¹H NMR (400 MHz, DMSO-d₆) δ=8.75 (d, J=2.0 Hz, 1H), 8.01 (d, J=8.4 Hz, 1H), 7.87-7.81 (m, 1H), 7.66-7.56 (m, 2H), 7.47 (d, J=8.4 Hz, 2H), 7.26 (s, 1H), 6.96 (s, 1H), 6.45-6.33 (m, 1H), 5.48-3.33 (m, 5H), 3.26-3.12 (m, 2H), 2.15-2.07 (m, 2H), 1.48-1.36 (m, 2H), 1.17 (s, 3H), 1.06 (s, 6H). LCMS: (ES⁺) m/z=560.3 (M+H).

(cxi) Compound 112:

To a solution of 1-[6-(4-chloroanilino)-2-(2-hydroxy-2-methyl-propoxy)-5-nitro-pyrimidin-4-yl]-3-methyl-azetidin-3-ol (Intermediate B-5(112), Example 4vi) (50 mg, 0.12 mmol, 1.0 eq.) and 5-formylpyridine-2-carbonitrile (78 mg, 0.59 mmol, 5.0 eq.) in ethanol (1 mL) and acetic acid (0.4 mL) was added iron (66 mg, 1.18 mmol, 10 eq.). The mixture was stirred at 80° C. for 1 hour. The reaction mixture was filtered and concentrated under vacuum. The residue was purified by prep-HPLC (column: Phenomenex luna C18 150*25 mm*10 μm; mobile phase: [water(formic acid)-ACN]; B %: 36%-66%, 10 min) to give the compound 5-[9-(4-chlorophenyl)-6-(3-hydroxy-3-methyl-azetidin-1-yl)-2-(2-hydroxy-2-methyl-propoxy)purin-8-yl]pyridine-2-carbonitrile (Compound 112, 24.97 mg, 48.8 μmol, 41.4% yield) as a yellow solid. ¹H NMR (400 MHz, CDCl₃-d) δ=8.84 (s, 1H), 7.83 (br d, J=7.8 Hz, 1H), 7.62 (br d, J=7.8 Hz, 1H), 7.51 (br d, J=7.6 Hz, 2H), 7.28-7.27 (m, 1H), 7.29-7.21 (m, 2H), 4.88-4.27 (m, 4H), 4.24-4.14 (m, 2H), 3.09-2.17 (m, 2H), 1.81-1.59 (m, 3H), 1.28 (s, 6H). LCMS: (ES⁺) m/z=506.2 (M+H).

(cxii) Compound 113:

A mixture of 1-[6-(4-chloroanilino)-2-(2-hydroxy-2-methyl-propoxy)-5-nitro-pyrimidin-4-yl]-3-methyl-azetidine-3-carbonitrile (Intermediate B-5(113), Example 4vii) (200 mg, 0.46 mmol, 1.0 eq.) and 5-formylpyridine-2-carbonitrile (305 mg, 2.31 mmol, 5.0 eq.) in ethyl alcohol (2 mL) was added iron (258 mg, 4.62 mmol, 10 eq.) and acetic acid (0.8 mL), the mixture was stirred at 80° C. for 1 hour. The reaction mixture was filtered and concentrated under reduced pressure to give a residue. The residue was purified by flash silica gel chromatography (ISCO®; 20 g SepaFlash® Silica Flash Column, Eluent of 0˜100% Ethyl acetate/Methanol @30 mL/min) and the organic phase was concentrated under reduced pressure to give the crude product. The residue was dissolved in N,N-dimethylformamide (2 mL) and purified by prep-HPLC (column: Phenomenex luna C18 150*25 mm*10 μm; mobile phase: [water (formic acid)-ACN]; B %: 43%-73%, 10 min) and the organic phase was concentrated under reduced pressure to remove acetonitrile, and the liquid was lyophilized to give 5-[9-(4-chlorophenyl)-6-(3-cyano-3-methyl-azetidin-1-yl)-2-(2-hydroxy-2-methyl-propoxy)purin-8-yl]pyridine-2-carbonitrile (Compound 113, 93.52 mg, 0.18 mmol, 39.0% yield) as a yellow solid. ¹H NMR (400 MHz, DMSO-d₆) δ=8.86-8.78 (m, 1H), 8.09-8.02 (m, 1H), 7.95-7.86 (m, 1H), 7.68-7.60 (m, 2H), 7.55-7.42 (m, 2H), 5.13-4.09 (m, 5H), 3.94 (s, 2H), 1.73 (s, 3H), 1.14 (s, 6H). LCMS: (ES⁺) m/z=515.2 (M+H).

(cxiii) Compound 114:

A mixture of 1-[4-(4-chloroanilino)-6-(3-methoxy-3-methyl-azetidin-1-yl)-5-nitro-pyrimidin-2-yl]oxy-2-methyl-propan-2-ol (Intermediate B-5(113), Example 4viii) (50 mg, 0.11 mmol, 1.0 eq.) and 5-formylpyridine-2-carbonitrile (75 mg, 0.57 mmol, 5.0 eq.) in ethyl alcohol (1 mL) was added iron (63 mg, 1.14 mmol, 10 eq.) and acetic acid (0.4 mL), the mixture was stirred at 80° C. for 1 hour. The reaction mixture was filtered and concentrated under reduced pressure to give a residue. The residue was purified by flash silica gel chromatography (ISCO®; 20 g SepaFlash® Silica Flash Column, Eluent of 0˜100% Ethyl acetate/Methanol@30 mL/min) and the organic phase was concentrated under reduced pressure to give the crude product. The crude product was purified by prep-HPLC (column: Phenomenex Luna C18 150*25 mm*10 μm; mobile phase: [water (formic acid)-ACN]; B %: 44%-74%, 10 min) and the organic phase was concentrated under reduced pressure to remove acetonitrile, and the liquid was lyophilized to give 5-[9-(4-chlorophenyl)-2-(2-hydroxy-2-methyl-propoxy)-6-(3-methoxy-3-methyl-azetidin-1-yl)purin-8-yl]pyridine-2-carbonitrile (Compound 114, 47.68 mg, 91.7 μmol, 80.3% yield) as a yellow solid. ¹H NMR (400 MHz, DMSO-d₆) δ=8.87-8.71 (m, 1H), 8.05 (d, J=8.0 Hz, 1H), 7.93-7.88 (m, 1H), 7.71-7.60 (m, 2H), 7.55-7.45 (m, 2H), 4.61 (s, 1H), 4.57-4.01 (m, 4H), 3.94 (s, 2H), 3.26 (s, 3H), 1.52 (s, 3H), 1.14 (s, 6H). LCMS: (ES⁺) m/z=520.2 (M+H).

(cxiv) Compound 115:

1-(9-(4-chlorophenyl)-8-(1,5-dimethyl-1H-imidazol-2-yl)-2-((2-hydroxyethyl)(methyl)amino)-9H-purin-6-yl)-4-methylpiperidine-4-carboxamide (Compound 115) was obtained following the synthetic procedure described for Compound 57. ¹H NMR (400 MHz, CDCl₃-d) δ=7.28 (d, J=8.7 Hz, 2H), 7.21-7.06 (m, 3H), 6.59 (s, 1H), 5.74-5.16 (m, 2H), 4.65-4.37 (m, 2H), 3.96-3.71 (m, 2H), 3.67 (br s, 2H), 3.66 (s, 3H), 3.59-3.51 (m, 2H), 3.04 (s, 3H), 2.10 (s, 3H), 2.04-1.96 (m, 2H), 1.53-1.48 (m, 2H), 1.19 (s, 3H). LCMS: (ES⁺) m/z=538.4 (M+H).

(cxv) Compound 116:

To a solution of 1-[6-(4-chloroanilino)-2-(2-hydroxy-2-methyl-propoxy)-5-nitro-pyrimidin-4-yl]-4-methyl-piperidine-4-carboxamide (Intermediate B-5(103), Example 4iii) (100 mg, 209 μmol, 1.0 eq.) and 6-(dimethylamino)pyridine-3-carbaldehyde (157 mg, 1.04 mmol, 5 eq.) in ethanol (2 mL) and acetic acid (0.8 mL) was added iron (117 mg, 2.09 mmol, 10 eq.). The mixture was stirred at 80° C. for 1 hour. The reaction mixture was filtered and concentrated under vacuum. The residue was purified by flash silica gel chromatography (ISCO®; 20 SepaFlash® Silica Flash Column, Eluent of 0˜15% Dichloromethane/Methanol @60 mL/min) to give a white solid (R_{f}=0.4). The desired mass was detected by LCMS (EW32208-162-P1A1). The white solid was purified by prep-HPLC (column: Phenomenex luna C18 150*25 mm*10 μm; mobile phase: [water(formic acid)-ACN]; B %: 19%-49%, 10 min) to give the compound 1-[9-(4-chlorophenyl)-8-[6-(dimethylamino)-3-pyridyl]-2-(2-hydroxy-2-methyl-propoxy)purin-6-yl]-4-methyl-piperidine-4-carboxamide (Compound 116, 8.67 mg, 15.0 μmol, 7.1% yield) as a white solid. ¹H NMR (400 MHz, DMSO-d₆) δ=8.07 (d, J=2.2 Hz, 1H), 7.66-7.58 (m, 2H), 7.51-7.38 (m, 3H), 7.27 (s, 1H), 6.97 (s, 1H), 6.61 (d, J=8.9 Hz, 1H), 4.58 (br s, 2H), 4.41-4.19 (m, 1H), 3.93 (s, 3H), 3.71-3.51 (m, 1H), 3.02 (s, 6H), 2.12 (br d, J=13.3 Hz, 2H), 1.49-1.38 (m, 2H), 1.23-1.08 (m, 9H). LCMS: (ES⁺) m/z=579.4 (M+H).

(cxvi) Compound 117:

A mixture of 1-[8-(2-chlorophenyl)-9-(4-chlorophenyl)-2-methylsulfonyl-purin-6-yl]-4-methyl-piperidine-4-carboxamide (Compound 68) (50 mg, 89.4 μmol, 1.0 eq.), [(2S)-1-methylpyrrolidin-2-yl]methanol (51 mg, 0.47 mmol, 0.05 mL, 5.0 eq.) and potassium carbonate (37 mg, 0.27 mmol, 3 eq.) in N,N-dimethylformamide (1 mL) was stirred at 100° C. for 12 hours. The reaction mixture was filtered and concentrated under reduced pressure to give a residue. The residue was dissolved in acetonitrile (2 mL) and purified by prep-HPLC (column: Waters Xbridge 150*25 mm*5 μm; mobile phase: [water (ammonium bicarbonate)-ACN]; B %: 51%-81%, 8 min) and the organic phase was concentrated under reduced pressure to remove acetonitrile, and the liquid was lyophilized to give 1-[8-(2-chlorophenyl)-9-(4-chlorophenyl)-2-[[(2S)-1-methylpyrrolidin-2-yl]methoxy]purin-6-yl]-4-methyl-piperidine-4-carboxamide (Compound 117, 22.70 mg, 38.2 μmol, 42.7% yield) as an off-white solid. ¹H NMR (400 MHz, DMSO-d₆) δ=7.69-7.66 (m, 1H), 7.54-7.40 (m, 5H), 7.33-7.23 (m, 3H), 6.96 (s, 1H), 5.19-4.65 (m, 1H), 4.29-4.25 (m, 2H), 3.99-3.95 (m, 1H), 3.92-3.39 (m, 2H), 2.98-2.88 (m, 1H), 2.29 (s, 3H), 2.25-1.95 (m, 4H), 1.94-1.84 (m, 1H), 1.70-1.52 (m, 3H), 1.49-1.36 (m, 2H), 1.16 (s, 3H). LCMS: (ES⁺) m/z=594.3 (M+H).

(cxvii) Compound 118:

A mixture of 1-[6-(4-chloroanilino)-2-(2-hydroxy-2-methyl-propoxy)-5-nitro-pyrimidin-4-yl]-4-methyl-piperidine-4-carboxylic acid (Intermediate B-5(118), Example 4ix) (190 mg, 0.40 mmol, 1.0 eq.) and 5-formylpyridine-2-carbonitrile (262 mg, 1.98 mmol, 5 eq.) and acetic acid (0.8 mL) in ethyl alcohol (2 mL) was added iron (221 mg, 3.96 mmol, 10 eq.), the mxiture was stirred at 80° C. for 1 hour. The reaction mixture was washed with water (20 mL) and extracted with ethyl acetate 60 mL (20 mL×3). The combined organic layers were washed with brine 30 mL (15 mL×2), dried over anhydrous sodium sulfate, filtered and concentrated under reduced pressure to give a residue. The residue was purified by flash silica gel chromatography (ISCO®; 20 g SepaFlash® Silica Flash Column, Eluent of 0˜100% Dichloromethane Methanol @30 mL/min) and the organic phase was concentrated under reduced pressure to give the crude product. The residue was dissolved in N,N-dimethylformamide (2 mL) and purified by prep-HPLC (column: Phenomenex luna C18 150*25 mm*10 μm; mobile phase: [water (formic acid)-ACN]; B %: 43%-73%, 10 min) and the organic phase was concentrated under reduced pressure to remove acetonitrile, and the liquid was lyophilized to give 1-[9-(4-chlorophenyl)-8-(6-cyano-3-pyridyl)-2-(2-hydroxy-2-methyl-propoxy)purin-6-yl]-4-methyl-piperidine-4-carboxylic acid (Compound 118, 64.27 mg, 114 μmol, 28.9% yield) as a yellow solid. ¹H NMR (400 MHz, CDCl₃-d₄) δ=8.85 (d, J=2.0 Hz, 1H), 7.97-7.92 (m, 1H), 7.81 (d, J=8.0 Hz, 1H), 7.63-7.54 (m, 2H), 7.45-7.38 (m, 2H), 5.36-4.95 (m, 2H), 4.12 (s, 2H), 3.93-3.45 (m, 2H), 2.26 (d, J=13.6 Hz, 2H), 1.65-1.46 (m, 2H), 1.30 (s, 3H), 1.27 (s, 6H). LCMS: (ES⁺) m/z=562.3 (M+H).

(cxviii) Compound 120:

A mixture of 1-[6-(4-chloroanilino)-2-(2-hydroxy-2-methyl-propoxy)-5-nitro-pyrimidin-4-yl]-4-ethoxy-piperidine-4-carboxamide (Intermediate B-5(120), Example 4x) (150 mg, 0.29 mmol, 1.0 eq.) and 5-formylpyridine-2-carbonitrile (195 mg, 1.47 mmol, 5.0 eq.) in ethyl alcohol (2.5 mL) was added iron (165 mg, 2.95 mmol, 10 eq.) and acetic acid (1 mL), the mixture was stirred at 80° C. for 1 hour. The reaction mixture was filtered and concentrated under reduced pressure to give a residue. The residue was purified by flash silica gel chromatography (ISCO®; 20 g SepaFlash® Silica Flash Column, Eluent of 0˜100% Ethyl acetate/Petroleum ether gradient @60 mL/min) and the organic phase was concentrated under reduced pressure to give the crude product. The residue was dissolved in acetonitrile (2 mL) and purified by prep-HPLC (column: Phenomenex Luna C18 150*25 mm*10 μm; mobile phase: [water (formic acid)-ACN]; B %: 43%-73%, 10 min) and the organic phase was concentrated under reduced pressure to remove acetonitrile, and the liquid was lyophilized to give 1-[9-(4-chlorophenyl)-8-(6-cyano-3-pyridyl)-2-(2-hydroxy-2-methyl-propoxy)purin-6-yl]-4-ethoxy-piperidine-4-carboxamide (Compound 120, 59.22 mg, 100 μmol, 34.0% yield) as a yellow solid. ¹H NMR (400 MHz, DMSO-d₆) δ=8.83-8.78 (m, 1H), 8.09-8.02 (m, 1H), 7.95-7.89 (m, 1H), 7.68-7.62 (m, 2H), 7.55-7.47 (m, 2H), 7.31 (s, 1H), 7.22 (s, 1H), 5.48 (s, 1H), 4.79-4.38 (m, 2H), 3.97 (s, 2H), 3.87-3.43 (m, 2H), 3.42-3.37 (m, 2H), 1.92 (s, 4H), 1.25-1.20 (m, 3H), 1.14 (s, 6H). LCMS: (ES⁺) m/z=591.3 (M+H).

(cxix) Compound 121:

A mixture of 1-[6-(4-chloroanilino)-2-(2-hydroxy-2-methyl-propoxy)-5-nitro-pyrimidin-4-yl]-4-methyl-piperidine-4-carboxamide (Intermediate B-5(103), Example 4iii) (140 mg, 0.29 mmol, 1.0 eq.) and 3-chloropyridine-4-carbaldehyde (207 mg, 1.46 mmol, 5.0 eq.) in ethyl alcohol (2 mL) was added iron (163 mg, 2.92 mmol, 10 eq.) and acetic acid (0.8 mL), the mixture was stirred at 80° C. for 1 hour. The reaction mixture was filtered and concentrated under reduced pressure to give a residue. The residue was dissolved in acetonitrile (2 mL) and purified by prep-HPLC (column: Phenomenex luna C18 150*25 mm*10 μm; mobile phase: [water (formic acid)-ACN]; B %: 39%-69%, 10 min) and the organic phase was concentrated under reduced pressure to remove acetonitrile, and the liquid was lyophilized to give 1-[9-(4-chlorophenyl)-8-(3-chloro-4-pyridyl)-2-(2-hydroxy-2-methyl-propoxy)purin-6-yl]-4-methyl-piperidine-4-carboxamide (Compound 121, 40.47 mg, 70.9 μmol, 24.3% yield) as a yellow solid. ¹H NMR (400 MHz, DMSO-d₆) δ=8.70 (s, 1H), 8.67-8.61 (m, 1H), 7.73-7.65 (m, 1H), 7.63-7.48 (m, 2H), 7.39-7.31 (m, 2H), 7.28 (s, 1H), 6.98 (s, 1H), 5.26-4.75 (m, 1H), 4.62 (s, 1H), 4.55-4.12 (m, 1H), 3.98 (s, 2H), 3.94-3.38 (m, 2H), 2.17-2.05 (m, 2H), 1.50-1.37 (m, 2H), 1.20-1.11 (m, 9H). LCMS: (ES⁺) m/z=570.2 (M+H).

(cxx) Compound 122:

A mixture of 1-[6-(4-chloroanilino)-2-(2-hydroxy-2-methyl-propoxy)-5-nitro-pyrimidin-4-yl]-4-methyl-piperidine-4-carboxamide (Intermediate B-5(103), Example 4iii) (210 mg, 0.44 mmol, 1.0 eq.) and 3-methylpyridine-4-carbaldehyde (266 mg, 2.19 mmol, 5.0 eq.) in ethyl alcohol (5 mL) was added iron (245 mg, 4.38 mmol, 10 eq.) and acetic acid (2 mL), the mixture was stirred at 80° C. for 1 hour. The reaction mixture was filtered and concentrated under reduced pressure to give a residue. The residue was purified by prep-HPLC (column: Phenomenex Luna C18 150*25 mm*10 μm; mobile phase: [water (formic acid)-ACN]; B %: 27%-57%, 10 min) and the organic phase was concentrated under reduced pressure to remove acetonitrile, and the liquid was lyophilized to give 1-[9-(4-chlorophenyl)-2-(2-hydroxy-2-methyl-propoxy)-8-(3-methyl-4-pyridyl)purin-6-yl]-4-methyl-piperidine-4-carboxamide (Compound 122, 97.22 mg, 176 μmol, 40.1% yield) as a yellow solid. ¹H NMR (400 MHz, DMSO-d₆) δ=8.53 (s, 1H), 8.36 (d, J=4.8 Hz, 1H), 7.53 (d, J=8.4 Hz, 2H), 7.36 (d, J=8.4 Hz, 2H), 7.27 (s, 1H), 7.15 (d, J=5.2 Hz, 1H), 6.97 (s, 1H), 5.26-3.99 (m, 5H), 3.97 (s, 2H), 2.25 (s, 3H), 2.13-2.08 (m, 2H), 1.49-1.35 (m, 2H), 1.15 (s, 9H). LCMS: (ES⁺) m/z=550.2 (M+H).

(cxxi) Compound 130:

A mixture of 1-[6-(4-chloroanilino)-2-[(2S)-2-(hydroxymethyl)pyrrolidin-1-yl]-5-nitro-pyrimidin-4-yl]-4-methyl-piperidine-4-carboxamide (Intermediate B-5(36), Example 4i) (800 mg, 1.63 mmol, 1.0 eq.), 5-formylpyridine-2-carbonitrile (2.16 g, 16.32 mmol, 10.0 eq.) and iron (912 mg, 16.32 mmol, 10.0 eq.) in N-methyl-2-pyrrolidone (10 mL) and acetic acid (10 mL) was stirred at 80° C. for 8 hours. The reaction mixture was cooled at room temperature and diluted with water (150 mL), the resulting mixture was filtered. The filter cake was concentrated under reduced pressure to give a residue. The residue was purified by flash silica gel chromatography (ISCO®; 40 g SepaFlash® Silica Flash Column, Eluent of 50˜100% Ethyl acetate/Petroleum ether gradient @40 mL/min). The cut fraction was concentrated under reduced pressure, the resulting residue was purified by reversed-phase HPLC (column: Phenomenex luna C18 150*25 mm*10 μm; mobile phase: [water(formic acid)-ACN]; B %: 41%-71%, 10 min) and the organic phase was concentrated under reduced pressure to remove acetonitrile, and the liquid was lyophilized to give the crude product. The crude product was purified by reversed-phase HPLC (column: Waters Xbridge 150*25 mm*5 μm; mobile phase: [water(ammonium bicarbonate)-ACN]; B %: 47%-77%, 8 min). The cut fraction was concentrated under reduced pressure to remove acetonitrile. The residue was lyophilized to give compound 1-[9-(4-chlorophenyl)-8-(6-cyano-3-pyridyl)-2-[(2S)-2-(hydroxymethyl)pyrrolidin-1-yl]purin-6-yl]-4-methyl-piperidine-4-carboxamide (Compound 130, 138.33 mg, 0.24 mmol, 14.7% yield) as a yellow solid. ¹H NMR (400 MHz, CDCl₃) δ=8.86-8.84 (m, 1H), 7.84-7.76 (m, 1H), 7.59 (d, J=8.4 Hz, 1H), 7.54 (d, J=8.4 Hz, 2H), 7.28-7.27 (m, 1H), 7.27-7.25 (m, 1H), 6.32-5.80 (m, 1H), 5.77-5.34 (m, 2H), 4.93-3.51 (m, 9H), 2.26-2.14 (m, 2H), 2.14-2.05 (m, 1H), 1.96-1.80 (m, 2H), 1.74-1.65 (m, 3H), 1.36 (s, 3H). LCMS: (ES⁺) m/z=572.3 (M+H).

(cxxii) Compound 131:

A mixture of 1-[6-(4-chloroanilino)-2-[(3R)-3-hydroxypyrrolidin-1-yl]-5-nitro-pyrimidin-4-yl]-4-methyl-piperidine-4-carboxamide (Intermediate B-5(131), Example 4xi) (800 mg, 1.68 mmol, 1.0 eq.), 5-formylpyridine-2-carbonitrile (2.22 g, 16.80 mmol, 10.0 eq.) and iron (939 mg, 16.80 mmol, 10.0 eq.) in N-methyl-2-pyrrolidone (12 mL) and acetic acid (12 mL) was stirred at 80° C. for 8 hours. The reaction mixture was cooled at room temperature and diluted with water (150 mL), the resulting mixture was filtered. The filter cake was concentrated under reduced pressure, the resulting residue was purified by flash silica gel chromatography (ISCO®; 40 g SepaFlash® Silica Flash Column, Eluent of 50-100% Ethyl acetate/Petroleum ether gradient @40 mL/min). The cut fraction was concentrated under reduced pressure, the resulting residue was purified by reversed-phase HPLC (column: Phenomenex luna C18 150*25 mm*10 μm; mobile phase: [water (formic acid)-ACN]; B %: 45%-75%, 10 min). The cut fraction was concentrated under reduced pressure to remove acetonitrile. The residue was lyophilized, the resulting crude product was purified by reversed-phase HPLC (column: Waters Xbridge 150*5 mm*5 μm; mobile phase: [water (ammonium bicarbonate)-ACN]; B %: 39%-69%, 8 min). The cut fraction was concentrated under reduced pressure to remove acetonitrile. The residue was lyophilized to give compound 1-[9-(4-chlorophenyl)-8-(6-cyano-3-pyridyl)-2-[(3R)-3-hydroxypyrrolidin-1-yl]purin-6-yl]-4-methyl-piperidine-4-carboxamide (Compound 131, 119 mg, 213 μmol, 13% yield) as a yellow solid. ¹H NMR (400 MHz, DMSO-d₆) δ=8.79-8.74 (m, 1H), 8.01 (d, J=8.4 Hz, 1H), 7.89-7.81 (m, 1H), 7.62 (d, J=8.8 Hz, 2H), 7.47 (d, J=8.8 Hz, 2H), 7.30-6.93 (m, 2H), 4.84 (d, J=2.8 Hz, 1H), 4.29 (s, 1H), 3.83-3.33 (m, 8H), 2.15-2.08 (m, 2H), 1.96-1.79 (m, 2H), 1.47-1.38 (m, 2H), 1.17 (s, 3H). LCMS: (ES⁺) m/z=558.3 (M+H).

(cxxiii) Compound 132:

A mixture of 1-[6-(4-chloroanilino)-2-[(2-hydroxy-2-methyl-propyl)amino]-5-nitro-pyrimidin-4-yl]-4-ethoxy-piperidine-4-carboxamide (Intermediate B-5(132), Example 4xii) (90 mg, 0.18 mmol, 1.0 eq.) and 5-formylpyridine-2-carbonitrile (234 mg, 1.77 mmol, 10 eq.) in ethyl alcohol (1 mL) was added iron (99 mg, 1.77 mmol, 10 eq.) and acetic acid (0.4 mL), the mixture was stirred at 80° C. for 1 hour. The reaction mixture was filtered and concentrated under reduced pressure to give a residue. The residue was dissolved in N,N-dimethylformamide (2 mL) and purified by prep-HPLC (column: Phenomenex Luna C18 150*25 mm*10 μm; mobile phase: [water (formic acid)-ACN]; B %: 35%-65%, 10 min) and the organic phase was concentrated under reduced pressure to remove acetonitrile, and the liquid was lyophilized to give 1-[9-(4-chlorophenyl)-8-(6-cyano-3-pyridyl)-2-[(2-hydroxy-2-methyl-propyl)amino]purin-6-yl]-4-ethoxy-piperidine-4-carboxamide (Compound 132, 18.23 mg, 0.03 mmol, 17.4% yield) as a yellow solid. ¹H NMR (400 MHz, CDCl₃-d) δ=8.83 (d, J=2.0 Hz, 1H), 7.80-7.75 (m, 1H), 7.58 (d, J=8.4 Hz, 1H), 7.51 (d, J=8.8 Hz, 2H), 7.27-7.23 (m, 3H), 6.41 (d, J=3.2 Hz, 1H), 5.34 (d, J=4.4 Hz, 1H), 5.22 (t, J=6.4 Hz, 1H), 4.81-3.63 (m, 2H), 3.62-3.19 (m, 6H), 2.24-2.11 (m, 2H), 2.01-1.94 (m, 2H), 1.33-1.29 (m, 3H), 1.21 (s, 6H). LCMS: (ES⁺) m/z=590.1 (M+H).

(cxxiv) Compound 133:

A mixture of 1-[6-(4-chloroanilino)-2-[(2S)-2-(hydroxymethyl)pyrrolidin-1-yl]-5-nitro-pyrimidin-4-yl]-4-ethoxy-piperidine-4-carboxamide (Intermediate B-5(133), Example 4xiii) (160 mg, 0.31 mmol, 1.0 eq.) and 5-formylpyridine-2-carbonitrile (203 mg, 1.54 mmol, 5.0 eq.) in ethyl alcohol (1.5 mL) was added iron (172 mg, 3.08 mmol, 10 eq.) and acetic acid (0.6 mL), the mixture was stirred at 80° C. for 1 hour. The reaction mixture was filtered and concentrated under reduced pressure to give a residue. The residue was purified by flash silica gel chromatography (ISCO®; 40 g SepaFlash® Silica Flash Column, Eluent of 0˜100% Dichloromethane/Methanol @40 mL/min) and the organic phase was concentrated under reduced pressure to give the crude product. The residue was dissolved in acetonitrile (2 mL) and purified by prep-HPLC (column: Phenomenex luna C18 150*25 mm*10 μm; mobile phase: [water (formic acid)-ACN]; B %: 36%-66%, 10 min) and the organic phase was concentrated under reduced pressure to remove acetonitrile, and the liquid was lyophilized to give 1-[9-(4-chlorophenyl)-8-(6-cyano-3-pyridyl)-2-[(2S)-2-(hydroxymethyl)pyrrolidin-1-yl]purin-6-yl]-4-ethoxy-piperidine-4-carboxamide (Compound 133, 55.14 mg, 86.4 μmol, 28.0% yield) as a yellow solid. ¹H NMR (400 MHz, DMSO-d₆) δ=8.78-8.74 (m, 1H), 8.01 (d, J=8.0 Hz, 1H), 7.86-7.81 (m, 1H), 7.60 (d, J=8.8 Hz, 2H), 7.46 (d, J=8.8 Hz, 2H), 7.34-7.27 (m, 1H), 7.22 (s, 1H), 5.62-5.15 (m, 1H), 4.97-4.29 (m, 2H), 4.20-3.86 (m, 1H), 3.83-3.34 (m, 8H), 1.98-1.76 (m, 8H), 1.22 (t, J=6.8 Hz, 3H). LCMS: (ES⁺) m/z=602.3 (M+H).

(cxxv) Compound 135:

A mixture of intermediate B-5(135) (100 mg, 0.19 mmol, 1.0 eq.) and 5-formylpyridine-2-carbonitrile (247 mg, 1.87 mmol, 10 eq.) in acetic acid (1 mL) was added iron (105 mg, 1.87 mmol, 10 eq.) and N-methylpyrrolidone (1 mL), the mixture was stirred at 80° C. for 5 hours. The mixture was added to water (50 mL), filtered to give a residue. The residue was purified by flash silica gel chromatography (ISCO®; 25 g SepaFlash® Silica Flash Column, Eluent of 0˜25% methanol/dichloromethane ether gradient @50 mL/min) and prep-HPLC (column: Phenomenex luna C18 150*25 mm*10 μm; mobile phase: [water(formic acid)-acetonitrile]; B %: 56%-86%, 10 min) to give compound 135 (23.42 mg, 38 μmol, 20% yield) as a yellow solid. ¹H NMR (400 MHz, DMSO-d₆) δ=8.85-8.73 (m, 1H), 8.05 (d, J=8.0 Hz, 1H), 7.96-7.88 (m, 1H), 7.68-7.60 (m, 2H), 7.54-7.45 (m, 2H), 7.37-7.28 (m, 1H), 7.26-7.13 (m, 1H), 5.31-5.03 (m, 1H), 4.78-4.53 (m, 1H), 4.50-4.25 (m, 1H), 4.21-4.01 (m, 1H), 3.96 (s, 2H), 3.83-3.72 (m, 1H), 3.68-3.48 (m, 1H), 2.03-1.81 (m, 4H), 1.17 (d, J=6.0 Hz, 6H), 1.14 (s, 6H). LCMS: (ES⁺) m/z=590.3 (M+H).

(cxxvi) Compound 136:

A mixture of 1-[6-(4-chloroanilino)-2-[(2S)-2-(hydroxymethyl)pyrrolidin-1-yl]-5-nitro-pyrimidin-4-yl]-4-isopropoxy-piperidine-4-carboxamide (Intermediate B-5(136), Example 4xv) (100 mg, 0.18 mmol, 1.0 eq.) and 5-formylpyridine-2-carbonitrile (247 mg, 1.87 mmol, 10 eq.) in acetic acid (1 mL) was added iron (105 mg, 1.87 mmol, 10 eq.) and N-methylpyrrolidone (1 mL), the mixture was stirred at 80° C. for 5 hours. The mixture was added to water (50 mL), filtered to give a residue. The residue was purified by flash silica gel chromatography (ISCO®; 25 g SepaFlash® Silica Flash Column, Eluent of 0˜25% methanol/dichloromethane ether gradient @50 mL/min) and prep-HPLC (column: Phenomenex luna C18 150*25 mm*10 μm; mobile phase: [water(formic acid)-acetonitrile]; B %: 56%-86%, 10 min) to give 1-[9-(4-chlorophenyl)-8-(6-cyano-3-pyridyl)-2-[(2S)-2-(hydroxymethyl)pyrrolidin-1-yl]purin-6-yl]-4-isopropoxy-piperidine-4-carboxamide (Compound 136, 23.42 mg, 38.0 μmol, 20% yield) as a yellow solid. ¹H NMR (400 MHz, DMSO-d₆) δ=8.79-8.74 (m, 1H), 8.01 (d, J=8.0 Hz, 1H), 7.88-7.81 (m, 1H), 7.60 (d, J=8.8 Hz, 2H), 7.46 (d, J=8.8 Hz, 2H), 7.30 (s, 1H), 7.19 (s, 1H), 5.53-4.84 (m, 1H), 4.70 (t, J=5.2 Hz, 1H), 4.48-3.33 (m, 9H), 1.99-1.76 (m, 8H), 1.16 (d, J=6.0 Hz, 6H). LCMS: (ES⁺) m/z=616.3 (M+H).

(cxxvii) Compound 158:

The 1-[8-(2-chlorophenyl)-9-(4-chlorophenyl)-2-methylsulfonyl-purin-6-yl]-4-methyl-piperidine-4-carboxamide (Compound 68) (90 μmol) was dissolved in N-methylethanolamine and the reaction mixture was stirred at 100° C. overnight. EtOAc (15 mL) was added and the solution was washed 5 times with saturated NH₄Cl, dried with anhydrous Na₂SO₄ and concentrated. Crude residue was purified (injection with DMSO) on Buchi semi-prep (C18 column) using 20-60% ACN in AmF. The fractions were lyophilized affording 1-[8-(2-chlorophenyl)-9-(4-chlorophenyl)-2-((2-hydroxyethyl)(methyl)amino)-purin-6-yl]-4-methyl-piperidine-4-carboxamide (Compound 158, 57% yield) as an off-white solid. ¹H NMR (400 MHz, DMSO-d₆) δ 7.70-7.60 (m, 1H), 7.51-7.41 (m, 5H), 7.32-7.22 (m, 3H), 6.94 (s, 1H), 4.60 (s, 1H), 3.77-3.64 (m, 1H), 3.60-3.44 (m, 6H), 3.06 (s, 3H), 2.12-1.99 (m, 2H), 1.46-1.33 (m, 2H), 1.15 (s, 3H). LCMS: (ES⁺) m/z=554.5 (M+H).

(cxxviii) Compound 160:

The 1-[8-(2-chlorophenyl)-9-(4-chlorophenyl)-2-methylsulfonyl-purin-6-yl]-4-methyl-piperidine-4-carboxamide (Compound 68) (90 μmol) was dissolved in (S)-2-pyrrolidinemethanol and the reaction mixture was stirred at 100° C. overnight. EtOAc (15 mL) was added and the solution was washed 5 times with saturated NH₄Cl, dried with anhydrous Na₂SO₄ and concentrated. Crude residue was purified (injection with DMSO) on Buchi semi-prep (C18 column) using 20-60% ACN in AmF. The fractions were lyophilized affording 1-[8-(2-chlorophenyl)-9-(4-chlorophenyl)-2-((2S)-pyrrolidinemethoxy)-purin-6-yl]-4-methyl-piperidine-4-carboxamide (Compound 160, 57% yield) as an off-white solid. ¹H NMR (400 MHz, DMSO-d₆) δ 7.68 (dd, J=7.4, 1.1 Hz, 1H), 7.56-7.40 (m, 5H), 7.35-7.24 (m, 3H), 6.98 (s, 1H), 5.01 (s, 2H), 4.33-4.18 (m, 3H), 3.91 (s, 1H), 3.71-3.59 (m, 1H), 3.54-3.38 (m, 1H), 3.10-2.93 (m, 2H), 2.18-2.04 (m, 2H), 2.01-1.90 (m, 1H), 1.89-1.69 (m, 2H), 1.58 (dq, J=15.9, 8.0 Hz, 1H), 1.48-1.35 (m, 2H), 1.16 (s, 3H). LCMS: (ES⁺) m/z=580.5 (M+H).

(cxxix) Compound 161:

The crude 8-(2-chlorophenyl)-9-(4-chlorophenyl)-2-methylsulfonyl-6-[4-(trifluoromethyl)-1-piperidyl]purine (Intermediate D-3) (90 μmol) was dissolved in N-methylethanolamine and the reaction mixture was stirred at 100° C. overnight. EtOAc (15 mL) was added and the solution was washed 5 times with saturated NH₄Cl, dried with anhydrous Na₂SO₄ and concentrated. Crude residue was purified (injection with DMSO) on a C18 semi-prep column using 20-60% ACN in AmF. The fractions were lyophilized affording 1-[[8-(2-chlorophenyl)-9-(4-chlorophenyl)-6-[4-(trifluoromethyl)-1-piperidyl]purin-2-yl]-methyl-amino]-ethan-2-ol (Compound 161, 29.0 mg, 57%) as an off-white solid. ¹H NMR (400 MHz, DMSO-d6) δ 7.66 (ddd, J=7.1, 1.7, 0.7 Hz, 1H), 7.51-7.40 (m, 5H), 7.31-7.25 (m, 2H), 5.42 (s, 1H), 4.60 (t, J=5.1 Hz, 1H), 3.57 (s, 4H) 3.07 (s, 5H), 2.79-2.68 (m, 1H), 1.94 (d, J=11.0 Hz, 2H), 1.45 (qd, J=12.6, 4.1 Hz, 2H).

(cxxx) Compound 162:

The crude 8-(2-chlorophenyl)-9-(4-chlorophenyl)-2-methylsulfonyl-6-[4-(trifluoromethyl)-1-piperidyl]purine (Intermediate D-3) (90 μmol) was dissolved in (S)-2-pyrrolidinemethanol and the reaction mixture was stirred at 100° C. overnight. EtOAc (15 mL) was added and the solution was washed 5 times with saturated NH₄Cl, dried with anhydrous Na₂SO₄ and concentrated. The crude residue was purified (injection with DMSO) on a C18 semi-prep column using 20-60% ACN in AmF. The fractions were lyophilized affording [(2S)-1-[8-(2-chlorophenyl)-9-(4-chlorophenyl)-6-[4-(trifluoromethyl)-1-piperidyl]purin-2-yl]pyrrolidin-2-yl]methanol (Compound 162, 57% yield) as an off-white solid. ¹H NMR (400 MHz, DMSO-d6) δ 7.66 (ddd, J=7.1, 1.7, 0.7 Hz, 1H), 7.52-7.40 (m, 5H), 7.31-7.23 (m, 2H), 5.43 (s, 1H), 4.71 (t, J=5.4 Hz, 1H), 4.04 (s, 1H), 3.63 (s, 1H), 3.50-3.34 (m, 2H), 3.32-3.29 (m, 1H), 3.07 (t, J=11.8 Hz, 2H), 2.81-2.69 (m, 1H), 2.02-1.72 (m, 6H), 1.45 (q, J=12.1 Hz, 2H).

### Example 10—Biological Properties

### a) Design of the Assay

### Day 1:

Using polyethylenimine (PEI) transfection agent, HEK293 cells were transfected in suspension with the human CB1 receptor and one of the following bioSensAll® assays: GAPL-Gi2, or β-arrestin plasma membrane (PM) translocation biosensor (+GRK2). Cells were directly seeded in 96-well plates immediately following transfection.

### Day 4 (˜65 Hours Post-Transfection):

Cells were incubated with coelenterazine (luciferase substrate) and different test compounds prior to the measurement of BRET signals.

### b) BioSensAll® Platform Description.

Used in this project are i) Gα plasma membrane (GAPL) biosensors and the ii) β-arrestin plasma membrane translocation biosensor.

i) GAPL sensors are used to monitor the activation of heterotrimeric G proteins at the plasma membrane upon receptor stimulation. Specifically, these multimolecular BRET sensors detect the plasma membrane recruitment of proteins that interact with active Gα subunits in a G protein family-selective manner. G protein activation following receptor stimulation generally leads to an increase in the BRET signal.

ii) The multimolecular β-arrestin membrane recruitment biosensors allow for real-time spatio-temporal monitoring of β-arrestin 1 and β-arrestin 2 following GPCR activation. Specifically, these β-arrestin sensors were designed to detect the recruitment of proteins to the plasma membrane (β-arrestin PM) with localization to compartment resulting in an increased BRET signal.

### c) Cell Culture Conditions

HEK293 cells were maintained in Dulbecco's Modified Eagle Medium (DMEM) (Wisent; cat #319-030-CL: without sodium pyruvate, with 4.5 g/L glucose, without L-glutamine) supplemented with 1% penicillin-streptomycin (Wisent; cat #450-201-EL) and 2 or 10% fetal bovine serum (Wisent cat #090150).

### d) Transfection and Cell Plating

HEK293 cells were co-transfected with hCB1 and one of the above-listed bioSensAll® assays. For each transfection condition:
the total amount of transfected DNA was kept constant at 1 μg per mL of cell culture to be transfected; whenever necessary, salmon sperm DNA (Invitrogen, cat #15632011) was used as ‘carrier’ DNA to supplement plasmid DNA (i.e., biosensor and receptor);
the PEI (polyethylenimine 25 kDa linear, PolyScience, cat #23966) to DNA ratio (ug:ug) was fixed at 3:1.

Transfections were carried out as follows:
DNA and PEI were first diluted separately in 150 mM NaCl. The volume of the diluent in each tube corresponds to 5% of the cell culture volume to be transfected.
Once DNA and PEI were diluted, the PEI-containing solution was added to the DNA solution and the DNA/PEI mixture immediately vortexed for 5 seconds.
The DNA/PEI mixture was incubated for at least 20 minutes at room temperature to allow for the formation of DNA/PEI complexes.
During the incubation, HEK293 cells were detached, counted and re-suspended in culture medium (composition specified above).
At the end of the incubation period, the DNA/PEI mixture was added to the cells.
Cells were finally distributed in 96-well plates (White Opaque 96-well/Microplates, Greiner, cat #655083 or CulturPlate-96, White Opaque 96-well Microplate, PerkinElmer, cat #6005688) at a density of 30,000 cells per well.

### e) BRET Assay

At 48 hours post-transfection:
Using 450-Select TS Biotek plate washer, culture medium was aspirated and replaced with 100 μL of Hank's Balanced Salt Solution buffer (HBSS) (Wisent, cat #319-067CL: without red phenol; with sodium bicarbonate, with calcium and magnesium, with HEPES) per well.
Plates were then incubated (equilibrated) at 37° C., 5% CO₂ for 60 minutes.
Increasing doses of test compounds were then added to each well using the HP D300e digital dispenser (Tecan). All compounds were assayed at the following 12 concentrations (in nM) with each biosensor: 0.000, 0.013, 0.040, 0.120, 0.320, 0.960, 2.80, 8.00, 24.0, 70.0, 205 and 600.
Three to 5 minutes later, an assay-specific effective concentration (EC)75-80 of agonist CP55,940 (i.e., 0.1 nM and 100 nM for G□i2-GAPL or □Arrestin2 (+GRK2) assay, respectively) was added to wells previously injected with test compound (i.e., previous step 3). Further, as internal plate controls, 2 wells were injected with an EC₇₅ of CP55,940 alone, 3 wells were injected with an Eₘₐₓ of CP55,940 alone and 3 wells were not injected with any compound.
Cells were then incubated overnight at 37° C., 5% CO₂.
The next day, 10 μL of 20 μM e-Coelenterazine Prolume Purple (Methoxy e-CTZ) (Nanolight, cat #369) was added to each well for 5 minutes (final concentration of 2 μM).
BRET readings were collected with a 0.4 seconds integration time on a Synergy NEO plate reader (BioTek Instruments, Inc., USA; filters: 400 nm/70 nm, 515 nm/20 nm)

### f) Calculations

BRET signals were determined by calculating the ratio of light emitted by GFP-acceptor (515 nm) over light emitted by luciferase-donor (400 nm). All BRET ratios were standardized using the equation below with pre-established BRET values for positive and negative controls. The standardized BRET ratio is referred to as universal BRET (uBRET).

$$uBRET = ( ( BRET ratio - A ) / ( B - A ) ) * 10 \; 000$$

### Where:

A=BRET ratio obtained from transfection of negative control
B=BRET ratio obtained from transfection of positive control

Resulting dose-response curves were fitted using the four-parameter logistic non-linear regression model in GraphPad Prism 9.

In vitro assay results for Compounds 1 to 240 as prepared in Example 9 are presented in Table 17.

TABLE 17
In vitro assay data

| | CB₁ wt Gi2 | CB₁ wt βarr2 |
|---|---|---|
| Compound | IC₅₀ (nM) | IC₅₀ (nM) |
| 1 | 4.3 | 15.2 |
| 2 | 10.5 | 63.5 |
| 3 | 1.38 | 12.2 |
| 4 | 2.03 | 10.4 |
| 5 | 0.961 | 16 |
| 6 | 0.861 | 4.78 |
| 7 | 0.417 | 3.45 |
| 8 | 0.922 | 2.74 |
| 9 | 0.398 | 2.92 |
| 10 | 1.47 | 6.8 |
| 11 | 2.01 | 30.1 |
| 12 | 0.5 | 3.09 |
| 13 | 7.02 | >5000 |
| 14 | 6.5 | 34.5 |
| 15 | 1.29 | 8.03 |
| 16 | 0.315 | 1.51 |
| 17 | 0.617 | 2.39 |
| 18 | 3.73 | 11.3 |
| 19 | 0.187 | 1.07 |
| 20 | 0.255 | 0.568 |
| 21 | 4.37 | 28.9 |
| 22 | 1.33 | 11.3 |
| 23 | 0.846 | 4.36 |
| 24 | 0.707 | 1.91 |
| 25 | 0.706 | 2.06 |
| 26 | 1.09 | 5.69 |
| 27 | 1.5 | 10.2 |
| 28 | 1.02 | 12.2 |
| 29 | 6.92 | >5000 |
| 30 | 14.4 | 60 |
| 31 | 2.76 | 21.8 |
| 32 | 0.509 | 3.02 |
| 33 | 1.42 | 4.98 |
| 34 | 6.62 | 17.6 |
| 35 | 0.687 | 3.3 |
| 36 | 0.686 | 1.52 |
| 37 | 7.39 | 60.5 |
| 38 | 1.67 | 6.48 |
| 39 | 3.29 | 16.4 |
| 40 | 0.971 | 15.3 |
| 41 | 1.65 | 7.33 |
| 42 | 0.695 | 2.39 |
| 43 | 1.37 | 5.27 |
| 44 | 2.1 | 10 |
| 45 | 5.85 | 39.9 |
| 46 | 1.82 | 9.64 |
| 47 | 1.87 | 6.65 |
| 48 | 1.2 | 5.91 |
| 49 | 2.6 | 19.3 |
| 50 | 8.41 | 47.8 |
| 51 | 10.1 | 64.3 |
| 52 | 2.06 | 29.1 |
| 53 | 0.966 | 13.1 |
| 54 | 0.427 | 1.12 |
| 55 | 0.647 | 4.58 |
| 56 | 0.83 | 5.41 |
| 57 | 41.2 | 121 |
| 58 | 6.91 | 515 |
| 59 | >5000 | >5000 |
| 60 | 6.96 | 19.3 |
| 61 | 0.645 | 16.7 |
| 62 | 6.14 | 7.62 |
| 63 | 9.9 | 65.1 |
| 64 | 2.45 | 27.9 |
| 65 | 0.534 | 1.70 |
| 66 | 0.586 | 1.45 |
| 67 | 1.27 | 4.38 |
| 68 | 1.69 | 10.7 |
| 69 | 41.9 | 117 |
| 70 | 64.6 | >5000 |
| 71 | 9.05 | NA |
| 72 | 1.1 | 3.81 |
| 73 | 0.556 | 1.77 |
| 74 | 0.574 | 3.02 |
| 75 | 0.399 | 0.834 |
| 76 | 0.51 | 1.18 |
| 77 | >5000 | >5000 |
| 78 | 0.995 | 5.68 |
| 79 | 0.868 | 4.37 |
| 80 | 1.02 | 5.09 |
| 81 | 0.896 | 4.9 |
| 82 | 0.753 | 4.49 |
| 83 | 1.16 | 6.26 |
| 84 | 2.1 | 11.7 |
| 85 | 0.754 | 6.76 |
| 86 | 1.38 | 9.49 |
| 87 | 3.1 | 21.6 |
| 88 | 1.37 | 10.3 |
| 89 | 0.789 | 2.31 |
| 90 | 0.52 | 1.36 |
| 91 | 0.801 | 2.56 |
| 92 | 1.71 | 7.93 |
| 93 | 0.606 | 1.43 |
| 94 | 0.82 | 2.41 |
| 95 | 13 | 72.4 |
| 96 | 3.43 | 5.68 |
| 97 | 25.8 | 123 |
| 98 | NA | >5000 |
| 99 | 4.51 | 36.2 |
| 100 | >5000 | >5000 |
| 101 | 1.53 | 16 |
| 102 | 50 | 40.1 |
| 103 | 131 | >5000 |
| 104 | 48.8 | >5000 |
| 105 | 17.8 | 760 |
| 106 | 14 | 50.7 |
| 107 | 27.7 | NA |
| 108 | 3.51 | 26.2 |
| 109 | 13.2 | 61.5 |
| 110 | 6.33 | 13 |
| 111 | 18.4 | 53.7 |
| 112 | 88.3 | 105 |
| 113 | 7.11 | 15.5 |
| 114 | 3.87 | 14.1 |
| 115 | 17.8 | 93.6 |
| 116 | 68.8 | >5000 |
| 117 | 1.64 | 3.76 |
| 118 | NA | >5000 |
| 119 | 3.09 | 21.6 |
| 120 | 16.9 | 25.4 |
| 121 | 2.6 | 17 |
| 122 | 14.6 | NA |
| 123 | 0.876 | 2.04 |
| 124 | 3.23 | 12.1 |
| 125 | 4.99 | 16.4 |
| 126 | 8.07 | 20.7 |
| 127 | 9.42 | 40.9 |
| 128 | 9.52 | 39.5 |
| 129 | 438 | NA |
| 130 | 0.592 | 1.2 |
| 131 | 0.713 | 2.95 |
| 132 | 17.8 | >5000 |
| 133 | 0.673 | 1.8 |
| 134 | NA | NA |
| 135 | 2.3 | 11.7 |
| 136 | 0.848 | 1.75 |
| 137 | NA | NA |
| 138 | 34.1 | >5000 |
| 139 | 7.55 | 58.8 |
| 140 | 1.22 | 8.69 |
| 141 | 0.982 | 2.28 |
| 142 | 0.892 | 2.6 |
| 143 | 0.837 | 1.59 |
| 144 | 1.35 | 6.22 |
| 145 | 0.973 | 3 |
| 146 | 0.718 | 1.62 |
| 147 | 4.1 | 88.8 |
| 148 | 1 | 1.49 |
| 149 | 8.93 | >5000 |
| 150 | 2.67 | 8.17 |
| 151 | 4.46 | 12.9 |
| 152 | 0.716 | 1.2 |
| 153 | 0.503 | 1.29 |
| 154 | 0.813 | 7.28 |
| 155 | 1.01 | 14.8 |
| 156 | 54.7 | >5000 |
| 157 | 0.938 | 6.04 |
| 158 | 0.555 | 0.931 |
| 160 | 0.868 | 21.7 |
| 161 | 0.95 | 1.53 |
| 162 | 1.3 | 1.83 |
| 163 | 0.491 | 2.78 |
| 164 | 1.66 | 22.8 |
| 165 | 0.413 | 2.03 |
| 166 | 0.308 | 1.4 |
| 167 | 0.421 | 4.39 |
| 168 | 1.93 | 12 |
| 169 | 0.532 | 2.04 |
| 170 | 0.597 | 3.49 |
| 171 | 0.962 | 5.27 |
| 172 | 0.887 | 14.3 |
| 173 | 0.419 | 1.51 |
| 174 | 0.645 | 3.94 |
| 175 | 5.3 | NA |
| 176 | 15.6 | >5000 |
| 177 | 0.436 | 1.88 |
| 178 | 0.544 | 4.04 |
| 179 | 0.244 | 1.92 |
| 180 | 0.226 | 1.21 |
| 181 | 0.181 | 0.592 |
| 182 | 0.175 | 0.661 |
| 183 | 0.167 | 0.785 |
| 184 | 0.24 | 0.889 |
| 185 | 0.148 | 0.894 |
| 186 | 0.407 | 4.92 |
| 187 | 3.97 | 40.4 |
| 188 | 0.31 | 1.84 |
| 189 | 0.419 | 1.35 |
| 190 | 2.66 | 23.7 |
| 191 | 0.418 | 0.626 |
| 192 | 0.752 | 4.75 |
| 193 | 1.23 | 15.8 |
| 194 | 0.445 | 6.02 |
| 195 | 0.394 | 3.88 |
| 196 | 0.186 | 1.42 |
| 197 | 0.161 | 1.19 |
| 198 | 7.69 | 31.5 |
| 199 | 0.732 | 3.27 |
| 200 | 1.72 | 25.2 |
| 201 | 1.88 | 14.8 |
| 202 | 5.91 | 38.8 |
| 203 | 1.39 | 6.49 |
| 204 | 3.96 | 16.6 |
| 205 | 6.04 | 54.6 |
| 206 | 0.782 | 6.99 |
| 207 | 0.652 | 2.02 |
| 208 | 16.8 | 141 |
| 209 | 1.34 | 14.7 |
| 210 | 1.56 | 18.7 |
| 211 | 0.943 | 6.86 |
| 212 | 2.51 | 25.9 |
| 213 | 1.85 | 7.84 |
| 214 | 1.09 | 6.06 |
| 215 | 0.707 | 1.64 |
| 216 | 1.77 | 8.61 |
| 217 | 27 | 73.7 |
| 218 | 7.82 | NA |
| 219 | 59.7 | NA |
| 220 | 3.47 | 23.2 |
| 221 | 4.04 | 30.6 |
| 222 | 1.56 | 7.91 |
| 223 | 0.916 | 2.8 |
| 224 | 1.27 | 7.99 |
| 225 | 0.881 | 2.57 |
| 226 | 0.767 | 2.31 |
| 227 | 4.78 | 11.7 |
| 228 | 1.2 | 2.29 |
| 229 | 3.65 | 12.5 |
| 230 | 0.895 | 2.76 |
| 231 | 1.02 | 1.56 |
| 232 | 0.934 | 1.94 |
| 233 | 2.37 | 22.3 |
| 234 | 0.49 | 1.27 |
| 235 | 1.31 | 28.5 |
| 236 | 2.89 | 22.8 |
| 237 | 0.61 | 1.93 |
| 238 | 13.8 | NA |
| 239 | 27.4 | NA |
| 240 | 5.79 | 12.2 |

NA: Not available

Numerous modifications could be made to any of the embodiments described above without departing from the scope of the present invention. Any references, patents or scientific literature documents referred to in the present document are incorporated herein by reference in their entirety for all purposes.

1.-119. (canceled)

120. A compound of Formula I: 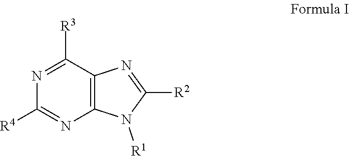
wherein,
R¹ is an optionally substituted C₆aryl group;
R² is an optionally substituted C₆aryl or C₅-C₆heteroaryl group;
R³ is an optionally substituted group selected from R⁷O—, N(R⁸)₂—, C₄₋₁₀heterocycloalkyl, and C₅₋₆heteroaryl;
R⁴ is an optionally substituted group selected from R⁵O— and N(R⁶)₂—;
R⁵ is a group selected from C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl;
R⁶ is H or a group selected from C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl, wherein at least one R⁶ is other than H, or two R⁶ groups are taken together with their adjacent nitrogen atom to form a C₄-C₁₀heterocycloalkyl or C₅-C₆heteroaryl group;
wherein at least one of said alkyl, cycloalkyl, heterocycloalkyl, or heteroaryl in R⁵ or R⁶ is substituted with at least one hydroxyl or hydroxy-substituted C₁-C₄alkyl group and is optionally further substituted with other substituents;
R⁷ is an optionally substituted C₁-C₆alkyl; and
R⁸ is independently in each occurrence selected from hydrogen, C₁-C₆alkyl, C₃-C₇cycloalkyl, and C₄-C₁₀heterocycloalkyl;
wherein each of the alkyl, cycloalkyl, heterocycloalkyl, aryl, and heteroaryl groups is optionally substituted;
or a pharmaceutically acceptable salt thereof.

121. The compound of claim 120, wherein said compound is of Formula II: 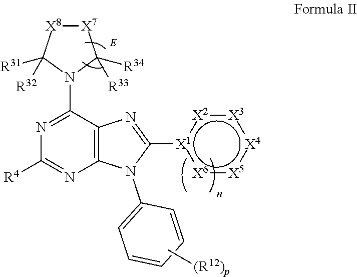
wherein,
R⁴ is an optionally substituted C₆aryl group;
X¹ is C and X² to X⁶ are each independently selected from N and CR²¹ and n is 1, wherein at most three of X² to X⁶ are N; or
X¹ is C or N and X² to X⁵ are each independently selected from CR²¹, O, S, N, or NR¹¹, n is zero and X⁶ is absent and replaced by a bond between X¹ and X⁵, wherein at most three of X¹ to X⁵ are other than C or CR²¹;
X⁷ and X⁸ are each independently selected from O, S, SO₂, NR³⁶, or C(R³⁵)₂, wherein when one of X⁷ and X⁸ is O, S, or NR³⁶, then the other is C(R³⁵)₂;
R⁹ is independently in each occurrence selected from optionally substituted C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, and C₅-C₆heteroarylC₁-C₃alkyl;
R¹⁰ is independently in each occurrence selected from H or a group selected from optionally substituted C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, and C₅-C₆heteroarylC₁-C₃alkyl, or two R¹⁰ groups are taken together with their adjacent nitrogen atom to form an optionally substituted C₄-C₁₀heterocycloalkyl or C₅-C₆heteroaryl;
R¹¹ is independently in each occurrence selected from H or a group selected from optionally substituted C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl;
R¹² is independently in each occurrence selected from F, Cl, CN, NH₂, N(H)C₁-C₃alkyl, N(C₁-C₃alkyl)₂, and C₁-C₃alkyl, and p is 0, 1, 2, or 3;
R²¹ is independently in each occurrence selected from hydrogen, halogen, OH, OR⁹, CN, NO₂, C(O)R⁹, C(O)N(R¹⁰)₂, C(R¹¹)═NR¹¹, SO₂R⁹, SO₂N(R¹⁰)₂, N(R¹¹)C(O)R⁹, N(R¹¹)SO₂R⁹, N(R¹¹)C(O)N(R¹⁰)₂, N(R¹¹)SO₂N(R¹⁰)₂, N(R¹⁰)₂, P(O)(R¹⁰)₂, P(O)(OR¹⁰)₂, B(OR¹⁰)₂, and optionally substituted C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl groups;
R³¹ to R³⁵ are independently in each occurrence selected from hydrogen, halogen, OH, OR⁹, CN, NO₂, C(O)OH, C(O)OR⁹, C(O)R⁹, C(O)N(R¹⁰)₂, C(R¹¹)═NR¹¹, SO₂R⁹, SO₂N(R¹⁰)₂, N(R¹¹)C(O)R⁹, N(R¹¹)SO₂R⁹, N(R¹¹)C(O)N(R¹⁰)₂, N(R¹¹)SO₂N(R¹⁰)₂, N(R¹⁰)₂, P(O)(R¹⁰)₂, P(O)(OR¹⁰)₂, B(OR¹⁰)₂, and optionally substituted C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl groups;
R³⁶ is selected from hydrogen, C(O)R⁹, C(O)OR⁹, C(O)N(R¹⁰)₂, C(R¹¹)═NR¹¹, SO₂R⁹, SO₂N(R¹⁰)₂, P(O)(R¹⁰)₂, P(O)(OR¹⁰)₂, B(OR¹⁰)₂, and optionally substituted C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl groups;
or two of R³¹ to R³⁶ are taken together with their adjacent atoms to form a cycle; and
m is 0, 1, 2, or 3;
wherein each of the alkyl, cycloalkyl, heterocycloalkyl, aryl, and heteroaryl groups is optionally substituted;
or a pharmaceutically acceptable salt thereof.

122. The compound of claim 120, wherein R³ is selected from groups C1-C7, C10, C15, C16, C18-C22, C24-C28, C32-C40, and C47-C69, or a pharmaceutically acceptable salt thereof.

123. The compound of claim 120, wherein said compound is of Formula III: 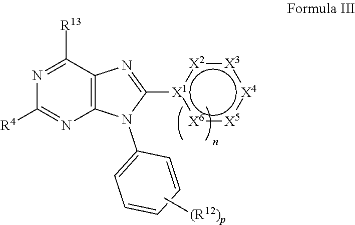
wherein,
R¹³ is an optionally substituted group selected from R⁷O—, N(R⁸)₂—, and C₅₋₆heteroaryl;
R⁴ is an optionally substituted group selected from R⁵O— and N(R⁶)₂—;
R⁷ is an optionally substituted C₁-C₆alkyl;
R⁸ is independently in each occurrence selected from hydrogen, C₁-C₆alkyl, C₃-C₇cycloalkyl, and C₄-C₁₀heterocycloalkyl; and
R¹² is independently in each occurrence selected from F, Cl, CN, NH₂, N(H)C₁-C₃alkyl, N(C₁-C₃alkyl)₂, and C₁-C₃alkyl, and p is 0, 1, 2, or 3;
X¹ is C and X² to X⁶ are each independently selected from N and CR²¹ and n is 1, wherein at most three of X² to X⁶ are N; or
X¹ is C or N and X² to X⁵ are each independently selected from CR²¹, O, S, N, or NR¹¹, n is zero and X⁶ is absent and replaced by a bond between X¹ and X⁵, wherein at most three of X¹ to X⁵ are other than C or CR²¹;
R¹¹ is independently in each occurrence selected from H or a group selected from optionally substituted C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl;
R²¹ is independently in each occurrence selected from hydrogen, halogen, OH, OR⁹, CN, NO₂, C(O)R⁹, C(O)N(R¹⁰)₂, C(R¹¹)═NR¹¹, SO₂R⁹, SO₂N(R¹⁰)₂, N(R¹¹)C(O)R⁹, N(R¹¹)SO₂R⁹, N(R¹¹)C(O)N(R¹⁰)₂, N(R¹¹)SO₂N(R¹⁰)₂, N(R¹⁰)₂, P(O)(R¹⁰)₂, P(O)(OR¹⁰)₂, B(OR¹⁰)₂, and optionally substituted C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl groups;
or a pharmaceutically acceptable salt thereof.

124. The compound of claim 123, wherein R¹³ is selected from C8, C9, C11-C14, C17, C23, C29-C31, and C41-C46; or a pharmaceutically acceptable salt thereof.

125. The compound of claim 121,
wherein R¹² is Cl and p is 1, forming a 4-chlorophenyl group; or
wherein p is zero and R¹² is absent, forming an unsubstituted phenyl group; or a pharmaceutically acceptable salt thereof.

126. The compound of claim 120, wherein R² is selected from groups B1 to B23;
or a pharmaceutically acceptable salt thereof.

127. The compound of claim 120,
wherein R⁴ is an R⁵O— group; and
wherein R⁵ is a group selected from C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl, and is substituted with at least one hydroxyl group or hydroxy-substituted C₁-C₄alkyl group; or a pharmaceutically acceptable salt thereof.

128. The compound of claim 127, wherein R⁵ is a C₂-C₆alkyl, C₄-C₁₀heterocycloalkyl, or C₄-C₁₀heterocycloalkylC₁-C₃alkyl group substituted with a hydroxy group and optionally other substituents; or a pharmaceutically acceptable salt thereof.

129. The compound of claim 120,
wherein R⁴ is an N(R⁶)₂-group; and
wherein at least one R⁶ is substituted with at least one hydroxyl group or hydroxy-substituted C₁-C₄alkyl group and optionally substituted with one or more other substituents; or a pharmaceutically acceptable salt thereof.

130. The compound of claim 129,
wherein one R⁶ is a C₂-C₆alkyl, C₃-C₇cycloalkyl, C₄-C₁₀heterocycloalkyl, C₃-C₇cycloalkylC₁-C₃alkyl, or C₄-C₁₀heterocycloalkylC₁-C₃alkyl group substituted with at least one hydroxyl or hydroxy-substituted C₁-C₄alkyl group, and the other R⁶ is a hydrogen or C₁-C₆alkyl and optionally substituted with one or more other substituents; or
wherein the two R⁶ groups are taken together with their adjacent nitrogen atom to form a C₄-C₁₀heterocycloalkyl or C₅heteroaryl group substituted with at least one hydroxyl or hydroxy-substituted C₁-C₄alkyl group and optionally substituted with one or more other substituents; or
a pharmaceutically acceptable salt thereof.

131. The compound of claim 130,
wherein one R⁶ is a C₂-C₆alkyl or C₃-C₇cycloalkylC₁-C₃alkyl group substituted with a hydroxyl or hydroxy-substituted C₁-C₄alkyl group, and the other R⁶ is a hydrogen or C₁-C₆alkyl; or
wherein the two R⁶ groups are taken together with their adjacent nitrogen atom to form a C₄-C₇heterocycloalkyl substituted with at least one hydroxyl or hydroxy-substituted C₁-C₄alkyl group and optionally substituted with one or more other substituents; or
wherein the two R⁶ groups are taken together with their adjacent nitrogen atom to form a C₅heteroaryl group substituted with at least one hydroxyl or hydroxy-substituted C₁-C₄alkyl group and optionally substituted with one or more other substituents;
or a pharmaceutically acceptable salt thereof.

132. The compound of claim 120, wherein R⁴ is selected from groups D5-D9, D12, D13, D19-D21, D23, D25, D27, D28, D30-D32, D36, D37, D43-D49, D52, D53, D67, D71, D75, D76, and D78-D81; or a pharmaceutically acceptable salt thereof.

133. A compound of Formula IV: 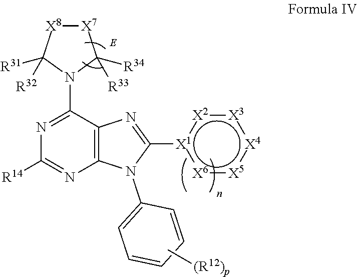
wherein,
m is 0, 1, 2, or 3;
n is 0 or 1;
p is 0, 1, 2, or 3;
X¹ is C and X² to X⁶ are each independently selected from N and CR²¹ and n is 1, wherein at most three of X² to X⁶ are N; or X¹ is C or N and X² to X⁵ are each independently selected from CR²¹, O, S, N, or NR¹¹, n is zero and X⁶ is absent and forms a bond between X¹ and X⁵, wherein at most three of X¹ to X⁵ are other than C or CR²¹;
X⁷ and X⁸ are each independently selected from O, S, SO₂, NR³⁶, or C(R³⁵)₂, wherein when one of X⁷ and X⁸ is O, S, or NR³⁶, then the other is C(R³⁵)₂;
R¹² is independently in each occurrence selected from F, Cl, CN, NH₂, N(H)C₁-C₃alkyl, N(C₁-C₃alkyl)₂, and C₁-C₃alkyl, and p is 0, 1, 2, or 3;
R¹⁴ is N(R¹⁶)₂— or optionally substituted C₅-C₆heteroaryl; and
R¹⁶ is selected from H or a group selected from optionally substituted C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl, or two R¹⁶ groups are taken together with their adjacent nitrogen atom to form an optionally substituted C₄-C₁₀heterocycloalkyl or C₅-C₆heteroaryl; and
R³¹ to R³⁴ are independently in each occurrence selected from hydrogen, halogen, OH, OR⁹, CN, NO₂, C(O)OH, C(O)OR⁹, C(O)R⁹, C(O)N(R¹⁰)₂, C(R¹¹)═NR¹¹, SO₂R⁹, SO₂N(R¹⁰)₂, N(R¹¹)C(O)R⁹, N(R¹¹)SO₂R⁹, N(R¹¹)C(O)N(R¹⁰)₂, N(R¹¹)SO₂N(R¹⁰)₂, N(R¹⁰)₂, P(O)(R¹⁰)₂, P(O)(OR¹⁰)₂, B(OR¹⁰)₂, and optionally substituted C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl groups;
or a pharmaceutically acceptable salt thereof.

134. The compound of claim 133, wherein R¹⁴ is N(R¹⁶)₂—, and
wherein one R¹⁶ is an optionally substituted C₁-C₆alkyl, C₃-C₇cycloalkyl, C₄-C₁₀heterocycloalkyl, C₃-C₇cycloalkylC₁-C₃alkyl, or C₄-C₁₀heterocycloalkylC₁-C₃alkyl group, and the other R¹⁶ is a hydrogen or C₁-C₆alkyl; or
wherein one R¹⁶ is an C₂-C₆alkyl group substituted with one or more substituents, and the other R¹⁶ is a hydrogen or C₁-C₆alkyl, wherein said substituent is selected from F, OH, CN, alkoxy, alkylcarbonylamino, akoxycarbonylamino, alkylsulfonamido, benzyl amino, aminocarbonyl, dialkylphosphino, phosphonato, dialkylamino, cycloalkyl, heterocycloalkyl, aryl, and heteroaryl; or
wherein the two R¹⁶ groups are taken together with their adjacent nitrogen atom to form an optionally substituted C₄-C₁₀heterocycloalkyl or C₅heteroaryl group connected through the nitrogen; or
wherein the two R¹⁶ groups are taken together with their adjacent nitrogen atom to form an optionally substituted C₄-C₇heterocycloalkyl connected through the nitrogen; or
wherein the two R¹⁶ groups are taken together with their adjacent nitrogen atom to form an optionally substituted C₅heteroaryl group connected through the nitrogen atom; or a pharmaceutically acceptable salt thereof.

135. The compound of claim 133, wherein R¹⁴ is an optionally substituted C₅-C₆heteroaryl; or wherein R¹⁴ is selected from D3-D5, D11-D16, D19-D28, D30-D32, D36, D37, D39-D41, D48-D54, D56, D57, D63-D67, D71, and D78-D81; or a pharmaceutically acceptable salt thereof.

136. A compound of Formula V: 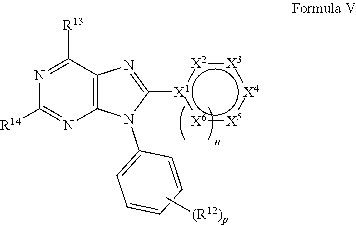
wherein,
n is 0 or 1;
p is 0, 1, 2, or 3;
X¹ is C and X² to X⁶ are each independently selected from N and CR²¹ and n is 1, wherein at most three of X² to X⁶ are N; or X¹ is C or N and X² to X⁵ are each independently selected from CR²¹, O, S, N, or NR¹¹, n is zero and X⁶ is absent and forms a bond between X¹ and X⁵, wherein at most three of X¹ to X⁵ are other than C or CR²¹;
R¹² is independently in each occurrence selected from F, Cl, CN, NH₂, N(H)C₁-C₃alkyl, N(C₁-C₃alkyl)₂, and C₁-C₃alkyl;
R¹³ is an optionally substituted group selected from R⁷O—, N(R⁸)₂—, and C₅₋₆heteroaryl; and
R¹⁴ is N(R¹⁶)₂— or optionally substituted C₅-C₆heteroaryl;
or a pharmaceutically acceptable salt thereof.

137. A compound of Formula VI: 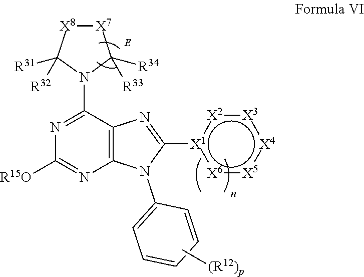
wherein,
m is 0, 1, 2, or 3;
n is 0 or 1;
p is 0, 1, 2, or 3;
X¹ is C and X² to X⁶ are each independently selected from N and CR²¹ and n is 1, wherein at most three of X² to X⁶ are N; or X¹ is C or N and X² to X⁵ are each independently selected from CR²¹, O, S, N, or NR¹¹, n is zero and X⁶ is absent and forms a bond between X¹ and X⁵, wherein at most three of X¹ to X⁵ are other than C or CR²¹;
X⁷ and X⁸ are each independently selected from O, S, SO₂, NR³⁶, or C(R³⁵)₂, wherein when one of X⁷ and X⁸ is O, S, or NR³⁶, then the other is C(R³⁵)₂;
R¹² is independently in each occurrence selected from F, Cl, CN, NH₂, N(H)C₁-C₃alkyl, N(C₁-C₃alkyl)₂, and C₁-C₃alkyl, and p is 0, 1, 2, or 3;
R¹⁵ is a group selected from C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl; and
R³¹ to R³⁴ are independently in each occurrence selected from hydrogen, halogen, OH, OR⁹, CN, NO₂, C(O)OH, C(O)OR⁹, C(O)R⁹, C(O)N(R¹⁰)₂, C(R¹¹)═NR¹¹, SO₂R⁹, SO₂N(R¹⁰)₂, N(R¹¹)C(O)R⁹, N(R¹¹)SO₂R⁹, N(R¹¹)C(O)N(R¹⁰)₂, N(R¹¹)SO₂N(R¹⁰)₂, N(R¹⁰)₂, P(O)(R¹⁰)₂, P(O)(OR¹⁰)₂, B(OR¹⁰)₂, and optionally substituted C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl groups;
or a pharmaceutically acceptable salt thereof.

138. The compound of claim 137, wherein R¹⁵ is a C₁-C₆alkyl group substituted with one or more substituents, for instance selected from F, OH, CN, alkoxy, alkylcarbonylamino, akoxycarbonylamino, alkylsulfonamido, benzyl amino, aminocarbonyl, dialkylphosphino, phosphonato, dialkylamino, cycloalkyl, heterocycloalkyl, aryl, and heteroaryl; or wherein R¹⁵ is an optionally substituted C₄-C₆heterocycloalkyl or C₄-C₆heterocycloalkylC₁-C₃alkyl group; or a pharmaceutically acceptable salt thereof.

139. The compound of claim 137, wherein the OR¹⁵ group is selected from D6-D10, D17, D29, D33-D35, D43-D47, D55, D68-D70, and D72-D77; or a pharmaceutically acceptable salt thereof.

140. A compound of Formula VII: 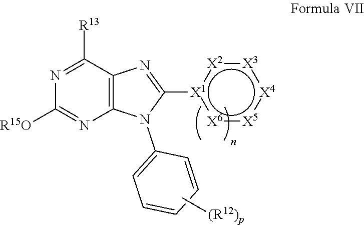
wherein,
n is 0 or 1;
p is 0, 1, 2, or 3;
X¹ is C and X² to X⁶ are each independently selected from N and CR²¹ and n is 1, wherein at most three of X² to X⁶ are N; or X¹ is C or N and X² to X⁵ are each independently selected from CR²¹, O, S, N, or NR¹¹, n is zero and X⁶ is absent and forms a bond between X¹ and X⁵, wherein at most three of X¹ to X⁵ are other than C or CR²¹;
R¹² is independently in each occurrence selected from F, Cl, CN, NH₂, N(H)C₁-C₃alkyl, N(C₁-C₃alkyl)₂, and C₁-C₃alkyl;
R¹³ is an optionally substituted group selected from R⁷O—, N(R⁸)₂—, and C₅₋₆heteroaryl; and
R¹⁵ is a group selected from C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl;
or a pharmaceutically acceptable salt thereof.

141. A compound of Formula VIII: 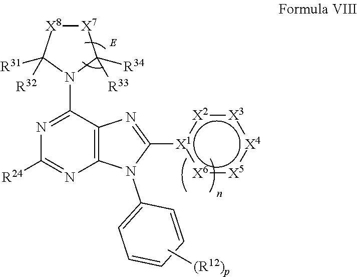
wherein,
m is 0, 1, 2, or 3;
n is 0 or 1;
p is 0, 1, 2, or 3;
X¹ is C and X² to X⁶ are each independently selected from N and CR²¹ and n is 1, wherein at most three of X² to X⁶ are N; or X¹ is C or N and X² to X⁵ are each independently selected from CR²¹, O, S, N, or NR¹¹, n is zero and X⁶ is absent and forms a bond between X¹ and X⁵, wherein at most three of X¹ to X⁵ are other than C or CR²¹;
X⁷ and X⁸ are each independently selected from O, S, SO₂, NR³⁶, or C(R³⁵)₂, wherein when one of X⁷ and X⁸ is O, S, or NR³⁶, then the other is C(R³⁵)₂;
R⁹ is independently in each occurrence selected from optionally substituted C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, and C₅-C₆heteroarylC₁-C₃alkyl;
R¹⁰ is independently in each occurrence selected from H or a group selected from optionally substituted C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, and C₅-C₆heteroarylC₁-C₃alkyl, or two R¹⁰ groups are taken together with their adjacent nitrogen atom to form an optionally substituted C₄-C₁₀heterocycloalkyl or C₅-C₆heteroaryl;
R¹¹ is independently in each occurrence selected from H or a group selected from optionally substituted C₁-C₆alkyl, C₃-C₇cycloalkyl, C₄-C₁₀heterocycloalkyl, C₅-C₆heteroaryl, C₃-C₇cycloalkylC₁-C₃alkyl, C₄-C₁₀heterocycloalkylC₁-C₃alkyl, C₅-C₆heteroarylC₁-C₃alkyl;
R¹² is independently in each occurrence selected from F, Cl, CN, NH₂, N(H)C₁-C₃alkyl, N(C₁-C₃alkyl)₂, and C₁-C₃alkyl;
R²⁴ is selected from Cl, CN, C(O)OH, R⁹C(O)N(R¹¹)—, R⁹C(O)NHC(NH)NH—, R⁹S(O)₂—, N(R¹⁰)₂C(O)—, and an optionally substituted C₁-C₆alkyl group; and
R³¹ to R³⁴ are independently in each occurrence selected from hydrogen, halogen, OH, OR⁹, CN, NO₂, C(O)OH, C(O)OR⁹, C(O)R⁹, C(O)N(R¹⁰)₂, C(R¹¹)═NR¹¹, SO₂R⁹, SO₂N(R¹⁰)₂, N(R¹¹)C(O)R⁹, N(R¹¹)SO₂R⁹, N(R¹¹)C(O)N(R¹⁰)₂, N(R¹¹)SO₂N(R¹⁰)₂, N(R¹⁰)₂, P(O)(R¹⁰)₂, P(O)(OR¹⁰)₂, B(OR¹⁰)₂, and optionally substituted C₁₋₆alkyl, C₂₋₆alkenyl, C₂₋₆alkynyl, C₆₋₁₀aryl, C₅₋₁₀heteroaryl, C₃₋₁₀cycloalkyl, and C₄₋₁₀heterocycloalkyl groups;
or a pharmaceutically acceptable salt thereof.

142. A compound of Formula IX: 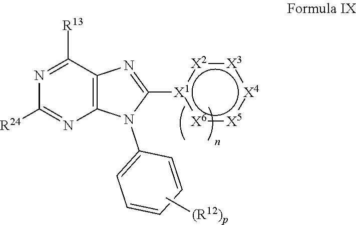
wherein,
X¹ is C and X² to X⁶ are each independently selected from N and CR²¹ and n is 1, wherein at most three of X² to X⁶ are N; or X¹ is C or N and X² to X⁵ are each independently selected from CR²¹, O, S, N, or NR¹¹, n is zero and X⁶ is absent and forms a bond between X¹ and X⁵, wherein at most three of X¹ to X⁵ are other than C or CR²¹;
R¹² is independently in each occurrence selected from F, Cl, CN, NH₂, N(H)C₁-C₃alkyl, N(C₁-C₃alkyl)₂, and C₁-C₃alkyl;
R¹³ is an optionally substituted group selected from R⁷O—, N(R⁸)₂—, and C₅₋₆heteroaryl; and
R²⁴ is selected from Cl, CN, C(O)OH, R⁹C(O)N(R¹¹)—, R⁹C(O)NHC(NH)NH—, R⁹S(O)₂—, N(R¹⁰)₂C(O)—, and an optionally substituted C₁-C₆alkyl group;
or a pharmaceutically acceptable salt thereof.

143. A compound of claim 141, wherein R²⁴ is selected from groups D1, D2, D18, D38, D42, and D58-D62; or a pharmaceutically acceptable salt thereof.

144. A compound selected from compounds 1 to 158 and 160 to 240, or a pharmaceutically acceptable salt thereof.

145. A pharmaceutical composition comprising a compound of claim 120, together with a pharmaceutically acceptable carrier, diluent or excipient.

146. A method for the treatment of a disorder selected from disorders related to appetite or their complications, disorders related to glucose regulation or their complications, fibrosis related disorders or their complications, disorders related to metabolism or their complications, disorders related to skin and hair growth and healing, disorders related to the GI tract, disorders related to obesity or their complications, or a combination thereof, comprising administering a compound of claim 120 to a subject in need thereof.

147. The method of claim 146, wherein said disorder is selected from diabetes Type I, diabetes Type II, nonalcoholic steatohepatitis (NASH), and chronic kidney disease.

148. The method of claim 146, wherein said fibrosis related disorder or one of its complications is selected from progressive fibrosis associated with interstitial lung disease, idiopathic pulmonary fibrosis (IPF), Hermansky-Pudlak syndrome pulmonary fibrosis (HPS-PF), cirrhosis and other liver fibrosis disorders (such as nonalcoholic steatohepatitis (NASH), primary sclerosing cholangitis, primary biliary cholangitis), skin fibrotic disorders (such as scleroderma), fibrotic renal diseases and chronic kidney diseases.
